SEQUENCE LISTING (37 sequences)

SEQ ID NO: 1 - DNA, 21 bp, Artificial Sequence
  Synthetic primer
gcttctaacaccggaggtctt

SEQ ID NO: 2 - DNA, 21 bp, Artificial Sequence
  Synthetic primer
ttatttgaggaactccgccgc

SEQ ID NO: 3 - DNA, 22 bp, Artificial Sequence
  Synthetic primer
catgcacaagaatggatcacaa

SEQ ID NO: 4 - DNA, 20 bp, Artificial Sequence
  Synthetic primer
ggggcgatttgcatttctat

SEQ ID NO: 5 - DNA, 19 bp, Artificial Sequence
  Synthetic primer
cggtcgttgaggaggttgg

SEQ ID NO: 6 - DNA, 22 bp, Artificial Sequence
  Synthetic primer
cattgacaactaccagccgtac

SEQ ID NO: 7 - DNA, 18 bp, Artificial Sequence
  Synthetic primer
gggcagcacatagcgtga

SEQ ID NO: 8 - DNA, 18 bp, Artificial Sequence
  Synthetic primer
gggagcctaaaggtcgtg

SEQ ID NO: 9 - DNA, 18 bp, Artificial Sequence
  Synthetic primer
gggttctcgggaaatgag

SEQ ID NO: 10 - DNA, 18 bp, Artificial Sequence
  Synthetic primer
ccccaactgacattaccc

SEQ ID NO: 11 - DNA, 18 bp, Artificial Sequence
  Synthetic primer
tcgaatacaacgcaccaa

SEQ ID NO: 12 - DNA, 22 bp, Artificial Sequence
  Synthetic primer
atggatgacgatatcgctgcgc

SEQ ID NO: 13 - DNA, 20 bp, Artificial Sequence
  Synthetic primer
gcagcacagggtgctcctca

SEQ ID NO: 14 - DNA, 19 bp, Artificial Sequence
  Synthetic primer
catctaccacttgcaatta

SEQ ID NO: 15 - DNA, 19 bp, Artificial Sequence
  Synthetic primer
gattatcagcaaaatcgag

SEQ ID NO: 16 - DNA, 19 bp, Artificial Sequence
  Synthetic primer
cctatgaaaggatgcaata

SEQ ID NO: 17 - DNA, 19 bp, Artificial Sequence
  Synthetic primer
gcaacagtatttcggtata

SEQ ID NO: 18 - DNA, 19 bp, Artificial Sequence
  Synthetic primer
aagcaggagacgctcacca

SEQ ID NO: 19 - DNA, 19 bp, Artificial Sequence
  Synthetic primer
cccggaccacgggtgacat

SEQ ID NO: 20 - DNA, 17 bp, Artificial Sequence
  Synthetic primer
cagcccgagactgttgc

SEQ ID NO: 21 - DNA, 18 bp, Artificial Sequence
  Synthetic primer
cagagcgtgggatgttag

SEQ ID NO: 22 - DNA, 17 bp, Artificial Sequence
  Synthetic primer
gcgaggaacagaagtgc

SEQ ID NO: 23 - DNA, 19 bp, Artificial Sequence
  Synthetic primer
gagttgtcggtgtagatgc

SEQ ID NO: 24 - DNA, 17 bp, Artificial Sequence
  Synthetic primer
acacccttctcccttcg

SEQ ID NO: 25 - DNA, 18 bp, Artificial Sequence
  Synthetic primer
ccgctccacatacagtcc

SEQ ID NO: 26 - protein, 15 aa, Homo sapiens
KCCSGAIIVLTKSGR

SEQ ID NO: 27 - DNA, 20 bp, Artificial Sequence
  Synthetic primer
cttctaacaccggaggtctt

SEQ ID NO: 28 - DNA, 21 bp, Artificial Sequence
  Synthetic primer
caacattcggaaagccttgtc

SEQ ID NO: 29 - DNA, 21 bp, Artificial Sequence
  Synthetic primer
gagcatcatgaagaagagtcc

SEQ ID NO: 30 - DNA, 18 bp, Artificial Sequence
  Synthetic primer
gggttcggaggtttgatg

SEQ ID NO: 31 - DNA, 17 bp, Artificial Sequence
  Synthetic primer
acggcggtggcttctgt

SEQ ID NO: 32 - DNA, 19 bp, Artificial Sequence
  Synthetic primer
ctggagaaacagccaaagg

SEQ ID NO: 33 - DNA, 20 bp, Artificial Sequence
  Synthetic primer
gccagactccgtcagaacta

SEQ ID NO: 34 - DNA, 23 bp, Artificial Sequence
  Synthetic primer
ccccggagcgactttcctcccag

SEQ ID NO: 35 - DNA, 23 bp, Artificial Sequence
  Synthetic primer
ccccggagctacttgtttcccag

SEQ ID NO: 36 - DNA, 21 bp, Artificial Sequence
  Synthetic primer
gcccgaaagggttccagctta

SEQ ID NO: 37 - DNA, 21 bp, Artificial Sequence
  Synthetic primer
aaggccgagtggcagttgagt

(19) United States
(12) Patent Application Publication
(10) Pub. No.: US 2015/0073033 A1
(43) Pub. Date: Mar. 12, 2015

(54) BIOMARKERS FOR CANCER CHARACTERIZATION AND TREATMENT

(71) Applicant: Board of Regents, The University of Texas System, Austin, TX (US)

(72) Inventors: Zhimin Lu, Houston, TX (US); W.K. Alfred Yung, Houston, TX (US)

(73) Assignee: Board of Regents, The University of Texas System, Austin, TX (US)

(21) Appl. No.: 14/355,116

(22) PCT Filed: Oct. 31, 2012
(86) PCT No.: PCT/US12/62693

§ 371 Date: Apr. 29, 2014

Related U.S. Application Data
(60) Provisional application No. 61/553,823, filed on Oct. 31, 2011.
(60) Provisional application No. 61/649,714, filed on May 21, 2012.

(51) Int. Cl. A61K 31/713; A61K 45/06; C12N 15/113; G01N 33/573; G01N 33/574

(52) U.S. Cl. CPC A61K 31/713; G01N 33/573; G01N 33/57496; C12N 15/1137; A61K 45/06; G01N 2500/02; C12N 2310/14
USPC 514/44 A; 435/7.92; 436/501

(57) ABSTRACT

Composition and methods for characterizing cancer cells by determining a marker of PKM2 activity. For example, methods are provided for predicting a patient response to a NF-κB, PKCε, PKM2, MEK/ERK, Pin1 or Src inhibitor therapy. Methods for treating patients with such therapies are likewise provided. Phosphorylation selective β-catenin, MLC2, histone H3, Bub3, and PKM2-binding antibodies are also provided.

This application claims the benefit of U.S. Provisional Patent Application Nos. 61/553,823, filed Oct. 31, 2011; and 61/649,714, filed May 21, 2012, each of which is incorporated herein by reference in its entirety.

The invention was made with government support under Grant Nos. 5R01CA109035, 5 P50 CA127001-03, and CA16672 awarded by the National Institutes of Health. The government has certain rights in the invention.

### BACKGROUND OF THE INVENTION

1. Field of the Invention

The present invention relates generally to the field of molecular biology, oncology and medicine. More particularly, it concerns methods and composition for characterizing cancer cells.

2. Description of Related Art

Tumor cells have elevated rates of glucose uptake and higher lactate production in the presence of oxygen. This phenomenon, known as aerobic glycolysis, or the Warburg effect, supports tumor cell growth (Vander Heiden et al., 2009). Pyruvate kinase regulates the rate-limiting final step of glycolysis, which catalyzes the transfer of a phosphate group from phosphoenolpyruvate (PEP) to ADP, yielding pyruvate and ATP. Four pyruvate kinase isoforms exist in mammals and are derived from two distinct genes, PKLR and PKM (formerly PKM2). The R and L isozymes are expressed in erythrocytes and the liver, respectively, and are encoded by the PKLR gene, arising through the use of different tissue-specific promoters (Mazurek et al., 2005). The M1 and M2 isoforms result from mutually exclusive alternative splicing of the PKM pre-mRNA, reflecting inclusion of either exon 9 (PKM1) or exon 10 (PKM2). PKM2 is essential for the Warburg effect. While PKM2 has a well-established role in aerobic glycolysis, the mechanism underlying nonmetabolic function of PKM2 remains elusive.

### SUMMARY OF THE INVENTION

In a first embodiment there is provided an isolated antibody, or an antigen-binding fragment thereof, wherein the antibody selectively binds to β-catenin protein that is phosphorylated at position Y333. For example, the antibody can have at least two, three, four, five or more-fold higher affinity to β-catenin protein that is phosphorylated at position Y333 than to β-catenin protein that is not phosphorylated at position Y333 (e.g., the antibody can exhibit essentially no binding to β-catenin protein that is not phosphorylated at position Y333).

In a further embodiment there is provided an isolated antibody, or an antigen-binding fragment thereof, wherein the antibody selectively binds to PKM2 protein that is phosphorylated at position S37. For example, the antibody can have at least two, three, four, five or more-fold higher affinity to PKM2 protein that is phosphorylated at position S37 than to PKM2 protein that is not phosphorylated at position S37 (e.g., the antibody can exhibit essentially no binding to PKM2 protein that is not phosphorylated at position S37).

In a still a further embodiment there is provided an isolated antibody, or an antigen-binding fragment thereof, wherein the antibody selectively binds to MLC2 protein that is phosphorylated at position Y118. For example, the antibody can have at least two, three, four, five or more-fold higher affinity to MLC2 protein that is phosphorylated at position Y118 than to MLC2 protein that is not phosphorylated at position Y118 (e.g., the antibody can exhibit essentially no binding to MLC2 protein that is not phosphorylated at position Y118).

In yet a further embodiment there is provided an isolated antibody, or an antigen-binding fragment thereof, wherein the antibody selectively binds to histone H3 protein that is phosphorylated at position T11. For example, the antibody can have at least two, three, four, five or more-fold higher affinity to histone H3 protein that is phosphorylated at position T11 than to histone H3 protein that is not phosphorylated at position T11 (e.g., the antibody can exhibit essentially no binding to histone H3 protein that is not phosphorylated at position T11).

Certain aspects of the embodiments concern β-catenin, histone H3, MLC2 and/or PKM2-binding antibodies, such as phosphorylation specific antibodies. As used herein an antibody can be a polyclonal or a monoclonal antibody or an antigen binding fragment of an antibody. For example, the antibody can be a humanized antibody, a chimeric antibody, a Fab, a Fab2, a ScFv, or a single domain antibody. In certain aspects, an antibody of the embodiments comprises a label, such as a radioactive, enzyme, fluorescent or affinity label.

In still a further embodiment there is a provided a kit comprising an antibody of the embodiments in a sealed container.

In some embodiments a method is provided for determining a prognosis in a patient having a cancer comprising determining whether cancer cells of the patient comprise (1) an elevated level of β-catenin activity (e.g., an elevated level of β-catenin Y333 phosphorylation); (2) an elevated level of PKM2 S37 phosphorylation; (3) elevated level of nuclear PKM2 expression; (4) an elevated level of histone H3 T11 phosphorylation; (5) an elevated level of histone H3 K9 acetylation; (6) an elevated level of Bub3 Y207 phosphorylation; (7) an elevated level of MLC2 Y118 phosphorylation; and/or (8) an elevated level of EGF-dependent NF-κB activation compared to a reference level, wherein if the cancer cells comprise (1) an elevated level of β-catenin activity (e.g., an elevated level of β-catenin Y333 phosphorylation); (2) an elevated level of PKM2 S37 phosphorylation; (3) elevated level of nuclear PKM2 expression; (4) an elevated level of histone H3 T11 phosphorylation; (5) an elevated level of histone H3 K9 acetylation; (6) an elevated level of Bub3 Y207 phosphorylation; (7) an elevated level of MLC2 Y118 phosphorylation; and/or (8) an elevated level of EGF-dependent NF-κB activation, then the patient is predicted to have an aggressive cancer. In some aspects, a method is provided for determining a prognosis in a patient having a cancer comprising (a) determining whether cancer cells of the patient comprise an elevated level of any of 1, 2, 3, 4, 5, 6, 7 or 8 compared to a reference level; and (b) identifying the patient as predicted to have an aggressive cancer, if cancer cells from the patient comprise an elevated level of any of 1, 2, 3, 4, 5, 6, 7 or 8; or identifying the patient as not predicted to have an aggressive cancer, if cancer cells from the patient do not comprise an elevated level of any of 1, 2, 3, 4, 5, 6, 7 or 8.

In a further embodiment a method is provided for determining a prognosis in a patient having a cancer comprising determining whether cancer cells of the patient comprise an elevated level of histone H3 T11 phosphorylation; an elevated level of Bub3 Y207 phosphorylation; an elevated level of MLC2 Y118 phosphorylation; and/or an elevated level of histone H3 K9 acetylation compared to a reference level, wherein if the cancer cells comprise an elevated level of histone H3 T11 phosphorylation; an elevated level of Bub3 Y207 phosphorylation; an elevated level of MLC2 Y118 phosphorylation; and/or an elevated level of histone H3 K9 acetylation, then the patient is predicted to have an aggressive cancer. Thus, in some aspects, a method is provided for determining a prognosis in a patient having a cancer comprising: (a) determining whether cancer cells of the patient comprise an elevated level of histone H3 T11 phosphorylation; an elevated level of Bub3 Y207 phosphorylation; an elevated level of MLC2 Y118 phosphorylation; or an elevated level of histone H3 K9 acetylation compared to a reference level; and (b) identifying the patient as predicted to have an aggressive cancer, if cancer cells from the patient comprise an elevated level of histone H3 T11 phosphorylation; an elevated level of Bub3 Y207 phosphorylation; an elevated level of MLC2 Y118 phosphorylation; or an elevated level of histone H3 K9 acetylation; or identifying the patient as not predicted to have an aggressive cancer, if cancer cells from the patient do not comprise an elevated level of histone H3 T11 phosphorylation or an elevated level of histone H3 K9 acetylation.

In some aspects, a method of determining a prognosis can comprise determining the grade of cancer or the probability that the cancer will metastasize. In certain aspects, a method of determining a prognosis further comprises reporting whether cancer cells from the patient comprise (1) an elevated level of β-catenin activity (e.g., an elevated level of β-catenin Y333 phosphorylation); (2) an elevated level of PKM2 S37 phosphorylation; (3) elevated level of nuclear PKM2 expression; (4) an elevated level of histone H3 T11 phosphorylation; (5) an elevated level of histone H3 K9 acetylation; (6) an elevated level of Bub3 Y207 phosphorylation; (7) an elevated level of MLC2 Y118 phosphorylation; and/or (8) an elevated level of EGF-dependent NF-κB activation compared to a reference level. In still further aspects, a method can comprise reporting whether a cancer is an aggressive cancer or reporting a grade for the cancer.

In yet a further embodiment, a method is provided for predicting the severity of a cancer in a patient comprising: (i) determining a level of β-catenin activity, a level of PKM2 S37 phosphorylation, a level of nuclear PKM2 expression, a level of histone H3 T11 phosphorylation, a level of histone H3 K9 acetylation, a level of Bub3 Y207 phosphorylation, a level of MLC2 Y118 phosphorylation; and/or a level of EGF-dependent NF-κB activation in a patient sample; and (ii) predicting the severity of cancer in the patient based on the level of β-catenin activity, the level of PKM2 S37 phosphorylation, the level of nuclear PKM2 expression, the level of histone H3 T11 phosphorylation, the level of histone H3 K9 acetylation, the level of Bub3 Y207 phosphorylation, the level of MLC2 Y118 phosphorylation, and/or the level of EGF-dependent NF-κB activation, wherein an elevated level of β-catenin activity, PKM2 S37 phosphorylation, nuclear PKM2 expression, histone H3 T11 phosphorylation, histone H3 K9 acetylation, Bub3 Y207 phosphorylation, MLC2 Y118 phosphorylation; and/or EGF-dependent NF-κB activation relative to a reference level indicates a more severe cancer. For example, determining the level of β-catenin activity in the sample can comprise determining the level of β-catenin Y333 phosphorylation (e.g., by contacting the sample with a phosphorylation specific antibody). In some aspects, a method of the embodiments involves determining a level of PKM2 S37 phosphorylation in the sample, such as by contacting the sample with a phosphorylation specific antibody. Likewise, in certain aspects, determining a level of histone H3 T11 phosphorylation, Bub3 Y207 phosphorylation or MLC2 Y118 phosphorylation in the sample comprises contacting the sample with a phosphorylation specific antibody to the indicated phosphoprotein.

In some embodiments, a method is provided for predicting a response to a MEK/ERK inhibitor; a Src inhibitor; a NF-κB inhibitor; a PKCε inhibitor; Pin1 inhibitor or a PKM2 inhibitor therapy in a patient having a cancer comprising determining whether cancer cells of the patient comprise (1) an elevated level of β-catenin activity (e.g., an elevated level of β-catenin Y333 phosphorylation); (2) an elevated level of PKM2 S37 phosphorylation; (3) elevated level of nuclear PKM2 expression; (4) an elevated level of histone H3 Ti phosphorylation; (5) an elevated level of histone H3 K9 acetylation; (6) an elevated level of Bub3 Y207 phosphorylation; (7) an elevated level of MLC2 Y118 phosphorylation; and/or (8) an elevated level of EGF-dependent NF-κB activation compared to a reference level, wherein if the cancer cells comprise (1) an elevated level of β-catenin activity (e.g., an elevated level of β-catenin Y333 phosphorylation); (2) an elevated level of PKM2 S37 phosphorylation; (3) elevated level of nuclear PKM2 expression; (4) an elevated level of histone H3 T11 phosphorylation; (5) an elevated level of histone H3 K9 acetylation; (6) an elevated level of Bub3 Y207 phosphorylation; (7) an elevated level of MLC2 Y118 phosphorylation; and/or (8) an elevated level of EGF-dependent NF-κB activation, then the patient is predicted to have a favorable response to a MEK/ERK inhibitor; a Src inhibitor; a NF-κB inhibitor; a PKCε inhibitor; Pin1 inhibitor or a PKM2 inhibitor therapy. Thus, in some aspects, a method is provided for predicting a response to a MEK/ERK inhibitor; a Src inhibitor; a NF-κB inhibitor; a PKCε inhibitor; Pin1 inhibitor or a PKM2 inhibitor therapy in a patient having a cancer comprising (a) determining whether cancer cells of the patient comprise an elevated level any of 1, 2, 3, 4, 5, 6, 7 and/or 8 compared to a reference level; and (b) identifying the patient as predicted to have a favorable response to a MEK/ERK inhibitor; a Src inhibitor; a NF-κB inhibitor; a PKCε inhibitor; Pin1 inhibitor or a PKM2 inhibitor therapy, if cancer cells from the patient comprise an elevated level of any of 1, 2, 3, 4, 5, 6, 7 and/or 8; or identifying the patient as not predicted to have a favorable response to a MEK/ERK inhibitor; a Src inhibitor; a NF-κB inhibitor; a PKCε inhibitor; Pin1 inhibitor or a PKM2 inhibitor therapy, if cancer cells from the patient do not comprise an elevated level of any of 1, 2, 3, 4, 5, 6, 7 and/or 8.

In some further embodiments a method is provided for predicting a response to a PKM2 inhibitor therapy in a patient having a cancer comprising determining whether cancer cells of the patient comprise an elevated level of histone H3 T11 phosphorylation; an elevated level of Bub3 Y207 phosphorylation; an elevated level of MLC2 Y118 phosphorylation or an elevated level of histone H3 K9 acetylation compared to a reference level, wherein if the cancer cells comprise an elevated level of histone H3 T11 phosphorylation; an elevated level of Bub3 Y207 phosphorylation; an elevated level of MLC2 Y118 phosphorylation or an elevated level of histone H3 K9 acetylation, then the patient is predicted to have a favorable response to a PKM2 inhibitor therapy. Thus, in some aspects, a method is provided for predicting a response to a PKM2 inhibitor therapy in a patient having a cancer comprising (a) determining whether cancer cells of the patient comprise an elevated level of histone H3 T11 phosphorylation; an elevated level of Bub3 Y207 phosphorylation; an elevated level of MLC2 Y118 phosphorylation; or an elevated level of histone H3 K9 acetylation compared to a reference level; and (b) identifying the patient as predicted to have a favorable response to a PKM2 inhibitor therapy, if cancer cells from the patient comprise an elevated level of histone H3 T11 phosphorylation; an elevated level of Bub3 Y207 phosphorylation; an elevated level of MLC2 Y118 phosphorylation; or an elevated level of histone H3 K9 acetylation; or identifying the patient as not predicted to have a favorable response to a PKM2 inhibitor therapy, if cancer cells from the patient do not comprise an elevated level of histone H3 T11 phosphorylation; an elevated level of Bub3 Y207 phosphorylation; an elevated level of MLC2 Y118 phosphorylation; or an elevated level of histone H3 K9 acetylation.

As used in the context of methods of the embodiments a “favorable response” to a therapy, such as a MEK/ERK inhibitor; a Src inhibitor; a NF-κB inhibitor; a PKCε inhibitor; Pin1 inhibitor and/or a PKM2 inhibitor therapy, can comprise reduction in tumor size or burden, blocking of tumor growth, reduction in tumor-associated pain, reduction in cancer associated pathology, reduction in cancer associated symptoms, cancer non-progression, increased disease free interval, increased time to progression, induction of remission, reduction of metastasis, increased patient survival and/or an increase in the sensitivity of the tumor to an anticancer therapy.

In some aspects, a method of predicting a response further comprises reporting whether cancer cells from the patient comprise (1) an elevated level of β-catenin activity (e.g., an elevated level of β-catenin Y333 phosphorylation); (2) an elevated level of PKM2 S37 phosphorylation; (3) elevated level of nuclear PKM2 expression; (4) an elevated level of histone H3 T11 phosphorylation; (5) an elevated level of histone H3 K9 acetylation; (6) an elevated level of Bub3 Y207 phosphorylation; (7) an elevated level of MLC2 Y118 phosphorylation; and/or (8) an elevated level of EGF-dependent NF-κB activation compared to a reference level. In still further aspects, a method can comprise reporting whether a cancer is predicted to respond to a MEK/ERK inhibitor; a Src inhibitor; a NF-κB inhibitor; a PKCε inhibitor; Pin1 inhibitor and/or a PKM2 inhibitor therapy.

In still a further embodiment there is provided a method of selecting a patient having a cancer for a MEK/ERK inhibitor; a Src inhibitor; a NF-κB inhibitor; a PKCε inhibitor; Pin1 inhibitor and/or a PKM2 inhibitor therapy comprising determining whether cancer cells of the patient comprise (1) an elevated level of β-catenin activity (e.g., an elevated level of β-catenin Y333 phosphorylation); (2) an elevated level of PKM2 S37 phosphorylation; (3) elevated level of nuclear PKM2 expression; (4) an elevated level of histone H3 T11 phosphorylation; (5) an elevated level of histone H3 K9 acetylation; (6) an elevated level of Bub3 Y207 phosphorylation; (7) an elevated level of MLC2 Y118 phosphorylation; and/or (8) an elevated level of EGF-dependent NF-κB activation compared to a reference level, wherein if the cancer cells comprise (1) an elevated level of β-catenin activity (e.g., an elevated level of β-catenin Y333 phosphorylation); (2) an elevated level of PKM2 S37 phosphorylation; (3) elevated level of nuclear PKM2 expression; (4) an elevated level of histone H3 T11 phosphorylation; (5) an elevated level of histone H3 K9 acetylation; (6) an elevated level of Bub3 Y207 phosphorylation; (7) an elevated level of MLC2 Y118 phosphorylation; and/or (8) an elevated level of EGF-dependent NF-κB activation compared to a reference level, then the patient is selected for a MEK/ERK inhibitor; a Src inhibitor; a NF-κB inhibitor; a PKCε inhibitor; Pin1 inhibitor and/or a PKM2 inhibitor therapy. Thus, in some aspects, a method is provided of selecting a patient having a cancer for a MEK/ERK inhibitor; a Src inhibitor; a NF-κB inhibitor; a PKCε inhibitor; Pin1 inhibitor and/or a PKM2 inhibitor therapy comprising: (a) determining whether cancer cells of the patient comprise an elevated level of any of 1, 2, 3, 4, 5, 6, 7 and/or 8; and (b) selecting a patient for a MEK/ERK inhibitor; a Src inhibitor; a NF-κB inhibitor; a PKCε inhibitor; Pin1 inhibitor and/or a PKM2 inhibitor therapy if cancer cells of the patient comprise an elevated level of any of 1, 2, 3, 4, 5, 6, 7 and/or 8.

In yet a further embodiment there is provided a composition for use in treating a patient having a cancer determined to comprise: an elevated level of histone H3 T11 phosphorylation; an elevated level of PKM2 S37 phosphorylation; an elevated level of nuclear PKM2 expression; an elevated level of Bub3 Y207 phosphorylation; an elevated level of MLC2 Y118 phosphorylation; and/or an elevated level of histone H3 K9 acetylation compared to a reference level. For example, such a composition can comprise an effective amount of a MEK/ERK inhibitor; a Src inhibitor; a NF-κB inhibitor; a PKCε inhibitor; Pin1 inhibitor, a PKM2 inhibitor or a combination thereof.

In still a further embodiment there is provided a method for treating a patient having a cancer comprising (i) selecting a patient whose cancer cells have been determined to comprise: an elevated level of EGF-dependent NF-κB activation; an elevated level of 3-catenin activity; an elevated level of PKM2 S37 phosphorylation; an elevated level of nuclear PKM2 expression; an elevated level of histone H3 T11 phosphorylation; an elevated level of Bub3 Y207 phosphorylation; an elevated level of MLC2 Y118 phosphorylation; or an elevated level of histone H3 K9 acetylation compared to a reference level and (ii) treating the selected patient with a PKM2 inhibitor therapy. Thus, in a related embodiment, a composition comprising a PKM2 inhibitor is provided for use in treating a patient having a cancer determined to comprise: an elevated level of EGF-dependent NF-κB activation; an elevated level of β-catenin activity; an elevated level of PKM2 S37 phosphorylation; an elevated level of nuclear PKM2 expression; an elevated level of histone H3 T11 phosphorylation; an elevated level of Bub3 Y207 phosphorylation; an elevated level of MLC2 Y118 phosphorylation; or an elevated level of histone H3 K9 acetylation compared to a reference level.

In a further embodiment, a method is provided of selecting a patient having a cancer for a PKM2 inhibitor therapy comprising determining whether cancer cells of the patient comprise an elevated level of histone H3 T11 phosphorylation or an elevated level of histone H3 K9 acetylation compared to a reference level, wherein if the cancer cells comprise an elevated level of histone H3 T11 phosphorylation or an elevated level of histone H3 K9 acetylation, then the patient is selected for a PKM2 inhibitor therapy. Thus, in some aspects, a method is provided of selecting a patient having a cancer for an PKM2 inhibitor therapy comprising (a) determining whether cancer cells of the patient comprise an elevated level of histone H3 T11 phosphorylation or an elevated level of histone H3 K9 acetylation compared to a reference level; and (b) selecting a patient for PKM2 inhibitor therapy if cancer cells of the patient comprise an elevated level of histone H3 T11 phosphorylation or an elevated level of histone H3 K9 acetylation.

In a further embodiment a method is provided of identifying a cancer patient that is a candidate for a therapy comprising: (i) determining a level of β-catenin activity in a patient sample; and (ii) identifying a cancer patient that is a candidate for a Src inhibitor therapy based on the level of β-catenin activity, wherein an elevated level of β-catenin activity relative to a reference level indicates that the patient is a candidate for said therapy. In still a further embodiment, a method is provided for identifying a cancer patient that is a candidate for a therapy comprising: (i) determining a level of PKM2 S37 phosphorylation, or a level of nuclear PKM2 expression in a patient sample; and (ii) identifying a cancer patient that is a candidate for a MEK/ERK inhibitor therapy based on the level of PKM2 S37 phosphorylation or the level of nuclear PKM2 expression, wherein an elevated level of PKM2 S37 phosphorylation or nuclear PKM2 expression relative to a reference level indicates that the patient is a candidate for said therapy.

In some aspects, a method of selecting a patient or identifying a candidate further comprises reporting whether cancer cells from the patient comprise (1) an elevated level of β-catenin activity (e.g., an elevated level of β-catenin Y333 phosphorylation); (2) an elevated level of PKM2 S37 phosphorylation; (3) elevated level of nuclear PKM2 expression; (4) an elevated level of histone H3 T11 phosphorylation and/or (5) an elevated level of histone H3 K9 acetylation compared to a reference level. In still further aspects, a method can comprise reporting whether a patient is selected for or is a candidate for a PKM2, MEK/ERK and/or Src inhibitor therapy.

In certain aspects, methods of the embodiments comprise reporting results, such as by providing a written, electronic or oral report. In some aspects, a report is provided to the patient. In still further aspects, the report is provided to a third party, such an insurance company or health care provider (e.g., a doctor or hospital).

In a further embodiment there is provided a method for treating a patient having a cancer comprising (i) selecting a patient whose cancer cells have been determined to comprise (1) an elevated level of β-catenin activity (e.g., an elevated level of β-catenin Y333 phosphorylation); (2) an elevated level of PKM2 S37 phosphorylation; (3) elevated level of nuclear PKM2 expression; (4) an elevated level of histone H3 T11 phosphorylation; (5) an elevated level of histone H3 K9 acetylation; (6) an elevated level of Bub3 Y207 phosphorylation; (7) an elevated level of MLC2 Y118 phosphorylation; and/or (8) an elevated level of EGF-dependent NF-κB activation compared to a reference level; and (ii) treating the patient with a MEK/ERK inhibitor; a Src inhibitor; a NF-κB inhibitor; a PKCε inhibitor; Pin1 inhibitor and/or a PKM2 inhibitor therapy.

In yet a further embodiment a method for treating a patient having a cancer is provided comprising: (i) selecting a patient whose cancer cells have been determined to comprise an elevated level of β-catenin activity relative to a reference level (e.g., a patient whose cancer cells comprise an elevated level of β-catenin Y333 phosphorylation); and (ii) treating the patient with a Src inhibitor therapy, a MEK/ERK inhibitor therapy and/or a PKM2 inhibitor therapy.

In still yet a further embodiment a method for treating a patient having a cancer is provided comprising: (i) selecting a patient whose cancer cells have been determined to comprise an elevated level of PKM2 S37 phosphorylation or nuclear PKM2 expression relative to a reference level; and (ii) treating the patient with a MEK/ERK inhibitor therapy, a Src inhibitor therapy and/or a PKM2 inhibitor therapy. In some specific aspects, a method comprises (i) selecting a patient whose cancer cells have been determined to comprise an elevated level of PKM2 S37 phosphorylation; and (ii) treating the patient with a MEK/ERK inhibitor therapy.

In still a further embodiment there is provided a method for treating a patient having a cancer comprising (i) selecting a patient whose cancer cells have been determined to comprise an elevated level of histone H3 T11 phosphorylation or an elevated level of histone H3 K9 acetylation compared to a reference level; and (ii) treating the patient with a MEK/ERK inhibitor; a Src inhibitor; a NF-κB inhibitor; a PKCε inhibitor; Pin1 inhibitor and/or a PKM2 inhibitor therapy. In certain aspects the selected patient is treated PKM2 inhibitor therapy, optionally in conjunction with a MEK/ERK inhibitor therapy and/or a Src inhibitor therapy.

In a further embodiment there is provided a method for treating a patient having a cancer comprising (i) selecting a patient whose cancer cells have been determined to comprise an elevated level of Bub3 Y207 and/or MLC2 Y118 phosphorylation; and (ii) treating the patient with a MEK/ERK inhibitor; a Src inhibitor; a NF-κB inhibitor; a PKCε inhibitor; Pin1 inhibitor and/or a PKM2 inhibitor therapy. In certain aspects the selected patient is treated PKM2 inhibitor therapy, optionally in conjunction with a MEK/ERK inhibitor therapy and/or a Src inhibitor therapy.

Aspects of the embodiments concern determining or obtaining a level of (1) β-catenin activity (e.g., an elevated level of β-catenin Y333 phosphorylation); (2) PKM2 S37 phosphorylation; (3) nuclear PKM2 expression; (4) histone H3 T11 phosphorylation and/or (5) a histone H3 K9 acetylation compared to a reference level. For example, in certain aspects, the reference level is level from a healthy patient or a non-cancer cell. In still further aspects, the reference level is a level determined or obtained from an early stage or low grade cancer cell.

Various aspects of the embodiments involve determining a level of β-catenin activity, a level of PKM2 S37 phosphorylation, a level of nuclear PKM2 expression, a level of histone H3 T11 phosphorylation, a level of histone H3 K9 acetylation, a level of Bub3 Y207 phosphorylation; a level of MLC2 Y118 phosphorylation and/or a level of EGF-dependent NF-κB activation. In certain aspects, this determining can comprise performing an ELISA, an immunoassay, a radioimmunoassay (RIA), Immunohistochemistry, an immunoradiometric assay, a fluoroimmunoassay, a chemiluminescent assay, a bioluminescent assay, a gel electrophoresis, a Western blot analysis, a southern blot, flow cytometry, in situ hybridization, positron emission tomography (PET), single photon emission computed tomography (SPECT) imaging) or a microscopic assay. For example, in some cases, a phosphorylation specific antibody is used to determine a level of β-catenin Y333, PKM2 S37, MLC2 Y118, Bub3 Y207 or histone H3 T11 phosphorylation. Likewise, in some aspects, an acetylation specific antibody is used to determine a level of histone H3 K9 acetylation. In some aspects, a method of the embodiments is defined as an in vitro method in other aspects a method may be performed in vivo (e.g., by in vivo imaging).

Some aspects of the embodiments involve a patient, such as a patient having a cancer. As used herein a patient can be human or non-human animal patient (e.g., a dog, cat, mouse, horse, etc). In certain aspects, the patient has a cancer, such as an oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, neuroendocrine tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In some aspects, the cancer is a glioma.

Some aspects of the embodiments concern patient samples, such as a tissue sample, a fluid sample (e.g., blood, urine or stool), or a tumor biopsy sample. Such a sample can be directly obtained from a patient or can be obtained by a third party.

As used herein, a Src inhibitor therapy is a therapy that comprises administration of a compound or prodrug of a compound that inhibits Src, such as by inhibiting Src kinase activity. Examples of such compounds include, but are not limited to, BMS-354825 (Dasatinib), SKI-606 (Bosutinib), AZD0530 (Saracatinib) and AP23451.

As used herein a MEK/ERK inhibitor therapy is a therapy that comprises administration of a compound or prodrug of a compound that inhibits MEK/ERK, such as by inhibiting MEK/ERK kinase activity. Examples of such compounds include, but are not limited to, U0126, AZD6244, PD98059, GSK1120212, GDC-0973, RDEA119, PD18416, CI1040 or FR180204.

As used herein, a PKM2 inhibitor therapy is a therapy that comprises administration of a compound (or prodrug of a compound) that inhibits PKM2, such as by inhibiting PKM2 kinase activity. In some aspects, the PKM2 inhibitor selectively inhibits PKM2 (relative to PKM1). Examples of PKM2 inhibitors include, without limitation, a polynucleotide complementary to all or part of a PKM2 gene (e.g., a PKM2-targeted shRNA, siRNA or miRNA) a small molecule inhibitor or a prodrug of such as small molecule inhibitor. Examples of small molecule PKM2 inhibitors for use according to the embodiments include the compounds detailed herein and those provided in U.S. Pat. Publn. 2010/0099726 and Vander Heiden et al., 2010, both of which are specifically incorporated herein by reference in their entirety.

In certain aspects, a method of the embodiments comprises administering a MEK/ERK inhibitor; a Src inhibitor; a NF-κB inhibitor; a PKCε inhibitor; Pin1 inhibitor and/or a PKM2 inhibitor therapy to a patient wherein the therapy is administered in conjunction with at least a second therapy. For example, the second therapy can be administered before, after or essentially simultaneously with the MEK/ERK inhibitor; a Src inhibitor; a NF-κB inhibitor; a PKCε inhibitor; Pin1 inhibitor and/or a PKM2 inhibitor therapy. Example of such second therapies include, without limitation, a surgical, radiation, hormonal, cancer cell-targeted or chemotherapeutic anticancer therapy.

In still a further embodiment a method for screening candidate anti-cancer agents (e.g., small molecule agents) is provided comprising determining the binding of PKM2 (or a fragment thereof) to β-catenin (or a fragment thereof); to Bub3 (or a fragment thereof); to MLC2 (or a fragment thereof) and/or histone H3 (or a fragment thereof) in the presence or absence of an agent, wherein an agent that disrupts binding of PKM2 to β-catenin and/or histone H3 is a candidate anti-cancer agent. Thus, in some aspects, a method comprises (a) determining the binding of PKM2 to β-catenin and/or histone H3 in the presence or absence of an agent; and (b) selecting an agent that disrupts binding of PKM2 to β-catenin; Bub3, MLC2 and/or histone H3 as a candidate anti-cancer agent.

In yet still a further embodiment there is provided a method for screening candidate PKM2 inhibitors or anti-cancer agents comprising determining PKM2 phosphorylation activity on histone H3 (or a fragment thereof, such as a fragment comprising threonine 11 of histone H3), Bub3 and/or MLC2 in the presence or absence of an agent, wherein an agent that disrupts phosphorylation of histone H3, Bub3 and/or MLC2 by PKM2 is a candidate PKM2 inhibitor or anti-cancer agent. Thus, in some aspects a method comprises (a) determining the binding of PKM2 to histone H3 and/or the phosphorylation of histone H3, Bub3 or MLC2 by PKM2 in the presence or absence of an agent; and (b) selecting a candidate PKM2 inhibitor or anti-cancer agent based on the agent disrupting the binding of PKM2 to histone H3 and/or disrupts phosphorylation of histone H3, Bub3 or MLC2 by PKM2. In some aspects, determining histone H3 phosphorylation comprises determining phosphorylation at threonine 11 of histone H3. Likewise, determining Bub3 or MLC2 phosphorylation can comprise determining phosphorylation at positions Y207 or Y118, respectively. In yet further aspects, histone H3 phosphorylation is determined indirectly, such as by determining histone H3 K9 acetylation.

In certain aspects, methods for screening of the embodiments can involve screening of small molecules, peptides and/or polypeptides (e.g., antibodies). In certain aspects, the screening methods can be in a cell-free system. In further aspects screening is performed in cells, such as cells comprised in an organism. Additional components can, in some cases, be included in the screening assay, such as without limitation, additional polypeptides, lipids, carbohydrates, ATP, buffers, chelating agents, etc.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: EGF induces the PKM2-β-catenin interaction in the nucleus. a: U87/EGFR cells were treated with or without EGF for 10 h. b: U87/EGFR cells with or without PKM2 depletion were plated and counted seven days after seeding. Data represent the mean±SD of three independent experiments. c, e: U87/EGFR cells with or without PKM2 depletion were treated with or without EGF for 24 h (c) or 10 h (e). d: U87/EGFR cells with or without PKM2 depletion were transfected with TOP-FLASH or FOP-FLASH, which was followed by EGF treatment for 10 h. Data represent the mean±SD of three independent experiments. f: Myc-TCF4 was immunoprecipitated from PKM2-depleted or PKM2-undepleted U87/EGFR cells treated with or without EGF for 10 h. g, h: PKM2 (g) or β-catenin (h) was immunoprecipitated from the indicated cell fractions of U87/EGFR cells treated with or without EGF for 6 h. i: β-catenin immunoprecipitated from U87/EGFR cells with or without EGF treatment for 6 h was incubated with or without CIP (10 U) for 30 min at 37° C. followed by three rounds of PBS washing. j, k: U87/EGFR cells stably expressing FLAG-tagged WT PKM2, PKM2 K433E (j), or PKM2 K367M (k) were treated with or without EGF for 6 h.

FIG. 2: c-Src phosphorylates β-catenin at Y333 upon EGFR activation. a: U87/EGFR cells were treated with SU6656 (4 μM) or an Abl inhibitor (0.2 μM) for 30 min before EGF treatment for 6 h. b: The indicated cells were treated with or without EGF for 6 h. c: β-catenin was immunoprecipitated from the nuclear fractions of U87/EGFR cells treated with or without EGF for 6 h. d, g: U87/EGFR cells transiently expressing the indicated FLAG-tagged β-catenin proteins were treated with or without EGF for 6 h. e: In vitro kinase assays were performed with purified active c-Src and purified β-catenin proteins. f: Immobilized GST-β-catenin proteins were mixed with purified His-PKM2 proteins in the presence or absence of active c-Src.

FIG. 3: The PKM2-β-catenin interaction is required for β-catenin-induced cyclin D1 expression. a, b: U87/EGFR cells transiently expressing FLAG-β-catenin proteins were treated with or without EGF for 10 h. c: β-catenin was depleted in U87/EGFRvIII cells, followed by reconstituted expression of β-catenin. d, g, h: U87/EGFR cells with or without depleted PKM2 and reconstituted expression of rPKM2 were treated with or without EGF for 24 h (d) or 10 h (g, h). e: U87/EGFR cells transiently expressing the indicated FLAG-tagged PKM2 proteins were treated with or without EGF for 10 h. f: U87/EGFR cells transiently expressing FLAG-PKM2 were treated with or without EGF for 10 h. 3-catenin was immunoprecipitated from the cell lysates, and the remaining supernatant was used for ChIP analyses.

FIG. 4: The PKM2-β-catenin interaction is required for tumor development. a, b: U87, U87/EGFRvIII cells with or without depleted β-catenin and reconstituted expression of rβ-catenin (a), or U87/EGFRvIII cells with or without depleted PKM2 and reconstitution of the expression of rPKM2 (b), were plated and counted seven days after seeding. Data represent the mean±SD of three independent experiments. c: U87 (bottom left panel), U87/EGFRvIII cells with or without depleted β-catenin and reconstituted expression of rβ-catenin (top panel), or U87/EGFRvIII cells with or without depleted PKM2 and reconstituted expression of rPKM2 (bottom right panel) were intracranially injected into athymic nude mice. After two weeks, tumor growth was examined. H & E-stained coronal brain sections show representative tumor xenografts. d: IHC staining with the indicated antibodies was performed on 55 GBM specimens. Representative photos of four tumors are shown. e, f: The survival time for 84 patients with low (0-5 staining scores, blue curve) versus high (6-8 staining scores, red curve) β-catenin Y333 phosphorylation (e: low, 28 patients; high, 56 patients) and nuclear PKM2 expression (f: low, 28 patients; high, 56 patients) were compared (bottom panel). The table (top panel) shows the multivariate analysis after adjustment for patient age, indicating the significance level of the association of Y133-phosphorylated β-catenin expression (e) or nuclear PKM2 expression (f) with patient survival. Empty circles represent deceased patients, and filled circles represent censored (alive at last clinical follow-up) patients.

FIG. 5: A mechanism for EGFR-induced β-catenin transactivation. EGFR activation results in the translocation of PKM2 and c-Src to the nucleus. c-Src phosphorylates β-catenin at Y333 in the nucleus, leading to the interaction between PKM2 K433 and the phosphorylated Y333 residue of β-catenin. The binding of PKM2 to the CCND1 promoter, which is likely guided by the associated β-catenin, is important for HDAC3 disassociation from the promoter and subsequent acetylation of histone H3. This protein complex, together with TCF/LEF, modulates cyclin D1 expression and promotes cell proliferation and tumorigenesis.

FIG. 6: EGFR activation induces nuclear translocation of PKM2. Immunoblotting analyses (b, b) were performed with the indicated antibodies. Nuclear PCNA and cytoplasmic tubulin were used as controls. a: The indicated cells were immunostained with an anti-PKM2 antibody. Nuclei were stained with Hoechst 33342. b: The nuclear fractions were prepared from DU145, MDA-MB-231 (left panel), and U87/EGFR cells (right panel), which were pretreated with or without cycloheximide (CHX) (100 μg/mL) for 30 min before being treated with or without EGF (100 ng/mL) for 10 h. c: The nuclear fractions were prepared from U87/EGFR cells, which had been treated with or without EGF (100 ng/mL) for 10 h.

FIG. 7: PKM2 is not required for Wnt-induced β-catenin transactivation. a: U87/EGFR cells were stably transfected with pGIPZ expressing a control or a PKM2 shRNA. b: U87/EGFR cells with or without PKM2 depletion were transfected with either TOP-FLASH or FOP-FLASH, which was followed by WNT3A (20 ng/mL) treatment for 10 h. The relative levels of luciferase activity were normalized to the levels of untreated cells and to the levels of luciferase activity of the Renilla control plasmid. Data represent the mean±SD of three independent experiments. c: U87/EGFR cells with or without PKM2 depletion were treated with or without WNT3A (20 ng/mL) for 24 h. d: U87/EGFR cells were treated with or without WNT3A (20 ng/mL) for 10 h. The nuclear fractions were prepared.

FIG. 8: PKM2 does not bind to β-catenin without post-translational modifications. A GST pull-down assay was performed by mixing purified GST-β-catenin on glutathione agarose beads with purified His-PKM2 (left lane). Purified His-PKM2 was used as a positive control for the immunoblotting analysis (right lane).

FIG. 9: PKM2 K433E has comparable enzyme activity to WT PKM2. The activity of bacterially purified WT PKM2 (0.1 g) and PKM2 K433E (0.1 g) toward PEP in the presence of a saturated amount of the PKM2 activator fructose-1,6-bisphosphate (FBP) was measured using a pyruvate kinase assay. Data represent the mean±SD of three independent experiments.

FIG. 10: EGF induces activation and nuclear translocation of c-Src and nuclear β-catenin phosphorylation. Immunoprecipitation and immunoblotting analyses were performed with the indicated antibodies. a: U87/EGFR cells were treated with SU6656 (4 VM) or an Abl inhibitor (0.2 VM) for 30 min before EGF (100 ng/mL) treatment for 6 h. b: The indicated cells were treated with or without EGF (100 ng/mL) for 6 h. c: Cytosolic or nuclear fractions of U87/EGFR cells treated with or without EGF (100 ng/mL) for 6 h were prepared. d: β-catenin was immunoprecipitated from the membrane, cytosol, or nuclear fractions of U87/EGFR cells treated with or without EGF (100 ng/mL) for 6 h.

FIG. 11: PKM2 K433E and the inactive PKM2 K367M mutants translocated into the nucleus upon EGF stimulation. U87/EGFR cells expressing FLAG-PKM2 K433E or FLAG-PKM2 K367M were treated with or without EGF (100 ng/mL) for 10 h. The nuclear fractions and total cell lysates were prepared. Immunoblotting analyses were performed with the indicated antibodies.

FIG. 12: Reconstituted expression of WT rPKM2, rPKM2 K367M, or rPKM2 K433E in U87/EGFR cells with depleted endogenous PKM2. Cell lysates were prepared from the indicated cells. Immunoblotting analyses were performed with the indicated antibodies.

FIG. 13: EGF induces the β-catenin downstream effector gene DKK1, but not AXIN2 and βTrCP, in a PKM2-dependent manner. U87/EGFR cells with or without PKM2 depletion were treated with or without EGF (100 ng/mL) for 24 h. mRNA expression levels of DKK1, AXIN2, and βTrCP were measured by real-time quantitative RT-PCR analysis. 3-actin mRNA from the same cDNA library was amplified as a control. The relative mRNA levels of DKK1, AXIN2, and βTrCP were normalized to the levels of untreated cells and P-actin mRNA. Data represent the mean±SD of three independent experiments.

FIG. 14A-H: β-catenin Y333 phosphorylation plays distinct roles in EGF and WNT-induced signaling and biological activities. a: β-catenin Y333F, like its WT counterpart, binds to APC, AXIN2, and E-cadherin. A vector expressing FLAG-β-catenin or FLAG-β-catenin Y333F was transiently transfected into A431 cells. Immunoprecipitation and immunoblotting analyses were performed with the indicated antibodies. b: β-catenin Y333F, like its WT counterpart, increases its association with TCF4 upon WNT1 expression. A vector expressing Myc-TCF4 was co-transfected with a vector expressing WT FLAG-β-catenin or FLAG-β-catenin Y333F into U87/EGFR cells. These cells were infected with or without lentiviruses expressing WNT1 for 72 h. Immunoprecipitation and immunoblotting analyses were performed with the indicated antibodies. c: β-catenin Y333F behaves similarly to WT β-catenin in WNT1-induced β-catenin transactivation. U87/EGFR cells with β-catenin depletion were reconstituted to express WT β-catenin or β-catenin Y333F. These cells were transfected with either TOP-FLASH or FOP-FLASH and infected with or without lentivirus expressing WNT1 for 72 h. The relative levels of luciferase activity were normalized to the levels of untreated cells and to the levels of luciferase activity of the Renilla control plasmid. Data represent the mean±SD of three independent experiments. d: β-catenin Y333F behaves similarly to WT β-catenin in WNT1-induced expression of downstream target genes, such as AXIN2, DKK1, and βTrCP. U87/EGFR cells depleted of endogenous β-catenin were reconstituted to express WT rβ-catenin or rβ-catenin Y333F. These cells were infected with or without lentivirus expressing WNT1 for 72 h. mRNA expression levels of DKK1, AXIN2, and βTrCP were measured by real-time quantitative RT-PCR analysis. β-actin mRNA from the same cDNA library was amplified as a control. The relative mRNA levels of DKK1, AXIN2, and βTrCP were normalized to the levels of untreated cells and β-actin mRNA. Data represent the mean±SD of three independent experiments. e: β-catenin Y333F co-localizes with E-cadherin as does its WT counterpart. Immunofluorescence analyses of A431 cells expressing WT FLAG-β-catenin or FLAG-β-catenin Y333F were performed with the indicated antibodies. Nuclei were stained with Hoechst 33342 (blue). f: β-catenin Y333F does not significantly alter focal adhesions, as detected by vinculin staining. Immunofluorescence analyses of NIH 3T3 cells expressing WT FLAG-β-catenin or FLAG-β-catenin Y333F were performed with the indicated antibodies. g: There are no differences in the effects of WT β-catenin or β-catenin Y333F expression on WNT3A-induced inhibition of nerve growth factor-stimulated neurite outgrowth of PC12 cells27. PC12 cells overexpressing WT FLAG-β-catenin or FLAG-β-catenin Y333 (immunoblotting analyses: left panel) were treated with or without WNT3A (20 ng/mL) and/or nerve growth factor (NGF) (100 ng/mL) for 6 d. Pictures were taken with a digital camera mounted on a microscope with 100× magnification (top right panel). Cells with neurite extensions longer than two cell diameter lengths were counted (bottom right panel). A total of 200 cells for each condition were examined. h: β-catenin Y333F blocks EGF- but not WNT3A-induced cell migration. U87/EGFR cells depleted of endogenous β-catenin were reconstituted to express WT rβ-catenin or rβ-catenin Y333F. These cells were plated at the top surface of the Matrigel in the absence or presence of WNT3A (20 ng/mL) or EGF (100 ng/mL). Twelve hours after plating, cells that had migrated to the opposite side of the insert were stained with crystal violet. Representative microphotographs are shown (left panel). The membranes with invaded cells were dissolved in 4% deoxycholic acid and read colorimetrically at 590 nm for quantification of invasion. Data represent the mean±standard deviation of three independent experiments (right panel).

FIG. 15: HDAC1 or HDAC2 is not prebound to the CCND1 promoter, and HDAC3 interacts with PKM2. a: U87/EGFR cells were treated with or without EGF (100 ng/mL) for 10 h. A ChIP assay was performed with antibodies for HDAC1 or HDAC2 for immunoprecipitation, followed by PCR with CCND1 promoter-specific primers. b: U87/EGFR cells transiently expressing FLAG-HDAC3 with WT HA-PKM2 or HA-PKM2 K367M were treated with or without EGF (100 ng/mL) for 10 h.

FIG. 16: EGF induces the nuclear translocation of PKM2, c-Src-dependent 3-catenin Y333 phosphorylation, and a c-Src-dependent interaction between PKM2 and β-catenin in primary GBM cells. Immunoprecipitation and immunoblotting analyses were performed with the indicated antibodies. Nuclear PCNA and cytoplasmic tubulin were used as controls. A: The total cell lysates and nuclear fractions were prepared from the indicated primary GBM cells, which were treated with or without EGF (100 ng/mL) for 10 h (top panel) or 24 h (bottom panel). B, C: The total cell lysates were prepared from the indicated primary GBM cells, which were pretreated with or without SU6656 (4 VM) for 30 min before being treated with or without EGF (100 ng/mL) for 6 h.

FIG. 17: The PKM2-β-catenin interaction is essential for cell cycle progression. a: Expression of WT rPKM2, rPKM2 K433E, or rPKM2 K367M was reconstituted in U87/EGFRvIII cells with depleted PKM2. Immunoblotting analyses were performed with the indicated antibodies. b: U87/EGFRvIII cells with or without depletion of β-catenin (left panel) and PKM2 (right panel) and reconstituted expression of WT rβ-catenin, rβ-catenin Y333F, WT rPKM2, or rPKM2 K433E were stained with propidium iodide and analyzed for DNA staining profiles by flow cytometry. Data represent the mean±SD of three independent experiments.

FIG. 18: PKM2-regulated β-catenin transactivation is required for brain tumor growth. a: GSC11 cells (5×10⁵) with or without RNAi-depleted β-catenin that were reconstituted to express WT rβ-catenin or rβ-catenin Y333F, or GSC11 cells (5×10⁵) with or without RNAi-depleted PKM2 that were reconstituted to express WT rPKM2 or rPKM2 K433E, were intracranially injected into athymic nude mice. After 30 days, the mice were sacrificed to examine tumor growth. H & E-stained coronal brain sections show representative tumor xenografts that were obtained for each group of mice. Immunoblotting analyses were performed with the indicated antibodies for the indicated cell lines (top panel). b: GSC11 cells (5×10⁵) that were infected with lentiviruses expressing PKM2 shRNA in a TRIPZ vector were intracranially injected into athymic nude mice for each group. After 14 d, seven mice were sacrificed to examine tumor growth; the remaining mice that were fed with drinking water with or without doxycycline (800 μg/mL) were sacrificed at day 30. H & E-stained coronal brain sections showed representative tumor xenografts (top panel). Tumor volumes (bottom left panel) were estimated by measuring two dimensions [length (a) and width (b)] and calculated using the equation: V=ab2/2. Immunoblotting analyses of protein lysates from the indicated tumor tissues were performed with the indicated antibodies (bottom right panel).

FIG. 19: c-Src inhibition suppresses tumorigenesis and reduces β-catenin Y333 phosphorylation and cyclin D1 expression in tumor tissues. a: U87/EGFRvIII (5×10⁵) cells were intracranially injected into athymic nude mice. After 5 d, seven mice were sacrificed to examine tumor growth; the remaining mice, whose tumors were intracranially injected with SU6656 (0.015 mg/kg in 5 μL of DMSO) or DMSO every three days, were sacrificed at day 15. H & E-stained coronal brain sections showed representative tumor xenografts. b: Tumor volumes were measured using length (a) and width (b) and calculated using the equation: V=ab2/2. C: Immunoblotting analyses of the indicated tumor tissues were performed with the indicated antibodies.

FIG. 20: WNT1 expression rescues PKM2-dependent tumorigenesis. a: U87/EGFRvIII-PKM2 shRNA cells with or without reconstituted expression of rPKM2 K433E were infected with lentiviruses expressing WNT1 for 72 h. Immunoblotting analyses were performed with the indicated antibodies. b, c: A total number of 5×10⁵ U87/EGFRvIII cells with or without RNAi-depleted PKM2 and reconstitution of rPKM2 K433E expression were infected with or without lentiviruses expressing WNT1 and intracranially injected into athymic nude mice. After two weeks, the mice were sacrificed to examine tumor growth. H & E-stained coronal brain sections showed representative tumor xenografts (b). c: Tumor volumes were measured using two dimensions [length (a) and width (b)] and calculated using the equation: V=ab2/2.

FIG. 21: EGFR expression is correlated with c-Myc and CCND1 expression in human GBM samples. Publicly available microarray datasets (Affymetrix, U133) from TCGA and other sources were analyzed for relationships between EGFR expression and the expression of c-Myc and CCND1. Normalized data for GBM samples were used to compare average gene expression levels of c-Myc and CCND1 in samples with an EGFR expression level of 5.0 or higher (n=204), versus expression of these genes in samples with EGFR expression levels of 2.0 or lower (n=403).

FIG. 22: The phosphorylation levels of β-catenin Y333 correlated with phosphorylation levels of activated c-Src in seven human primary GBM cell lines. Immunoblotting analyses of lysates from seven lines of human primary GBM cells were performed with the indicated antibodies.

FIG. 23: Validation of antibody specificities. Immunofluorescence and IHC analyses were performed with the indicated antibodies. a: Immunofluorescence analyses of c-SRC+/+ and c-SRC−/− cells were performed with the indicated antibodies. EGF induced phosphorylation of c-Src Y418 and β-catenin Y333 in c-SRC+/+, but not in c-SRC−/−, cells. b: Immunofluorescence analyses of U87/EGFR with or without expression of PKM2 shRNA were performed with an anti-PKM2 antibody. c: IHC analyses of human GBM tissues were performed with the indicated antibodies in the presence or absence of specific blocking peptides.

FIG. 24: The levels of c-Src Y418 phosphorylation, β-catenin Y333 phosphorylation, and nuclear expression of PKM2 are correlated with each other. IHC staining with anti-phospho-c-Src Y418, anti-phospho-β-catenin Y333, and anti-PKM2 antibodies was performed on 55 GBM specimens. Semiquantitative scoring was performed (Pearson product moment correlation test; left panel, r=0.81, P<0.00001; middle panel, r=0.70, P<0.0001; right panel, r=0.74, P<0.0001). Note that some of the dots on the graphs represent more than one specimen (some scores overlapped).

FIG. 25: The levels of c-Src Y418 phosphorylation correlate with grades of glioma malignancy. Immunohistochemical staining of 30 diffuse astrocytoma specimens with anti-phospho-β-catenin Y333 antibody was performed and analyzed by comparing it with the staining of 88 GBM specimens (Student's t-test, two tailed, P<0.001).

FIG. 26: β-catenin Y333 is phosporylated in MDA-MB-231 (breast cancer) and DU145 (prostate cancer) cells. Immunoblot shows expression Y333 phosphorylated β-catenin in the indicated cell lines.

FIG. 27: EGF-induced and PKM2-dependent phosphorylation of histone H3 at T11 is required for acetylation of histone H3 at K9. Immunoprecipitation and immunoblotting analyses were performed with the indicated antibodies. A: U87/EGFR cells expressing FLAG-tagged WT H3, H3-K4R, and K9R were treated with or without EGF (100 ng/mL) for 6 h. B, F: U87/EGFR and U251 cells expressing a control or PKM2 shRNA were treated with or without EGF (100 ng/mL) for 6 h. Endogenously expressed histone H3 was examined. Data represent the mean±SD of three independent experiments (F). C: U87/EGFR were treated with or without EGF (100 ng/mL) or 20% serum with calyculin A (25 nM) for 6 h. D: U87/EGFR cells with or without expressing PKM2 shRNA were treated with or without EGF (100 ng/mL) for 6 h. Endogenously expressed histone H3 was immunoprecipitated. E: U87/EGFR cells expressing FLAG-tagged WT H3, H3-T3A, H3-T6A, and H3-T11A were treated with or without EGF (100 ng/mL) for 6 h. G: U87/EGFR cells expressing FLAG-tagged WT H3 or H3-T11A were treated with or without EGF (100 ng/mL) for 6 h. H: U87/EGFR cells expressing a control shRNA or shRNA against Chk1, DAPK3, or PKN1 mRNA were analyzed by immunoblotting analysis with the indicated antibodies. I: U87/EGFR cells expressing a control shRNA or shRNA against Chk1, DAPK3, or PKN1 mRNA were treated with or without EGF (100 ng/mL) for 6 h and analyzed by immunoblotting analysis with the indicated antibodies.

FIG. 28: PKM2 directly Interacts with Histone H3 and Phosphorylates H3-T11. Immunoprecipitation and immunoblotting analyses were performed with the indicated antibodies. A: Pull-down analyses were performed by mixing purified immobilized His-PKM2 on nickel agarose beads with purified non-tagged recombinant histone H3 or histone H2A. B: U87/EGFR cells were treated with or without EGF (100 ng/mL) for 6 h. C: In vitro phosphorylation was analyzed by mixing recombinant WT PKM2 or PKM2 K367M with purified recombinant WT H3 or H3-T11A in the presence of PEP or ATP. D: Purified recombinant His-histone H3 was phosphorylated by PKM2 in vitro and was analyzed by mass spectrometry. Mass spectrometric analysis of a tryptic fragment at m/z 533.258 (mass error was −0.98 ppm) matched to the doubly-charged peptide 9-KSTGGKAPR-17, suggesting that T11 was phosphorylated. The Sequest score for this match was Xcorr=2.74; Mascot score was 46, expectation value 5.1×10⁻⁴; pRS score was 116; site probability was 99.1%. The presence of the b₂⁺ ion at 258.2 indicates the S10 residue is unmodified; the presence of the y₇⁺ at 808.2 is in agreement with the assignment of the phosphorylation site to T11. E, F: U87/EGFR cells expressing PKM2 shRNA were reconstituted by the expression of WT rPKM2, rPKM2 K367M, or rPKM2 K433E (D) and treated with or without EGF (100 ng/mL) for 6 h (E).

FIG. 29: PKM2-dependent H3-T11 phosphorylation promotes the disassociation of HDAC3 from CCND1 and MYC promoter. Immunoblotting analyses were performed with the indicated antibodies. A, B: WT rH3 or rH3-T11A expression was reconstituted in endogenous H3-depleted U87/EGFR cells (A), which were then treated with or without EGF (100 ng/mL) for 6 h. ChIP analyses with a HDAC3 antibody were performed (B). C: GST-HDAC3 pull-down assay was performed by incubation of 100 ng of purified recombinant His-tagged WT histone H3 or H3-T11A mutant with or without immobilized GST-HDAC3, which was followed by incubation with 200 ng of purified recombinant WT His-PKM2 or His-PKM2 K367M in the presence of PEP.

FIG. 30: PKM2-dependent H3-T11 phosphorylation promotes EGF-induced expression of cyclin D1 and c-Myc. Immunoprecipitation, immunoblotting, and ChIP analyses were performed with the indicated antibodies. A: U87/EGFR cells with or without depletion of endogenous PKM2 and reconstituted expression of WT rPKM2 or rPKM2 K367M were treated with or without EGF (100 ng/mL) for 6 h. B: U87/EGFR cells expressing FLAG-tagged WT H3 or H3-T11A were treated with EGF (100 ng/mL) for 6 h. C, D, E: U87/EGFR cells with depleted endogenous histone H3 and reconstituted expression of WT rH3 or rH3-T11A were treated with or without EGF (100 ng/mL) for 6 h. ChIP analyses were performed with an anti-PKM2 (C) or an anti-acetyl-H3K9 antibody (D). E: Quantitative real time polymerase chain reaction (PCR) was performed with specific primers for CCND1 (left panel) or MYC mRNA (right panel). F: U87/EGFR cells with depleted endogenous histone H3 and reconstituted expression of WT rH3, rH3-T11A, or rH3-K9R were treated with or without EGF (100 ng/mL) for 6 h. G: U87/EGFR cells with endogenous PKM2 depletion and reconstituted expression of WT rPKM2 or rPKM2 K367M were treated with or without EGF (100 ng/mL) for 6 h.

FIG. 31: PKM2-dependent H3-T11 phosphorylation is required for cell cycle progression, cell proliferation, and tumor development. A: WT rH3 or rH3-T11A expression was reconstituted in U87/EGFRvIII cells with depleted endogenous H3. Immunoblotting analyses were performed with the indicated antibodies. B: U87/EGFRvIII cells with depleted PKM2 or endogenous H3 and reconstituted expression of WT rH3 or rH3-T11A were stained with propidium iodide and analyzed for DNA staining profiles by flow cytometry. Data represent the mean±SD of three independent experiments. C: A total number of 2×10⁴ U87/EGFRvIII cells with depleted PKM2 or endogenous H3 and reconstituted expression of WT rH3 or rH3-T11A were plated and counted seven days after seeding in DMEM with 2% bovine calf serum. Data represent the mean±SD of three independent experiments. D, E, F: A total of 5×10⁵ endogenous histone H3-depleted U87/EGFRvIII (D) or GSC11 (F) cells with reconstituted expression of WT rH3 or rH3-T11A were intracranially injected into athymic nude mice for each group. The mice were sacrificed and examined for tumor growth. H & E-stained coronal brain sections show representative tumor xenografts. Tumor volumes were measured by using length (a) and width (b) and calculated using the equation: V=ab²/2. Data represent the mean±SD of seven mice. E: Immunoblotting analysis with anti-phospho-H3-T11 antibody was performed on lysates of the tumor tissue derived from the mice injected with U87/EGFvIII cells with reconstituted expression of WT histone H3 and the counterpart tissue derived form the mice injected with U87/EGFvIII cells with reconstituted expression of histone H3 T11A mutant.

FIG. 32: H3-T11 phosphorylation positively correlates with the level of nuclear PKM2 expression and with grades of glioma malignancy and prognosis. A, B: Immunohistochemical staining with anti-phospho-EGFR Y1172, anti-phospho-H3-T11, and anti-PKM2 antibodies was performed on 45 GBM specimens. Representative photos of four tumors are shown (A). Semiquantitative scoring was performed (Pearson product moment correlation test; r=0.704, P<0.0001, left panel; r=0.86, P<0.001, right panel). Note that some of the dots on the graphs represent more than one specimen (some scores overlapped) (B). C: The survival times for 85 patients with low (0-4 staining scores, blue curve) versus high (4.1-8 staining scores, red curve) H3-T11 phosphorylation (low, 16 patients; high, 69 patients) were compared. The table (top panel) shows the multivariate analysis after adjustment for patient age, indicating the significance level of the association of H3-T11 phosphorylation (P=0.01349038) with patient survival. Empty circles represent deceased patients, and filled circles represent censored (alive at last clinical follow-up) patients. D: Thirty diffuse astrocytoma specimens were immunohistochemically stained with anti-phospho-H3-T11 antibody, and specimens were compared with 45 stained GBM specimens (Student's t-test, two tailed, P<0.001).

FIG. 33: PKM2 regulates gene expression by H3-T11 phosphorylation. EGFR activation results in nuclear translocation of PKM2 that binds to gene promoter regions, where PKM2 phosphorylates H3-T11, leading to HDAC3 disassociation from the promoters and subsequent acetylation of histone H3, transcription of genes, cell cycle progression, and cell proliferation.

FIG. 34: EGF induces PKM2-dependent histone H3 acetylation at K9 and phosphorylation at T11. A, B: U87/EGFR cells with or without PKM2 depletion were treated with EGF (100 ng/mL) for 6 h. LC-MS/MS analyses of a tryptic digest of immunoprecipitated endogenous histone H3 revealed acetylation of H3 at K9 in peptide 9-KSTGGKAPR-17 in a PKM2-dependent manner. The immunoprecipitated H3 was acetylated by d6-acetic anhydride and digested by trypsin. A characteristic pattern is observed in the mass spectrum for partly acetylated sites: lysines not acetylated react with the d6-acetic anhydride while endogenously acetylated lysines do not. For example, a peptide with one partly acetylated lysine gives rise to two MS peaks, separated by about 3 d [the mass difference between three ¹H hydrogens and three deuteriums (²H)]. The peak for the mono-isotopic ion of the endogenously acetylated peptide (observed at 494.7 d) is indicated by an asterisk (A). The average ratio of acetylated to non-acetylated H3 K9 was quantified from two separated experiments (B).

FIG. 35: Depletion of PKM2 and PKM1 blocks EGF-induced phosphorylation of histone H3 at T11. U87/EGFR cells expressing a control shRNA or shRNA against a coding sequence for both PKM2 and PKM1 were treated with or without EGF (100 ng/mL) for 6 h. Immunoblotting analysis of endogenous histone H3 was performed with the indicated antibodies.

FIG. 36: EGF-induced histone H3-T11 phosphorylation does not affect H3 trimethylation at K36. U87/EGFR cells expressing FLAG-tagged WT H3 or H3-T11A were treated with or without EGF (100 ng/mL) for 6 h. Immunoprecipitation and immunoblotting analyses were performed with the indicated antibodies.

FIG. 37: Chk1, DAPK3, or PKN1 is not involved in EGF-induced histone H3-T11 phosphorylation. A: U251 cells expressing a control shRNA or shRNA against Chk1, DAPK3, or PKN1 mRNA were analyzed by immunoblotting analysis with the indicated antibodies. B: U251 cells expressing a control shRNA or shRNA against Chk1, DAPK3, or PKN1 mRNA were treated with or without EGF (100 ng/mL) for 6 h and analyzed by immunoblotting analysis with the indicated antibodies.

FIG. 38: PKM2 phosphorylates histone H3 in a large fraction and PKM2 K367M and PKM1 were able to interact with histone H3. A: In vitro phosphorylation was analyzed by mixing recombinant PKM2 with purified histone H3 in the presence of PEP. The phosphorylated and non-phosphorylated histone H3 proteins were separated by immunoprecipitation with a saturated amount of anti-phospho-H3 T11 and non-phospho-H3 antibodies. Immunoblotting analysis was performed with the indicated antibodies. B: Pull-down analyses were performed by mixing immobilized recombinant His-K367M and PKM1 on nickel agarose beads with purified non-tagged histone H3. Immunoblotting analysis was performed with the indicated antibodies.

FIG. 39: Phosphorylated histone H3 by PKM2 lost its ability to interact with HDAC3. Purified His-tagged WT histone H3 was mixed with purified WT His-PKM2 in the presence or absence of PEP. Phosphosphorylated H3 from the sample with PEP and non-phosphosphorylated H3 from the sample without PEP were immunoprecipitated with anti-phospho-H3 T11 and regular H3 antibody, respectively, which was followed by incubation with soluble GST-HDAC3. Immunoblotting analysis was performed with the indicated antibodies.

FIG. 40: PKM2-dependent H3-T11 phosphorylation is required for EGF-induced H3-K9 acetylation and subsequent expression of cyclin D1 and c-Myc. A: U251 cells with depleted endogenous histone H3 and reconstituted expression of WT rH3 or rH3-T11A were treated with or without EGF (100 ng/mL) for 6 h. ChIP analyses were performed with an anti-acetyl-H3K9 antibody. B: U251 cells with depleted endogenous histone H3 and reconstituted expression of WT rH3, rH3-T11A, or rH3-K9R were treated with or without EGF (100 ng/mL) for 6 h. Immunoblotting analysis was performed with the indicated antibodies.

FIG. 41: Depletion of PKM2 and PKM1 blocked EGF-induced cell cycle progression, cell proliferation, and brain tumorigenesis. A: U87/EGFRvIII cells expressing a control shRNA or shRNA against a coding sequence for both PKM2 and PKM1. Immunoblotting analysis was performed with the indicated antibodies. B: U87/EGFRvIII cells expressing a control shRNA or shRNA against a coding sequence for both PKM2 and PKM1 were stained with propidium iodide and analyzed for DNA staining profiles by flow cytometry. Data represent the mean±SD of three independent experiments. C: A total of 2×10⁴ U87/EGFRvIII cells expressing a control shRNA or shRNA against a coding sequence for both PKM2 and PKM1 were plated and counted seven days after seeding in DMEM with 2% bovine calf serum. Data represent the mean±SD of three independent experiments. D: A total of 5×10⁵ U87/EGFRvIII cells expressing a control shRNA or shRNA against a coding sequence for both PKM2 and PKM1 were intracranially injected into athymic nude mice for each group. After two weeks, the mice were sacrificed and examined for tumor growth. H & E-stained coronal brain sections show representative tumor xenografts.

FIG. 42: ERK is required for PKM2 nucleus translocation. Immunoblotting and immunoprecipitation analyses were performed with the indicated antibodies. a: Nuclear fractions were prepared from U87/EGFR cells pretreated with LY290042 (30 μM), SU6656 (4 μM), SP600125 (25 μM), and U0126 (20 μM) for 30 min before EGF (100 ng/mL) for 6 h. Nuclear PCNA and cytoplasmic tubulin were used as controls. b: U87/EGFR cells were pretreated with or without U0126 (20 μM) for 30 min before EGF (100 ng/mL) for 6 h. Immunofluorescence analyses were performed with the indicated antibodies. Nuclei were stained with Hoechst 33342 (blue). c: U251 cells were stably transfected with a vector with or without expressing FLAG-ERK2 K52R (left panel) or transiently transfected with vectors expressing MEK1 Q56P and indicated FLAG-tagged ERK2 proteins (right panel). The cells were treated with or without EGF (100 ng/mL) for 6 h, and the total cell lysates and nuclear fractions were prepared. d: FLAG-PKM2 or FLAG-PKM1 was immunoprecipitated with an anti-FLAG antibody from U87/EGFR cells treated with or without EGF (100 ng/mL) for 30 min. e: Bacterially-purified GST-ERK2 immobilized on glutathione agarose beads was mixed with or without purified His-PKM2. GST pull-down analyses were performed. Ten percent of total His-PKM2 was used as an input. f: U87/EGFR cells transfected with vectors expressing the indicated FLAG-tagged ERK proteins were treated with or without EGF (100 ng/mL) for 30 min. g: U87/EGFR cells expressing the indicated FLAG-PKM2 proteins were treated with or without EGF (100 ng/mL) for 30 min.

FIG. 43: ERK2 phosphorylates PKM2 S37. Immunoblotting and immunoprecipitation analyses were performed with the indicated antibodies. a: In vitro kinase assays were performed with purified active ERK2, WT PKM2, and PKM2 S37A mutant. b: FLAG-PKM1 or FLAG-PKM2 was immunoprecipitated with anti-FLAG antibody from U87/EGFR cells pretreated with or without U0126 (20 μM) for 30 min before EGF (100 ng/mL) for 30 min. c: MEK1 Q56P and the indicated ERK2 proteins were transiently expressed in U87/EGFR cells. d, e, f: Total cell lysates (d) or nuclear fractions (e) were prepared from U87/EGFR cells expressing FLAG-tagged WT PKM2, PKM2 S37A, or PKM2 S37D, which had been treated with or without EGF (100 ng/mL) for 6 h. Immunofluorescence analyses were performed with an anti-FLAG antibody (f). Nuclei were stained with Hoechst 33342 (blue).

FIG. 44: PKM2 S37 phosphorylation recruits PIN1. Immunoprecipitation and immunoblotting analyses were performed with the indicated antibodies. a: His-PIN1 immobilized on nickel agarose beads was incubated with lysate of U87/EGFR cells treated with or without U0126 (20 μM) for 30 min before EGF (100 ng/mL) stimulation for 30 min. b: U87/EGFR cells transiently expressing WT FLAG-PKM2 or FLAG-PKM2 S37A were treated with or without EGF (100 ng/mL) for 30 min. His-PIN1 immobilized on nickel agarose beads was incubated with the cell lysates. c: His-PIN1 or His-PIN1 WW domain mutant (with substitutions at W11A, W34A, R14A, and R17A) immobilized on nickel agarose beads was incubated with lysate of U87/EGFR cells treated with or without EGF (100 ng/mL) for 30 min. d: Purified GST-PIN1 on glutathione agarose beads was mixed with purified His-PKM2 with or without purified active ERK2 (left panel) or incubated with purified His-PKM2 S37A or His-PKM2 S37D mutant (right panel). e: PIN1 was immunoprecipitated from U87/EGFR cells pretreated with or without U0126 (20 μM) for 30 min before EGF (100 ng/mL) stimulation for 30 min (left panel) or from U87/EGFR cells expressing FLAG-PKM2 or FLAG-PKM2 S37D (right panel). f: Cis-trans isomerization assays were performed by mixing synthesized phosphorylated or nonphosphorylated oligopeptide of PKM2 containing the S37P motif with purified WT GST-PIN 1 or GST-PIN 1 C113A mutant. Data represent the mean±SD of three independent experiments. g: PIN1^{-/-} cells were reconstituted to express the indicated PIN1 proteins (left panel). The total cell lysates and nuclear fractions were prepared from the indicated cells treated with or without EGF (100 ng/mL) for 6 h (right panel). h: Total cell lysates and nuclear fractions of PIN1^{-/-} cells or PIN^{-/-} cells expressing FLAG-PKM2 S37D were prepared.

FIG. 45: PIN1 regulates binding of PKM2 to importin α5. Immunoprecipitation and immunoblotting analyses were performed with the indicated antibodies. a: U87/EGFR cells expressing WT FLAG-PKM2 or the indicated FLAG-PKM2 mutants were treated with or without EGF (100 ng/mL) for 6 h, and the total cell lysates and nuclear fractions were prepared. b: Immunofluorescence analyses of U87/EGFR cells expressing FLAG-PKM2 R399/400A were performed with an anti-FLAG antibody. Nuclei were stained with Hoechst 33342. c: The indicated FLAG-importin α proteins and EGFR were coexpressed in 293T cells, which were treated with EGF (100 ng/mL) for 30 min. FLAG-importin α proteins were immunoprecipitated with an anti-FLAG antibody. d: U87/EGFR cells expressing WT FLAG-PKM2 or FLAG-PKM2 R399/400A were treated with or without EGF (100 ng/mL) for 30 min. The cell lysate was mixed with purified GST-importin α5 for a GST pull-down assay. e: U87/EGFR cells with or without depletion of importin α5 were treated with or without EGF (100 ng/mL) for 6 h. The cytosolic and nuclear fractions were prepared. f: Purified His-PKM2 S37D on nickel agarose beads was mixed with purified GST-importin α5 in the presence or absence of purified WT His-PIN1 or His-PIN 1 C113A. A GST pull-down assay was performed.

FIG. 46: Nuclear PKM2 regulates glycolytic gene expression. Immunoblotting analyses were performed with the indicated antibodies. a, c: U87/EGFR cells with or without PKM2 depletion and reconstituted expression of the indicated PKM2 proteins (a) or U87/EGFR cells with or without PIN1 depletion (c) were transfected with either TOP-FLASH or FOP-FLASH (control vector), which was followed by EGF (100 ng/mL) treatment for 12 h. The relative levels of luciferase activity were normalized to the levels of untreated cells and to the levels of luciferase activity of the Renilla control plasmid. Data represent the mean±SD of three independent experiments. b: U87/EGFR cells with or without PKM2 depletion and reconstituted expression of the indicated PKM2 proteins were treated with or without EGF (100 ng/mL) for 12 h. ChIP assay was performed with an anti-β-catenin antibody for immunoprecipitation followed by PCR with MYC promoter-specific primers. d: U87/EGFR cells with or without PKM2 depletion and reconstituted expression of WT rPKM2 or rPKM2 S37A mutant were treated with or without EGF (100 ng/mL) for 24 h. e: U87/EGFR cells expressing FLAG-PKM2 S37A or FLAG-PKM2 R399/400A were treated with or without EGF (100 ng/mL) for 24 h. f: U87/EGFR cells with or without c-Myc depletion were treated with or without EGF (100 ng/mL) for 24 h.

FIG. 47: Nuclear PKM2 is required for the Warburg effect. Immunoblotting analyses were performed with the indicated antibodies. a, b: U87/EGFRvIII cells with or without PKM2 depletion and reconstituted expression of WT rPKM2 or rPKM2 S37A mutant (a) were cultured in no-serum DMEM. The media were collected for analysis of glucose consumption (b, left panel) or lactate production (b, right panel). Data represent the mean±SD of three independent experiments. *P<0.01: statistically significant value in relation with U87/EGFRvIII cells without PKM2 depletion. #P<0.01: statistically significant value in relation with U87/EGFRvIII cells with PKM2 depletion and reconstituted expression of WT rPKM2. c: U87/EGFRvIII cells (5×10⁵) with or without RNAi-depleted PKM2 and reconstituted expression of WT rPKM2 or rPKM2 S37A were intracranially injected into athymic nude mice. After two weeks, mice were sacrificed for examining the tumor growth. H & E-stained coronal brain sections are representative tumor xenografts. d: U87/EGFRvIII (5×10⁵) cells expressing luciferase were intracranially injected into athymic nude mice. After three days, selumetinib (50 mg/mL in 5 μL of DMSO) or DMSO was intracranially and intratumorally injected every three days. Bioluminescence imaging of mice was taken at day 9. e: Tumor volumes were measured using length (a) and width (b) and calculated using the equation: V=ab²/2. Data represent the mean±SD of three independent experiments. f: Immunoblotting analyses of the tumor tissues received with or without selumetinib treatment were performed with the indicated antibodies. g: Lactate production of the tumor tissues received with or without selumetinib treatment was analyzed. Data represent the mean±SD of three separate samples.

FIG. 48: Levels of PKM2 S37 phosphorylation correlate with EGFR and ERK1/2 activity in human GBM specimens. a, b: IHC staining with anti-phospho-EGFR Y1172, anti-phospho-ERK1/2, and anti-phospho-PKM2 S37 antibodies was performed on 48 GBM specimens. Representative photos of four tumors are shown (a). Semi-quantitative scoring (using a scale from 0-8) was performed (Pearson product moment correlation test, r=0.77, P<0.001, top panel; r=0.78, P<0.001, bottom panel). Note that some of the dots on the graphs represent more than one specimen (i.e., some scores overlapped) (b). c: A mechanism of PKM2-regulated Warburg effect. EGFR activation in human cancer cells induces PKM2 nuclear translocation, which is mediated by post-translational modification of PKM2 by ERK-dependent phosphorylation and PIN1-regulated cis-trans isomerization and subsequent importin α5 binding. Nuclear PKM2 is required for EGF-induced β-catenin transactivation, which in turn induces c-Myc expression and thereby upregulatesing GLUT1, LDHA, and PTB-dependent PKM2 expression. Nuclear PKM2-dependent expression of these glycolytic enzymes promotes the Warburg effect.

FIG. 49: ERK activation is required for nuclear translocation of PKM2. a: U251 cells were treated with or without EGF (100 ng/mL) for 6 h. Immunofluorescence analyses of endogenous PKM2 or transiently expressed FLAG-PKM1 were performed with the indicated antibodies. Nuclei were stained with Hoechst 33342 (blue). b: Total cell lysates and nuclear fractions were prepared from U87/EGFR cells pretreated with or without AG1478 (1 μM) for 30 min before EGF (100 ng/mL) for 6 h. Immunoblotting analyses were performed with the indicated antibodies. Nuclear PCNA was used as controls for immunoblotting analyses. c: An equal volume of cytosolic and nuclear fractions were prepared from U87/EGFR cells treated with or without EGF (100 ng/mL) for 6 h. Ten microliters of protein from cytosolic and nuclear fractions were used for immunoblotting analyses with the indicated antibodies. d: Nuclear fractions were prepared from U251 cells pretreated with LY290042 (30 μM), SU6656 (4 μM), SP600125 (25 μM), and U0126 (20 μM) for 30 min before EGF (100 ng/mL) for 6 h. Immunoblotting analyses were performed with the indicated antibodies. Nuclear PCNA and cytoplasmic tubulin were used as controls. e: U87/EGFR cells were pretreated with LY290042 (30 μM), SU6656 (4 μM), SP600125 (25 μM), and U0126 (20 μM) for 30 min before EGF (100 ng/mL) for 6 h. Immunoblotting analyses were performed with the indicated antibodies.

FIG. 50: ERK2 and PIN1 regulate nuclear translocation of PKM2. a: U87/EGFR cells were pretreated with or without U0126 (20 μM) for 30 min before EGF (100 ng/mL) for 6 h. Cytosolic and nuclear fractions were prepared. Immunoblotting analyses were performed with the indicated antibodies. b: U87/EGFR cells with depleted endogenous PKM2 and reconstituted expression of RNAi-resistant WT rPKM2 or rPKM2 S37A were treated with or without EGF (100 ng/mL) for 6 h. Total cell lysates (left panel) and nuclear fractions (right panel) were prepared. Immunoblotting analyses were performed with the indicated antibodies. c: PIN1^{-/-} cells were reconstituted to express WT PIN1 or PIN1 L60/61A mutant (left panel), and the nuclear fractions of the cells were prepared (right panel). d: Cis-trans isomerization assays were performed by mixing synthesized phosphorylated oligopeptide of PKM2 containing the S37P motif with purified WT GST-PIN1 or GST-PIN1 L60/61A mutant. Data represent the mean±SD of three independent experiments. e: The indicated cells expressing FLAG-PKM2 were treated with or without EGF (100 ng/mL) for 6 h, and the nuclear fractions were prepared. Immunoblotting analyses were performed with the indicated antibodies. f: U87/EGFR cells expressing a control shRNA or expressing HIF1α shRNA (left panel) were treated with or without EGF (100 ng/mL) for 6 h. The nuclear fractions were prepared (right panel). Immunoblotting analyses were performed with the indicated antibodies.

FIG. 51: Replacement of PKM2 with PKM1 fails to promote EGF-induced β-catenin transactivation and expression of c-Myc, GLUT 1, and LDHA. A: U87/EGFR cells with PKM2 depletion and reconstituted expression of FLAG-rPKM2 and the indicated FLAG-PKM1 proteins were transfected with either TOP-FLASH or FOP-FLASH, which were followed by EGF (100 ng/mL) treatment for 12 h. Immunoblotting analyses were performed with the indicated antibodies. The relative levels of luciferase activity were normalized to the levels of untreated cells and to the levels of luciferase activity of the Renilla control plasmid. Data represent the mean±SD of three independent experiments. B: β-catenin does not bind to RPL30 exon, which is not regulated by PKM2 expression. U87/EGFR cells with or without PKM2 depletion and reconstituted expression of the indicated PKM2 proteins were treated with or without EGF (100 ng/mL) for 12 h. ChIP assay was performed with an anti-β-catenin antibody for immunoprecipitation followed by PCR with RPL30 exon two-specific primers. C: U87/EGFR cells with PKM2 depletion and reconstituted expression of WT FLAG-rPKM2, WT FLAG-PKM1, or FLAG-PKM1 S37A mutant were treated with or without EGF (100 ng/mL) for 24 h. Immunoblotting analyses were performed with the indicated antibodies.

FIG. 52: Replacement of PKM2 with PKM1 fails to promote EGF-induced Warburg effect. a: Endogenous PKM2-depleted U87/EGFRvIII cells with reconstituted expression of FLAG-rPKM2 or FLAG-PKM1 were analyzed by immunoblotting analyses with the indicated antibodies. b: Endogenous PKM2-depleted U87/EGFRvIII cells with reconstituted expression of FLAG-rPKM2 or FLAG-PKM1 were cultured in no-serum DMEM. The media were collected for analysis of glucose consumption (left panel) or lactate production (right panel). Data represent the mean±SD of three independent experiments. c: WT PKM2 and PKM2 S37A have comparable enzymatic activity. The activity of 0.1 g of bacterially purified WT PKM2 (set to 1) or PKM2 S37A toward PEP was measured using a pyruvate kinase assay. Data represent the mean±SD of three independent experiments. d: U87/EGFRvIII cells were infected with lentiviruses expressing control shRNA or shRNA of GLUT1, LDHA, and PTB. Immunoblotting analyses were performed with the indicated antibodies. e, f: Depletion of GLUT1, LDHA, and PTB reduces glucose consumption and lactate production. U87/EGFRvIII cells with or without depletion of GLUT1, LDHA, and PTB were cultured in no-serum DMEM. The media were collected for analysis of glucose consumption (d) or lactate production (e). Data represent the mean±SD of three independent experiments. *P<0.05: statistically significant value in relation with U87/EGFRvIII cells without depletion of GLUT1, LDHA, and PTB.

FIG. 53: PDGF induces nuclear translocation of PKM2 and the Warburg effect in an ERK1/2 activity-dependent manner. a: U87 cells were treated with or without U0126 (20 μM) for 30 min before PDGF-AA (50 ng/mL) for 6 h. Nuclear PCNA. Immunoblotting analyses were performed with the indicated antibodies. b: U87 cells were cultured in no-serum DMEM and treated with or without U0126 (20 μM) for 30 min before PDGF-AA (50 ng/mL) for 20 h. The media were collected for analysis of glucose consumption (left panel) or lactate production (right panel). Data represent the mean±SD of three independent experiments.

FIG. 54: PKM2, but not PKM1, promotes EGFR-induced brain tumorigenesis. a: Luciferase-expressing U87/EGFRvIII cells with PKM2 depletion and reconstituted expression of rPKM2 or rPKM2 S37A were intracranially injected into athymic nude mice. Time course illustrates the representative tumor progression in mice via bioluminescence imaging. Data represent the mean±SD of three independent experiments. b: Endogenous PKM2-depleted U87/EGFRvIII cells with reconstituted expression of FLAG-rPKM2 or overexpression of WT FLAG-PKM1 were intracranially injected into athymic nude mice. After two weeks, mice were sacrificed to examine for tumor growth. H & E-stained coronal brain sections are representative tumor xenografts.

FIG. 55: Nuclear translocation of PKM2 is required for the Warburg effect and brain tumor development. a: GSC11 cells with or without PKM2 depletion and reconstituted expression of WT rPKM2 or rPKM2 S37A mutant were analyzed by immunoblotting analyses with the indicated antibodies. b: GSC11 cells with or without PKM2 depletion and reconstituted expression of WT rPKM2 or rPKM2 S37A mutant were cultured in no-serum DMEM. The media were collected for analysis of glucose consumption (left panel) or lactate production (right panel). Data represent the mean±SD of three independent experiments. c: GSC11 cells (5×10⁵) with or without PKM2 depletion and reconstituted expression of WT rPKM2 or rPKM2 S37A were intracranially injected into athymic nude mice. After four weeks, mice were sacrificed to examine tumor growth. H & E-stained coronal brain sections are representative tumor xenografts. d: Depletion of GLUT1, LDHA, and PTB inhibits EGFRvIII-induced brain tumor growth. U87/EGFRvIII cells (5×10⁵) with or without depletion of GLUT1, LDHA, and PTB were intracranially injected into athymic nude mice. After two weeks, mice were sacrificed to examine tumor growth. H & E-stained coronal brain sections show representative tumor xenografts. e: Validation of antibody specificities. IHC analyses of human GBM tissues were performed with the indicated antibodies in the presence or absence of specific blocking peptides.

FIG. 56: EGFR activation results in upregulation of PKM2 expression. A-D: Immunoblotting analyses were performed with or without the indicated antibodies. A: The indicated cell lines were treated with EGF (100 ng/mL) for 12 h. B: U87 cells were stably transfected with plasmids expressing EGFR or EGFRvIII. C: U87/EGFR cells were pretreated with or without AG1478 (1 μM) for 30 min before EGF (100 ng/mL) treatment for 12 h. D: U87/EGFR cells were pretreated with or without cycloheximide (CHX) (200 μg/mL) for 30 min before EGF (100 ng/mL) treatment for 12 h. E: mRNA expression levels of PKM2 and PKM1 in U87/EGFR cells treated with or without EGF (100 ng/mL) for 12 h were measured by real-time quantitative RT-PCR analysis. β-actin mRNA from the same cDNA library was amplified as a control. The relative mRNA levels of PKM2 and PKM1 were normalized to the levels of untreated cells and β-actin mRNA. Data represent the mean±SD of three independent experiments.

FIG. 57: EGF increases PKM2 expression in a PKC- and NF-κB-dependent manner. A-D, H: Immunoblotting analyses were performed with the indicated antibodies. A: U87/EGFR cells were pretreated with Bis-I (2 μM), Go6976 (2 μM), NF-κB activation inhibitor II (7 μM), an AKT inhibitor (10 μM), or TBB (50 μM), followed by EGF (100 ng/mL) stimulation for 12 h. B: 293T cells transiently transfected with pCep4 EGFR and vectors expressing IKKβ S177/181A were treated with or without EGF (100 ng/mL) for 12 h. C: U87/EGFR cells stably transfected with pGIPZ expressing a control or a RelA shRNA were treated with or without EGF (100 ng/mL) for 12 h. D: RelA^{+/+}, RelA^{-/-}, or RelA^{-/-} fibroblasts with reconstituted RelA expression were treated with or without EGF (100 ng/mL) for 12 h. E: U87/EGFR cells treated with or without EGF (100 ng/mL) for 12 h. ChIP assay was performed with an anti-RelA antibody for immunoprecipitation, followed by PCR with PKM promoter-specific primers. F: An oligonucleotide containing the putative WT or mutated NF-κB binding sequence was labeled using [γ-³²P] ATP and T4 polynucleotide kinase. Nuclear extracts of U87/EGFR cells treated with or without EGF (100 ng/mL) for 12 h were incubated with the ³²P-labeled probe in the presence or absence of an anti-RelA antibody or unlabeled oligonucleotide. Samples were subjected to 5% polyacrylamide gel electrophoresis, and the dried gel was exposed to x-ray film. G: The luciferase reporter vector pGL3-promoter containing either the WT or a mutated PKM promoter fragment was transfected into U87/EGFR cells (left panel), or RelA^{+/+}, RelA^{-/-}, or RelA^{-/-} fibroblasts with reconstituted RelA expression (right panel), which were treated with or without EGF (100 ng/mL) for 12 h. The relative levels of luciferase activity were normalized to the levels of untreated cells and to the levels of luciferase activity of the Renilla control plasmid. Data represent the mean±standard deviation of three independent experiments. H: U251 cells infected with lentiviruses expressing a control or a PTBP1 shRNA were treated with or without EGF (100 ng/mL) for 12 h.

FIG. 58: PKCε downstream from PLCγ1, rather than TAK1, activates IKKβ and subsequently increases PKM2 expression. Immunoprecipitation and immunoblotting analyses were performed with the indicated antibodies. A: The indicated fibroblasts were treated with or without EGF (100 ng/mL) for 12 h. B: U87/EGFR cells were treated with TNFα (10 ng/mL) for the indicated time. C: U87/EGFR cells were treated with or without EGF (100 ng/mL) or TNFα (10 ng/mL) for 4 h. D: U87/EGFR cells were treated with or without EGF (100 ng/mL) for 12 h. HIF1α was immunodepleted from the cell lysates by incubation with an anti-HIF1α antibody, which was followed by a ChIP assay with an anti-FLAG antibody for immunoprecipitation of FLAG-RelA and PCR analysis with PKM promoter-specific primers. E: U87/EGFR cells transiently transfected with a control or a HIF1α siRNA were treated with or without EGF (100 ng/mL) for 12 h. F: 293T cells were transiently transfected with constitutively active (+) or kinase-dead (−) PKC mutants. G: U87/EGFR cells stably transfected with pGIPZ expressing a control or a PKCε shRNA were reconstituted with or without expression of rPKCε and were treated with or without EGF (100 ng/mL) for 12 h. H, I: 293T cells were transiently transfected with the indicated plasmids. J, K: U87/EGFR cells pretreated with or without U73122 (2 μM) for 30 min (J) or stably expressed PLCγ1 H59Q (K) were treated with or without EGF (100 ng/mL) for 12 h.

FIG. 59: PKCε phosphorylates IKKβ at Ser177 and activates IKKβ. A, B, D-G: Immunoprecipitation and immunoblotting analyses were performed with the indicated antibodies. A: The indicated cells were treated with or without EGF (100 ng/mL) for 30 min. B: Bacterially purified His-IKKβ on nickel agarose beads was mixed with purified active GST-PKCε. A His-protein pull-down assay with nickel agarose beads incubated with GST-PKCε as a control was performed. C: U87/EGFR cells were treated with EGF (100 ng/mL) for 30 min and immunostained with the indicated antibodies. Nuclei were stained with Hoechst 33342 (blue). D: In vitro kinase assays were performed with purified active PKCε and bacterially purified WT His-IKKβ or different His-IKKβ mutants. E: 293T cells were transiently transfected with FLAG-PKCε AE3 or FLAG-PKCε knAE1. F: U87/EGFR cells stably transfected with pGIPZ expressing a control or a PKCε shRNA were reconstituted with or without expression of rPKCε and treated with or without EGF (100 ng/mL) for 30 min. G: IKKβ^{+/+} and IKKβ^{-/-} fibroblasts with reconstituted expression of WT or S177A mutant of IKKβ were treated with or without EGF (100 ng/mL) for 30 min (top three panels) or 12 h (bottom two panels).

FIG. 60: Binding of NEMO zinc finger to monoubiquitylated PKCε at Lys321 regulates the interaction between PKCε and IKKβ. Immunoprecipitation and immunoblotting analyses were performed with the indicated antibodies. A, B: The indicated cells were treated with or without EGF (100 ng/mL) for 12 h (A) or 30 min (B). C: U87/EGFR cells were treated with or without EGF (100 ng/mL) for 30 min. D: Nickel agarose beads were mixed with lysates of 293T cells transiently transfected with plasmids expressing EGFR and His-ubiquitin and treated with or without EGF (100 ng/mL) for 30 min. E: 293T cells were transiently transfected with plasmids expressing EGFR and Myc-NEMO and treated with or without EGF (100 ng/mL) for 30 min. F: A vector expressing FLAG-PKCε AE3 was cotransfected with or without vectors expressing WT FLAG-RINCK1, FLAG-RINCK1 C20A, WT-RINCK2, HA-HOIL-1L, or FLAG-HOIP into 293T cells. F: U87/EGFR cells expressing FLAG-PKCε AE3 were stably transfected with a vector expressing a control shRNA or RINCK1 shRNA. H, I: 293T cells were transiently transfected with plasmids expressing FLAG-PKCεAE3 or the indicated PKCε AE3 mutants. J: 293T cells were transiently transfected with plasmids expressing Myc-NEMO and FLAG-PKCε AE3 or the PKCε AE3 K321R mutant. K: U87/EGFR cells stably transfected with WT or the indicated NEMO mutants were treated with or without EGF (100 ng/mL) for 30 min. L: FLAG-PKCε WT was transiently transfected into NEMO^{+/+} and NEMO^{-/-} fibroblasts with reconstituted expression of WT or the indicated NEMO mutants, and the cells were treated with or without EGF (100 ng/mL) for 30 min. M: U87/EGFR cells with or without expression of PLCγ1 H59Q were treated with or without EGF (100 ng/mL) for 30 min.

FIG. 61: EGF promotes glycolysis and tumorigenesis by PKCε- and NF-κB-dependent PKM2 upregulation. A, B: The indicated cells in no-serum DMEM were treated with or without EGF (100 ng/mL) for 20 h. The media were collected for analysis of glucose consumption (A) or lactate production (B), which was normalized by cell numbers (per 10⁶). Data represent the mean±SD of three independent experiments. C: Immunoblotting analyses of lysates of U87/EGFR cells stably transfected with pGIPZ expressing a control or a PKM2 shRNA with or without reconstituted expression of rPKM2 at different levels were performed with the indicated antibodies (left panel). These cells were treated with or without EGF (100 ng/mL) for 20 h. The media were collected for analysis of glucose consumption (middle panel) or lactate production (right panel), which was normalized by cell numbers (per 10⁶). Data represent the mean±SD of three independent experiments. L, low expression. H, high expression. D: U87/EGFR cells with or without PKCε or RelA depletion were treated with or without EGF (100 ng/mL) for 20 h. The media were collected for analysis of glucose consumption (left panel) or lactate production (right panel), which was normalized by cell numbers (per 10⁶). Data represent the mean±SD of three independent experiments. E: Immunoblotting analyses of lysates of U87/EGFRvIII cells with or without RelA depletion and U87/EGFRvIII cells with or without PKM2 depletion and reconstituted expression of rPKM2 were performed with the indicated antibodies (top panel). A total number of 2×10⁴ cells from each cell line were plated and counted seven days after seeding in DMEM with 2% bovine calf serum (bottom panel). Data represent the mean±SD of three independent experiments. L, low expression. H, high expression. F: U87/EGFRvIII cells (5×10⁵), with or without RNAi-depleted RelA or PKM2 or combined expression of rPKM2, were intracranially injected into athymic nude mice. After two weeks, the mice were sacrificed and tumor growth was examined. H & E-stained coronal brain sections show representative tumor xenografts (top panel). Tumor volumes were calculated (bottom panel). L, low expression. H, high expression. Data represent the mean±SD of seven mice.

FIG. 62: Levels of PKM2 correlate with activity levels of EGFR and IKKβ in human GBM and with grades of glioma malignancy and prognosis. A, B: IHC staining with anti-phospho-EGFR Y1172, anti-phospho-IKKβ S177/181, and anti-PKM2 antibodies was performed on 55 GBM specimens. Representative photos of four tumors are shown (A). Semi-quantitative scoring was performed (Pearson product moment correlation test, r=0.88, P<0.001, top panel; r=0.89, P<0.001, bottom panel). Note that some of the dots on the graphs represent more than one specimen (some scores overlapped) (B). C: Overall survival time of 55 patients with GBM by low (20 patients) and high (35 patients) PKM2 expression (P<0.001). Data represent the mean±SD of survival time (months) of 20 patients with low PKM2 expression and 35 patients with high PKM2 expression. D: Immunohistochemical staining of 27 diffuse astrocytoma specimens with a PKM2 antibody was performed and analyzed by comparing it with the staining of 38 GBM specimens (Student's t-test, two-tailed, P<0.001). Data represent the mean±SD of the staining scores for PKM2 from 27 astrocytoma specimens and 38 GBM specimens. E: A mechanism for EGFR-induced PKM2 upregulation. EGFR activation results in the binding of the SH2 domain of PLCγ1 to autophosphorylated EGFR and activation of PLCγ1. Diacylglycerol generated by PLCγ1 will activate PKCε, which results in RINCK1-dependent monoubiquitylation of PKCε at K321 and subsequent recruitment of the NEMO/IKKβ complex. PKCε phosphorylates IKKβ at S177 and activates IKKβ, leading to RelA/HIF1α-dependent transcriptional upregulation of PKM2.

FIG. 63: PKC, NF-κB, PI3-K, and CK2 are inhibited by their inhibitors. A, B: U87/EGFR cells were treated with Bis-I (2 μM) (A) or Go6976 (2 μM) (B) for 30 min before 12-O-tetradecanoylphorbol-13-acetate (TPA) (400 nm) for 8 h. Immunoblotting analyses were performed with the indicated antibodies. C: U87/EGFR cells transiently expressing a luciferase reporter vector containing the IκBα promoter were pretreated with or without NF-κB activation inhibitor II (7 μM) for 30 min before TNFα (10 ng/mL) for 8 h. The relative levels of luciferase activity were normalized to the levels of untreated cells and to the levels of luciferase activity of the Renilla control plasmid. Data represent the mean±standard deviation of three independent experiments. D, E: U87/EGFR cells were pretreated with an AKT inhibitor (10 μM) (D) and TBB (50 μM) (E) for 30 min before EGF (100 ng/mL) stimulation for 30 min. Immunoblotting analyses were performed with the indicated antibodies.

FIG. 64: EGF increases PKM2 expression in a RelA- and PTBP1-dependent manner. A: EGF treatment resulted in increased IKKβ activity. In vitro kinase assays were performed by incubation of bacterially purified His-IkBα with immunoprecipitated IKKβ from U87/EGFR cells treated with or without EGF (100 ng/mL) for 1 h. Data represent the mean±standard deviation of three independent experiments. B: EGF treatment resulted in IkBα S32 phosphorylation and degradation. U87/EGFR cells were treated with or without EGF (100 ng/mL) for 15 min. Immunoblotting analyses were performed with the indicated antibodies. C: RelA deficiency inhibits EGF-increased mRNA levels of PKM2, but not of PKM1. mRNA expression levels of PKM2 and PKM1 in RelA^{+/+} and RelA^{-/-} fibroblasts treated with or without EGF (100 ng/mL) for 8 h were measured by real-time quantitative RT-PCR analysis. β-actin mRNA from the same cDNA library was amplified as a control. The relative mRNA levels of PKM2 (left panel) and PKM1 (right panel) were normalized to the levels of untreated cells and β-actin mRNA. Data represent the mean±SD of three independent experiments. D: PTBP1 depletion blocked EGF-induced PKM2 mRNA expression. U251 cells infected with lentiviruses expressing a control or a PTBP1 shRNA were pretreated with or without NF-κB activation inhibitor II (7 μM) for 30 min before EGF (100 ng/mL) stimulation for 8 h. mRNA expression levels of PKM2 (middle panel) and PKM1 (right panel) in these cells were measured by real-time quantitative RT-PCR analysis. β-actin mRNA from the same cDNA library was amplified as a control. The relative mRNA levels of PKM2 and PKM1 were normalized to the levels of untreated cells and β-actin mRNA. Data represent the mean±SD of three independent experiments. E: EGF does not enhance IκBα promoter activity. U87/EGFR cells transiently expressing a luciferase reporter vector containing the IκBα promoter were treated with or without TNFα (10 ng/mL) or EGF (100 ng/mL) for 8 h. The relative levels of luciferase activity were normalized to the levels of untreated cells and to the levels of luciferase activity of the Renilla control plasmid. Data represent the mean±standard deviation of three independent experiments.

FIG. 65: HIF1α is a coactivator for RelA in response to EGF to induce PKM2 expression. A: RelA^{+/+} and RelA^{-/-} fibroblasts were treated with EGF (100 ng/mL) for 4 h. ChIP analyses were performed with an anti-HIF1α antibody for immunoprecipitation and PKM promoter-specific primers for a real-time quantitative PCR. B: U87/EGFR cells in normoxic condition were treated with or without EGF (100 ng/mL) for 4 h. HIF1α was immunodepleted from the cell lysates by incubating with an anti-HIF1α antibody, which was followed by a ChIP assay with an anti-RelA antibody for immunoprecipitation and a real-time quantitative PCR with PKM promoter-specific primers (left panel). A control ChIP analysis was performed with anti-RelA and anti-HIF1α antibodies for immunoprecipitation and RPL30 exon 3-specific primers for PCR (right panel). C: U87/EGFR cells were treated with or without EGF (100 ng/mL) for 4 h. HIF1α was immunodepleted from the cell lysates by incubating with an anti-HIF1α antibody, which was followed by a ChIP assay with an anti-acetylated histone H3 antibody for immunoprecipitation and a real-time quantitative PCR with PKM promoter-specific primers. D: U87/EGFR cells were treated with EGF (100 ng/mL) for 4 h in normoxic conditions (20% O₂) or hypoxic conditions (1% O₂). Immunoblotting analyses were performed with the indicated antibodies. E: U87/EGFR cells were treated with NF-κB activation inhibitor II (7 μM) for 30 min before EGF (100 ng/mL) for 24 h. Immunoblotting analyses were performed with the indicated antibodies. F: U87/EGFR cells were treated with or without EGF (100 ng/mL) for 4 h. HIF1α was immunodepleted from the cell lysates by incubating with an anti-HIF1α antibody, which was followed by a ChIP assay with an anti-RelA antibody for immunoprecipitation and a real-time quantitative PCR with HIF1α promoter-specific primers.

FIG. 66: PKCε, but not PKCζ, is involved in PKM2 upregulation. Immunoprecipitation and immunoblotting analyses were performed with the indicated antibodies. A: U87/EGFR cells were transiently transfected with constitutively active (+) or kinase-dead (−) PKCζ mutants. B: U87/EGFR cells stably transfected with a vector expressing PKCεknAE1 were treated with or without EGF (100 ng/mL) for 8 h.

FIG. 67: PDGF treatment induces PKM2 expression in a RelA-dependent manner. A: U87/EGFR cells were treated with U73122 (2 μM), Bis-I (2 μM), or NF-κB activation inhibitor II (7 μM) for 30 min before PDGF-AA (50 ng/mL) stimulation for 24 h. Immunoblotting analyses were performed with the indicated antibodies. B: U87/EGFR cells with or without expressing RelA shRNA were treated with or without PDGF-AA (50 ng/mL) for 24 h. Immunoblotting analyses were performed with the indicated antibodies.

FIG. 68: PKCα and PKCζ do not interact with IKKβ. Immunoprecipitation and immunoblotting analyses were performed with the indicated antibodies. A: U87/EGFR cells were treated with or without EGF (100 ng/mL) for 30 min. B: Immunoblotting analyses were performed by using NEMO^{+/+} and NEMO^{-/-} fibroblasts with or without reconstituted expression of WT or the indicated NEMO mutants.

FIG. 69: PKM2 overexpression enhances glycolysis and tumorigenesis. A: U87/EGFR cells were stably transfected with vectors expressing with or without FLAG-tagged PKM2 or PKM1 and were treated with or without EGF (100 ng/mL) for 20 h. Immunoblotting analyses were performed with the indicated antibodies. B, C: U87/EGFR cells with or without overexpressing FLAG-tagged PKM2 or PKM1 were incubated in no-serum DMEM for 20 h. The media were collected for analysis of glucose consumption (B) or lactate production (C), which was normalized by cell numbers (per 10⁶). Data represent the mean±SD of three independent experiments. Immunoblotting analyses were performed with the indicated antibody. D: U87/EGFR cells (5×10⁵) with or without overexpression of PKM2 or PKM1 were intracranially injected into athymic nude mice for each group. After two weeks, mice were sacrificed for examining the tumor growth. H & E-stained coronal brain sections show representative tumor xenografts.

FIG. 70: PKM2 is required for the fidelity of chromosome segregation and kinetochore localization of Bub3 and Bub1. Immunoblotting (B, C) or immunofluorescence (A, D, E, F) analyses were performed with the indicated antibodies. Nuclei were stained with DAPI (blue). A: HeLa cells in different phases of the cell cycle were immunostained with the indicated antibodies. B: HeLa cells synchronized by thymidine double block (2 mM) were released for the indicated periods of time (left panel) or 6 h followed by nocodazole (100 ng/mL) treatment for 12 h (right panel) with or without removing nocodazole for 2 h thereafter. Chromatin extract or cell lysates were prepared. C: HeLa cells with or without PKM2 depletion were reconstituted with or without the expression of WT rPKM2 or rPKM2 K367M. D: HeLa cells with or without PKM2 depletion were stained with the indicated antibodies. One hundred mitotic cells in each indicated subphase of mitosis were analyzed. Data represent the mean±SD of three independent experiments. The white arrows point to the fragmented chromatin or lagging chromosomes at the metaphase plate. E: HeLa cells with or without PKM2 depletion in interphase or prometaphase were immunostained with the indicated antibodies. F: HeLa cells with or without PKM2 depletion and reconstituted expression of WT rPKM2 or rPKM2 K367M were immunostained with the indicated antibodies. One hundred cells in prometaphase were analyzed. Data represent the mean±SD of three independent experiments.

FIG. 71: PKM2, but not PKM1, interacts with and phosphorylates Bub3 at Y207. Immunoblotting, immunoprecipitation (A-H, J), or immunofluorescence (I) analyses were performed with the indicated antibodies. Nuclei were stained with DAPI (blue). A: HeLa cells synchronized by thymidine double block (2 mM) with or without release for 6 h were treated with nocodazole (100 ng/mL) for 12 h with or without release for 2 h. I, interphase; M, mitosis. B: Purified recombinant GST-Bub1 or GST-Bub3 was mixed with purified His-PKM2 or His-PKM1. A GST pull-down assay was performed. C: HeLa cells with depleted Bub1 or Bub3 were synchronized by thymidine double block (2 mM) with or without release for 9 h. I, interphase; M, mitosis. D: In vitro phosphorylation analyses were performed by mixing purified WT His-PKM2, His-PKM2 K367M, or His-PKM1 with purified GST-Bub3 in the presence of PEP or ATP. E: In vitro phosphorylation analyses were performed by mixing purified His-PKM2 with the indicated recombinant GST-Bub3 proteins. F: HeLa cells synchronized by thymidine double block (2 mM) were released for the indicated periods of time. G: Bub3 was depleted from HeLa cells with expression of Bub3 shRNA and reconstituted with expression of the indicated Bub3 proteins. H: HeLa cells with depleted Bub3 and reconstituted expression of the indicated Bub3 proteins were synchronized by thymidine double block (2 mM) with or without release for 9 h. I, interphase; M, mitosis. I: HeLa cells with or without PKM2 depletion were immunostained with the indicated antibodies. The cells in interphase and prometaphase were examined. J: HeLa cells with depleted PKM2 and reconstituted expression of WT PKM2 or PKM2 K367M were synchronized by thymidine double block (2 mM) with or without release for 9 h.

FIG. 72: PKM2-dependent Bub3 Y207 phosphorylation is required for recruitment of Bub3 and Bub1 to kinetochores and accurate chromosome segregation. Immunofluorescence (A, D, E) and immunoblotting (B, C) analyses were performed with the indicated antibodies. Nuclei were stained with DAPI (blue). A, D: HeLa cells with depleted Bub3 and reconstituted expression of WT rBub3 and rBub3 Y207F were immunostained with the indicated antibodies. The cells in prometaphase were examined. B: Purified recombinant GST or GST-Bub3 proteins were mixed with purified recombinant His-Bub1. A GST pull-down assay was performed. C: HeLa cells with Bub3 depletion and reconstituted expression of FLAG-tagged WT rBub3 and rBub3 Y207F (left panel) or with or without PKM2 depletion and reconstituted expression of WT rPKM2 or rPKM2 K367M (right panel) or were synchronized by thymidine double block (2 mM) and released for 9 h. Immunoprecipitation analyses were performed with the indicated antibodies. E: HeLa cells with depleted Bub3 and reconstituted expression of WT rBub3 and rBub3 Y207F were immunostained with the indicated antibodies. The cells in metaphase and telophase were examined (left panel). One hundred cells in mitosis were analyzed. Data represent the mean±SD of three independent experiments (right panel).

FIG. 73: PKM2-dependent Bub3 Y207 phosphorylation is required for recruitment of Bub3 and Bub1 to Blinkin. Immunoblotting (A-C) or immunofluorescence (D, E) analyses were performed with the indicated antibodies. Nuclei were stained with DAPI (blue). A: HeLa cells with or without PKM2 depletion and reconstituted expression of WT rPKM2 or rPKM2 K367M were synchronized by thymidine double block (2 mM) and released for 9 h Immunoprecipitation of endogenous Blinkin was performed. B: Purified recombinant GST or GST-Bub3 proteins were mixed with or without purified recombinant His-PKM2 for an in vitro phosphorylation reaction, which was followed by incubation with the lysates of endogenous PKM2-depleted HeLa cells synchronized by thymidine double block (2 mM) and arrested at mitosis by nocodazole (100 ng/mL) treatment for 9 h. C: HeLa cells with Bub3 depletion and reconstituted expression of FLAG-tagged Bub3 proteins were synchronized by thymidine double block (2 mM) and released for 9 h. Immunoprecipitation analyses were performed with an anti-Blinkin antibody. D: HeLa cells with depleted Bub3 and reconstituted expression of WT rBub3 or rBub3 Y207F were immunostained with the indicated antibodies. The cells in prometaphase were examined. E: HeLa cells with depleted Bub3 and reconstituted expression of WT rBub3 or rBub3 Y207F or with depleted PKM2 were immunostained with the indicated antibodies.

FIG. 74: PKM2-dependent Bub3 Y207 phosphorylation is required for spindle assembly checkpoint, cell survival and proliferation, and tumorigenesis. A: HeLa cells with PKM2 depletion and reconstituted expression of WT rPKM2 or rPKM2 K367M (left panel) or with Bub3 depletion and reconstituted expression of WT rBub3 or rBub3 Y207F (right panel) were synchronized by thymidine double block (2 mM) and released for the indicated period of time. Immunoblotting analyses were performed with the indicated antibodies, and the intensity of H3 pS10 was quantified. Data represent the mean±SD of three independent experiments. B, C: HeLa cells with PKM2 depletion and reconstituted expression of WT rPKM2 or rPKM2 K367M (2nd and 3rd panel, respectively) or with Bub3 depletion and reconstituted expression of WT rBub3 or rBub3 Y207F (4th and 5th panel, respectively) were synchronized by thymidine double block (2 mM) and released for 6 h, followed by nocodazole (100 ng/mL) treatment for 36 h. Flow cytometric analyses of mitotic cells (B, lower panel) or apoptotic cells (C) were performed. Data represent the mean±SD of three independent experiments. D: HeLa cells (2×10⁴) with depleted Bub3 and reconstituted expression of WT rBub3 or rBub3 Y207F were plated and counted seven days after seeding in DMEM with 2% bovine calf serum. Data represent the mean±SD of three independent experiments. E: A total of 5×10⁵ U87/EGFRvIII cells with PKM2 depletion and reconstituted expression of WT rPKM2 or rPKM2 K367M or with Bub3 depletion and reconstituted expression of WT rBub3 or rBub3 Y207F were intracranially injected into athymic nude mice. The mice were sacrificed and examined for tumor growth. H & E-stained coronal brain sections show representative tumor xenografts. Tumor volumes were measured by using length (a) and width (b) and calculated using the equation: V=ab²/2. Data represent the mean±SD of seven mice.

FIG. 75: Bub3 Y207 phosphorylation positively correlates with the level of H3-S10 phosphorylation. A, B: Immunohistochemical staining with anti-phospho-Bub3 Y207 and anti-phospho-H3-S10 antibodies was performed on 50 GBM specimens. Representative photos of four tumors are shown (A). The inventors quantitatively scored the tissue sections by counting positively-stained cells in 10 microscopic fields. (Pearson product moment correlation test; r=0.78, P<0.0001). Note that some of the dots on the graphs represent more than one specimen (some scores overlapped) (B). C: PKM2 regulates chromosome segregation and mitotic checkpoint by Bub3 Y207 phosphorylation. PKM2 binds to Bub3 and phosphorylates Bub3 Y207 in the prometaphase, leading to recruitment of the Bub3-Bub1 complex to kinetochores and interaction with Blinkin. These interactions are required for proper chromosome segregation, mitotic checkpoint, and cell survival and proliferation.

FIG. 76: PKM2 is required for the fidelity of chromosome segregation and kinetochore localization of Bub3 and Bub1. A, C: HeLa cells with or without PKM2 depletion and reconstituted expression of WT PKM2 or PKM2 K367M were immunostained with the indicated antibodies. One hundred of the cells in prometaphase were analyzed. Data represent the mean±SD of three independent experiments. B: U87/EGFRvIII cells with or without PKM2 depletion were immunostained with the indicated antibodies. The cells in prometaphase were examined. D: U87/EGFRvIII cells with or without PKM2 depletion and reconstituted expression of WT rPKM2 or rPKM2 K367M were examined by immunoblotting analyses with the indicated antibodies. E: U87/EGFRvIII cells with or without PKM2 depletion and reconstituted expression of WT rPKM2 or rPKM2 K367M were immunostained with the indicated antibodies. One hundred of the cells in prometaphase were analyzed. Data represent the mean±SD of three independent experiments.

FIG. 77: PKM2, but not PKM1, interacts with and phosphorylates Bub3 at Y207. A: Bub3 was depleted from U87/EGFRvIII cells with expression of Bub3 shRNA, and expression of the indicated Bub3 proteins was reconstituted. Immunoblotting analyses were performed with the indicated antibodies. B: U87/EGFRvIII with depleted Bub3 and reconstituted expression of the indicated Bub3 proteins were synchronized by thymidine double block (2 mM) and released for 9 h. Immunoblotting analyses were performed with the indicated antibodies. I, interphase; M, mitosis. C: U87/EGFRvIII cells with or without PKM2 depletion were synchronized by thymidine double block (2 mM) with or without release for 9 h. Immunoblotting analyses were performed with the indicated antibodies.

FIG. 78: PKM2-dependent Bub3 Y207 phosphorylation is required for accurate chromosome segregation. U87/EGFRvIII cells with depleted Bub3 and reconstituted expression of WT rBub3 and rBub3 Y207F were immunostained with the indicated antibodies. The cells in metaphase and telophase were examined (left panel). One hundred of the cells in mitosis were analyzed. Data represent the mean±SD of three independent experiments (right panel).

FIG. 79: PKM2-dependent Bub3 Y207 phosphorylation is required for the association of Bub3 and Bub1 with Blinkin. U87/EGFRvIII cells with Bub3 depletion and reconstituted expression of the indicated FLAG-tagged Bub3 proteins were synchronized by thymidine double block (2 mM) and released for 9 h. Immunoprecipitation and immunoblotting analyses were performed with the indicated antibodies.

FIG. 80: PKM2-dependent Bub3 Y207 phosphorylation is required for spindle assembly checkpoint and tumorigenesis. A: U87/EGFRvIII cells with PKM2 depletion and reconstituted expression of WT rPKM2 or rPKM2 K367M (left panel) or with Bub3 depletion and reconstituted expression of WT rBub3 or rBub3 Y207F (right panel) were synchronized by thymidine double block (2 mM) and released for the indicated period of time. Immunoblotting analyses were performed with the indicated antibodies, and the intensity of H3 pS10 was quantified. Data represent the mean±SD of three independent experiments. I, interphase; M, mitosis. B: GSC11 cells with Bub3 depletion were reconstituted with the expression of WT rBub3 or rBub3 Y207F. Immunoblotting analyses were performed with the indicated antibodies. C: A total of 5×10⁵ GSC11 cells with Bub3 depletion and reconstituted expression of WT rBub3 or rBub3 Y207F were intracranially injected into athymic nude mice. The mice were sacrificed and examined for tumor growth. H & E-stained coronal brain sections show representative tumor xenografts. Tumor volumes were measured by using length (a) and width (b) and calculated using the equation: V=ab²/2. Data represent the mean±SD of seven mice.

### DESCRIPTION OF ILLUSTRATIVE EMBODIMENTS

### I. The Present Invention

The embryonic pyruvate kinase M2 (PKM2) isoform is highly expressed in human cancer. In contrast to the established role of PKM2 in aerobic glycolysis or the Warburg effect, its nonmetabolic functions remain elusive. Here it is demonstrated that EGFR activation induces translocation of PKM2, but not PKM1, into the nucleus, where K433 of PKM2 binds to c-Src-phosphorylated Y333 of β-catenin. This interaction is required for both proteins to be recruited to the CCND1 promoter, leading to HDAC3 removal from the promoter, histone H3 acetylation, and cyclin D1 expression. PKM2-dependent β-catenin transactivation is instrumental in EGFR-promoted tumor cell proliferation and brain tumor development. In addition, positive correlations have been identified among EGF-dependent NF-κB activation (via a PKCε-dependent mechanism), c-Src activity, β-catenin Y333 phosphorylation, and PKM2 nuclear accumulation in human glioblastoma specimens. Furthermore, levels of PKM2 expression and activation as well as β-catenin phosphorylation and nuclear PKM2 have been correlated with grades of glioma malignancy and prognosis. These findings reveal that EGF induces β-catenin transactivation via a mechanism distinct from that induced by Wnt/wingless and highlight the essential nonmetabolic functions of PKM2 in EGFR-promoted β-catenin transactivation, cell proliferation, and tumorigenesis.

Further studies detailed here demonstrate that EGFR-activated ERK2 binds directly to PKM2 I429/L431 via the ERK2 docking groove and phosphorylates PKM2 Ser37 but not PKM1. Phosphorylated PKM2 Ser37 recruits PIN1 for cis-trans isomerization of PKM2, which leads to PKM2 binding to importin α5 and nuclear translocation. Nuclear PKM2, acting as a coactivator of β-catenin, induces c-Myc expression, resulting in the upregulation of GLUT1, LDHA, and, in a positive feedback loop, PTB-dependent PKM2 expression. Replacement of wild type PKM2 with a nuclear translocation-deficient mutant (S37A) blocks the EGFR-promoted Warburg effect and brain tumor development. In addition, levels of PKM2 S37 phosphorylation correlate with EGFR and ERK1/2 activity in human glioblastoma specimens. These findings highlight the importance of nuclear functions of PKM2 in the Warburg effect and tumorigenesis.

It has now further been demonstrated that PKM2, functioning as a protein kinase, interacts with histone H3 and phosphorylates H3-T11, which leads to HDAC3 removal from CCND1 and MYC promoter regions and subsequently to K9 acetylation and gene transcription. Thus, PKM2 plays two integrated functions in tumor development: 1) PKM2 act as a glycolytic enzyme transferring a phosphate group from PEP to ADP for ATP generation and pyruvate production. It is also a rate-limiting controller of glycolysis needed for generation of glucose metabolites to synthesize amino acids, phospholipids, and nucleic acids, which are building blocks for cell growth and cell proliferation (Hsu and Sabatini, 2008; Koppenol et al., 2011; Vander Heiden et al., 2009). 2) PKM2 acts as a protein kinase phosphorylating histone for gene transcription, which directly controls cell cycle progression and cell proliferation (Yang et al., 2011). This line of evidence establishes PKM2 as a unique and key regulator of cancer development by virtue of its coordination of ATP generation, macromolecular syntheses, and gene transcription via both metabolic and nonmetabolic functions.

These studies thereby provide a range of biomarkers that can be used to determine cancer prognosis and to identify cancers that can be treated with therapeutics that target aspects of the PKM2 pathway. In particular, cancer cells can be assessed for elevated (1) β-catenin activity (such as by assessing Y333 phosphorylation); (2) elevated PKM2 S37 phosphorylation; (3) elevated nuclear PKM2 expression; (4) elevated histone H3 T11 phosphorylation; (5) elevated histone H3 K9 acetylation; (6) elevated Bub3 Y207 phosphorylation; (7) elevated MLC2 Y118 phosphorylation and/or (8) elevated EGF-dependent NF-κB activity, each of which are markers of cancer aggressiveness, and can be used to provide a grade of the individual cancer. Moreover, elevated levels of these markers indicate cells that are likely to respond to a PKM2, ERK/MEK, PKCε, NF-κB, Pin1 and/or Src-targeted therapies. Thus, the biomarkers and methods detailed here provide the ability to individualize cancer diagnosis and to tailor anti-cancer therapy based on the individual biomarker profile of the cancer.

### II. Targeted Therapies of the Embodiments

Certain aspects of the embodiments concern administering a targeted therapy to a patient determined to comprise one or more biomarkers of the embodiments. In some aspects, a patient identified to have a cancer expressing activated PKM2 (or a biomarker thereof) is administered one or more of a NF-κB, PKCε, MEK/ERK, Src or PKM2 inhibitor therapy. Some specific targeted therapies for use according to the embodiments are provided below.

A. PKM2 Inhibitors

Certain aspects of the embodiments concern PKM2 inhibitors. For example, in some aspects, the PKM2 inhibitor is a protein inhibitor, such as a peptide that binds to PKM2 and competes with a PKM2 binding partner or phosphorylation substrate. In further aspects, the PKM2 inhibitor can be a small molecules inhibitor. For example, the small molecule inhibitor can be Alkannin, Shikonin or a derivative or prodrug thereof (see, e.g., Chen et al., 2011. Further examples of small molecule PKM2 inhibitor for use according to the embodiments include, without limitation, compounds according the structures (I)-(VIII), below.

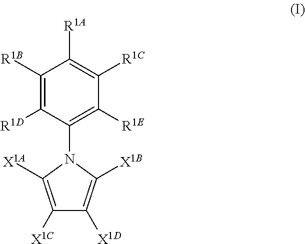

wherein each of R^{1A}, R^{1B}, R^{1C}, R^{1D}, R^{1E}, X^{1A}, X^{1B}, X^{1C}, and X^{1D} is independently, selected from H, halide, nitro, C₁₋₄ alkyl, C₂₋₄ alkenyl, C₂₋₄ alkynyl, OR^{1K}, OC(O)R^{1L}, NR^{1M}R^{1N}, NHC(O)R^{1O}, NHC(S)R^{1P}, NHC(O)OR^{1Q}, NHC(S)OR^{1R}, NHC(O)NHR^{1S}, NHC(S)NHR^{1T}, NHC(O)SR^{1U}, NHC(S)SR^{1V}, NHS(O)₂R^{1W}, C(O)OR^{1X}, C(O)NHR^{1Y}, (CH₂)₁₋₄OH, C(O)R^{1Z}, CH₂R^{1AA}, SO₃H, SO₂R^{1BB}, S(O)R^{1CC}, SR^{1DD}, SO₂NHR^{1EE}, and S(CH₂)₁₋₄C(O)OH; and each of R^{1K}, R^{1L}, R^{1M}, R^{1N}, R^{1O}, R^{1P}, R^{1Q}, R^{1R}, R^{1S}, R^{1T}, R^{1U}, R^{1V}, R^{1W}, R^{1X}, R^{1Y}, R^{1Z}, R^{1AA}, R^{1BB}, R^{1CC}, R^{1DD}, and R^{1EE} is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₄ heteroalkyl, and salts thereof. In one particular embodiment, X^{1A} and X^{1B} are both methyl, X^{1C} and X^{1D} are both H, and each of R^{1A}, R^{1B}, R^{1C}, R^{1D}, and R^{1E} is, independently, selected from H, halide, nitro, C₁₋₄ alkyl, C₂₋₄ alkenyl, C₂₋₄ alkynyl, OR^{1K}, OC(O)R^{1L}, NR^{1M}R^{1N}, NHC(O)R^{1O}, NHC(S)R^{1P}, NHC(O)OR^{1Q}, NHC(S)OR^{1R}, NHC(O)NHR^{1S}, NHC(S)NHR^{1T}, NHC(O)SR^{1U}, NHC(S)SR^{1V}, NHS(O)₂R^{1W}, C(O)OR^{1X}, C(O)NHR^{1Y}, (CH₂)₁₋₄OH, C(O)R^{1Z}, CH₂R^{1AA}, SO₃H, SO₂R^{1BB}, S(O)R^{1CC}, SR^{1DD}, SO₂NHR^{1EE}, and S(CH₂)₁₋₄C(O)OH; and each of R^{1K}, R^{1L}, R^{1M}, R^{1N}, R^{1O}, R^{1P}, R^{1Q}, R^{1R}, R^{1S}, R^{1T}, R^{1U}, R^{1V}, R^{1W}, R^{1X}, R^{1Y}, R^{1Z}, R^{1AA}, R^{1BB}, R^{1CC}, R^{1DD}, and R^{1EE} is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₄ heteroalkyl, and salts thereof.

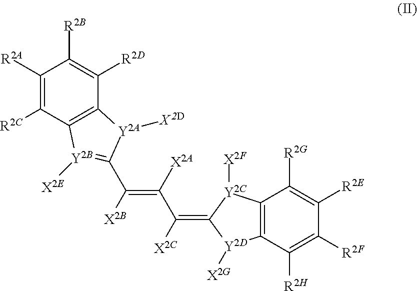

wherein each of X^{2A}, X^{2B}, X^{2C}, X^{2D}, X^{2E}, X^{2F}, and X^{2G} is, independently, selected from H, C₁₋₈ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₈ heteroalkyl; and each of Y^{2A}, Y^{2C}, and Y^{2D} is, independently, selected from N and CH; and Y^{2B} is, independently, selected from N+ and C; and each of R^{2A}, R^{2B}, R^{2C}, R^{2D}, R^{2E}, R^{2F}, R^{2G}, and R^{2H} is, independently, selected from H, halide, nitro, C₁₋₄ alkyl, C₂₋₄ alkenyl, C₂₋₄ alkynyl, OR^{2K}, OC(O)R^{2L}, NR^{2M}R^{2N}, NHC(O)R^{2O}, NHC(S)R^{2P}, NHC(O)OR^{2Q}, NHC(S)OR^{2R}, NHC(O)NHR^{2S}, NHC(S)NHR^{2T}, NHC(O)SR^{2U}, NHC(S)SR^{2V}, NHS(O)₂R^{2W}, C(O)OR^{2X}, C(O)NHR^{2Y}, (CH₂)₁₋₄OH, C(O)R^{2Z}, CH₂R^{2AA}, SO₃H, SO₂R^{2BB}, S(O)R^{2CC}, SR^{2DD}, SO₂NHR^{2EE}, and S(CH₂)₁₋₄C(O)OH; and each of R^{2K}, R^{2L}, R^{2M}, R^{2N}, R^{2O}, R^{2P}, R^{2Q}, R^{2R}, R^{2S}, R^{2T}, R^{2U}, R^{2V}, R^{2W}, R^{2X}, R^{2Y}, R^{2Z}, R^{2AA}, R^{2BB}, R^{2CC}, R^{2DD}, and R^{2EE} is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₄ heteroalkyl, and salts thereof. In one particular embodiment, each of X^{2A}, X^{2B}, X^{2C}, R^{2C}, R^{2D}, R^{2G}, and R^{2H} is H; and each of Y^{2A}, Y^{2C}, and Y^{2D} is N; and Y^{2B} is N+; and each of X^{2D}, X^{2E}, X^{2F}, and X^{2G} is, independently, selected from H, C₁₋₈ alkyl, C₂₋₄ alkenyl, C₂₋₄ alkynyl, and C₁₋₈ heteroalkyl; and each of R^{2A}, R^{2B}, R^{2E}, and R^{2F} is, independently, selected from H, halide, nitro, C₁₋₄ alkyl, C₂₋₄ alkenyl, C₂₋₄ alkynyl, OR^{2K}, OC(O)R^{2L}, NR^{2M}R^{2N}, NHC(O)R^{2O}, NHC(S)R^{2P}, NHC(O)OR^{2Q}, NHC(S)OR^{2R}, NHC(O)NHR^{2S}, NHC(S)NHR^{2T}, NHC(O)SR^{2U}, NHC(S)SR^{2V}, NHS(O)₂R^{2W}, C(O)OR^{2X}, C(O)NHR^{2Y}, (CH₂)₁₋₄OH, C(O)R^{2Z}, CH₂R^{2AA}, SO₃H, SO₂R^{2BB}, S(O)R^{2CC}, SR^{2DD}, SO₂NHR^{2EE}, and S(CH₂)₁₋₄C(O)OH; and each of R^{2K}, R^{2L}, R^{2M}, R^{2N}, R^{2O}, R^{2P}, R^{2Q}, R^{2R}, R^{2S}, R^{2T}, R^{2U}, R^{2V}, R^{2W}, R^{2X}, R^{2Y}, R^{2Z}, R^{2AA}, R^{2BB}, R^{2CC}, R^{2DD}, and R^{2EE} is, independently, selected from H, C₁₋₄ alkyl, and salts thereof.

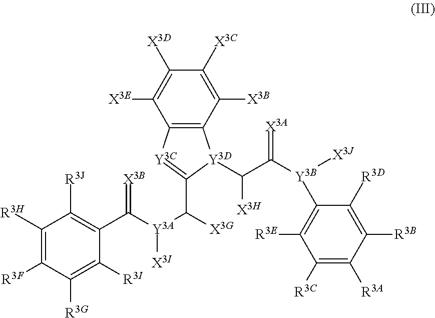

wherein each of X^{3A} and X^{3B} is, independently, selected from S, O, NH, and CH₂; and each of X^{3G} and X^{3H} is, independently, selected from H, C₁₋₈ alkyl, C₂₋₄ alkenyl, C₂₋₄ alkynyl, and C₁₋₈ heteroalkyl; and each of Y^{3A} and Y^{3B} is, independently, selected from O, CH, N, and S; and X^{3I} is empty when Y^{3A} is S or O, X^{3J} is empty when Y^{3B} is S or O, otherwise each of X^{3I} and X^{3J} is, independently, selected from H, C₁₋₈ alkyl, C₂₋₄ alkenyl, C₂₋₄ alkynyl, and C₁₋₈ heteroalkyl; and each of Y^{3C} and Y^{3D} is, independently, selected from CH and N; and each of R^{3A}, R^{3B}, R^{3C}, R^{3D}, R^{3E}, R^{3F}, R^{3G}, R^{3H}, R^{3I}, R^{3J}, X^{3C}, X^{3D}, X^{3E}, and X^{3F} is, independently, selected from H, halide, nitro, C₁₋₄ alkyl, C₂₋₄ alkenyl, C₂₋₄ alkynyl, OR³K, OC(O)R^{3L}, NR^{3M}R^{3N}, NHC(O)R^{3O}, NHC(S)R^{3P}, NHC(O)OR^{3Q}, NHC(S)OR^{3R}, NHC(O)NHR^{3S}, NHC(S)NHR^{3T}, NHC(O)SR^{3U}, NHC(S)SR^{3V}, NHS(O)₂R^{3W}, C(O)OR^{3X}, C(O)NHR^{3Y}, (CH₂)₁₋₄OH, C(O)R^{3Z}, CH₂R^{3AA}, SO₃H, SO₂R^{3BB}, S(O)R^{3CC}, SR^{3DD} SO₂NHR^{3EE}, and S(CH₂)₁₋₄C(O)OH; and each of R^{3K}, R^{3L}, R^{3M}, R^{3N}, R^{3O}, R^{3P}, R^{3Q}, R^{3R}, R^{3S}, R^{3T}, R^{3U}, R^{3V}, R^{3W}, R^{3X}, R^{3Y}, R^{3Z}, R^{3AA}, R^{3BB}, R^{3CC}, R^{3DD}, and R^{3EE} is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₄ heteroalkyl, and salts thereof. In one particular embodiment, each of X^{3A} and X^{3B} is, independently, selected from S and O; and each of X^{3G}, X^{3H}, X^{3I}, and X^{3J} is H; and each of Y^{3A}, Y^{3B}, Y^{3C}, and Y^{3D} is, independently, selected from CH and N; and each of R^{3A}, R^{3B}, R^{3C}, R^{3D}, R^{3E}, R^{3F}, R^{3G}, R^{3H}, R^{3I}, R^{3J}, X^{3C}, X^{3D}, X^{3E}, and X^{3F} is, independently, selected from H, halide, nitro, C₁₋₄ alkyl, C₂₋₄ alkenyl, C₂₋₄ alkynyl, OR³K, OC(O)R^{3L}, NR^{3M}R^{3N}, NHC(O)R^{3O}, NHC(S)R^{3P}, NHC(O)OR^{3Q}, NHC(S)OR^{3R}, NHC(O)NHR^{3S}, NHC(S)NHR^{3T}, NHC(O)SR^{3U}, NHC(S)SR^{3V}, NHS(O)₂R^{3W}, C(O)OR^{3X}, C(O)NHR^{3Y}, (CH₂)₁₋₄OH, C(O)R^{3Z}, CH₂R^{3AA}, SO₃H, SO₂R^{3BB}, S(O)R^{3CC}, SR^{3DD}, SO₂NHR^{3EE}, and S(CH₂)₁₋₄C(O)OH; and each of R^{3K}, R^{3L}, R^{3M}, R^{3N}, R^{3O}, R^{3P}, R^{3Q}, R^{3R}, R^{3S}, R^{3T}, R^{3U}, R^{3V}, R^{3W}, R^{3X}, R^{3Y}, R^{3Z}, R^{3AA}, R^{3BB}, R^{3CC}, R^{3DD}, and R^{3EE} is, independently, selected from H and C₁₋₄ alkyl, and salts thereof.

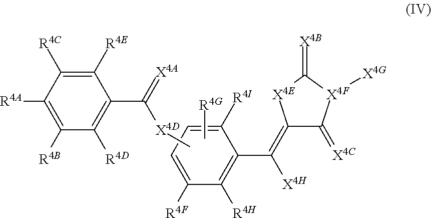

wherein each of X^{4A}, X^{4B}, and X^{4C} is, independently, selected from S, O, NH, CH₂, and two hydrogen atoms; and each of X^{4G} and X^{4H} is, independently, selected from H, C₁₋₈ alkyl, C₂₋₄ alkenyl, C₂₋₄ alkynyl, and C₁₋₈ heteroalkyl; and each of X^{4D} and X^{4E} is, independently, selected from O, CH₂, NH, and S; and X^{4F} is, independently, selected from CH and N; and each of R^{4A}, R^{4B}, R^{4C}, R^{4D}, R^{4E}, R^{4F}, R^{4G}, R^{4H}, and R^{4I}, is, independently, selected from H, halide, nitro, C₁₋₄ alkyl, C₂₋₄ alkenyl, C₂₋₄ alkynyl, OR^{4K}, OC(O)R^{4L}, NR^{4M}R^{4N}, NHC(O)R^{4O}, NHC(S)R^{4P}, NHC(O)OR^{4Q}, NHC(S)OR^{4R}, NHC(O)NHR^{4S}, NHC(S)NHR^{4T}, NHC(O)SR^{4U}, NHC(S)SR^{4V}, NHS(O)₂R^{4W}, C(O)OR^{4X}, C(O)NHR^{4Y}, (CH₂)₁₋₄OH, C(O)R^{4Z}, CH₂R^{4AA}, SO₃H, SO₂R^{4BB}, S(O)R^{4CC}, SR^{4DD}, SO₂NHR^{4EE}, and S(CH₂)₁₋₄C(O)OH; and each of R^{4K}, R^{4L}, R^{4M}, R^{4N}, R^{4O}, R^{4P}, R^{4Q}, R^{4R}, R^{4S}, R^{4T}, R^{4U}, R^{4V}, R^{4W}, R^{4X}, R^{4Y}, R^{4Z}, R^{4AA}, R^{4BB}, R^{4CC}, R^{4DD}, and R^{4EE} is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₄ heteroalkyl, and salts thereof. In one particular embodiment, each of X^{4A}, X^{4B}, and X^{4C} is, independently, selected from S, O, and two hydrogen atoms; and X^{4G} is, independently, selected from H, C₁₋₈ alkyl, and C₁₋₈ heteroalkyl; and X^{4H} is H; and each of X^{4D} and X^{4E} is, independently, selected from O, CH₂, NH, and S; and X^{4F} is, independently, selected from CH and N; and each of R^{4A}, R^{4B}, R^{4C}, R^{4D}, R^{4E}, R^{4F}, R^{4G}, R^{4H}, and R^{4I}, is, independently, selected from H, halide, nitro, C₁₋₄ alkyl, C₂₋₄ alkenyl, C₂₋₄ alkynyl, OR^{4K}, OC(O)R^{4L}, NR^{4M}R^{4N}, NHC(O)R^{4O}, NHC(S)R^{4P}, NHC(O)OR^{4Q}, NHC(S)OR^{4R}, NHC(O)NHR^{4S}, NHC(S)NHR^{4T}, NHC(O)SR^{4U}, NHC(S)SR^{4V}, NHS(O)₂R^{4W}, C(O)OR^{4X}, C(O)NHR^{4Y}, (CH₂)₁₋₄OH, C(O)R^{4Z}, CH₂R^{4AA}, SO₃H, SO₂R^{4BB}, S(O)R^{4CC}, SR^{4DD}, SO₂NHR^{4EE}, and S(CH₂)₁₋₄C(O)OH; and each of R^{4K}, R^{4L}, R^{4M}, R^{4N}, R^{4O}, R^{4P}, R^{4Q}, R^{4R}, R^{4S}, R^{4T}, R^{4U}, R^{4V}, R^{4W}, R^{4X}, R^{4Y}, R^{4Z}, R^{4AA}, R^{4BB}, R^{4CC}, R^{4DD}, and R^{4EE} is, independently, selected from H and C₁₋₄ alkyl, and salts thereof.

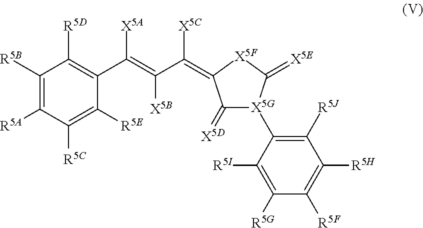

wherein each of X^{5A}, X^{5B}, and X^{5C} is, independently, selected from H, C₁₋₈ alkyl, C₂₋₄ alkenyl, C₂₋₄ alkynyl, and C₁₋₈ heteroalkyl; and each of X^{5D} and X^{5E} is, independently, selected from S, NH, O, and CH₂; and X^{5F} is, independently, selected from O, NH, CH₂, and S; and X^{5G} is, independently, selected from CH and N; and each of R^{5A}, R^{5B}, R^{5C}, R^{5D}, R^{5E}, R^{5F}, R^{5G}, R^{5H}, R^{5I}, and R^{5J}, is, independently, selected from H, halide, nitro, C₁₋₄ alkyl, C₂₋₄ alkenyl, C₂₋₄ alkynyl, OR^{5K}, OC(O)R^{5L}, NR^{5M}R^{5N}, NHC(O)R^{5O}, NHC(S)R^{5P}, NHC(O)OR^{5Q}, NHC(S)OR^{5R}, NHC(O)NHR^{5S}, NHC(S)NHR^{5T}, NHC(O)SR^{5U}, NHC(S)SR^{5V}, NHS(O)₂R^{5W}, C(O)OR^{5X}, C(O)NHR^{5Y}, (CH₂)₁₋₄OH, C(O)R^{5Z}, CH₂R^{5AA}, SO₃H, SO₂R^{5BB}, S(O)R^{5CC}, SR^{5DD}, SO₂NHR^{5EE}, and S(CH₂)₁₋₄C(O)OH; and each of R^{5K}, R^{5L}, R^{5M}, R^{5N}, R^{5O}, R^{5P}, R^{5Q}, R^{5S}, R^{5T}, R^{5U}, R^{5V}, R^{5W}, R^{5X}, R^{5Y}, X^{5Z}, R^{5AA}, R^{5BB}, R^{5CC}, R^{5DD}, and R^{5EE} is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₄ heteroalkyl, and salts thereof. In one particular embodiment, each of X^{5A}, X^{5B}, and X^{5C} is H; and each of X^{5D} and X^{5E} is, independently, selected from S and O; and X^{5F} is, independently, selected from O, NH, CH₂, and S; and X^{5G} is, independently, selected from CH and N; and each of R^{5A}, R^{5B}, R^{5C}, R^{5D}, R^{5E}, R^{5F}, R^{5G}, R^{5H}, R^{5I}, and R^{5J}, is, independently, selected from H, halide, nitro, C₁₋₄ alkyl, C₂₋₄ alkenyl, C₂₋₄ alkynyl, OR^{5K}, OC(O)R^{5L}, NR^{5M}R^{5N}, NHC(O)R^{5O}, NHC(S)R^{5P}, NHC(O)OR^{5Q}, NHC(S)OR^{5R}, NHC(O)NHR^{5S}, NHC(S)NHR^{5T}, NHC(O)SR^{5U}, NHC(S)SR^{5V}, NHS(O)₂R^{5W}, C(O)OR^{5X}, C(O)NHR^{5Y}, (CH₂)₁₋₄OH, C(O)R^{5Z}, CH₂R^{5AA}, SO₃H, SO₂R^{5BB}, S(O)R^{5CC}, SR^{5DD}, SO₂NHR^{5EE}, and S(CH₂)₁₋₄C(O)OH; and each of R^{5K}, R^{5L}, R^{5M}, R^{5N}, R^{5O}, R^{5P}, R^{5Q}, R^{5R}, R^{5S}, R^{5T}, R^{5U}, R^{5V}, R^{5W}, R^{5X}, R^{5Y}, R^{5Z}, R^{5AA}, R^{5BB}, R^{5CC}, R^{5DD}, and R^{5EE} is, independently, selected from H and C₁₋₄ alkyl, and salts thereof.

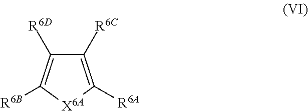

wherein X^{6A} is, independently, selected from S, NH, and O; and each of R^{6A}, R^{6B}, R^{6C}, and R^{6D}, is, independently, selected from H, halide, nitro, C₁₋₄ alkyl, C₂₋₄ alkenyl, C₂₋₄ alkynyl, OR^{6K}, OC(O)R^{6L}, NR^{6M}R^{6N}, NHC(O)R^{6O}, NHC(S)R^{6P}, NHC(O)OR^{6Q}, NHC(S)OR^{6R}, NHC(O)NHR^{6S}, NHC(S)NHR^{6T}, NHC(O)SR^{6U}, NHC(S)SR^{6V}, NHS(O)₂R^{6W}, C(O)OR^{6X}, C(O)NHR^{6Y}, (CH₂)₁₋₄OH, C(O)R^{6Z}, CH₂R^{6AA}, SO₃H, SO₂R^{6BB}, S(O)R^{6CC}, SR^{6DD}, SO₂NHR^{6EE}, and S(CH₂)₁₋₄C(O)OH; and each of R^{6K}, R^{6L}, R^{6M}, R^{6N}, R^{6O}, R^{6P}, R^{6Q}, R^{6R}, R^{6S}, R^{6T}, R^{6U}, R^{6V}, R^{6W}, R^{6X}, R^{6Y}, R^{6Z}, R^{6AA}, R^{6BB}, R^{6CC}, R^{6DD}, and R^{6EE} is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₄ heteroalkyl, and salts thereof. In one particular embodiment, X^{6A} is, independently, selected from S and O; and each of R^{6C} and R^{6D} is H; and each of R^{6A} and R^{6B}, is, independently, selected from H, halide, nitro, C₁₋₄ alkyl, C₂₋₄ alkenyl, C₂₋₄ alkynyl, OR^{6K}, OC(O)R^{6L}, NR^{6M}R^{6N}, NHC(O)R^{6O}, NHC(S)R^{6P}, NHC(O)OR^{6Q}, NHC(S)OR^{6R}, NHC(O)NHR^{6S}, NHC(S)NHR^{6T}, NHC(O)SR^{6U}, NHC(S)SR^{6V}, NHS(O)₂R^{6W} C(O)OR^{6X}, C(O)NHR^{6Y}, (CH₂)₁₋₄OH, C(O)R^{6Z}, CH₂R^{6AA}, SO₃H, SO₂R^{6BB}, S(O)R^{6CC}, SR^{6DD}, SO₂NHR^{6EE}, and S(CH₂)₁₋₄C(O)OH; and each of R^{6K}, R^{6L}, R^{6M}, R^{6N}, R^{6O}, R^{6P}, R^{6Q}, R^{6R}, R^{6S}, R^{6T}, R^{6U}, R^{6V}, R^{6W}, R^{6X}, R^{6Y}, R^{6Z}, R^{6AA}, R^{6BB}, R^{6CC}, R^{6DD}, and R^{6EE} is, independently, selected from H and C₁₋₄ alkyl, and salts thereof.

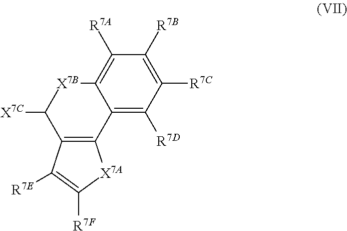

wherein each of X^{7A} and X^{7B} is, independently, selected from S, NH, and O; and X^{7C} is, independently, selected from S, NH, CH₂, and O; and each of R^{7A}, R^{7B}, R^{7C}, R^{7D}, R^{7E}, and R^{7F} is, independently, selected from H, halide, nitro, C₁₋₄ alkyl, C₂₋₄ alkenyl, C₂₋₄ alkynyl, OR^{7K}, OC(O)R^{7L}, NR^{7M}R^{7N}, NHC(O)R^{7O}, NHC(S)R^{7P}, NHC(O)OR^{7Q}, NHC(S)OR^{7R}, NHC(O)NHR^{7S}, NHC(S)NHR^{7T}, NHC(O)SR^{7U}, NHC(S)SR^{7V}, NHS(O)₂R^{7W}, C(O)OR^{7X}, C(O)NHR^{7Y}, (CH₂)₁₋₄OH, C(O)R^{7Z}, CH₂R^{7AA}, SO₃H, SO₂R^{7BB}, S(O)R^{7CC}, SR^{7DD}, SO₂NHR^{7EE}, and S(CH₂)₁₋₄C(O)OH; and each of R^{7K}, R^{7L}, R^{7M}, R^{7N}, R^{7O}, R^{7P}, R^{7Q}, R^{7R}, R^{7S}, R^{7T}, R^{7U}, R^{7V}, R^{7W}, R^{7X}, R^{7Y}, R^{7Z}, R^{7AA}, R^{7BB}, R^{7CC}, R^{7DD}, and R^{7EE} is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₄ heteroalkyl, and salts thereof. In one particular embodiment, each of X^{7A} and X^{7B} is, independently, selected from S, NH, and O; and X^{7C} is, independently, selected from S, NH, CH₂, and O; and each of R^{7A}, R^{7B}, R^{7C}, R^{7D}, and R^{7E} is, independently, selected from H, halide, nitro, C₁₋₄ alkyl, C₂₋₄ alkenyl, C₂₋₄ alkynyl, OR^{7K}, OC(O)R^{7L}, NR^{1M}R^{7N}, NHC(O)R^{7O}, NHC(S)R^{7P}, NHC(O)OR^{7Q}, NHC(S)OR^{7R}, NHC(O)NHR^{7S}, NHC(S)NHR^{7T}, NHC(O)SR^{7U}, NHC(S)SR^{7V}, NHS(O)₂R^{7W}, C(O)OR^{7X}, C(O)NHR^{7Y}, (CH₂)₁₋₄OH, C(O)R^{7Z}, CH₂R^{7AA}, SO₃H, SO₂R^{7BB}, S(O)R^{7CC}, SR^{7DD}, SO₂NHR^{7EE}, and S(CH₂)₁₋₄C(O)OH; and each of R^{7K}, R^{7L}, R^{7M}, R^{7N}, R^{7O}, R^{7P}, R^{7Q}, R^{7R}, R^{7S}, R^{7T}, R^{7U}, R^{7V}, R^{7W}, R^{7X}, R^{7Y}, R^{7Z}, R^{7AA}, R^{7BB}, R^{7CC}, R^{7DD}, and R^{7EE} is, independently, selected from H, C₁₋₄ alkyl; and R^{7F} is, independently, selected from OC(O)R^{7FF}, NHC(O)R^{7FF}, NHC(S)R^{7FF}, NHC(O)OR^{7FF}, NHC(S)OR^{7FF}, NHC(O)NHR^{7FF}, NHC(S)NHR^{7FF}, NHC(O)SR^{7FF}, NHC(S)SR^{7FF}, NHS(O)₂R^{7FF}, C(O)OR^{7FF}, C(O)NHR^{7FF}, C(O)R^{7FF}, SO₂R^{7FF}, S(O)R^{7FF}, and SO₂NHR^{7FF}, where R^{7FF} is selected from H and C₁₋₄ alkyl, and salts thereof.

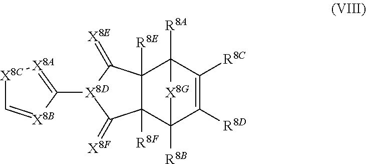

wherein X^{8C} is, independently, selected from NH, CH═CH, or N═CH, and each of X^{8A}, X^{8B}, and X^{8D} is, independently, selected from CH and N; and each of X^{8E}, X^{8F}, and X^{8G} is, independently, selected from S, NH, CH₂, and O; and each of R^{8A}, R^{8B}, R^{8C}, R^{8D}, R^{8E}, and R^{8F} is, independently, selected from H, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, and C₁₋₄ heteroalkyl, and salts thereof. In one particular embodiment, X^{8C} is, independently, selected from NH, CH═CH, or N═CH; and each of X^{8A}, X^{8B}, and X^{8D} is, independently, selected from CH and N; and each of X^{8E} and X^{8F} is, independently, selected from S and O; X^{8G} is CH₂; and each of R^{8A}, R^{8B}, R^{8C}, R^{8D}, R^{8E}, and R^{8F} is, independently, selected from H and C₁₋₄ alkyl, and salts thereof.

In still further aspects, a PKM2 inhibitor can be a compound that stabilizes the teterameric form for PKM2, which may reduce or prevent nuclear import of PKM2. Examples of such compounds are provided in Anastasiou et al., 2012 and in WO 2012/056319, each of which are incorporated herein by reference.

B. MEK/ERK Kinase Inhibitors

MEK inhibitors, which include inhibitors of mitogen-activated protein kinase kinase (MAPK/ERK kinase or MEK) or its related signaling pathways like MAPK cascade, may be used in certain aspects of the embodiments. Mitogen-activated protein kinase kinase (sic) is a kinase enzyme which phosphorylates mitogen-activated protein kinase. It is also known as MAP2K. Extracellular stimuli lead to activation of a MAP kinase via a signaling cascade (“MAPK cascade”) composed of MAP kinase, MAP kinase kinase (MEK, MKK, MEKK, or MAP2K), and MAP kinase kinase kinase (MKKK or MAP3K).

A MEK inhibitor herein refers to MEK inhibitors in general. Thus, a MEK inhibitor refers to any inhibitor of a member of the MEK family of protein kinases, including MEK1, MEK2 and MEK5. Reference is also made to MEK1, MEK2 and MEK5 inhibitors. Examples of suitable MEK inhibitors, already known in the art, include the MEK1 inhibitors PD184352 and PD98059, inhibitors of MEK1 and MEK2 U0126 and SL327, and those discussed in Davies et al. (2000).

In particular, PD184352 and PD0325901 have been found to have a high degree of specificity and potency when compared to other known MEK inhibitors (Bain et al., 2007). Other MEK inhibitors and classes of MEK inhibitors are described in Zhang et al. (2000).

Inhibitors of MEK can include antibodies to, dominant negative variants of, and siRNA and antisense nucleic acids that suppress expression of MEK. Specific examples of MEK inhibitors include, but are not limited to, PD0325901 (see, e.g., Rinehart et al., 2004), PD98059 (available, e.g., from Cell Signaling Technology), U0126 (available, for example, from Cell Signaling Technology), SL327 (available, e.g., from Sigma-Aldrich), ARRY-162 (available, e.g., from Array Biopharma), PD184161 (see, e.g., Klein et al., 2006), PD184352 (CI-1040) (see, e.g., Mattingly et al., 2006), sunitinib (AZD6244/ARRY-142886/ARRY-886; see, e.g., Voss, et al., US2008004287 incorporated herein by reference), sorafenib (see, Voss supra), Vandetanib (see, Voss supra), pazopanib (see, e.g., Voss supra), Axitinib (see, Voss supra), PTK787 (see, Voss supra), refametinib (BAY-86-9766/RDEA-119), Pimasertib (also known as AS703026 or MSC1936369B), and trametinib (GSK-1120212).

Currently, several MEK inhibitors are undergoing clinical trial evaluations. CI-1040 has been evaluated in Phase I and II clinical trials for cancer (see, e.g., Rinehart et al., 2004). Other MEK/ERK inhibitors being evaluated (e.g., in clinical trials) include PD 184352 (see, e.g., English et al., 2002), BAY 43-9006 (see, e.g., Chow et al., 2001), PD-325901 (also PD0325901), ARRY-438162, RDEA1 19, RDEA-436, RO5126766, XL518, AZD8330 (also ARRY-704), GDC-0973, RDEA1 19, PD18416, SCH 900353, RG-7167, WX-554, E-6201, AS-703988, BI-847325, TAK-733, RG-7304, or FR180204.

C. NF-κB Inhibitors

Certain aspects of the embodiments concern inhibitors of NF-κB or the NF-κB pathway. In particular aspect, NF-κB-pathway inhibitors for use according to the embodiments are those that inhibit the EGF-dependent NF-κB pathway. Examples of inhibitors of IKKβ activity include those listed in Table A, below. In some aspects, the inhibitor is Bay 11-7082 or sulfasalazine, which have been shown to inhibit both IKKα and IKKβ. Another useful inhibitor is glycosylated indolocarbazol, EC-7014, which has been recently been identified as a potent and selective inhibitor of IKKβ. Likewise, IKK inhibitors, such as SAR113945, maybe used to inhibit the pathway. SAR113945, for example, is a small molecule inhibitor from Sanofi-Aventis that is being evaluated in patients with knee osteoarthritis. In further aspects the NF-κB-pathway can be indirectly inhibited by use of a proteasome inhibitor. Proteasome inhibitor s, such as PS-341 (bortezomib, velcade), selectively and reversibly inhibit the 26S proteasome and prevent the breakdown of many regulatory proteins including IKB.

TABLE A
Small molecules IKKβ inhibitors

| INHIBITOR | *IC50 | | REFERENCE** |
|---|---|---|---|
| BMS-345541 | 0.3 | μM | Burke et al., J Biol Chem. 278: 1450-6, |
| | | | 2003. |
| IMD-0354 | 0.28-3.0 | μM | Tanaka et al., Blood. 105: 2324-31, 2005. |
| TPCA-1 | 0.018 | μM | J Pharmacol Exp Ther. 2005; 312: 373-81 |
| PS1145 | 0.088 | μM | J Biol Chem. 2005; 280: 20442-8 |
| MLN120B | 0.06-1.0 | μM | Nagashima et al., Blood. 107: 4266-73, |
| | | | 2006. |
| IKI-1 | 0.07 | μM | Cancer Res. 2008; 68: 9519-24 |
| KINK-1 | 2.8-21 | μM | J Natl Cancer Inst. 2008; 100: 862-75 |
| NSC 676914 | 17 | μM | Mol Cancer Ther. 2009; 8: 571-81 |
| PF-184 | 0.037 | μM | J Pharmacol Exp Ther. 2009; 330: 377-88 |
| VH01 | 20.3 | μM | BMC Bioinformatics. 2010; Suppl 7: S15 |
| LASSBio-1524 | 20 | μM | Eur J Med Chem. 2011; 46: 1245-53 |

*half maximal inhibitory concentration;
**each of which is incorporated herein by reference.

D. PKCε inhibitors

Certain aspects of the embodiments concern PKC inhibitors and the administration of such inhibitors. In preferred aspects, the PKC inhibitors are specific for PKCε. Inhibitors of PKC can include, without limitation, Midostaurin (PKC412) and sotrastaurin (AEB071). Peptide inhibitors of PKCε include, for example, the Cys-conjugated peptide: EAVSLKPTC, which inhibits PKCε interaction with the anchoring protein ERACK.

E. Src Inhibitors

Examples of Src inhibitors for use according to the embodiments include, without limitation, BMS-354825 (Dasatinib), SKI-606 (Bosutinib), AZD0530 (Saracatinib), AP23451, saracatinib (AZD-0530), ponatinib (AP-24534), KX-01, NS-018, KD-020, BGB-102, XL-228, KD-019, AZD0424, KX2-391, and XL999.

F. Pin1 Inhibitors

Further aspects of the embodiments concern Pin1 inhibitors and the administration of such inhibitors. Examples of inhibitors of Pin1 include, without limitation, TME-001 (2-(3-chloro-4-fluoro-phenyl)-isothiazol-3-one; see, Mori et al., 2011), 5′-nitro-indirubinoxime (Yoon et al., 2012) and cyclohexyl ketone substrate analogue inhibitors, such as Ac-pSer-Ψ[C═OCH]-Pip-tryptamine (Xu et al., 2012). Xu et al. (2011) also describe a Pin1 inhibitor having the structure R-pSer-Ψ[CH₂N]-Pro-2-(indol-3-yl)ethylamine, wherein R is fluorenylmethoxycarbonyl (Fmoc) or Ac. Peptides such as, disulfide-cyclized peptides, have also been demonstrated as an effective Pin1 inhibitors and may be used in accordance with the present embodiments (see, e.g., Duncan et al. 2011, incorporated herein by reference).

G. Additional Targeted Inhibitors

Targeted inhibition can likewise be achieved using targeted inhibitory RNA therapies (e.g., through the administration or expression of micro RNAs (miRNAs) or small interfering RNAs (siRNAs) to a particular gene or pathway). Inhibition of, for example, PKM2, NF-κB, Src, PKCε or MEK/ERKs can be conveniently achieved using RNA-mediated interference. Typically, a double-stranded RNA molecule complementary to all or part of a target mRNA is introduced into cancer cells, thus promoting specific degradation of mRNA molecules. This post-transcriptional mechanism results in reduced or abolished expression of the targeted mRNA and the corresponding encoded protein.

Moreover a number of assays for identifying new targeted inhibitor, including e.g., PKM2, Pin1, Src, PKCε or MEK inhibitors, are known. For example, Davies et al. (2000) describes kinase assays in which a kinase is incubated in the presence of a peptide substrate and radiolabeled ATP. Phosphorylation of the substrate by the kinase results in incorporation of the label into the substrate. Aliquots of each reaction are immobilized on phosphocellulose paper and washed in phosphoric acid to remove free ATP. The activity of the substrate following incubation is then measured and provides an indication of kinase activity. The relative kinase activity in the presence and absence of candidate kinase inhibitors can be readily determined using such an assay. Downey et al. (1996) also describes assays for kinase activity which can be used to identify kinase inhibitors that may be used in accordance with the embodiments.

H. Prodrugs

Compounds, such as targeted inhibitors of the present embodiments may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. In general, such prodrugs will be functional derivatives of the metabolic pathway inhibitors of the embodiments, which are readily convertible in vivo into the active inhibitor. Conventional procedures for the selection and preparation of suitable prodrug derivatives are described, for example, in “Design of Prodrugs”, ed. H. Bundgaard, Elsevier, 1985; Huttunen et al., 2011; and Hsieh et al., 2009, each of which is incorporated herein by reference in its entirety.

A prodrug may be a pharmacologically inactive derivative of a biologically active inhibitor (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality. Thus, prodrugs of the compounds employed in the embodiments may be prepared by modifying functional groups present in the compound in such a way that the modifications are cleaved, either in routine manipulation or in vivo, to the parent compound. Prodrugs include, for example, compounds described herein in which a hydroxy, amino, or carboxy group is bonded to any group that, when the prodrug is administered to a subject, cleaves to form a hydroxy, amino, or carboxylic acid, respectively. Thus, the invention contemplates prodrugs of compounds of the present invention as well as methods of delivering prodrugs.

### III. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Nuclear PKM2 Regulates 1-Catenin Transactivation Upon EGFR Activation

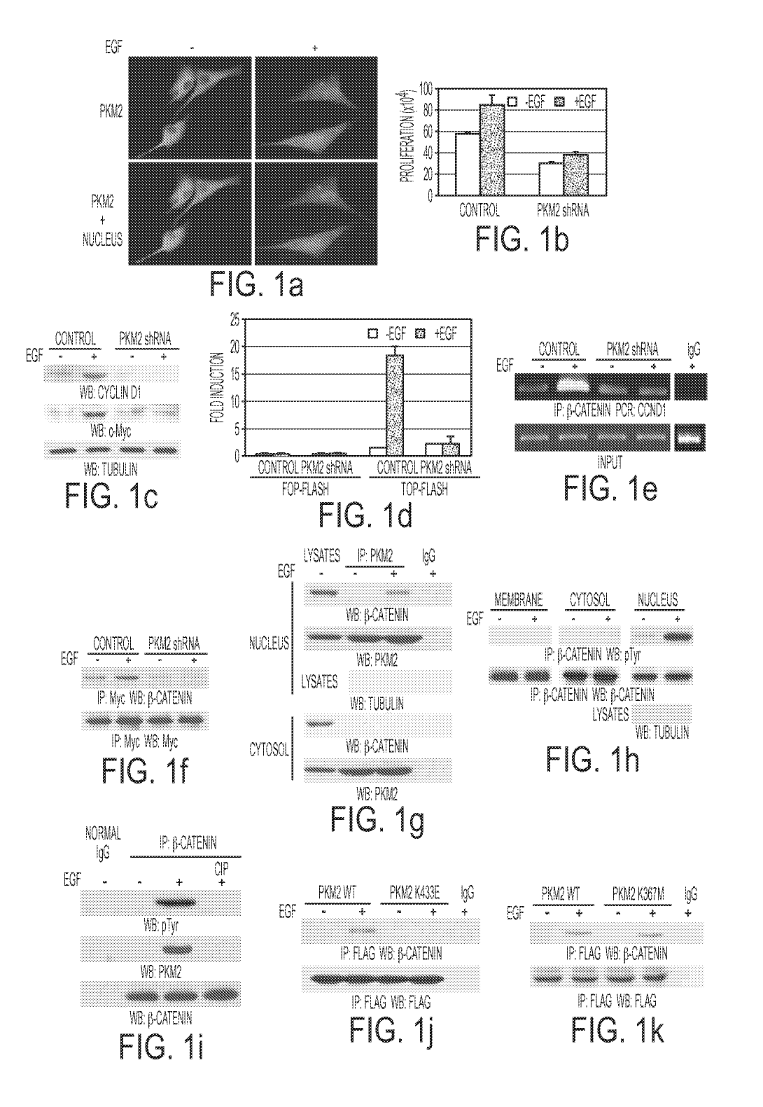

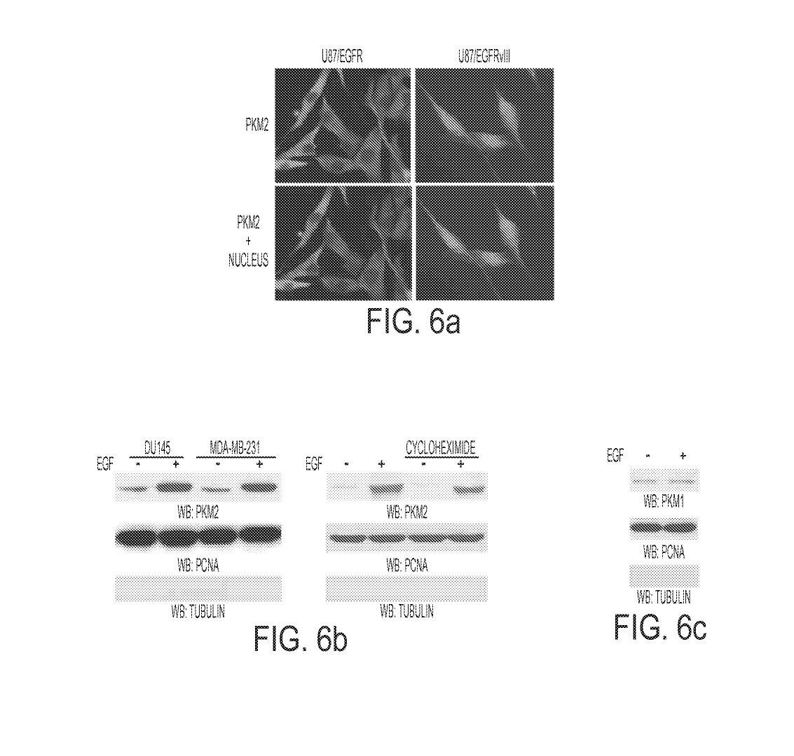

Since both EGFR activation and PKM2 expression are instrumental in tumorigenesis a (Lu et al., 2001; Wykosky et al., 2011; Christofk et al., 2008), it was examined whether EGFR activation regulates PKM2 functions in a subcellular compartment-dependent manner. Immunofluorescence analysis showed that EGF treatment resulted in the nuclear accumulation of PKM2 in U87/EGFR human glioblastoma (GBM) cells (FIG. 1a). In addition, expression of the constitutively active EGFRvIII mutant in U87 cells had a higher amount of nuclear PKM2 than did EGF-untreated U87/EGFR cells (FIG. 6a). The finding that EGF induces nuclear translocation of PKM2 was further supported by cell fractionation analysis of DU 145 prostate cancer cells, MDA-MB-231 breast cancer cells, and U87/EGFR cells (FIG. 6b). In addition, PKM1 failed to translocate into the nucleus upon EGF stimulation (FIG. 6c), indicating that EGF specifically regulates the subcellular distribution of PKM2 in multiple types of cancer cells.

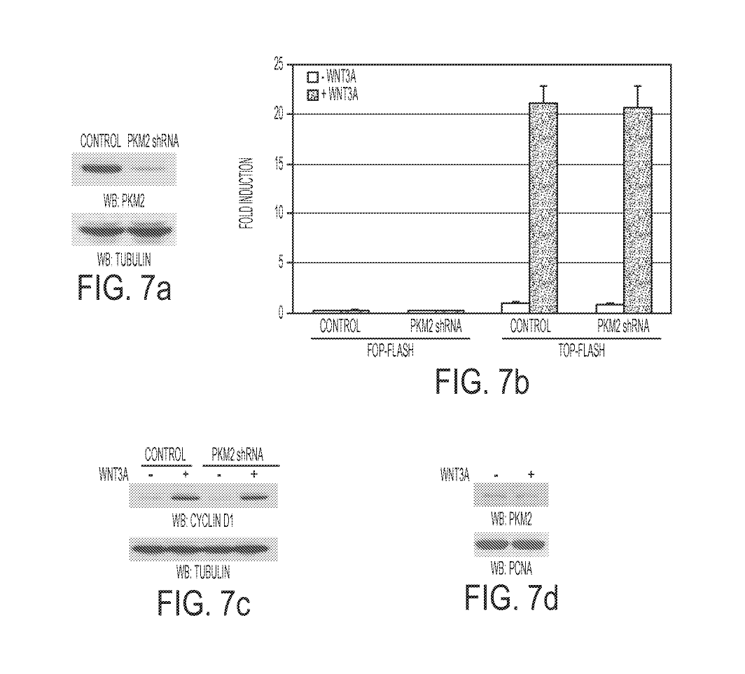

To examine whether PKM2 directly regulates gene transcription and cell proliferation, PKM2 shRNA was expressed in U87/EGFR cells (FIG. 7a). PKM2 depletion largely reduced both basal and EGF-induced tumor cell proliferation (FIG. 1b) and blocked EGF-enhanced expression of cyclin D1 and c-Myc (FIG. Ic), which are known important regulators of cell proliferation and downstream genes of β-catenin transactivation (Yochum et al., 2007). To examine whether these PKM2-dependent effects were mediated by β-catenin, TCF/LEF-1 luciferase reporter analyses were performed, showing that PKM2 depletion significantly inhibited EGF-induced β-catenin transactivation (FIG. ID). In addition, chromatin immunoprecipitation (ChIP) analyses showed that EGFR activation resulted in increased binding of β-catenin to the promoter region of CCND1 (coding for cyclin D1) (FIG. 1e) and c-myc, which was blocked by PKM2 depletion. In addition, coimmunoprecipitation (co-IP) analyses showed that PKM2 depletion inhibited EGF-induced interaction between β-catenin and Myc-tagged TCF4 (FIG. If). However, PKM2 depletion failed to inhibit Wnt3a- or Wnt1-induced β-catenin transactivation (FIG. 7b) and cyclin D1 expression (FIG. 7c). In addition, Wnt3a did not induce PKM2 nuclear translocation (FIG. 7d). These results indicate that EGF induces β-catenin transactivation via a mechanism distinct from that induced by Wnt/wingless (Lu and Hunter, 2004) and that PKM2 expression plays a pivotal role in EGF-, but not Wnt-induced β-catenin transactivation.

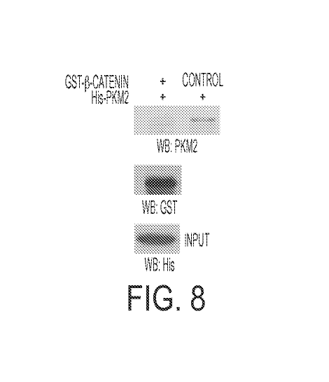

To examine the mechanism underlying PKM2-regulated β-catenin transactivation, co-IP analyses were performed, showing that EGF stimulation resulted in an interaction between endogenous PKM2 and β-catenin in the nuclear, but not cytosolic, fraction of U87/EGFR cells (FIG. 1g). However, an in vitro glutathione S-transferase (GST) pull-down assay showed that purified GST-β-catenin failed to bind to purified His-PKM2 (FIG. 8). These results suggest that the interaction of these two proteins might require post-translational modifications of the proteins.

PKM2 binds to tyrosine-phosphorylated peptides, and expression of the phosphotyrosine-binding form is required for cancer cell growth (Christofk et al., 2008). To examine whether β-catenin is tyrosine-phosphorylated, immunoblotting analyses were performed with a phospho-Tyr antibody, showing that EGF stimulation induced Tyr phosphorylation of immunoprecipitated β-catenin in the nucleus, but not in the cytosol or at the plasma membrane (FIG. 1h). Treatment of the immunoprecipitated β-catenin with calf intestinal alkaline phosphatase (CIP) resulted in β-catenin dephosphorylation and abrogation of the PKM2-β-catenin interaction (FIG. 1i). Thus, EGF-induced Tyr-phosphorylation of β-catenin is required for the PKM2-β-catenin interaction.

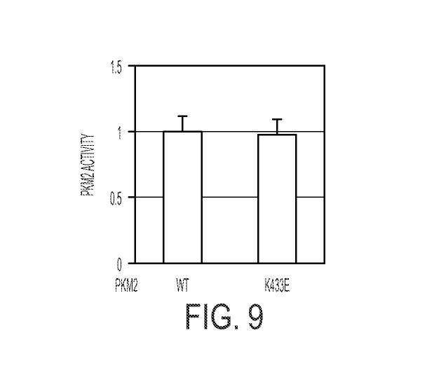

PKM2 K433E mutant, which fails to bind to tyrosine-phosphorylated peptides (Christofk et al., 2008), had similar glycolytic enzyme activity to its WT counterpart (FIG. 9) (Christofk et al., 2008). Co-IP analyses showed that EGF treatment induced the binding of β-catenin to FLAG-tagged wild-type (WT) PKM2, but not to the PKM2 K433E mutant (FIG. 1j). In contrast, a kinase-dead FLAG-PKM2 K367M (Le Mellay et al., 2002; Mazurek, 2005), acting like its WT counterpart, binds to β-catenin (FIG. 1k). These results indicate that the K433 binding residue of PKM2, but not its catalytic activity, is critical for the PKM2-β-catenin interaction.

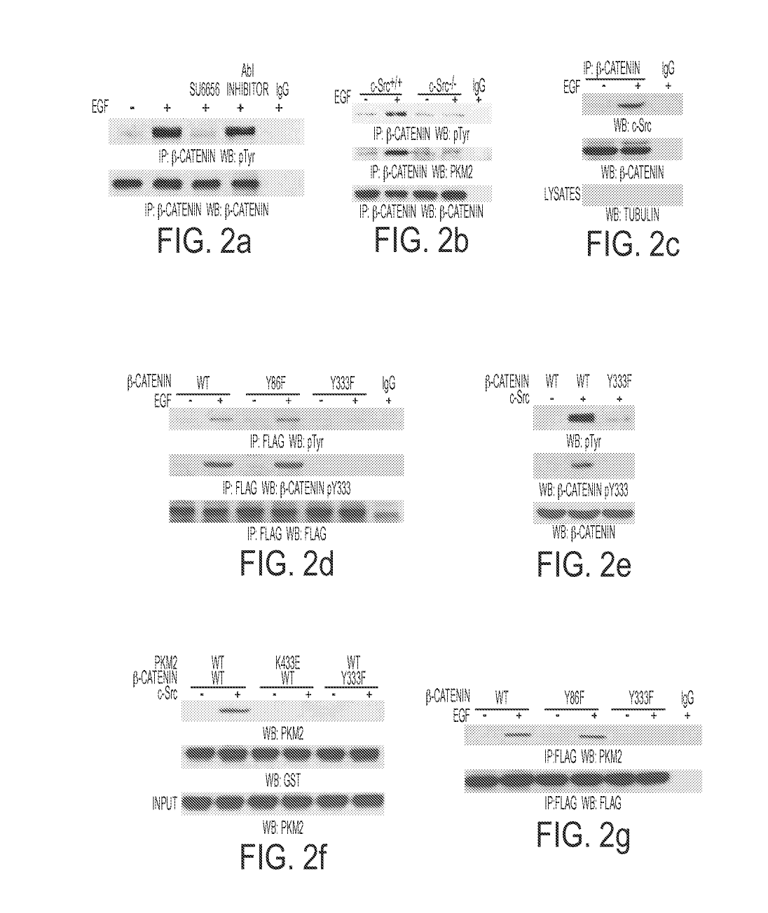

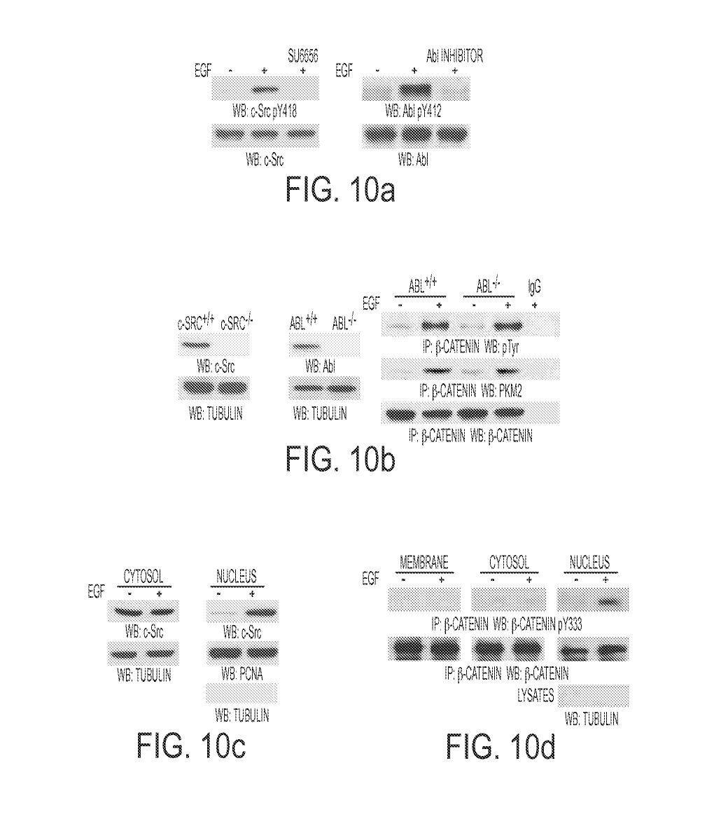

ABL and Src have been reported to phosphorylate β-catenin (Coluccia et al., 2007; Miravet et al., 2003). Pretreatment with SU6656 (Src inhibitor) or an ABL inhibitor completely abrogated EGF-induced activation of c-Src or ABL, as shown by the reduced levels of c-Src (Y418) or ABL (Y412) phosphorylation (FIG. 10a). However, inhibition of c-Src, but not ABL, blocked EGF-induced Tyr phosphorylation of β-catenin (FIG. 2a). In addition, deficiency of c-Src (FIG. 2b), but not of ABL (FIG. 10b), abrogated EGF-induced β-catenin Tyr-phosphorylation and the PKM2-β-catenin interaction. These results indicate that c-Src, a downstream effector of EGFR, phosphorylates β-catenin, which is required for the PKM2-β-catenin interaction.

To examine the subcellular compartment in which c-Src phosphorylates β-catenin, fractionation analyses were conducted. It was found that EGF stimulation resulted in the nuclear translocation of c-Src (FIG. 10c). In addition, co-IP analyses showed that EGF treatment induced an enhanced interaction between β-catenin and c-Src in nuclear fractions (FIG. 2c). These results, combined with the evidence that β-catenin is phosphorylated in the nucleus (FIG. 1h), strongly suggest that c-Src translocates into the nucleus and subsequently interacts with and phosphorylates β-catenin.

The Y86 residue of β-catenin has been shown to be phosphorylated by Src as well as Bcr-ABL (Coluccia et al., 2007; Miravet et al., 2003), and analysis of the amino acid sequence identified an additional potential Src phosphorylation site at Y333. Immunoblotting analysis showed that EGF stimulation resulted in tyrosine phosphorylation of FLAG-tagged WT β-catenin and β-catenin Y86F, but not β-catenin Y333F, which was further validated by immunoblotting with a phospho-β-catenin Y333 antibody (FIG. 2d). In addition, cell fractionation analysis demonstrated that EGF induced β-catenin Y333 phosphorylation primarily in the nucleus (FIG. 10d). Furthermore, an in vitro protein kinase assay showed that active c-Src was able to phosphorylate WT β-catenin (FIG. 2e), but the 3-catenin Y333F mutation largely reduced total Tyr-phosphorylation levels and completely abrogated the Y333-phosphorylation. These results reveal that c-Src binds and phosphorylates β-catenin at Y333 in vitro and in vivo.

To examine whether phosphorylation of β-catenin Y333 regulates its binding to PKM2, a GST pull-down assay was performed by mixing purified GST-β-catenin and His-PKM2 with or without purified active c-Src. FIG. 2f shows that WT PKM2 did not bind to unphosphorylated WT β-catenin. However, the presence of c-Src enabled the binding of WT PKM2 to β-catenin, which was abrogated by mutation of β-catenin at Y333 or PKM2 at K433. These in vitro results were further validated by co-IP analyses, showing that in contrast to FLAG-tagged WT β-catenin or β-catenin Y86F, FLAG-β-catenin Y333F failed to bind to endogenous PKM2 (FIG. 2g). These results indicate that c-Src-mediated β-catenin Y333 phosphorylation is required for the PKM2-β-catenin interaction.

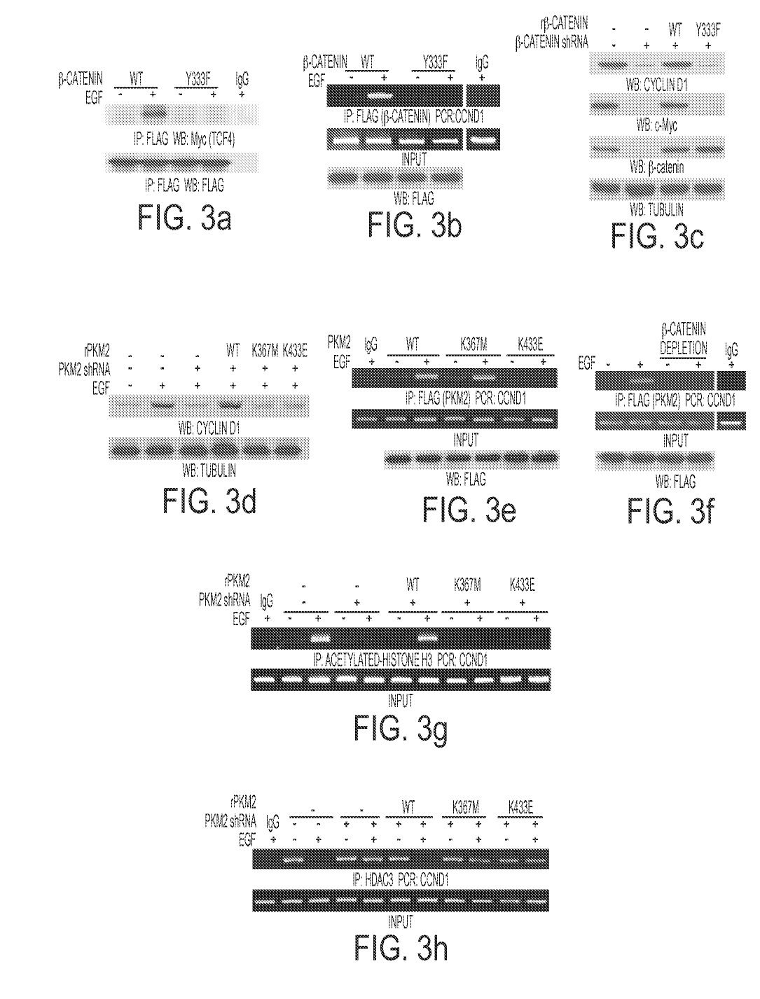

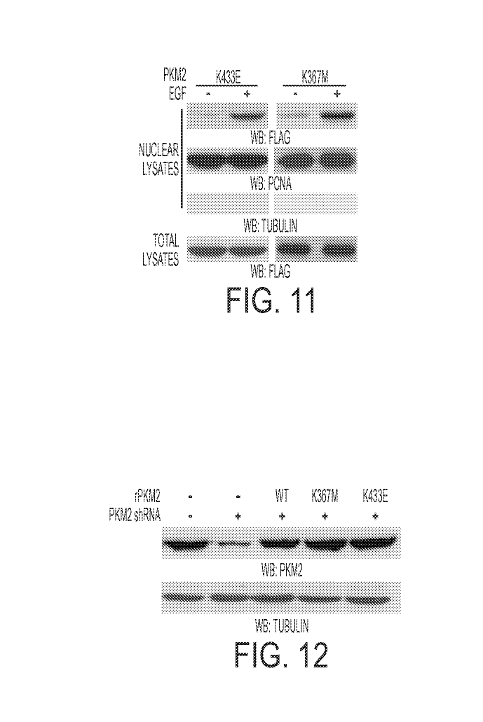

Next it was examined the significance of the PKM2-β-catenin interaction in β-catenin transactivation. FIG. 3a shows that FLAG-β-catenin Y333F failed to interact with Myc-tagged TCF4 upon EGF stimulation, in contrast to its WT counterpart. ChIP analyses demonstrated that FLAG-tagged WT β-catenin bound to the CCND1 promoter region upon EGFR activation, which was abrogated by the Y333F mutation (FIG. 3b). In addition, reconstituted expression of the RNAi-resistant β-catenin (rβ-catenin) Y333F mutant, but not of WT rβ-catenin, in endogenous β-catenin-depleted U87/EGFRvIII cells failed to induce cyclin D1 and c-Myc expression (FIG. 3c). Furthermore, FLAG-PKM2 K433E and the inactive FLAG-PKM2 K367M mutant translocated into the nucleus upon EGF stimulation (FIG. 11), but reconstituted expression of these mutants (FIG. 12) failed to induce cyclin D1 expression as did WT rPKM2 expression (FIG. 3d). Thus, both PKM2 catalytic activity and the PKM2-β-catenin interaction are required for cyclin D1 expression upon EGFR activation.

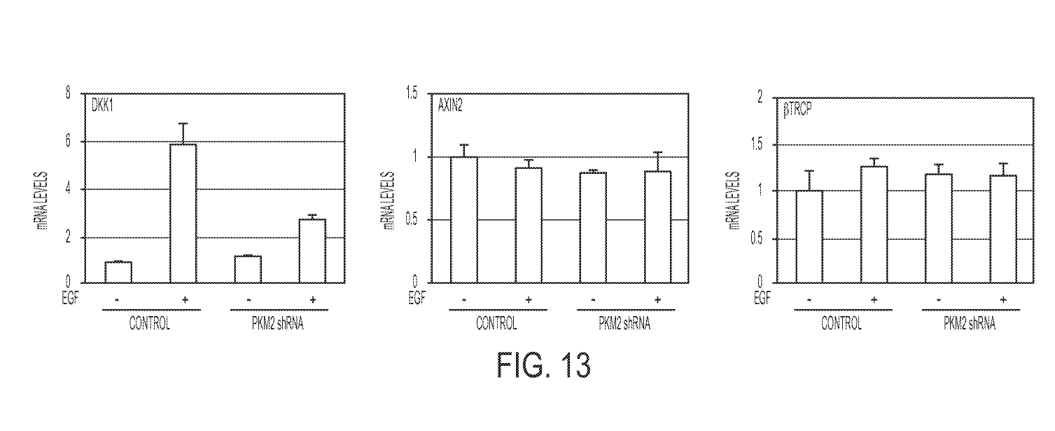

To compare downstream targets of EGF and Wnt signaling, the expression of other Wnt/β-catenin downstream genes: AXIN2, DKK1, and βTrCP were examined (Yochum et al., 2007). Quantitative RT-PCR analysis showed that EGF treatment increased mRNA levels of DKK1, but not of AXIN2 or (3TrCP, which was blocked by PKM2 depletion (FIG. 13). These results indicate that EGF-induced and PKM2-dependent β-catenin transactivation induced transcription of a set of genes, which do not completely overlap with those induced by Wnt signaling.

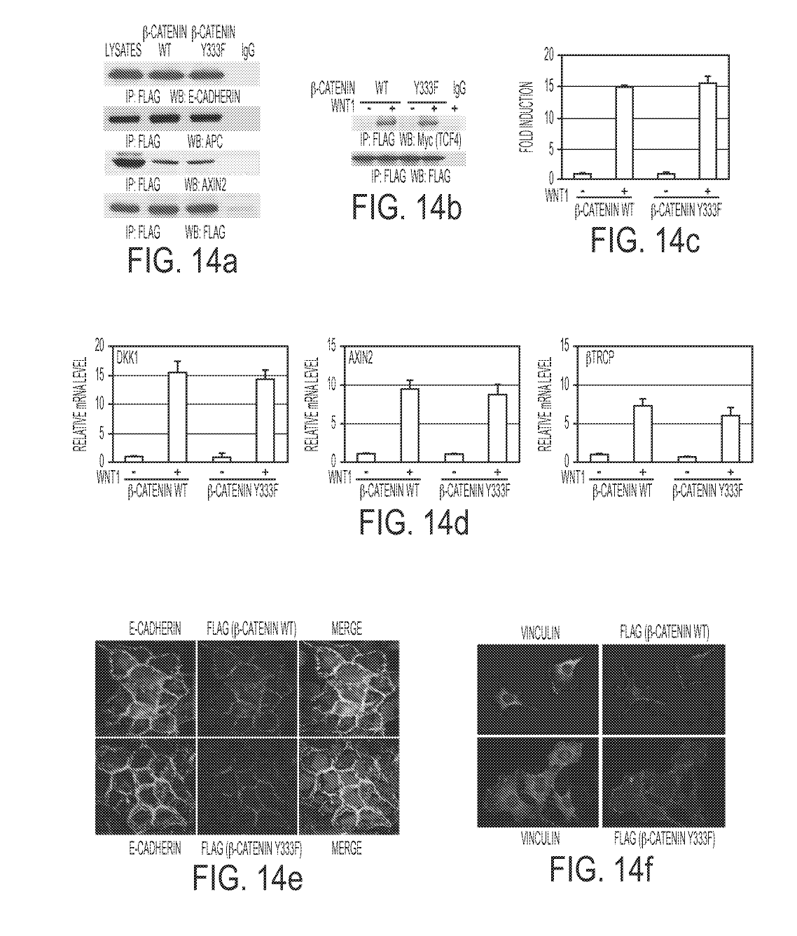

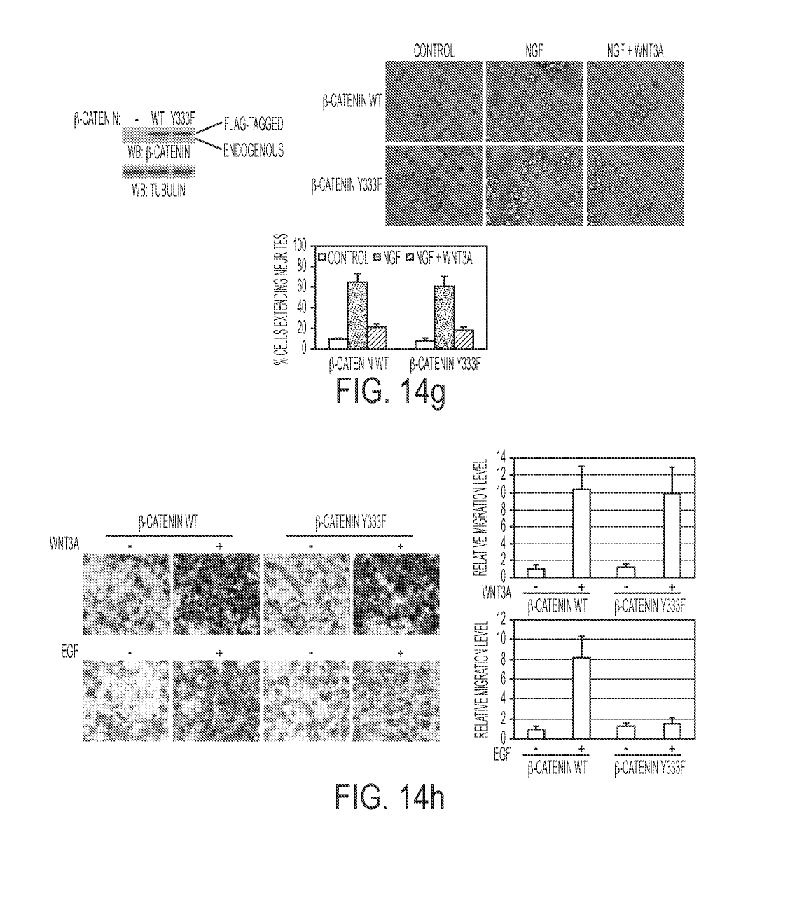

The findings that PKM2 was not required for Wnt3a-induced β-catenin transactivation (FIG. 7b) suggested that c-Src-dependent β-catenin Y333 phosphorylation is not involved in Wnt-induced signaling or cell adhesion. This assumption was supported by the results, showing that β-catenin Y333F behaved similarly to WT β-catenin in binding to APC, AXIN2, and E-cadherin and in Wnt-induced β-catenin transactivation and cellular functions (FIG. 14 a-g). In contrast, expression of β-catenin Y333F blocked EGF- but not Wnt3a-induced cell migration (FIG. 14h).

To investigate the mechanisms underlying PKM2- and β-catenin-dependent cyclin D1 expression, ChIP analyses were performed. FIG. 3e shows that EGF induced an enhanced binding of WT FLAG-PKM2 and FLAG-PKM2 K367M, but not of FLAG-PKM2 K433E, to the CCND1 promoter region. In addition, binding of FLAG-PKM2 to the promoter region was not detected in ChIP analyses after β-catenin had been immunodepleted from cell lysates (FIG. 3f). These results indicate that the PKM2-β-catenin interaction, but not PKM2 kinase activity, is required for both proteins to bind to the CCND1 promoter region.

Next it was examined whether PKM2 binding to the CCND1 promoter region regulates histone H3 acetylation, which is important for gene transcription. ChIP analyses showed that EGF treatment resulted in a significant increase of histone H3 acetylation in the CCND1 promoter region, which was blocked by PKM2 depletion (FIG. 3g). Reconstituted expression of rPKM2 K433E and the inactive rPKM2 K367M mutant failed to restore EGF-induced histone H3 acetylation, compared with the WT protein.

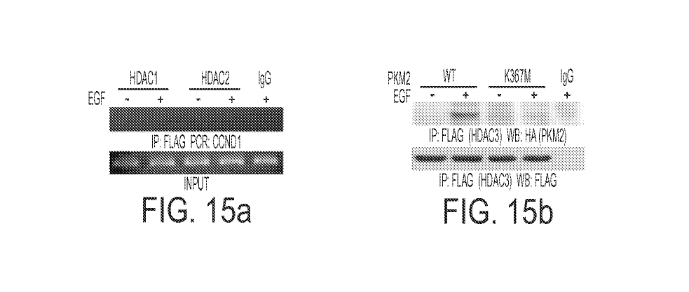

To further understand the mechanism underlying PKM2-regulated histone H3 acetylation, ChIP analyses were performed with antibodies against ubiquitously expressed histone deacetylase (HDAC)1, HDAC2, and HDAC3 (Xia et al., 2007). FIG. 3h shows that HDAC3, but not HDAC1 or HDAC2 (FIG. 15a), was prebound to the CCND1 promoter. EGF treatment resulted in the disassociation of HDAC3 from the CCND1 promoter, and this disassociation was blocked by PKM2 depletion. Furthermore, reconstituted expression of WT rPKM2, but not of rPKM2 K367M or rPKM2 K433E mutants, was able to restore EGF-induced disassociation of HDAC3 from the promoter (FIG. 3h). These results indicate that the kinase activity of PKM2 and its binding with β-catenin to the CCND1 promoter region are required for HDAC3 removal from the promoter. An additional co-IP analysis showed that WT PKM2, but not PKM2 K367M, interacted with HDAC3 upon EGF treatment (FIG. 15b). These results suggest that PKM2 can interact with HDAC3 in an active conformation, which facilitates HDAC3 removal from the CCND1 promoter, acetylation of histone H3, and cyclin D1 expression.

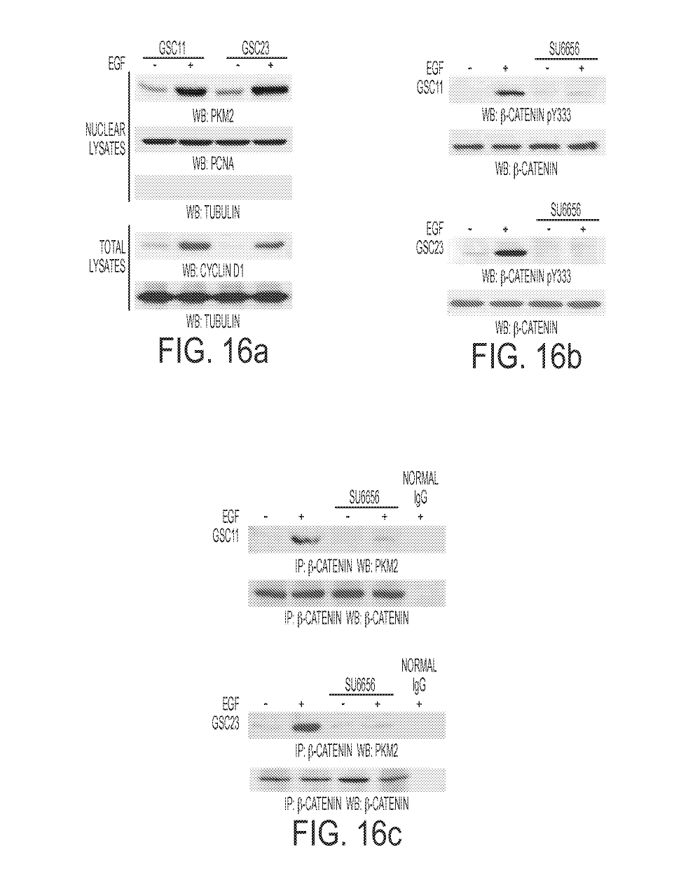

To support the findings that EGF-induced and c-Src-dependent PKM2-β-catenin interaction and subsequent cyclin D1 expression are not cell line-specific, GSC11 and GSC23 human primary GBM cells were treated with EGF. FIG. 16a shows that EGF treatment results in nuclear translocation of PKM2 and cyclin D1 expression in these cells. In addition, EGF-induced phosphorylation of β-catenin Y333 (FIG. 16b) and its association with PKM2 (FIG. 16c) were blocked by pretreatment with SU6656.

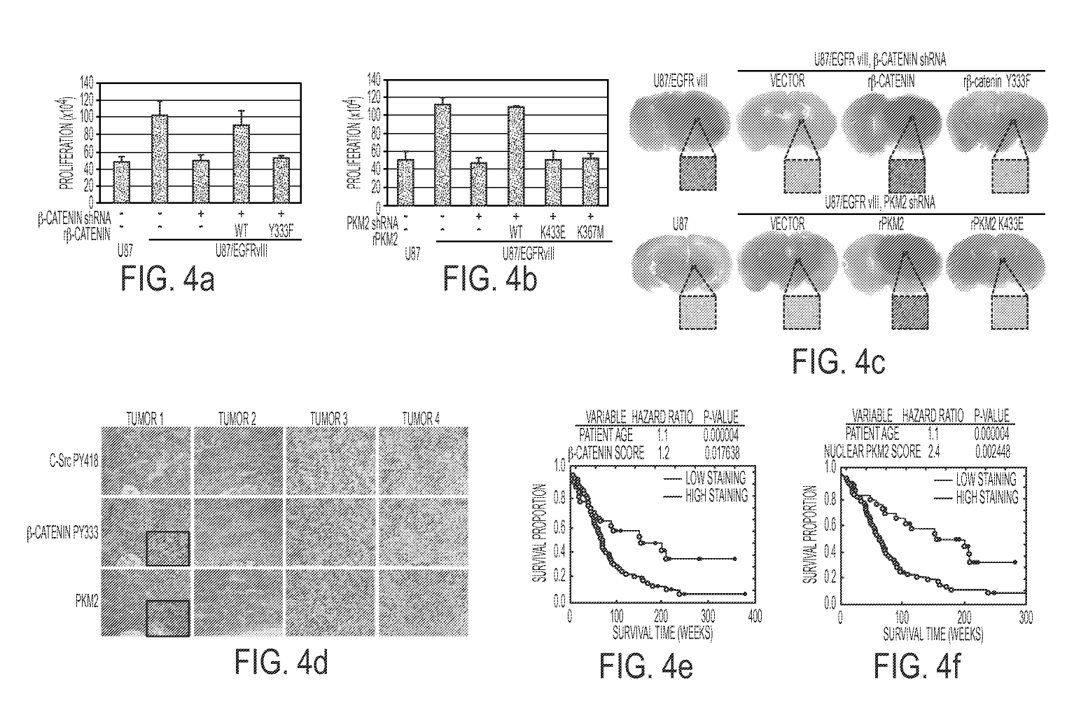

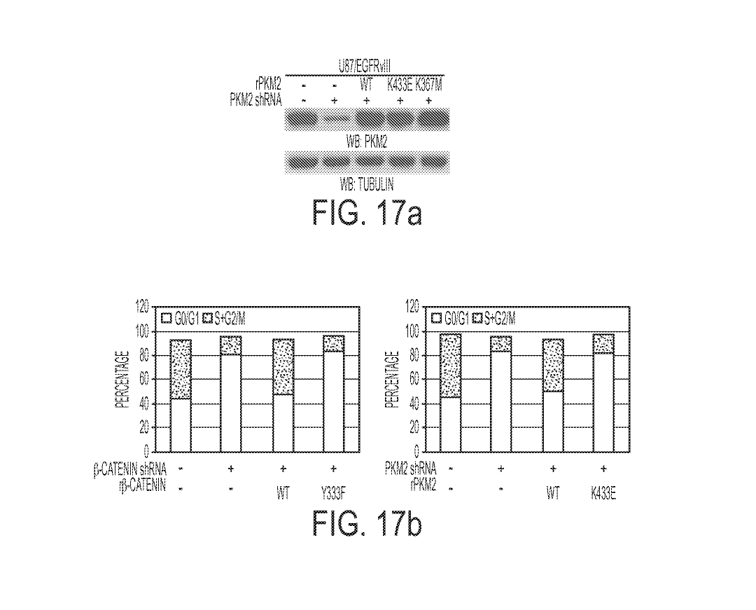

Next the significance of the PKM2-β-catenin interaction in tumor cell proliferation was examined. FIG. 4a shows that U87/EGFRvIII cells proliferated much faster than did parental U87 cells. Expression of β-catenin shRNA (FIG. 3c) largely inhibited U87/EGFRvIII cell proliferation, which was rescued by reconstituted expression of WT rβ-catenin, but not of the rβ-catenin Y333F mutant (FIG. 4a). In addition, EGFRvIII-promoted cell proliferation was similarly inhibited by PKM2 depletion, which was rescued by the reconstituted expression of WT rPKM2, but not of the rPKM2 K433E or rPKM2 K367M mutant (FIG. 17a and FIG. 4b).

β-catenin-regulated cyclin D1 expression is critical for G1-S phase transition and cell cycle progression (Tetsu and McCormick, 1999). Depletion of β-catenin or PKM2 resulted in an accumulation of U87/EGFRvIII cells in G0/G1 phase, which was rescued by reconstituted expression of WT rβ-catenin or WT rPKM2, but not of the rβ-catenin Y333F or rPKM2 K433E mutant (FIG. 17b). Thus, the PKM2-β-catenin interaction is essential for cell cycle progression.

To determine the role of PKM2-dependent β-catenin transactivation in brain tumor development, U87 or U87/EGFRvIII cells were intracranially injected into athymic nude mice. U87 cells did not form a detectable tumor two weeks after injection (FIG. 4c, bottom left panel). In contrast, U87/EGFRvIII cells elicited rapid tumorigenesis (FIG. 4c, top left panel). Notably, depletion of β-catenin (top panel) or PKM2 (bottom panel) abrogated EGFRvIII-driven tumor growth, which was rescued by expression of WT rβ-catenin or WT rPKM2, but not of the rβ-catenin Y333F or rPKM2 K433E mutant. Similar results were obtained using GSC11 cells (FIG. 18a). In addition, adding doxycycline to the drinking water 14 d after intracranial injection of GSC11 cells that expressed a tetracycline-inducible PKM2 shRNA partially depleted PKM2 expression in tumor tissues and inhibited tumor growth (FIG. 18b). Thus, the PKM2-β-catenin interaction and PKM2 expression is instrumental in tumor growth.

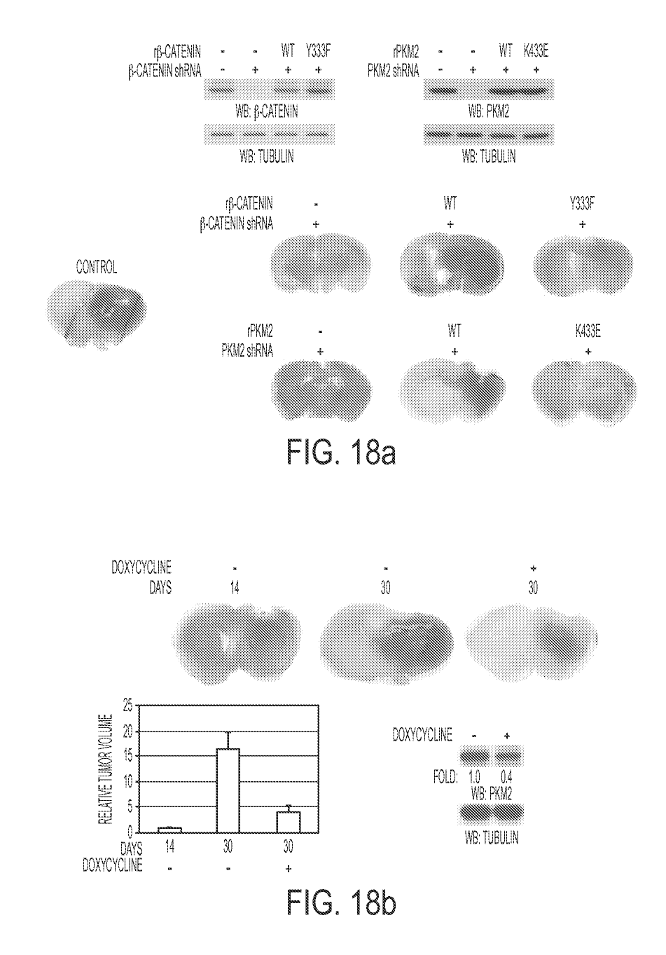

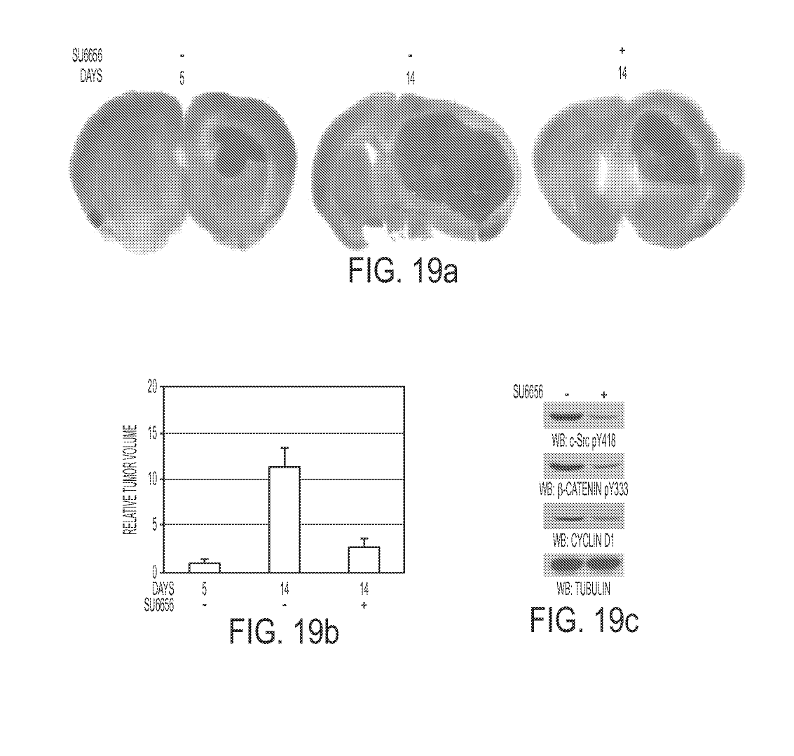

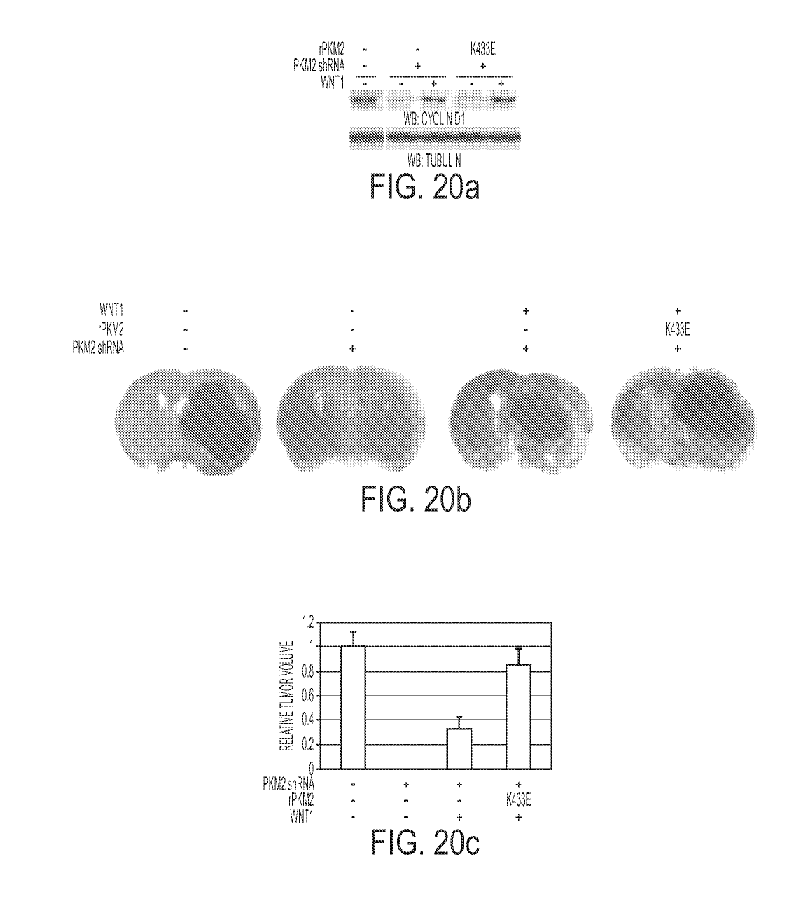

To further support the role of c-Src in EGFR-induced β-catenin transactivation in vivo, SU6656 was injected intratumorally, which significantly blocked tumor growth (FIGS. 19a and 19b), reduced the phosphorylation levels of c-Src Y418 and β-catenin Y333, and inhibited cyclin D1 expression in tumor tissue (FIG. 19c). The requirement of β-catenin transactivation in tumorigenesis was also examined by expression of Wnt1 in U87/EGFRvIII-PKM2 shRNA cells. Wnt1 expression resulted in the induction of cyclin D1 (FIG. 20a) and largely rescued PKM2 depletion-blocked tumorigenesis (FIGS. 20b and 20c). In addition, this effect was further enhanced using Wnt1-expressing U87/EGFRvIII cells with reconstituted expression of rPKM2 K433E, which retains its catalytic activity for glycolysis. These results suggest that, while the metabolic function of PKM2 plays a critical role in aerobic glycolysis (Christofk et al., 2008) and tumorigenesis, brain tumor development promoted by EGFR requires PKM2-modulated β-catenin transactivation.

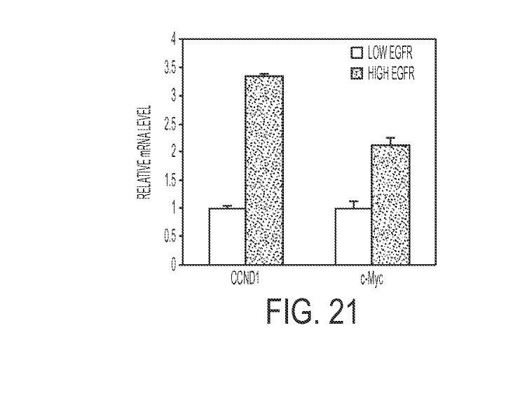

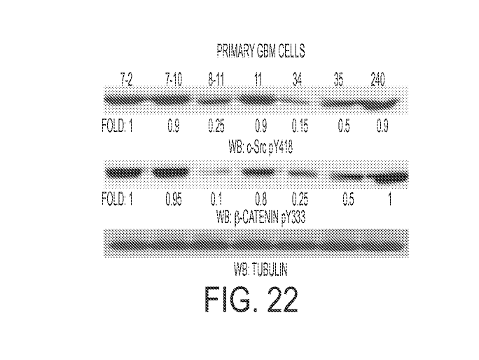

Analysis of publicly available microarray datasets (Affymetrix, U133) from The Cancer Genome Atlas (TCGA) and other sources (Freije et al., 2004; Gravendeel et al., 2009; Petalidis et al., 2008; Phillips et al., 2006) revealed a correlation of c-myc and CCND1 expression with EGFR expression in GBM samples (FIG. 21). In addition, phosphorylation levels of β-catenin Y333 correlated with phosphorylation levels of activated c-Src in seven human primary GBM cell lines (FIG. 22).

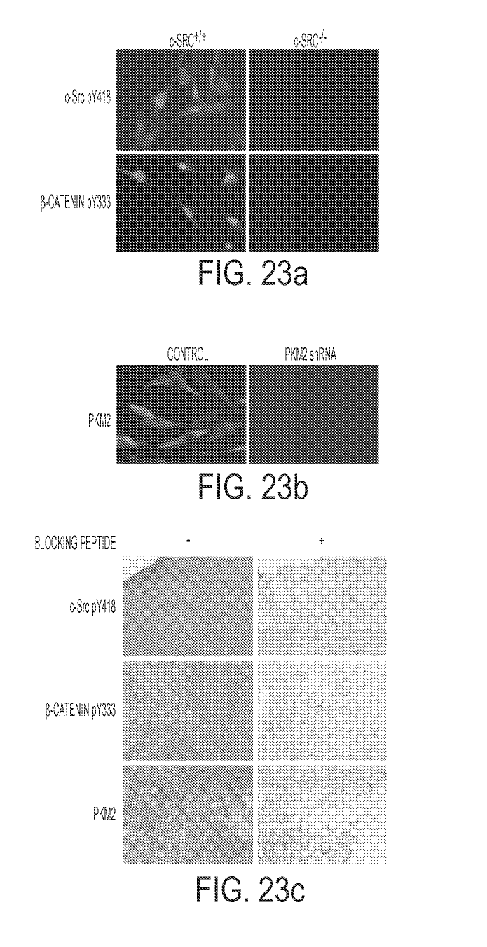

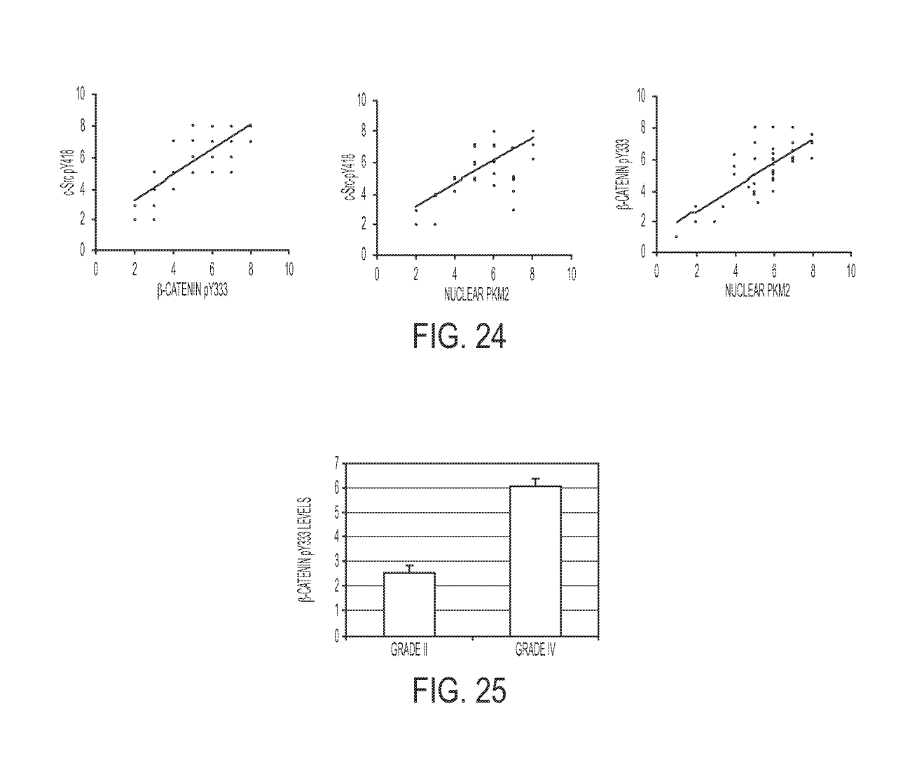

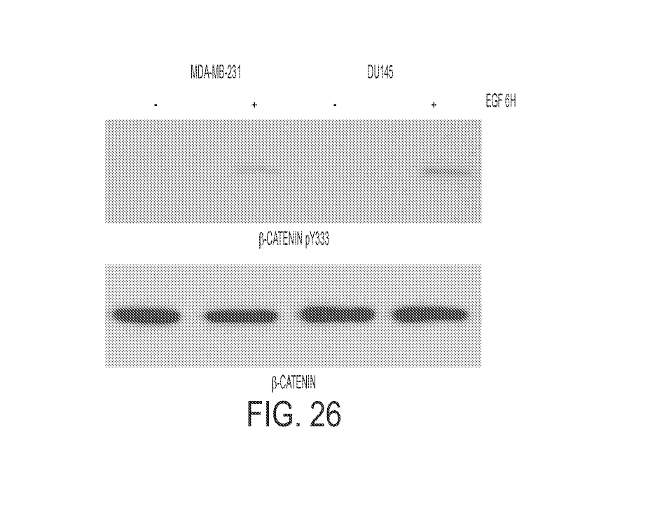

Immunohistochemical (IHC) analyses were next performed to examine c-Src activity, β-catenin Y333 phosphorylation, and PKM2 nuclear localization in serial sections of 55 human primary GBM specimens by using antibodies with validated specificities (FIG. 23). FIG. 4d shows that the levels of c-Src Y418 phosphorylation, 3-catenin Y333 phosphorylation, and nuclear PKM2 expression were correlated with each other. In addition, β-catenin Y333 phosphorylation accumulated in the nuclei of a large percentage of tumor cells (FIG. 4d, left panel). Quantification of the staining on a scale of 0 to 8.0 showed that these correlations were significant (FIG. 24). The survival durations of 84 patients, all of whom received standard adjuvant radiotherapy after surgery, followed by treatment with an alkylating agent (temozolomide in the majority of cases), with low (0-5 staining) versus high (5.1-8 staining) β-catenin Y333 phosphorylation and nuclear PKM2 expression were compared. Patients whose tumors had low β-catenin Y333 phosphorylation or nuclear PKM2 expression had a median survival of 185.2 and 130.0 weeks, respectively. The median survival of patients decreased to 69.4 and 82.5 weeks, respectively, when their tumors showed high levels of β-catenin Y333 phosphorylation or nuclear PKM2 expression. In a Cox multivariate model, the IHC score of β-catenin phosphorylation (FIG. 4e) and nuclear PKM2 expression (FIG. 4f) were independent predictors of glioblastoma patient survival, after adjusting for the age of the patient, a relevant clinical covariate. These results support the role of PKM2 association with c-Src-phosphorylated β-catenin Y333 in the clinical behavior of human GBM and reveal a relationship between β-catenin Y333 phosphorylation/nuclear PKM2 localization and clinical aggressiveness of the tumor. These findings were further supported by IHC analyses, showing significantly lower levels of 3-catenin Y333 phosphorylation in low-grade diffuse astrocytoma (30 cases) (World Health Organization [WHO] grade II; median survival time >5 years) than were present in GBM specimens (WHO grade IV) (Furnari et al., 2007) (FIG. 25).

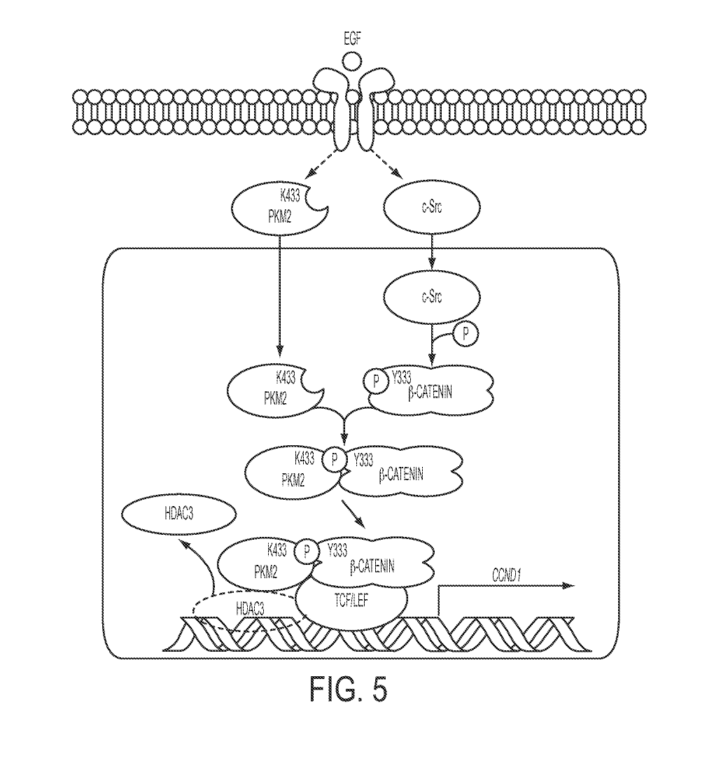

It was shown that GSK-3β-independent transactivation of β-catenin by growth factor receptor occurs by mechanisms distinct from Wnt-dependent canonical signaling (Lu and Hunter, 2004; Ji et al., 2009; Fang et al., 2007; Lu et al., 2003). In the studies detailed above, an important and previously unknown mechanism underlying EGFR activation-induced β-catenin transactivation through interaction with PKM2 was described, which plays a critical role in transcription of CCND1 and c-myc (FIG. 5). The understanding that phosphorylation of β-catenin Y333 and its interaction with PKM2 are required for tumor cell proliferation and tumor development, and that the levels of β-catenin Y333 phosphorylation and nuclear PKM2 correlate with grades of glioma malignancy and prognosis, may provide a molecular basis for improved diagnosis and treatment of tumors with activated EGFR and upregulated PKM2. In summary, these findings, in combination with previous reports (Christofk et al., 2008; Luo et al., 2011), delineate two essential mechanisms underlying tumor development by regulation of metabolic and non-metabolic functions of PKM2: 1) PKM2 enhances aerobic glycolysis (Christofk et al., 2008; Luo et al., 2011) and 2) PKM2 promotes tumor cell proliferation by binding to and transactivating Y333-phosphorylated β-catenin. Thus, PKM2 has dual roles that are essential for tumorigenesis: regulating cancer cell metabolism and gene transcription required for cell proliferation. The coordinated control of metabolism and proliferation by PKM2 is essential for tumorigenesis.

### Discussion

The studies above demonstrate that PKM2, but not PKM1, in response to EGF, translocates into the nucleus and binds to c-Src-phosphorylated β-catenin at Y333, thereby directly regulating β-catenin-dependent gene expression (FIG. 5). Pretreatment with cycloheximide, which blocks protein translation and thereby excludes the potential effect of transcriptionally regulated protein expression on subcellular redistribution of proteins, did not inhibit EGF-induced PKM2 nuclear accumulation (FIG. 6b, right panel). These results suggest that EGF induces the nuclear translocation of PKM2 independent of changes in PKM2 expression. To examine whether cytokines or other growth factors have a similar effect to that of EGF stimulation, U87 cells were treated with interleukin 3, fibroblast growth factor, and plateletderived growth factor. These treatments induced STAT3 phosphorylation, but failed to induce β-catenin Y333 phosphorylation or the PKM2-β-catenin interaction, suggesting that IL3, FGF, and PDGF do not regulate β-catenin similarly to EGF. β-catenin can be phosphorylated at multiple tyrosine residues by distinct protein kinases (Lilien and Balsamo, 2005). Protein tyrosine kinase 6 (PTK6) primarily phosphorylates β-catenin at Y64, which exerts no effect on PTK6-inhibited β-catenin transcriptional activity (Palka-Hamblin et al., 2010). Bcr-ABL phosphorylates β-catenin at Y86 and Y654 and prevents 3-catenin from binding to axin/GSK-3P3 and subsequent phosphorylation by GSK-3P3, thereby enhancing the stability of β-catenin (Coluccia et al., 2007). In an in vitro experiment, purified β-catenin can be phosphorylated by recombinant c-Src, primarily at Y86, in addition to Y654. Phosphorylation of Y654, but not Y86, prevents β-catenin from binding to E-cadherin in vitro (Roura et al., 1999) or in response to UV irradiation (Jean et al., 2009). However, c-Src-dependent β-catenin phosphorylation at these residues has not been validated in vivo. These results show that c-Src phosphorylates β-catenin at Y333 in vitro and that 3-catenin Y333F is largely resistant to c-Src-mediated phosphorylation. In addition, inhibition or deficiency of c-Src or mutation of β-catenin Y333, but not of Y86, blocked EGF-induced nuclear β-catenin phosphorylation. The Src-dependent phosphorylation of β-catenin at Y333 in the nucleus was further validated by experiments using an antibody that could specifically recognize the phosphorylated Y333 of β-catenin. In combination with the evidence that EGF treatment resulted in nuclear translocation of c-Src and an increase in binding of c-Src to nuclear β-catenin, these results indicate that nuclear β-catenin is a physiological substrate of c-Src in response to EGF stimulation. Since co-IP experiments did not detect any associated PKM2 and c-Src, it suggests that c Src disassociates from β-catenin after β-catenin phosphorylation and does not complex with PKM2.

Reconstituted expression of PKM2 K433E and β-catenin Y333F failed to induce tumorigenesis. PKM2 K433E mutation may affect the status of PKM2 oligomerization in cells and the ability of the cells to modulate glycolytic flux (Christofk et al., 2008; Jurica et al., 1998; Mellati et al., 1992). Given that binding of phosphorylated β-catenin Y333 to PKM2 likely contributes to the release of PKM2 allosteric activator FBP and reduces PKM2 activity (Christofk et al., 2008) in the nucleus, expression of β-catenin Y333F could also affect glycolysis if unbound and more active PKM2 exits the nucleus. That PKM2 modulates histone H3 acetylation, thereby downstream gene expression, may indirectly regulate cancer cell metabolism that also contributes to tumorigenesis. However, the defect of PKM2 K433E in promoting tumorigenesis was rescued by Wnt1 expression, supporting the essential role of PKM2-regulated β-catenin transactivation in EGFR-promoted tumor development. The finding of phosphorylation of β-catenin Y333 by c-Src in the nucleus with subsequent interaction with PKM2 indicates that subcellular compartment-specific modification of β-catenin defines its interacting proteins and thereby its functions. β-catenin Y333 phosphorylation is an independent predictor of glioma malignancy, and GBM patient survival distinguishes it as a potential biomarker for both prognosis and selection of GBM treatment with Src inhibitors in clinical practice.

### Materials and Methods

### Cells and Cell Culture Conditions

U87, U87/EGFR, and U251 GBM cells; DU145 prostate cancer cells; MDA-MB-231 breast cancer cells; A431 epidermoid carcinoma cells and NIH3T3, 293T, c-Src^{+/+}, c-Src^{-/-}, Abl^{+/+}, and Abl^{-/-} cells were maintained in Dulbecco's modified Eagle's medium (DMEM) supplemented with 10% bovine calf serum (HyClone, Logan, Utah). Human primary GBM cells were maintained in DMEM/F-12 50/50 supplemented with B27, EGF (10 ng/mL), bFGF (10 ng/mL). Rat pheochromocytoma PC12 cells were maintained in DMEM supplemented with 10% horse serum and 5% fetal bovine serum. Cell cultures were made quiescent by growing them to confluence, and the medium was replaced with fresh medium containing 0.5% serum for 1 d. EGF with 100 ng/mL final concentration was used for cell stimulation.

### Materials

Rabbit polyclonal antibodies recognizing phospho-β-catenin Y333, PKM1, PKM2, c-Src, Abl, phospho-Abl Y412, and c-Myc were obtained from Signalway Biotechnology (Pearland, Tex.). A mouse antibody recognizing phospho-tyrosine was obtained from BD Biosciences (Bedford, Mass.). A monoclonal antibody against phospho-c-Src Y418 was purchased from Millipore (Billerica, Mass.). Polyclonal antibodies for cyclin D1, and PCNA and monoclonal antibodies for β-catenin and Myc tag were purchased from Santa Cruz Biotechnology (Santa Cruz, Calif.). A polyclonal antibody of acetylated histone H3 and a monoclonal antibody for HDAC3 were obtained from Upstate Biotechnology (Lake placid, NY). EGF and mouse monoclonal antibodies for FLAG, GST, His, and tubulin were purchased from Sigma (St. Louis, Mo.). Hygromycin, puromycin, G418, SU6656, Abl inhibitor, cycloheximide, DNase-free RNase A, and propidium iodide were purchased from EMD Biosciences (San Diego, Calif.). Active c-Src was obtained from Signalchem (Richmond, Canada). Hoechst 33342 and Alexa Fluor 488 goat anti-rabbit antibody was from Molecular Probes (Eugene, Oreg.). HyFect transfection reagents were from Denville Scientific (Metuchen, N.J.). GelCode Blue Stain Reagent was obtained from Pierce (Rockford, Ill.).

### Transfection

Cells were plated at a density of 4×10⁵/60 mm dish 18 h prior to transfection. Transfection was performed, as previously described (Xia et al., 2007).

### Immunoprecipitation and Immunoblotting Analysis

Extraction of proteins with a modified buffer from cultured cells was followed by immunoprecipitation and immunoblotting with corresponding antibodies, as described previously (Lu et al., 1998).

### Cell Proliferation Assay

2×10⁴ cells were plated and counted seven days after seeding in DMEM with 0.5% bovine calf serum. Data represent the mean±SD of three independent experiments.

### DNA Constructs and Mutagenesis

PCR-amplified human PKM1 was cloned into pcDNA3.1/hygro (+) vector between BamH I and Xho I. PCR-amplified human PKM2 was cloned into pcDNA3.1/hygro (+) vector between BamH I and Xba I. β-catenin was subcloned into pGEX-4T-1 vector between BamH-I and Not I. pcDNA 3.1/hygro (+)-PKM2, -K433E, -K367M, pcDNA 3.1/hygro (+)-β-catenin Y86F, and -β-catenin Y333F were made using the QuikChange site-directed mutagenesis kit (Stratagene, La Jolla, Calif.). pCDNA 3.1-rPKM2 contains mutations of C1192T and C1194G.

The pGIPZ control was generated with a control oligonucleotide 5′-GCTTCTAACACCGGAGGTCTT-3′ (SEQ ID NO: 1). pGIPZ PKM2 shRNA and pTRIPZ PKM2 shRNA were generated with 5′-TTATTTGAGGAACTCCGCCGC-3′ (SEQ ID NO: 2) oligonucleotide targeting exon 10 of the PKM2 transcript. pGIPZ β-catenin shRNA was generated with 5′-CATGCACAAGAATGGATCACAA-3′ (SEQ ID NO: 3).

### Flow Cytometry Analysis

On million treated cells were fixed in cold 70% ethanol for 3 h, spun down, and incubated for 1 h at 37° C. in PBS with DNase-free RNase A (100 μg/mL) and propidium iodide (50 μg/mL). Cells were then analyzed by fluorescence-activated cell sorting (FACS).

### Purification of Recombinant Proteins

The WT and mutants of GST-β-catenin were expressed in bacteria and purified, as described previously (Xia et al., 2007).

### In Vitro Kinase Assays

The kinase reactions were performed, as described previously (Fang et al., 2007).

### Luciferase Reporter Gene Assay

The transcriptional activation of β-catenin in 293T cells was measured, as previously described (Fang et al., 2007). The relative levels of luciferase activity were normalized to the levels of untreated cells and to the levels of luciferase activity of the Renilla control plasmid.

### ChIP Assay

ChIP was performed using an Upstate Biotechnology kit. Chromatin prepared from cells (in a 10 cm dish) was used to determine total DNA input and for overnight incubation with the specific antibodies or with normal rabbit or mouse immunoglobulin G. The human CCND1 promoter-specific primers used in PCR were 5′-GGGGCGATTTGCATTTCTAT-3′ (SEQ ID NO: 4) (forward) and 5′-CGGTCGTTGAGGAGGTTGG-3′ (SEQ ID NO:5) (reverse).

### Immunofluorescence Analysis

Immunofluorescence analysis was performed, as described previously (Fang et al., 2007).

### Subcellular Fractionation

Nuclei, cytosol, and cell membranes were isolated using the Nuclear Extract Kit from Active Motif North America (Carlsbad, Calif.) and the ProteoExtract Subcellular Proteome Extraction Kit from Calbiochem (San Diego, Calif.).

### Immunohistochemical Analysis

Mouse tumor tissues were fixed and prepared for staining. The specimens were stained with Mayer's hematoxylin and subsequently with eosin (Biogenex Laboratories, San Ramon, Calif.). Afterwards, the slides were mounted using Universal Mount (Research Genetics).

The tissue sections from paraffin-embedded human GBM specimens were stained with antibodies against phospho-c-Src Y418, phospho-β-catenin Y333, PKM2, or nonspecific IgG as a negative control. The tissue sections were quantitatively scored according to the percentage of positive cells and staining intensity, as previously defined (Ji et al., 2009). The following proportion scores were assigned: 0 if 0% of the tumor cells showed positive staining, 1 if 0% to 1% of cells were stained, 2 if 1% to 10% stained, 3 if 11% to 30% stained, 4 if 31% to 70% stained, and 5 if 71% to 100% stained. Intensity of staining was rated on a scale of 0 to 3: 0, negative; 1, weak; 2, moderate; and 3, strong. The proportion and intensity scores were then combined to obtain a total score (range, 0-8), as described previously (Ji et al., 2009). Scores were compared with overall survival, defined as the time from date of diagnosis to death or last known date of follow-up. All patients received standard adjuvant radiotherapy after surgery, followed by treatment with an alkylating agent (temozolomide in the majority of cases). The use of human brain tumor specimens and the database was approved by the institutional review board at MD Anderson Cancer Center.

### Intracranial Injection

GBM cells (5×10⁵) were intracranially injected (in 5 μL of DMEM per mouse), with or without regulation of β-catenin or PKM2 expression, into 4-week-old female athymic nude mice. The intracranial injections were performed, as described in a previous publication (Gomez-Manzano et al., 2006). Seven mice per group in each experiment were included. Animals injected with U87/EGFRvIII or GSC 11 cells were sacrificed 2 weeks or 30 days after glioma cell injection, respectively. The brain of each mouse was harvested, fixed in 4% formaldehyde, and embedded in paraffin. Tumor formation and phenotype were determined by histologic analysis of H & E-stained sections.

For doxycycline induction studies, seven mice were sacrificed 14 days after GBM cell injection to examine tumor growth, whereas the remaining 14 mice in two groups were fed with or without water containing 800 μg/mL doxycycline. The water containing doxycycline was changed every three days. The mice in these two groups were sacrificed at day 30. For the SU6656 treatment studies, seven mice were sacrificed five days after U87/EGFRvIII cell injection to examine tumor growth, whereas the remaining 14 mice in two groups were treated with either DMSO or SU6656. SU6656 (0.015 mg/kg in 5 μL of DMSO) was intracranially injected into the tumor every three days, and the mice in both groups were sacrificed at day 14 after GBM cell injection.

### Neurite Extension Assay

Mouse NGF (Upstate Biotechnology, Lake Placid, N.Y., USA) was added at 100 ng/mL to PC12 cells in culture, and the medium was changed every three days. Two hundred cells from 10 randomly chosen microscopic fields for each condition were examined. A cell was considered to respond to NGF if it extended neurites at least two cell bodies long after the incubation duration.

### Pyruvate Kinase Assay

The activity of bacterially purified WT PKM2 (0.1 μg) and PKM2 K433E (0.1 μg) toward PEP was measured by a pyruvate kinase assay (BioVision, Moutain View, Calif.), according to the manufacturer's instruction. Data represent the mean±SD of three independent experiments.

### Cell Migration Assay

Matrigel-transwell assay was performed, as described previously (Fang et al., 2007).

### Lentivirus Preparation

293T cells were transfected in 150-mm plates with 12 μg pFU-CGW Wnt1, 4 μg μMDL, 4 μg pRSV-Rev, and 4 μg pCMV-VSVG, a plasmid encoding the G-protein of the vesicular stomatitis virus (VSV-G) envelope. The medium was changed the next day. The medium containing lentivirus was harvested at 48 h and 72 h after transfection. Virus particles were concentrated and purified by ultra-high-speed centrifugation (25,000 g for 2 hours at 4° C.). Cells were infected with lentivirus (1×10⁶) in the presence of 6 μg/mL polybrene (Sigma, St. Louis, Mo.).

### Quantitative Real-time PCR

Total RNA was extracted using a RNA High-purity Total RNA Rapid Extraction Kit (Signalway Biotechnology, TX). cDNA was prepared using oligonucleotide (dT), random primers, and a Thermo Reverse Transcription kit (Signalway Biotechnology). Quantitative real-time PCR analysis was performed using 2×SIBR real-time PCR Premixture (Signalway Biotechnology) under the following conditions: 5 min at 95° C. followed by 40 cycles at 95° C. for 30 s, 55° C. for 40 s, and 72° C. for 1 min using an ABI Prism 7700 sequence detection system. Data were normalized to expression of a control gene (3-actin) for each experiment.

The following primer pairs were used for quantitative real-time PCR:

DKK1,
(forward)
(SEQ ID NO: 6)
5′-CATTGACAACTACCAGCCGTAC-3′
and
(reverse)
(SEQ ID NO: 7)
5′-GGGCAGCACATAGCGTGA-3′;
AXIN2,
(forward)
(SEQ ID NO: 8)
5′-GGGAGCCTAAAGGTCGTG-3′
and
(reverse)
(SEQ ID NO: 9)
5′-GGGTTCTCGGGAAATGAG-3′;
βTRCP,
(forward)
(SEQ ID NO: 10)
5′-CCCCAACTGACATTACCC-3′
and
(reverse)
(SEQ ID NO: 11)
5′-TCGAATACAACGCACCAA-3;
β-actin,
(forward)
(SEQ ID NO: 12)
5′-ATGGATGACGATATCGCTGCGC-3′
and
(reverse)
(SEQ ID NO: 13)
5′-GCAGCACAGGGTGCTCCTCA-3′.

### Example 2

### PKM2, Functioning as a Protein Kinase, Phosphorylates Histone H3 and Promotes Gene Transcription and Tumorigenesis

### EGF-Induced and PKM2-Dependent Phosphorylation of Histone H3 at T11 is Required for Acetylation of Histone H3 at K9

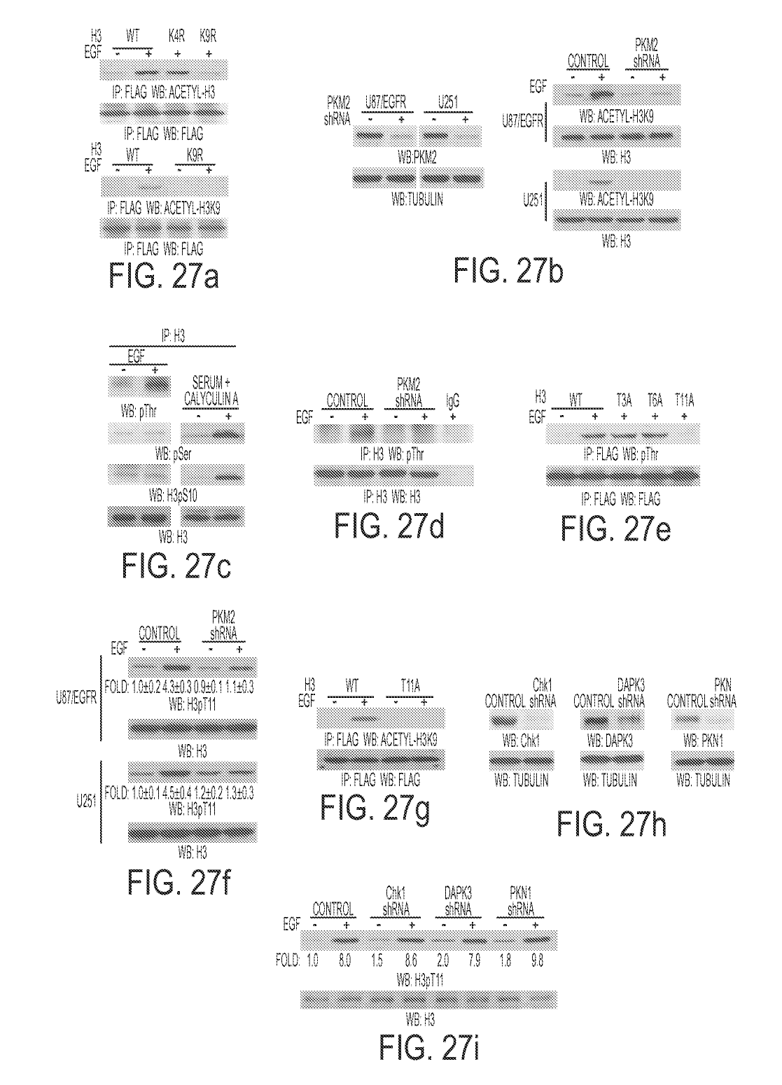

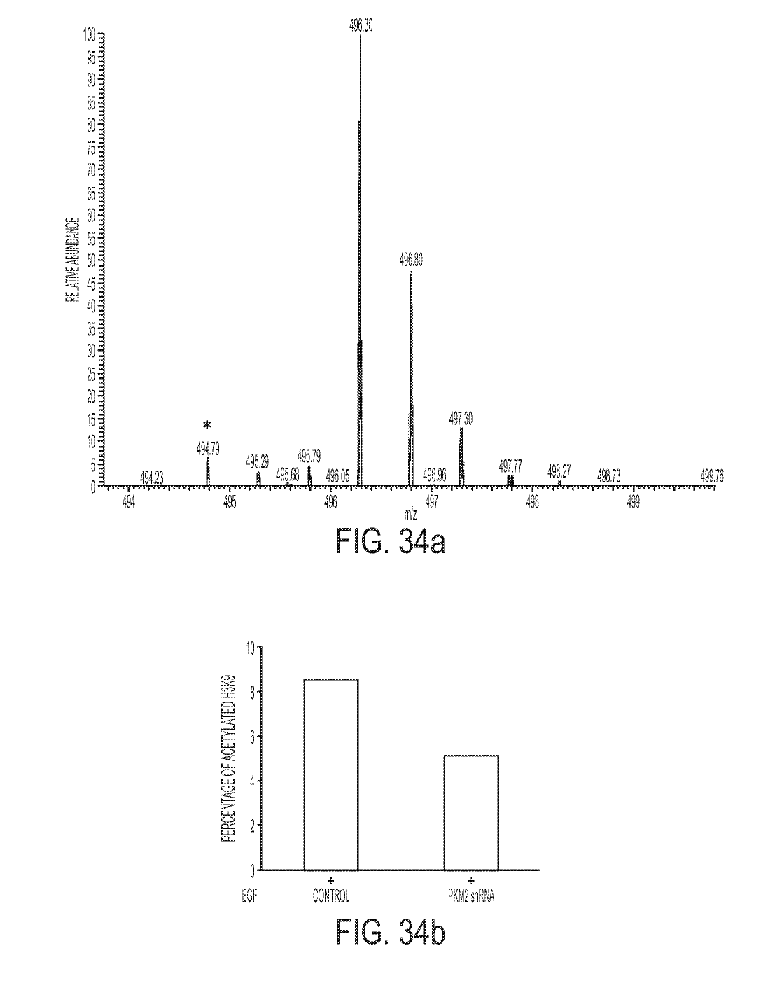

EGFR activation results in PKM2-dependent acetylation of histone H3, which was detected by an anti-acetylated histone H3 antibody recognizing acetylated K4 and K9 (Yang et al., 2011) (FIG. 27A). To identify the Lys residue in histone H3 acetylated upon EGFR activation, FLAG-tagged K4R or K9R mutants of histone H3, in which the individual lysines were mutated into arginine, were expressed in U87/EGFR human glioblastoma multiforme (GBM) cells. Immunoblotting analysis with an anti-acetylated H3 antibody showed that histone H3 K9R, but not histone H3 K4R, was resistant to acetylation induced by EGF stimulation (FIG. 27A). In addition, histone H3 K9R mutation abrogated EGF-induced H3-K9 acetylation recognized by a specific H3-K9 acetylation antibody (FIG. 27A). shRNA-induced depletion of PKM2 (FIG. 27B) in U87/EGFR and/or U251 GBM cells blocked EGF-induced H3-K9 acetylation, as detected by immunoblotting analysis, which was further validated by liquid chromatography-coupled mass spectrometric (LC-MS/MS) analyses of a tryptic digest of immunoprecipitated endogenous histone H3 (FIG. 34). These results indicate that PKM2 is required for EGF-induced H3-K9 acetylation.

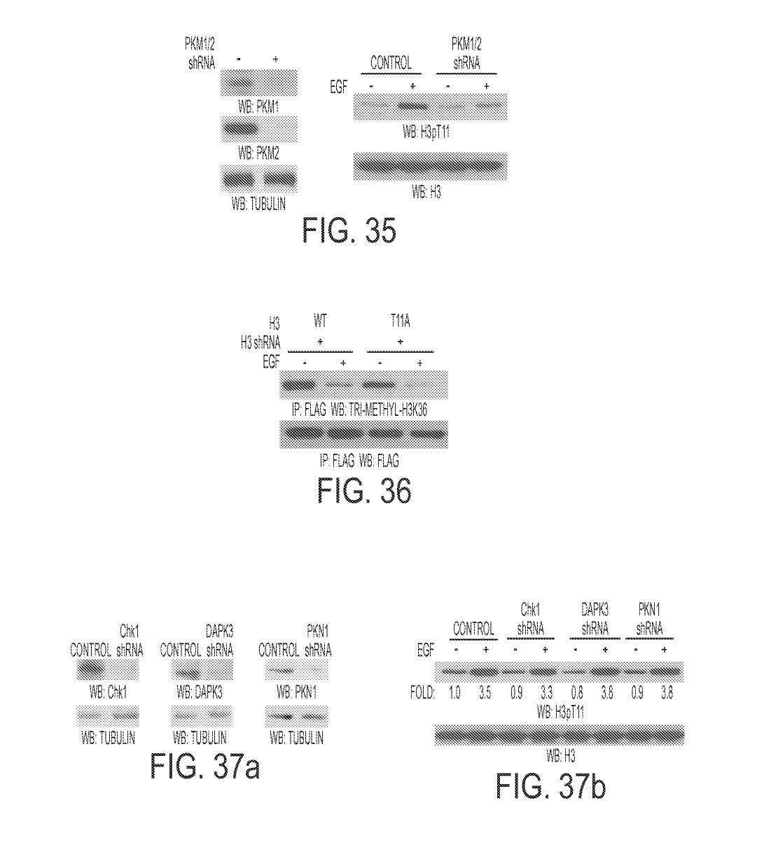

Histones can undergo several different posttranslational modifications, including acetylation, phosphorylation, methylation, and ubiquitylation. Histone modifications can influence one another, such that one modification is required for the generation of a different modification for subsequent gene transcription regulation (Lee et al., 2010; Suganuma and Workman, 2008). Given that phosphorylation of a histone H3 serine or threonine residue can lead to acetylation of its adjacent Lys (Baek, 2011; Perez-Cadahia et al., 2009; Shimada and Nakanishi, 2008; Shimada et al., 2008), next it was examined whether EGF induces histone H3 phosphorylation, which may be essential for H3-K9 acetylation. Immunoblotting analyses of immunoprecipitated histone H3 with antibodies for phospho-threonine, phospho-serine, or phospho-histone H3-S10 showed that EGFR activation increased total levels of phosphorylated threonine (FIG. 27C); however, EGF stimulation, unlike treatment with serum and calyculin A (a serine/threonine phosphatase inhibitor), failed to increase total levels of phosphorylated serine or S10 in histone H3. Notably, PKM2 depletion prevented EGF-induced Thr-phosphorylation of histone H3 (FIG. 27D). Mutation of T3, T6, and T11 of histone H3, which lie close to K9, into alanine (Ala, A) showed that the H3-T11A mutant, but not the H3-T3A or H3-T6A mutant, was resistant to Thr-phosphorylation induced by EGF stimulation (FIG. 27E). Furthermore, EGF-induced H3-T11 phosphorylation, detected with a phospho-H3-T11-specific antibody, was reduced by PKM2 depletion (FIG. 27F) or depletion of both PKM2 and PKM1 (FIG. 35). Immunoblotting analyses of immunoprecipitated wild-type FLAG-histone H3 and FLAG-histone H3-T11A with an anti-acetylated H3-K9 antibody showed that the T11A mutation abrogated EGF-induced histone H3 acetylation at K9 (FIG. 27G) without affecting the status of K36 trimethylation (FIG. 36). These results, which are in line with a previous finding that H3-T11 phosphorylation is required for K9 acetylation (Shimada and Nakanishi, 2008), indicate that PKM2-dependent H3-T11 phosphorylation primes K9 acetylation upon EGFR activation.

Chk1 (Shimada et al., 2008), death-associated protein (DAP)-like kinase (Dlk, also termed DAPK3 and ZIPK) (Preuss et al., 2003), and protein-kinase-C-related kinase 1 (PRK1/PKN1) (Metzger et al., 2008) are reported to phosphorylate H3-T11. To examine the potential involvement of these protein kinases in EGF-regulated H3-K9 acetylation, EGF was used to treat U87/EGFR (FIG. 27H) or U251 (FIG. 37A) cells with or without expressing shRNA against Chk1, DAPK3, or PKN1 (encoding PRK1). As shown in FIGS. 27I and 37B, depletion of mRNA expression of Chk1, DAPK3, and PKN1 did not affect EGF-induced H3-T11 phosphorylation, further supporting the finding that PKM2 specifically regulates H3-T11 phosphorylation and subsequent H3-K9 acetylation upon EGFR activation.

PKM2 Directly Interacts with Histone H3 and Phosphorylates H3-T11

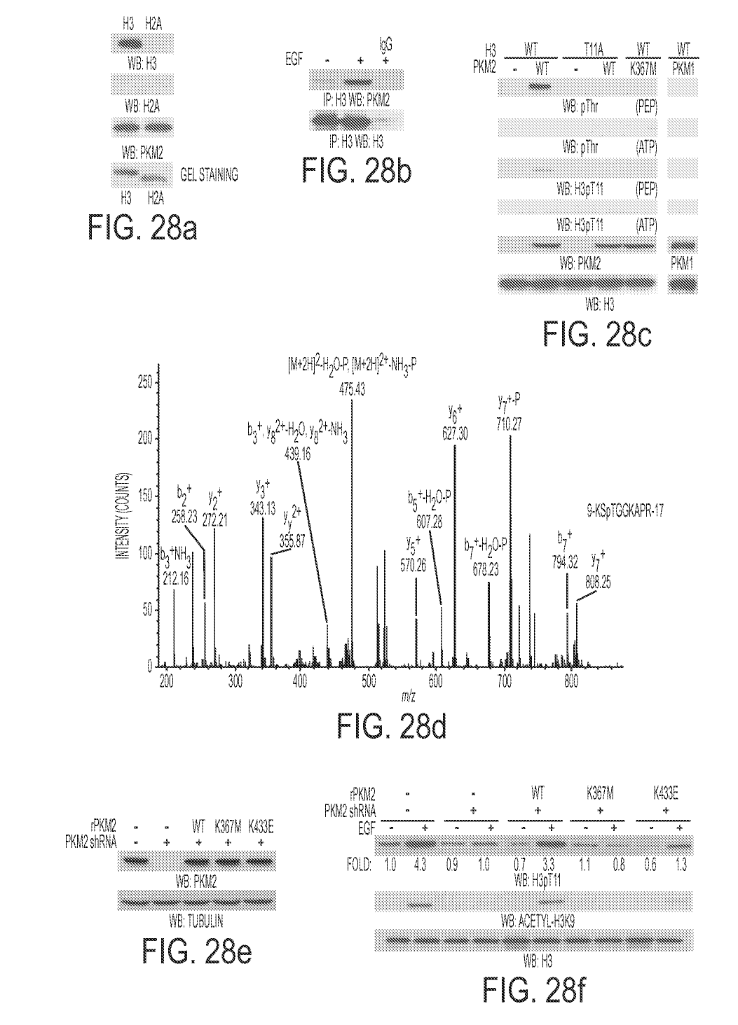

To further determine the relationship between PKM2 and phosphorylation of H3-T11, the interaction between these two proteins was examined. Pull-down analyses by mixing purified recombinant His-PKM2 on nickel agarose beads with purified recombinant histone H3 or histone H2A showed that PKM2 directly bound to histone H3 but not to histone H2A (FIG. 28A). Immunoblotting analyses of immunoprecipitated endogenous histone H3 with an anti-PKM2 antibody showed that EGF stimulation resulted in increased binding of PKM2 to histone H3 (FIG. 28B). These results indicate that PKM2 interacts with histone H3 both in vitro and in cells.

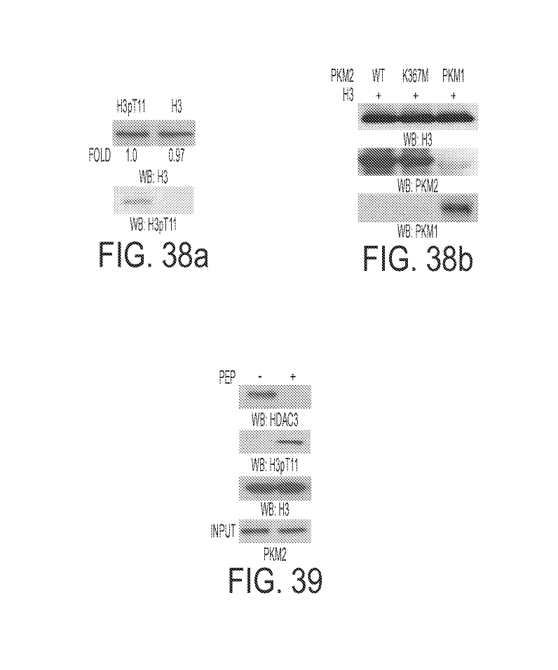

Next it was examined whether histone H3 might be directly phosphorylated by the catalytic activity of PKM2. An in vitro phosphorylation analysis using ATP as the phosphate group donor did not detect any histone H3 phosphorylation by recombinant PKM2, as detected by immunoblotting with an anti-phospho-Thr antibody or a phospho-H3-T11-specific antibody. However, incubation of PKM2 with histone H3 in the presence of PEP, the physiological phosphate group donor of PKM2, showed that WT PKM2, but not PKM2 K367M kinase-dead mutant (Yang et al., 2011) or PKM1, phosphorylated WT histone H3 but not H3-T11A (FIG. 28C and FIG. 38A), although recombinant PKM2 K367M and PKM1 were able to interact with histone H3 (FIG. 38B). Furthermore, LC-MS/MS analysis identified T11, but not S10, as a residue phosphorylated by PKM2 (FIG. 28D). In addition, reconstituted expression of RNAi-resistant inactive rPKM2 K367M mutant, unlike the re-expression of its WT counterpart (WT rPKM2), in endogenous PKM2-depleted U87/EGFR cells (FIG. 28E), failed to rescue EGF-induced H3-T11 phosphorylation or H3-K9 acetylation (FIG. 28F). Intriguingly, reconstituted expression of a PKM2 K433E mutant, which loses its binding ability to tyrosine-phosphorylated proteins including β-catenin and promoter regions, such as CCND1 and MYC (Christofk et al., 2008b; Yang et al., 2011), also largely failed to restore EGF-induced H3-T11 phosphorylation or H3-K9 acetylation (FIGS. 28E and 28F). These results indicate that PKM2 directly binds to histone H3 and phosphorylates histone H3 at T11, which is required for subsequent H3-K9 acetylation.

PKM2-Dependent H3-T11 Phosphorylation Promotes the Disassociation of HDAC3 from CCND1 and MYC Promoter

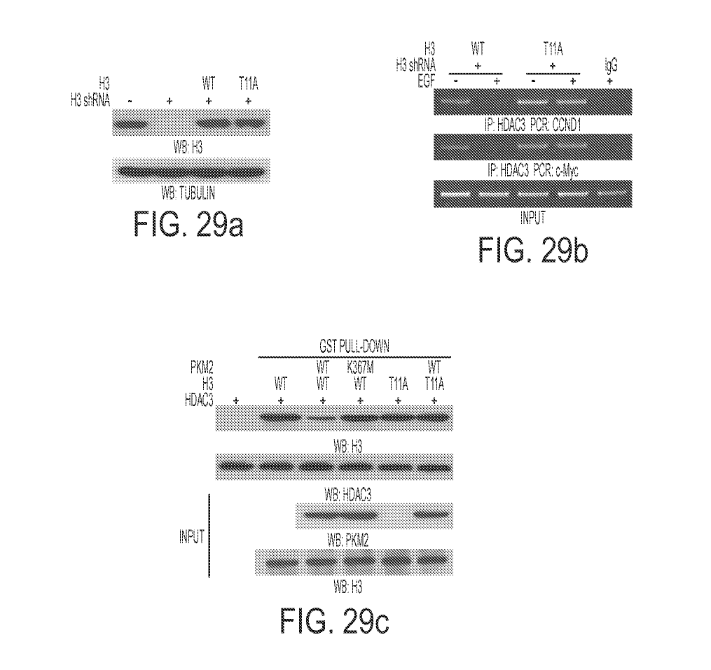

As demonstrated above, the binding of PKM2 to the CCND1 promoter is required for the dissociation of HDAC3 from the promoter (Yang et al., 2011). To examine whether PKM2-regulated H3-K9 acetylation is mediated by HDAC3 dissociation from the CCND1 promoter, which, in turn, requires prior H3-T11 phosphorylation, chromatin immunoprecipitation (ChIP) analyses were performed with an HDAC3 antibody. As shown in FIG. 29, reconstituted expression of RNAi-resistant histone rH3-T11A, compared with re-expression of its WT counterpart in endogenous histone H3-depleted U87/EGFR cells (FIG. 29A), blocked EGF-induced HDAC3 dissociation from the CCND1 and MYC promoters (FIG. 29B). (The histone H3-depleted U87/EGFR cell line without reconstituted H3 expression was not stable, and H3 expression in these cells recovered after prolonged cultures; these cells were not used for further experiments.) To further support the finding that PKM2-dependent H3-T11 phosphorylation promotes the dissociation of HDAC3 from histone H3, in vitro binding analyses were performed by mixing purified recombinant GST-HDAC3 on agarose beads and purified recombinant WT histone H3 or histone H3-T11A mutant, which was followed by incubation with or without purified recombinant WT PKM2 or PKM2 K367M mutant in a PEP-containing kinase buffer. As shown in FIG. 29C, GST-HDAC3 interacted with both WT histone H3 and histone H3-T11A. Intriguingly, the presence of WT PKM2, but not of PKM2 K367M, resulted in the dissociation of HDAC3 from WT histone H3 but not from histone H3-T11A. In addition, pre-incubation of histone H3 with recombinant PKM2 in the presence or absence of PEP before incubating with GST-HDAC3 showed that histone H3 phosphorylated by PKM2 lost its ability to interact with HDAC3 (FIG. 39). These results indicate that PKM2-dependent H3-T11 phosphorylation promotes HDAC3 dissociation from histone H3 and facilitates subsequent H3-K9 acetylation.

PKM2-Dependent H3-T11 Phosphorylation Promotes EGF-Induced Expression of Cyclin D1 and c-Myc

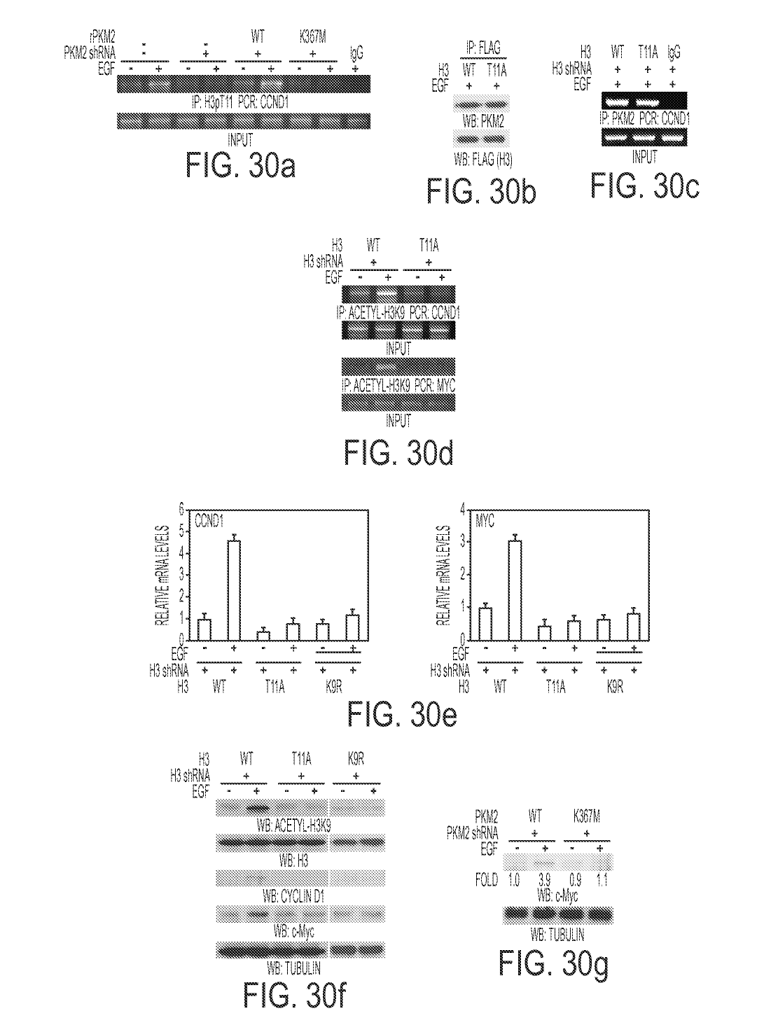

EGFR activation results in complex formation between PKM2 and β-catenin, which leads to binding of the complex to the CCND1 and MYC promoter regions and subsequent histone H3 acetylation at the promoters (Yang et al., 2011). To determine whether PKM2 regulates cyclin D1 and c-Myc expression via modulating H3-T11 phosphorylation at the promoter regions, ChIP analyses were performed with anti-phospho-H3-T11. As shown in FIG. 30A, EGF treatment resulted in enhanced H3-T11 phosphorylation at the CCND1 promoter, which was prevented by PKM2 depletion. Reconstituted expression of RNAi-resistant rPKM2 K367M in U87/EGFR cells (FIG. 28D), unlike its WT counterpart, failed to rescue EGF-induced H3-T11 phosphorylation at the CCND1 promoter (FIG. 30A). Given that WT PKM2 and PKM2 K367M have comparable affinity for CCND1 promoter regions (Yang et al., 2011), these results indicate that the kinase activity of PKM2 is required for EGF-induced H3-T11 phosphorylation at the CCND1 promoter.

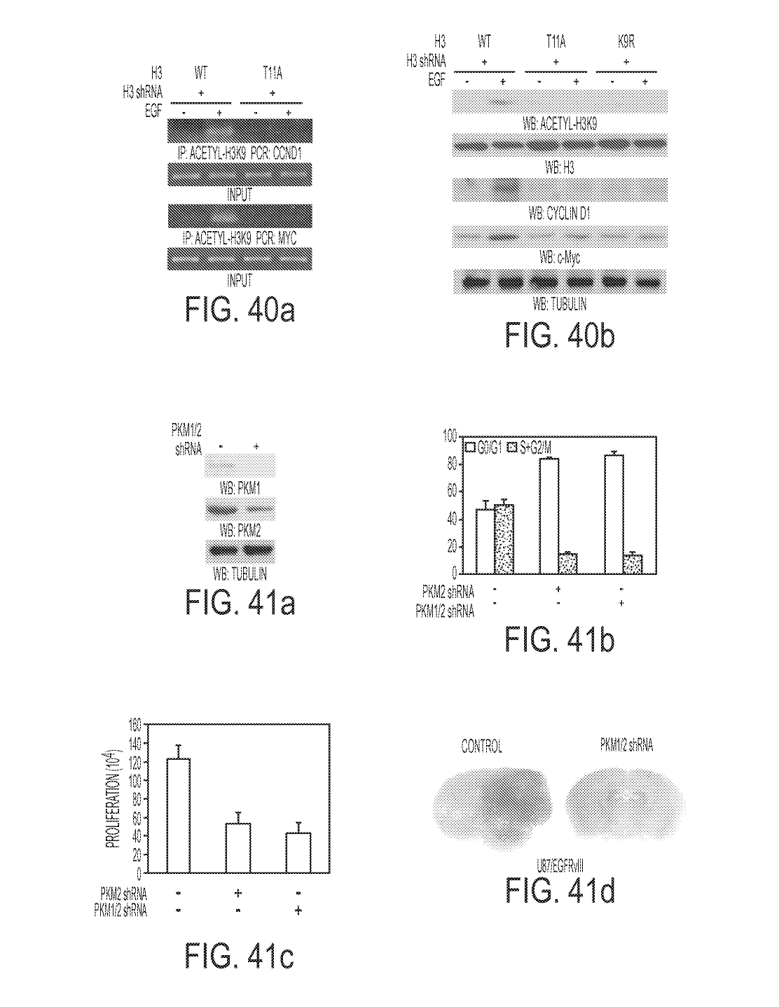

Next the significance of H3-T11 phosphorylation in EGF-induced cyclin D1 and c-Myc expression was investigated by reconstituting the expression of WT histone rH3 and histone rH3-T11A in endogenous histone H3-depleted U87/EGFR (FIG. 29A) and U251 (FIG. 40B) cells. Immunoblotting analyses of immunoprecipitated FLAG-tagged histone H3 with an anti-PKM2 antibody showed that FLAG-tagged histone H3-T11A, acting like its WT counterpart, binds to PKM2 upon EGF stimulation (FIG. 30B). ChIP analyses with a PKM2 antibody demonstrated that PKM2 binds to CCND1 promoter regions to a similar degree in WT histone H3-expressing and histone H3-T11A-expressing cells (FIG. 30C). However, expression of histone H3-T11A blocked EGF-induced H3-K9 acetylation at CCND1 and MYC promoter regions in both U87/EGFR (FIG. 30D) and U251 cells (FIG. 40A), as demonstrated by ChIP analyses with an anti-acetylated H3-K9 antibody. In addition, H3-T11A expression abrogated EGF-enhanced mRNA levels of CCND1 and MYC (FIG. 30E), H3-K9 acetylation, and expression of cyclin D1 and c-Myc in both U87/EGFR (FIG. 30F) and U251 (FIG. 40B) cells. Furthermore, reconstituted expression of H3-K9R blocked EGF-induced expression of cyclin D1 and c-Myc at both mRNA and protein expression levels (FIGS. 30E and 30F). In line with the finding that PKM2 kinase activity is required for EGF-induced cyclin D1 expression (Yang et al., 2011), reconstituted expression of PKM2 K367M, compared with re-expression of its WT counterpart in U87/EGFR cells with depleted endogenous PKM2 (FIG. 28D), blocked EGF-induced c-Myc expression (FIG. 30G). These results indicate that PKM2 phosphorylates H3-T11 at CCND1 and MYC promoter regions, which is required for subsequent H3-K9 acetylation and transcription of the genes.

### PKM2-Dependent H3-T11 Phosphorylation is Required for Cell Cycle Progression, Cell Proliferation, and Tumorigenesis

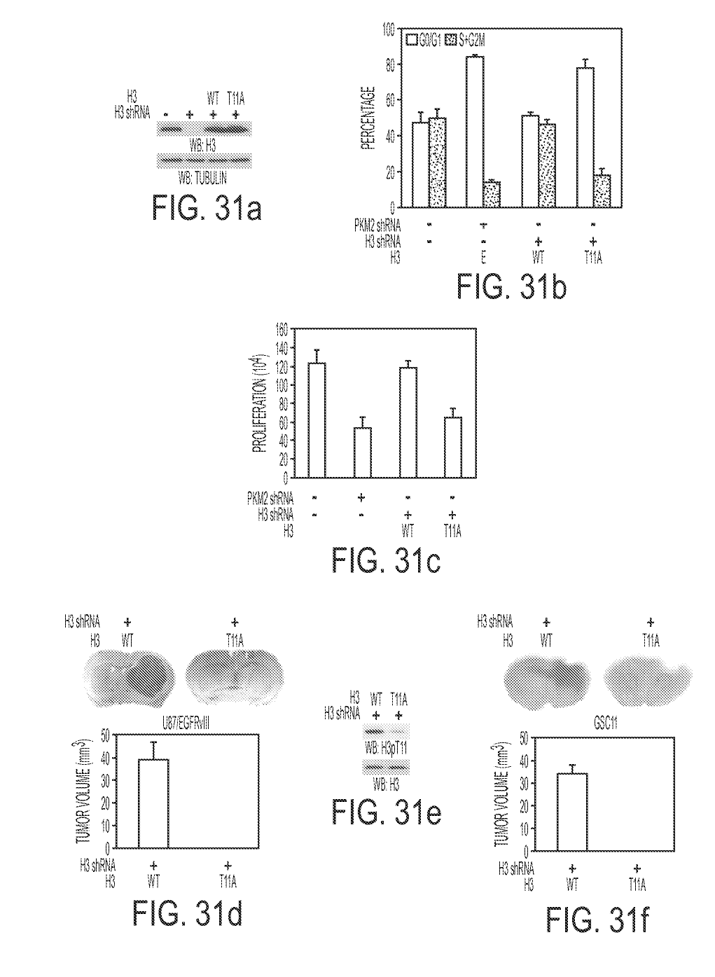

Cyclin D1 expression is required for the G1-S phase transition (Resnitzky and Reed, 1995). To examine whether PKM2-dependent H3-T11 phosphorylation, which promotes cyclin D1 expression, regulates the G1-S phase transition, the expression of RNAi-resistant WT histone rH3 or rH3-T11A in endogenous histone H3-depleted U87 cells was reconstituted by expressing a constitutively active EGFRvIII mutant (FIG. 31A). As shown in FIG. 31B, expression of histone rH3-T11A, compared with expression of WT histone rH3, resulted in accumulation of U87/EGFRvIII cells in the G0/G1 phase, as determined by flow cytometric analyses. In addition, expression of histone rH3-T11A, in contrast to expression of its WT counterpart, inhibited cell proliferation (FIG. 31C). The inhibitory effect on cell cycle progression and cell proliferation was also observed by depletion of PKM2 (FIG. 31B-C) (Yang et al., 2011) or depletion of both PKM2 and PKM1 (FIG. 41A-C). These results strongly suggest that PKM2-dependent H3-T11 phosphorylation is required for cell cycle progression and cell proliferation.

Depletion of PKM2 (Yang et al., 2011) or PKM1/2 abrogated brain tumorigenesis induced by intracranial injection of U87/EGFRvIII cells (FIG. 41D). To determine the role of PKM2-dependent H3-T11 phosphorylation in brain tumor development, endogenous histone H3-depleted U87/EGFRvIII cells with reconstituted expression of WT histone rH3 or histone rH3-T11A mutant were intracranially injected. U87/EGFRvIII cells expressing WT histone rH3 elicited rapid tumorigenesis (FIG. 31D). In addition, the levels of phosphorylated histone H3 at T11 were higher in the tumor tissue derived from the mice injected with U87/EGFvIII cells with reconstituted expression of WT histone H3 than in the counterpart tissue derived form the mice injected with U87/EGFvIII cells with reconstituted expression of histone H3 T11A (FIG. 31E). In contrast, histone rH3-T11A expression abrogated EGFRvIII-driven tumor growth. Similar results were obtained by using GSC11 human primary GBM cells with endogenous histone H3 depletion and reconstituted expression of WT histone rH3 or rH3-T11A (FIG. 31F). These results indicate that PKM2-dependent H3-T11 phosphorylation is instrumental in EGFR-promoted tumor development.

H3-T11 Phosphorylation Positively Correlates with the Level of Nuclear PKM2 Expression and with Grades of Glioma Malignancy and Prognosis

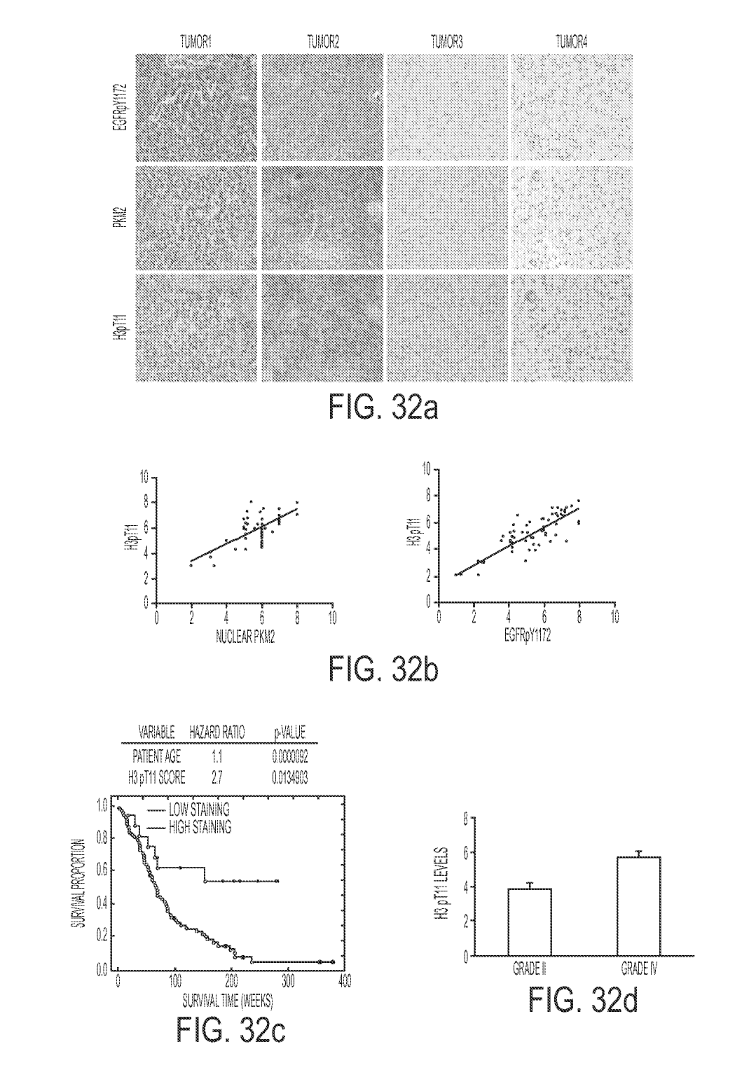

The nuclear expression level of PKM2 correlates with poor GBM prognosis (Yang et al., 2011). To further define the clinical relevance of the finding that nuclear PKM2 phosphorylates H3-T11 upon EGFR activation, IHC analyses were used to examine the activity levels of EGFR reflected by their phosphorylation levels, H3-T11 phosphorylation, and PKM2 nuclear localization in serial sections of 45 human primary GBM specimens (World Health Organization [WHO] grade IV). The antibody specificities were validated by using IHC analyses with specific blocking peptides. As shown in FIG. 32A, levels of H3-T11 phosphorylation, nuclear PKM2 expression, and EGFR activity were correlated with each other. Quantification of the staining on a scale of 0 to 8.0 showed that these correlations were significant (FIG. 32B).

Survival durations of 85 patients were compared, all of whom received standard adjuvant radiotherapy after surgery, followed by treatment with an alkylating agent (temozolomide in most cases), with low (0-4 staining) versus high (4.1-8 staining) H3-T11 phosphorylation. Patients whose tumors had low H3-T11 phosphorylation (16 cases) had a median survival that was not reached; those whose tumors had high levels of H3-T11 phosphorylation (69 cases) had a significantly lower median survival duration of 77 weeks. In a Cox multivariate model, the IHC score of H3-T11 phosphorylation (FIG. 32C, P=0.013490) was an independent predictor of GBM patient survival, after adjusting for patient age, a relevant clinical covariate. These results support a role for PKM2-dependent H3-T11 phosphorylation in the clinical behavior of human GBM and reveal a relationship between H3-T11 phosphorylation and clinical aggressiveness of the tumor. To further explore this relationship, the levels of H3-T11 phosphorylation were examined to determine whether the levels correlated with the grades of glioma malignancy. Levels of H3-T11 phosphorylation in samples from patients (30 cases) with low-grade diffuse astrocytoma (WHO grade II; median survival time >5 years) were compared with those from patients with high-grade GBM (Furnari et al., 2007). IHC analysis showed significantly lower levels of H3-T11 phosphorylation in low-grade tumors than were present in GBM specimens (FIG. 32D).

### Discussion

The mechanisms underlying PKM2-regulated transcriptional control of gene expression were not previously known. The studies here demonstrate that PKM2, functioning as a protein kinase, interacts with histone H3 and phosphorylates H3-T11, which leads to HDAC3 removal from CCND1 and MYC promoter regions and subsequently to K9 acetylation and gene transcription.

These findings, significantly enrich the understanding of the physiological role of PKM2 in tumor development by revealing its two integrated functions: 1) PKM2 act as a glycolytic enzyme transferring a phosphate group from PEP to ADP for ATP generation and pyruvate production. It is also a rate-limiting controller of glycolysis needed for generation of glucose metabolites to synthesize amino acids, phospholipids, and nucleic acids, which are building blocks for cell growth and cell proliferation (Hsu and Sabatini, 2008; Koppenol et al., 2011; Vander Heiden et al., 2009). 2) PKM2 acts as a protein kinase phosphorylating histone for gene transcription, which directly controls cell cycle progression and cell proliferation. This line of evidence establishes PKM2 as a unique and key regulator of cancer development by virtue of its coordination of ATP generation, macromolecular syntheses, and gene transcription via both metabolic and nonmetabolic functions.

EGFR activation results in the nuclear translocation of PKM2, but not PKM1, which restricts the accessibility of PKM1 to histone. PEP participates in the phosphorylation of H11 in phosphoglycerate mutase (PGAM1), but not through PKM2 acting as a PGAM1 kinase (Vander Heiden et al., 2010). It has been reported that histone H3 T11 can be phosphorylated by several protein kinases (Shimada et al., 2008; Metzger et al., 2008; Preuss et al., 2003). Dlk/DAPK3/ZIPK phosphorylates H3-T11 in mitosis. However, the role of H3-T11 phosphorylation in mitosis is not clear (Preuss et al., 2003). Basal Chk1 activity was reported for phosphorylation of H3-T11 in interphase, and DNA damage, which phosphorylates and activates Chk1, causes the dissociation of Chk1 from chromatin and H3-T11 dephosphorylation (Shimada et al., 2008). In addition, androgen stimulation enhances H3-T11 phosphorylation in prostate cancer cells in a PRK1/PKN1-dependent manner (Metzger et al., 2008). However, depletion of DAPK3, Chk1, or PRK1/PKN1 did not affect EGF-induced H3-T11 phosphorylation, further supporting the idea that nuclear translocation of PKM2 induced by EGFR activation plays a critical role in H3-T11 phosphorylation, which promotes G1-S phase transition and cell cycle progression.

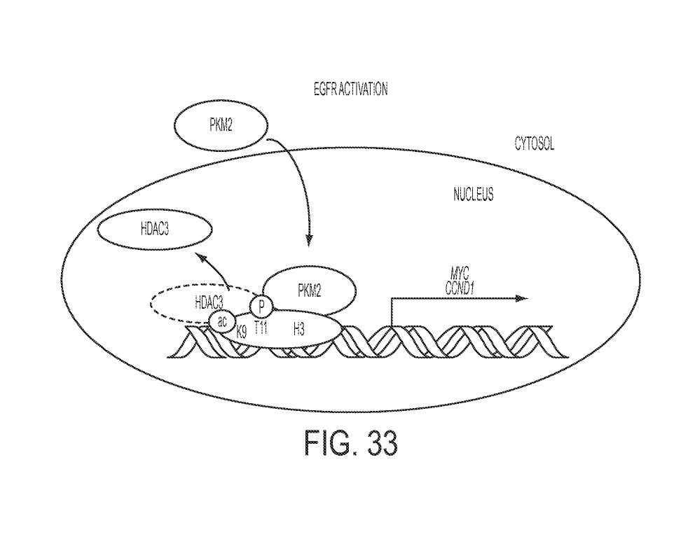

EGFR activation results in GSK-3β-independent β-catenin transactivation by mechanisms distinct from Wnt-dependent canonical signaling (Fang et al., 2007; Ji et al., 2009; Lu et al., 2003; Lu and Hunter, 2004; Yang et al., 2011). EGFR activation results in nuclear translocation of PKM2, which interacts with Y333-phosphorylated β-catenin (Yang et al., 2011). This protein complex binds to the CCND1 and MYC promoter regions, where PKM phosphorylates H3-T11, leading to HDAC3 disassociation from the promoters and subsequent acetylation of histone H3, transcription of genes, and cell cycle progression (FIG. 33). The finding that PKM2-dependent H3-T11 phosphorylation, which regulates total cellular histone H3 acetylation levels, is required for tumor cell proliferation and tumorigenesis, and that the levels of H3-T11 phosphorylation and nuclear PKM2 correlate with grades of glioma malignancy and prognosis, may provide a molecular basis for improved diagnosis and treatment of tumors with activated EGFR and upregulated PKM2.

### Materials and Methods

### Materials

Rabbit polyclonal antibodies recognizing phospho-histone H3 T11, phospho-histone H3 S10, phospho-EGFR Y1172, PKM1, PKM2, and c-Myc were obtained from Signalway Biotechnology (Pearland, Tex.). Rabbit polyclonal antibodies recognizing histone H3, histone H2A, tri-methyl-histone H3 K36, tri-methyl-histone H3 K79, and phospho-histone H3 T11 were obtained from Abcam (Cambridge, Mass.). Mouse antibodies recognizing phospho-tyrosine and phospho-serine were obtained from BD Biosciences (Bedford, Mass.). Polyclonal antibodies for Chk1, PKN1, cyclin D1, and PCNA and a monoclonal antibody for phospho-threonine were purchased from Santa Cruz Biotechnology (Santa Cruz, Calif.). EGF and mouse monoclonal antibodies for FLAG, His, DAPK3, and tubulin were purchased from Sigma (St. Louis, Mo.). A polyclonal antibody specific for acetylated histone H3 K9, a monoclonal antibody for HDAC3, hygromycin, puromycin, G418, DNase-free RNase A, and propidium iodide were purchased from EMD Biosciences (San Diego, Calif.). HyFect transfection reagents were from Denville Scientific (Metuchen, N.J.). GelCode Blue Stain Reagent was obtained from Pierce (Rockford, Ill.). Purified histone H3 was from New England Biolab (Ipswich, Mass.).

### Cells and Cell Culture Conditions

U87, U87/EGFR, and U251 GBM cells and 293T cells were maintained in Dulbecco's modified Eagle's medium (DMEM) supplemented with 10% bovine calf serum (HyClone, Logan, Utah). Human primary GSC11 GBM cells were maintained in DMEM/F-12 50/50 supplemented with B27, EGF (10 ng/mL), and bFGF (10 ng/mL). Cell cultures were made quiescent by growing them to confluence, and the medium was replaced with fresh medium containing 0.5% serum for 1 d. EGF at a final concentration of 100 ng/mL was used for cell stimulation.

### Transfection

Cells were plated at a density of 4×10⁵/60-mm dish at 18 h before transfection. Transfection was performed as previously described (Xia et al., 2007).

### Mass Spectrometry Analysis

An in vitro PKM2-phosphorylated sample of purified H3 was exhaustively acetylated with acetic anhydride and triethylamine in acetonitrile, evaporated to dryness, then resuspended in 50 mM ammonium bicarbonate buffer containing Rapigest (Waters Corp, MA). The sample was heated to 95° C. for 10 min and then allowed to cool; 100 ng of sequencing-grade modified trypsin (Promega, Madison, Wis.) was added. The digestion proceeded overnight at 37° C. and was analyzed by LC-MS/MS on an Obitrap-XL mass spectrometer (Thermo Fisher Scientific, Waltham, Mass.).

Proteins were identified by a database search of the fragment spectra against the National SwissProt protein database (EBI) using Mascot v.2.3 (Matrix Science, London, UK) and Sequest (v.1.20) via Proteome Discoverer v.1.3 (Thermo Fisher Scientific). Phosphopeptide matches were analyzed by using PhosphoRS implemented in Proteome Discoverer and manually curated (Taus et al., 2011).

### Immunoprecipitation and Immunoblotting Analysis

Extraction of proteins with a modified buffer from cultured cells was followed by immunoprecipitation and immunoblotting with corresponding antibodies as described previously (Lu et al., 1998).

### Cell Proliferation Assay

A total of 2×10⁴ cells were plated and counted seven days after seeding in DMEM with 0.5% bovine calf serum. Data represent the mean±SD of three independent experiments.

### DNA Constructs and Mutagenesis

Polymerase chain reaction (PCR)-amplified human PKM2 was cloned into pcDNA3.1/hygro (+) vector between BamH I and Not I. pcDNA 3.1/hygro (+)-PKM2 K367M, pcDNA 3.1/hygro (+)-Histone H3 K4R, -K9R, -T3A, -T6A, and -T11A were made by using the QuikChange site-directed mutagenesis kit (Stratagene, La Jolla, Calif.). pcDNA 3.1-rPKM2 contains non-sense mutations of C1170T, C1173T, T1174C, and G1176T.

The pGIPZ control was generated with control oligonucleotide 5′-GCTTCTAACACCGGAGGTCTT-3′ (SEQ ID NO: 1). pGIPZ PKM2 shRNA was generated with CATCTACCACTTGCAATTA (SEQ ID NO: 14) oligonucleotide targeting exon 10 of the PKM2 transcript. pGIPZ PKM1/2 shRNA was generated with 5′-GATTATCAGCAAAATCGAG-3′ (SEQ ID NO: 15). pGIPZ Histone H3 shRNA was generated with 5′-CCTATGAAAGGATGCAATA-3′ (SEQ ID NO: 16). pGIPZ Chk1 shRNA was generated with 5′-GCAACAGTATTTCGGTATA-3′ (SEQ ID NO: 17). pGIPZ DAPK3 shRNA was generated with 5′-AAGCAGGAGACGCTCACCA-3′ (SEQ ID NO: 18). pGIPZ PKN1 shRNA was generated with 5′-CCCGGACCACGGGTGACAT-3′ (SEQ ID NO: 19).

### Flow Cytometric Analysis

A total of 1×10⁶ treated cells were fixed in cold 70% ethanol for 3 h, spun down, and incubated for 1 h at 37° C. in PBS with DNase-free RNase A (100 μg/mL) and propidium iodide (50 μg/mL). Cells were then analyzed with use of a fluorescence-activated cell sorter (FACS).

### Purification of Recombinant Proteins

The WT and mutants of His-PKM2, His-PKM1, and His-histone H3 and GST-HDAC3 were expressed in bacteria and purified as described previously (Xia et al., 2007).

### In Vitro Kinase Assays

The kinase reactions were performed as described previously (Fang et al., 2007; Vander Heiden et al., 2010). In brief, the bacterially purified recombinant PKM2 (200 ng) were incubated with histone H3 (100 ng) with kinase buffer (50 mM Tris-HCl [pH 7.5], 100 mM KCl, 50 mM MgCl₂, 1 mM Na₃VO4, 1 mM DTT, 5% glycerol, 0.5 mM PEP, 0.05 mM FBP) in 25 μL at 25° C. for 1 h. The reactions were terminated by the addition of sodium dodecyl sulfate-polyacrylamide gel electrophoresis (SDS-PAGE) loading buffer and heated to 100° C. The reaction mixtures were then subjected to SDS-PAGE analyses.

### ChIP Assay

ChIP was performed by using SimpleChIP® Enzymatic Chromatin IP Kits. Chromatin prepared from cells (in a 10-cm dish) was used to determine total DNA input and for overnight incubation with the specific antibodies or with normal rabbit or mouse immunoglobulin G. The human CCND1 promoter-specific primers used in PCR were 5′-GGGGCGATTTGCATTTCTAT-3′ (SEQ ID NO: 4) (forward) and 5′-CGGTCGTTGAGGAGGTTGG-3′ (SEQ ID NO: 5) (reverse). MYC promoter-specific primers were 5′-CAGCCCGAGACTGTTGC-3′ (SEQ ID NO: 20) (forward) and 5′-CAGAGCGTGGGATGTTAG-3′ (SEQ ID NO: 21) (reverse).

### Immunofluorescence Analysis

Immunofluorescence analyses were performed as described previously (Fang et al., 2007).

### Immunohistochemical Analysis

Mouse tumor tissues were fixed and prepared for staining. The specimens were stained with Mayer's hematoxylin and subsequently with eosin (Biogenex Laboratories, San Ramon, Calif.). Afterward, the slides were mounted with use of a Universal Mount (Research Genetics Huntsville, Ala.).

The tissue sections from paraffin-embedded human GBM specimens were stained with antibodies against phospho-histone H3 T11, PKM2, or nonspecific IgG as a negative control. The tissue sections were quantitatively scored according to the percentage of positive cells and staining intensity, as previously defined (Ji et al., 2009). The following proportion scores were assigned: 0 if 0% of the tumor cells showed positive staining, 1 if 0% to 1% of cells were stained, 2 if 2% to 10% were stained, 3 if 11% to 30% were stained, 4 if 31% to 70% were stained, and 5 if 71% to 100% were stained. The intensity of staining was rated on a scale of 0 to 3: 0, negative; 1, weak; 2, moderate; and 3, strong. The proportion and intensity scores were then combined to obtain a total score (range, 0-8), as described previously (Ji et al., 2009). Scores were compared with overall survival, defined as the time from date of diagnosis to death or last known date of follow-up. All patients received standard adjuvant radiotherapy after surgery, followed by treatment with an alkylating agent (temozolomide in most cases). The use of human brain tumor specimens and the database was approved by the Institutional Review Board at MD Anderson Cancer Center.

### Intracranial Injection

GBM cells (5×10⁵ in 5 μL of DMEM per mouse) with endogenous histone H3 depletion and reconstituted expression of histone H3 WT or T11V were intracranially injected into 4-week-old female athymic nude mice. The intracranial injections were performed as described in a previous publication (Gomez-Manzano et al., 2006). Seven mice per group in each experiment were included. Animals injected with U87/EGFRvIII or GSC 11 cells were sacrificed 14 or 30 days after glioma cell injection, respectively. The brain of each mouse was harvested, fixed in 4% formaldehyde, and embedded in paraffin. Tumor formation and phenotype were determined by histologic analysis of H & E-stained sections.

### Quantitative Real-Time PCR

Total RNA was extracted with use of an RNA High-purity Total RNA Rapid Extraction Kit (Signalway Biotechnology). cDNA was prepared by using oligonucleotide (dT), random primers, and a Thermo Reverse Transcription kit (Signalway Biotechnology). Quantitative real-time PCR analysis was performed using 2×SIBR real-time PCR Premixture (Signalway Biotechnology) under the following conditions: 5 min at 95° C. followed by 40 cycles at 95° C. for 30 s, 55° C. for 40 s, and 72° C. for 1 min using an ABI Prism 7700 sequence detection system. Data were normalized to expression of a control gene (β-actin) for each experiment.

The following primer pairs were used for quantitative real-time PCR:

CCND1,
(forward)
(SEQ ID NO: 22)
5′-GCGAGGAACAGAAGTGC-3′
and
(reverse)
(SEQ ID NO: 23)
5′-GAGTTGTCGGTGTAGATGC-3′;
MYC,
(forward)
(SEQ ID NO: 24)
5′-ACACCCTTCTCCCTTCG-3′
and
(reverse)
(SEQ ID NO: 25)
5′-CCGCTCCACATACAGTCC-3′;
β-actin,
(forward)
(SEQ ID NO: 12)
5′-ATGGATGACGATATCGCTGCGC-3′
and
(reverse)
(SEQ ID NO: 13)
5′-GCAGCACAGGGTGCTCCTCA-3′.

### Example 3

### ERK1/2-Dependent Phosphorylation and Nuclear Translocation of PKM2 Promotes the Warburg Effect

The studies presented below demonstrate that extracellular signal-regulated kinase (ERK) phosphorylation-dependent nuclear translocation of PKM2 is required for the autoregulation of PKM2 expression and PKM2-dependent expression of glycolytic genes, which are essential for the EGFR-promoted Warburg effect and tumorigenesis.

### Results

### ERK is Required for PKM2 Nucleus Translocation

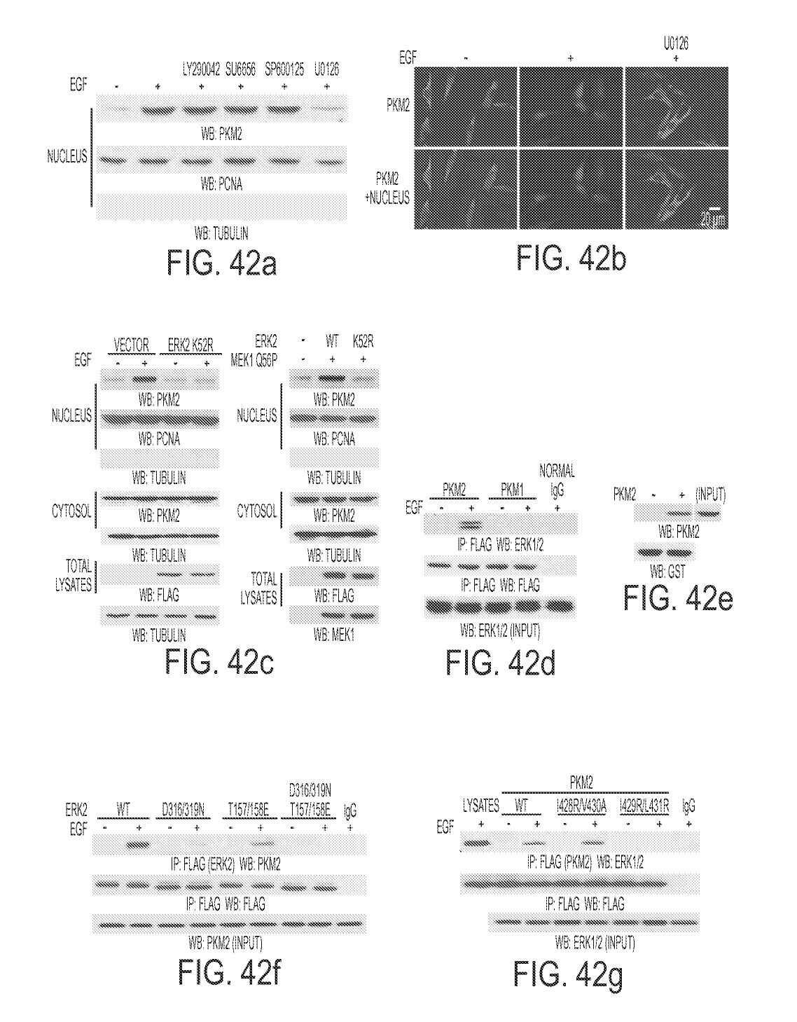

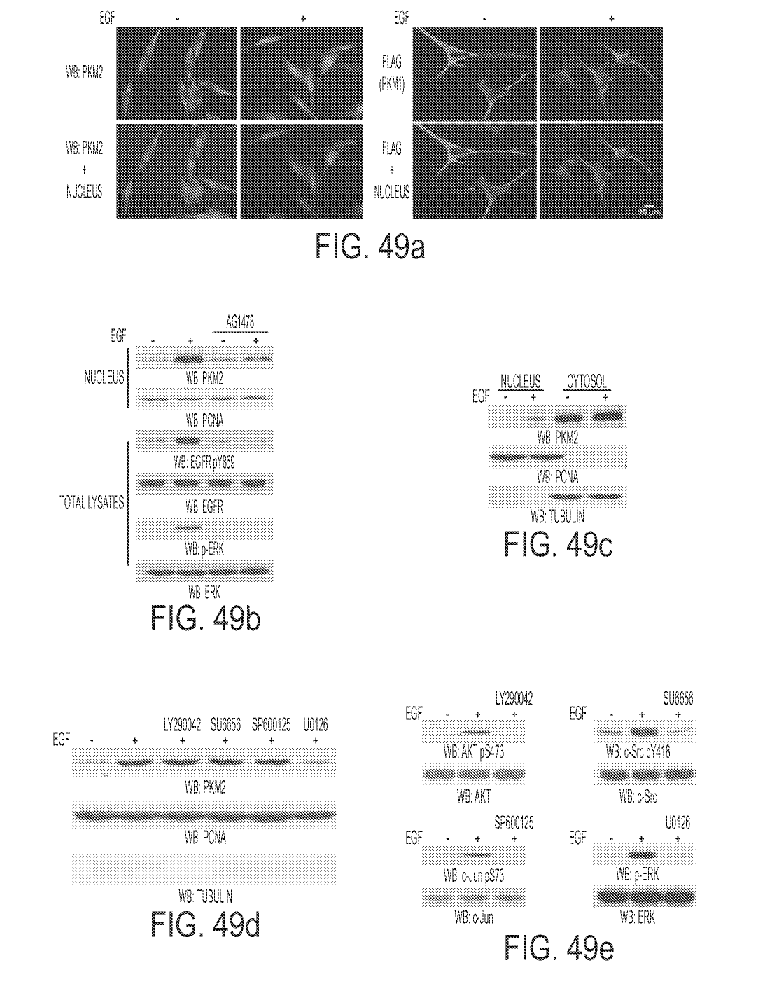

To understand the mechanism of PKM2 accumulation in the nucleus, the inventors performed immunofluorescence analysis and showed that PKM2, a primarily cytosolic protein, translocated into the nucleus upon EGF stimulation in U251 human glioblastoma multiforme (GBM) cells (FIG. 49a, left panel). This result is in line with the inventors findings that EGFR activation by EGF or expression of the constitutively activated EGFRvIII mutant promotes nuclear translocation of PKM2 (Yang et al., 2011). In contrast, FLAG-PKM1 did not show any subcellular redistribution upon EGF stimulation (FIG. 49a, right panel). Treatment with EGFR inhibitor AG1478, which blocked EGF-induced phosphorylation of EGFR and ERK1/2, abrogated EGF-induced nuclear accumulation of PKM2 (FIG. 49b). Compared to the amount of cytosolic PKM2, nuclear PKM2 is a small portion (FIG. 49c), which may account for no reduction of cytoplasmic PKM2 expression upon EGF treatment. Pretreatment of U87/EGFR (FIG. 42a) and U251 cells (FIG. 49d) with the phosphoinositide 3-kinase inhibitor LY290042, Src inhibitor SU6656, JNK inhibitor SP600125, and MEK/ERK inhibitor U0126 blocked EGF-induced phosphorylation of AKT, c-Src, c-Jun, and ERK1/2, respectively (FIG. 49e). Immunoblotting analyses showed that only inhibition of MEK/ERK abrogated EGF-induced nuclear translocation of PKM2 (FIG. 42a; FIG. 49d). These results were further supported by immunofluorescence analyses (FIG. 42b). In addition, expression of the FLAG-ERK2 K52R kinase-dead mutant blocked EGF-induced nuclear accumulation of PKM2 (FIG. 42c, left panel). Furthermore, coexpression of the constitutively active MEK1 Q56P mutant with FLAG-tagged wild-type (WT) ERK2 or ERK2 K52R in U251 cells (FIG. 42c, right panel) showed that expression of WT ERK2, but not ERK2 K52R, induced nuclear translocation of PKM2. These results indicate that ERK activation is required for EGF-induced nuclear translocation of PKM2.

To further determine the relationship between ERK1/2 and PKM2, the inventors performed a co-immunoprecipitation assay and revealed that EGF treatment resulted in ERK1/2 binding to FLAG-PKM2 but not FLAG-PKM1 (FIG. 42d). Moreover, an in vitro GST pull-down assay with mixed purified GST-ERK2 and His-PKM2 showed that these two proteins interacted directly (FIG. 42e). MAP kinases bind to their substrates through a docking groove comprised of an acidic common docking (CD) domain and glutamic acid-aspartic acid (ED) pockets (Lu and Xu, 2006). Immunoblotting of the immunoprecipitated FLAG-ERK2 proteins with an anti-PKM2 antibody showed that mutation of either the ERK2 CD domain (D316/319N) or the ED pocket (T157/158E) reduced binding to endogenous PKM2, as compared with the WT ERK2 control. Combined mutations at both the CD domain and ED pocket (T/E-D/N) abrogated the binding of ERK2 to PKM2 entirely (FIG. 42f), indicating that ERK2 binds to PKM2 through its docking groove.

ERK substrates often have a docking (D) domain, which is characterized by a cluster of basic residues followed by an LXL motif (L represents Leu, but can also be Ile or Val; X represents any amino acid) (Lu and Xu, 2006). Analysis of the PKM2 amino acid sequence with the Scansite program identified the putative ERK-binding sequence 422-KCCSGAIIVLTKSGR-436 (SEQ ID NO: 26) in the aa 380-434 region, which contains LXL motifs at I428/V430 and I429/L431. This series of amino acids is encoded by the PKM2-specific exon 10 and is thus unique to PKM2. Immunoblotting of the immunoprecipitated FLAG-PKM2 proteins with an anti-ERK1/2 antibody showed that a PKM2 I429/L431A mutant, but not a PKM2 I428R/V430A mutant, drastically reduced its binding to ERK1/2 (FIG. 42g). These results indicate that the ERK2 docking groove binds to a D domain in PKM2 at I429/L431.

### ERK2 Phosphorylates PKM2 S37

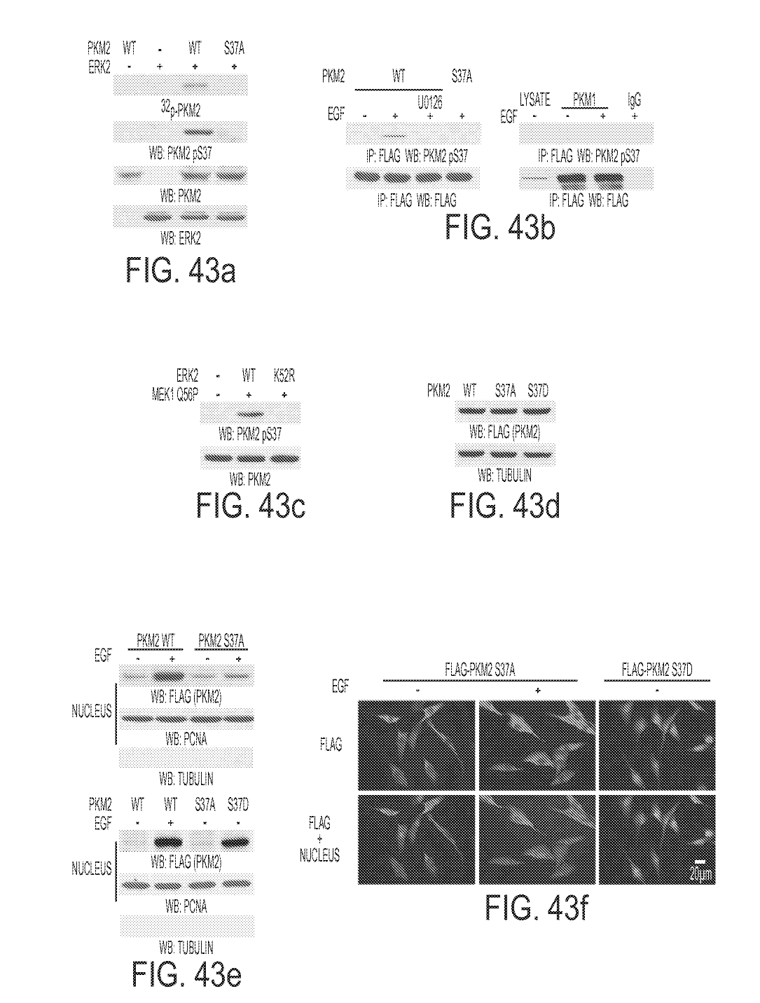

The inventors performed an in vitro kinase assay by mixing purified PKM2 with active ERK2 and showed that ERK2 phosphorylated PKM2 (FIG. 43a). Sequence analysis of PKM2 revealed that it contains an ERK consensus phosphorylation motif (Ser-Pro) (Lu and Xu, 2006) at the S37/P38 residues. The S37A mutation completely abrogated the ERK2-dependent phosphorylation of PKM2 in vitro; this finding was further validated by a specific phospho-PKM2 S37 antibody (FIG. 43a).

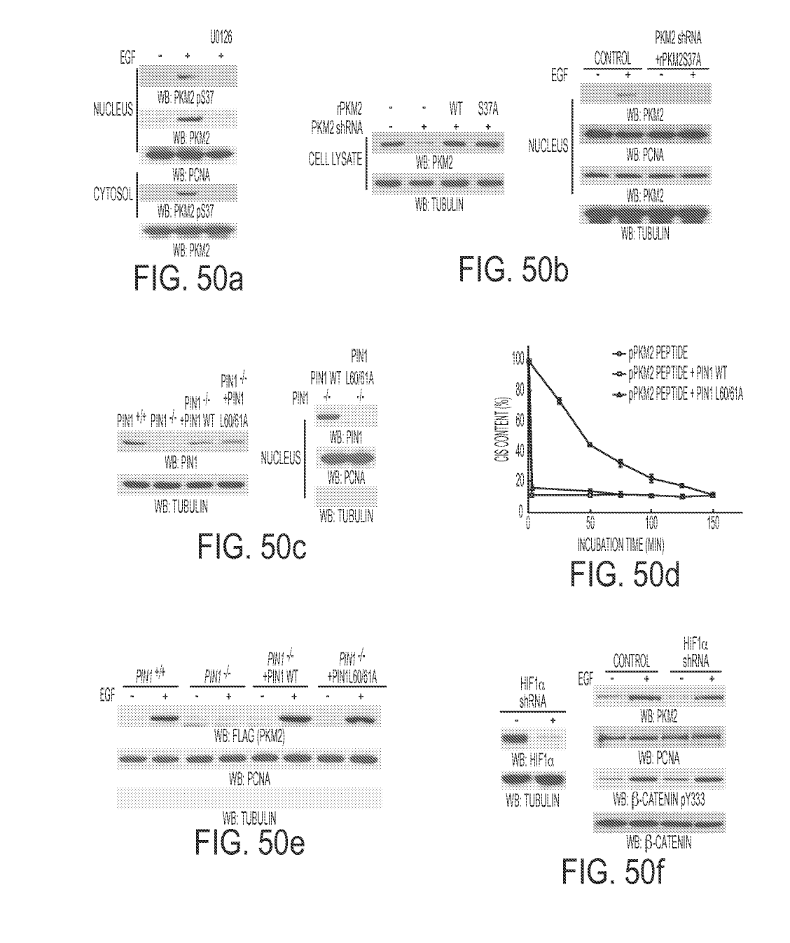

Because PKM1 and PKM2 share the identical N-terminal amino acid sequence (including S37), the inventors then tested whether ERK2-mediated phosphorylation of S37 is restricted to PKM2. Immunoblotting of the immunoprecipitated FLAG-tagged PKM1 or PKM2 with the anti-phospho-PKM2 S37 antibody showed that EGF treatment resulted in phosphorylation of WT PKM2 (FIG. 43b, left panel) but not PKM1 (FIG. 43b, right panel) or the PKM2 S37A mutant. Consistently, pretreatment with U0126 blocked EGF-induced S37 phosphorylation and nuclear translocation of PKM2 (FIG. 43b, left panel; FIG. 50a). In addition, expression of constitutively active MEK1 Q56P with WT ERK2, but not with the ERK2 K52R mutant, induced PKM2 S37 phosphorylation (FIG. 43c). These results indicate that ERK2 specifically phosphorylates PKM2 but not PKM1. This observation is consistent with the presence of the D domain in PKM2 (but not in PKM1) that allows unique ERK1/2 binding (FIGS. 42d,g).

Expression of FLAG-tagged WT PKM2, the PKM2 S37A mutant, or a phosphorylation-mimic PKM2 S37D mutant in U87/EGFR cells (FIG. 43d) showed that the FLAG-PKM2 S37A mutant was resistant to EGF-induced nuclear translocation, as determined by immunoblotting analyses (FIG. 43e, top panel) or immunofluorescence analysis (FIG. 43f, left and middle panels). Similar results were observed in U87/EGFR cells with depleted endogenous PKM2 and reconstituted expression of RNAi-resistant rPKM2 S37A (FIG. 50b). In contrast, the phosphorylation-mimic PKM2 S37D mutant had a higher level of nuclear accumulation in the absence of EGF treatment than did WT PKM2 or the S37A mutant (FIG. 43e, bottom panel; FIG. 43f, right panel). These results indicate that PKM2 phosphorylation at S37 is required for nuclear translocation of PKM2.

### PKM2 S37 Phosphorylation Recruits PIN1

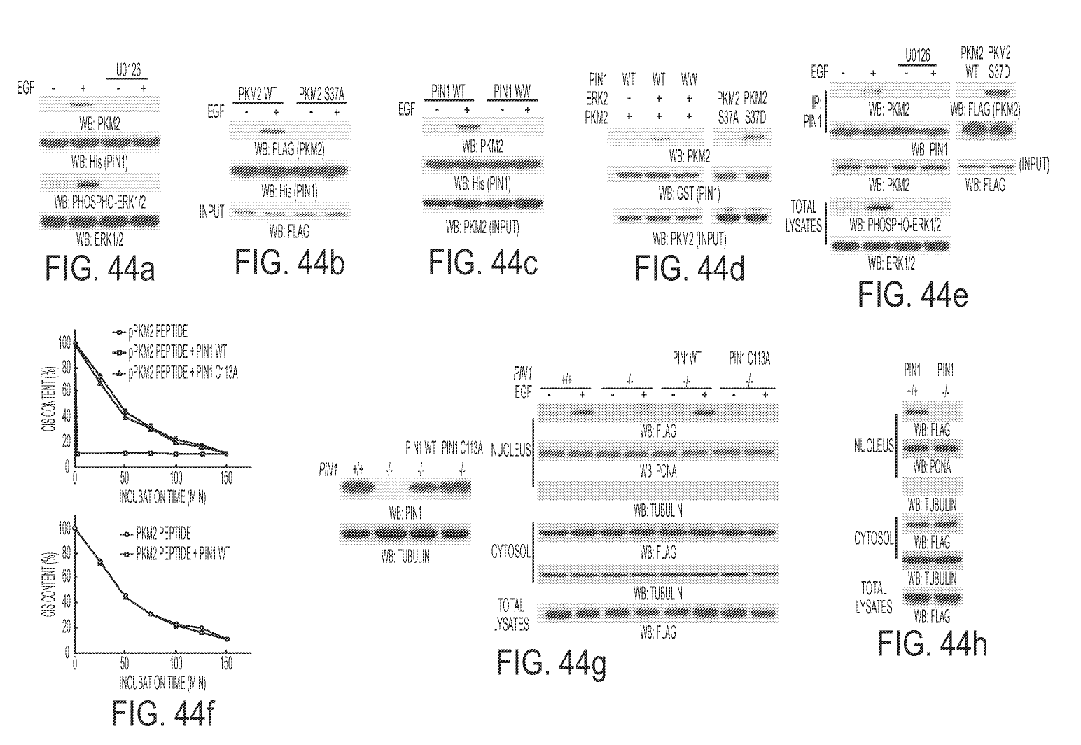

The peptidyl-proline isomerase protein interacting with never in mitosis A (NIMA)-1 (PIN1) recognizes phosphorylated pS/TP-peptide sequences and catalyzes their cis-trans isomerization (Lu and Zhou, 2007; Zheng et al., 2009). EGF treatment induced a strong binding of endogenous PKM2 to His-PIN1 immobilized on nickel agarose beads (FIG. 44a), whereas this binding was abrogated by pretreatment of cells with U0126 (FIG. 44a) or S37A mutation of PKM2 (FIG. 44b). In addition, a His-PIN1 WW domain mutant, which prevents the binding of PIN1 to a pS/TP substrate (Zheng et al., 2009), failed to bind to endogenous PKM2 in U87/EGFR cells upon EGF treatment (FIG. 44c). These results indicate that the PIN1 WW domain binds to the ERK1/2-phosphorylated S37 of PKM2.

To determine whether ERK1/2-dependent PKM2 phosphorylation is sufficient for PIN1 binding to PKM2, the inventors mixed His-PKM2 with WT GST-PIN1 or a GST-PIN1 WW domain mutant in the presence or absence of active ERK2. As shown in FIG. 44d (left panel), phosphorylated PKM2, but not its nonphosphorylated counterpart, bound to WT PIN1, but not the PIN1 WW domain mutant. In addition, the purified phosphorylation-mimic His-PKM2 S37D mutant, but not His-PKM2 S37A, was able to interact with GST-PIN1 in the absence of E2 (FIG. 44d, right panel). These in vitro results were validated by a co-immunoprecipitation assay showing that EGF stimulation greatly increased the binding of endogenous PIN1 to endogenous PKM2, which was blocked by U0126 pretreatment (FIG. 44e, left panel). In contrast to WT FLAG-PKM2, the FLAG-PKM2 S37D mutant was co-immunoprecipitated with PIN1 in the absence of EGF treatment (FIG. 44e, right panel). These results indicate that PKM2 S37 phosphorylation is sufficient for PIN1 binding to PKM2.

To further examine whether the phosphorylated S37/P38 motif of PKM2 is a PIN1 substrate, the inventors synthesized oligopeptides of PKM2 containing phosphorylated or nonphosphorylated S37/P38. As demonstrated in FIG. 44f, GST-WT PIN1 isomerized the phosphorylated S37/P38 peptide much more efficiently than did a catalytically inactive GST-PIN1 C113A mutant (left panel), whereas WT PIN1 failed to isomerize the nonphosphorylated counterpart (right panel). These results strongly suggest that PIN1 specifically isomerizes the phosphorylated S37/P38 within PKM2.

To determine the role of PIN1 in nuclear translocation of PKM2, the inventors used EGF to treat PIN1^{+/+}, PIN1^{-/-}, or PIN1^{-/-} mouse embryonic fibroblasts (MEFs) with reconstituted expression of WT PIN1 or a PIN1 C113A mutant (FIG. 44g, left panel). PIN1 deficiency completely blocked EGF-induced nuclear translocation of human FLAG-PKM2 whereas this block was rescued by re-expression of WT PIN1 but not by expression of the PIN1 C113A mutant (FIG. 44g, right panel). These results indicate that the catalytic activity of PIN1 is required for EGF-induced nuclear translocation of PKM2. Furthermore, the nuclear translocation of the phosphorylation-mimic FLAG-PKM2 S37D mutant (FIG. 43e) was inhibited by PIN1 deficiency (FIG. 44h). This observation indicate that phosphorylation of PKM2 by itself is not sufficient for PKM2 nuclear translocation, further supporting a role for PIN1 in EGF-induced nuclear translocation of PKM2.

### PIN1 Regulates Binding of PKM2 to Importin α5

PIN1 contains an NLS (Lufei and Cao, 2009). The inventors next tested whether PKM2 translocates together with PIN1 into the nucleus in a PIN1 NLS-dependent manner. A PIN1 NLS (L60/61A) mutant, which was expressed in PIN1^{-/-} cells, failed to accumulate in the nucleus (FIG. 50c). However, expression of this PIN1 mutant, whose catalytic activity was intact (FIG. 50d), still permitted EGF-induced nuclear translocation of FLAG-PKM2 (FIG. 50e). These results indicate that the nuclear translocation of PKM2 is not mediated by the NLS of PIN1 and suggest that these two proteins separately translocate into the nucleus. Furthermore, depletion of HIF1α, which is a PKM2-associated protein (Luo et al., 2011), did not affect EGF-induced nuclear translocation of PKM2 and β-catenin Y333 phosphorylation (which is required for β-catenin transactivation) (Yang et al., 2011) (FIG. 50f), suggesting separate nuclear translocation processes for these two proteins and β-catenin regulation independent of HIF1α.

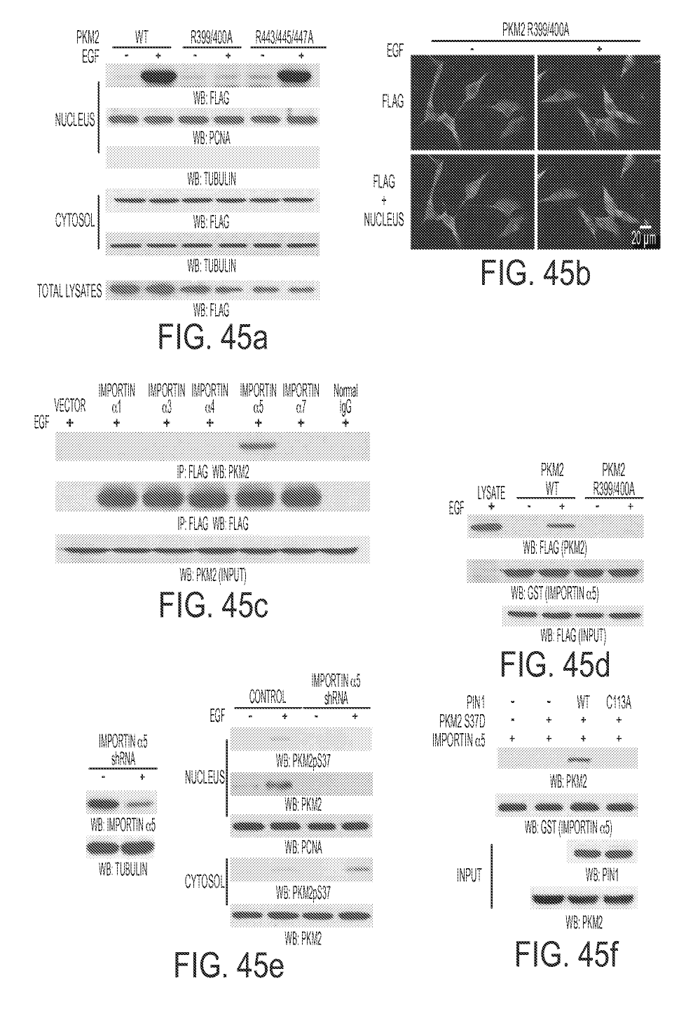

To test whether PKM2 contains a sterically inaccessible NLS that is exposed for importin binding after PIN1-mediated cis-trans isomerization, the inventors mutated the R399/400 and R443/445/447 residues in the putative NLS sequences of the C-domain (aa 393-531) encoded by PKM2-specific exon 10 (Jans et al., 2000) into alanine. Cell fractionation (FIG. 45a) and immunofluorescence (FIG. 45b) analyses showed that FLAG-PKM2 R399/400A, unlike the R443/445/447A mutant or WT PKM2, was unable to translocate into the nucleus upon EGF treatment. These results indicate that the NLS containing R399/400 in PKM2, but not in PKM1, is essential for EGF-induced nuclear translocation of PKM2.

Importin a functions as an adaptor and links NLS-containing proteins to importin β, which then docks the ternary complex at the nuclear-pore complex (NPC) to facilitate the translocation of these proteins across the nuclear envelope. Six importin a family members (α1, α3, α4, α5, α6 and α7) have been identified in humans (Mason et al., 2009). FIG. 45c shows that EGF treatment resulted in endogenous PKM2 binding to importin α5, but not to α1, α3, α4, and α5 family members. In addition, purified GST-importin α5 was able to pull down WT FLAG-PKM2, but not FLAG-PKM2 R399/400A, from EGF-treated U87/EGFR cells (FIG. 45d). Furthermore, depletion of importin α5 with KPNA1 (coding for importin α5) shRNA (FIG. 45e) largely blocked EGF-induced nuclear translocation of PKM2 and resulted in accumulated phosphorylated PKM2 S37 in the cytosol (FIG. 45e).

To determine whether PIN1 plays a role in the binding of PKM2 to importin α5, the inventors mixed purified phosphorylation-mimic His-PKM2 S37D and GST-importin α5 in the presence or absence of WT His-PIN1 or the His-PIN1 C113A mutant. As shown in FIG. 45f, PKM2 S37D alone did not bind to importin α5. However, inclusion of WT PIN1, but not of the inactive PIN1 C113A mutant, enabled PKM2 S37D to interact with importin α5. These results strongly suggest that phosphorylation of PKM2 at S37, which by itself is not sufficient for PKM2 nuclear translocation (FIG. 44h), leads to cis-trans isomerization of PKM2 by PIN1 to expose intermolecularly- or intramolecularly-masked R399/400 of the PKM2 NLS for binding to importin α5. In combination with the finding that the recruitment of PIN1 to PKM2 is mediated by both the binding of the ERK2 docking groove to PKM2-specific exon 10-encoded I429/L431 region and the phosphorylation of PKM2 at S37, these results highlight the significance of precise and sequential post-translational modifications of PKM2 and its interactions with ERK1/2, PIN1, and importin α5 in its nuclear translocation.

### Nuclear PKM2 Regulates Glycolytic Gene Expression

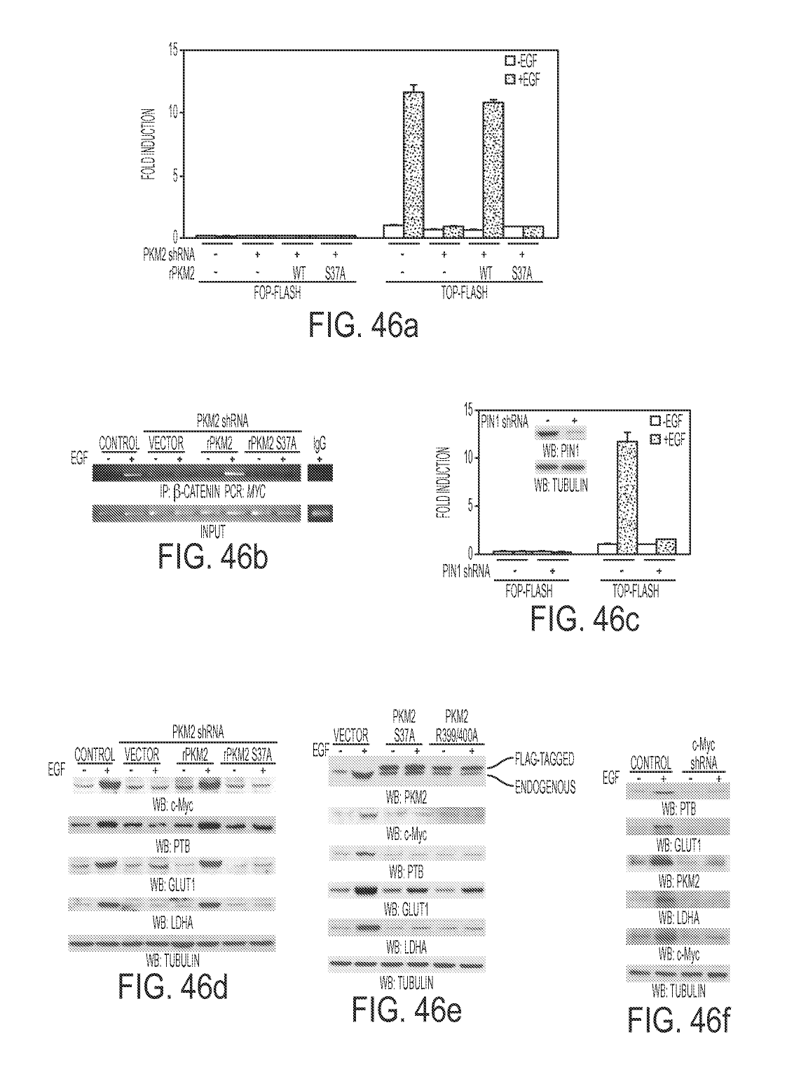

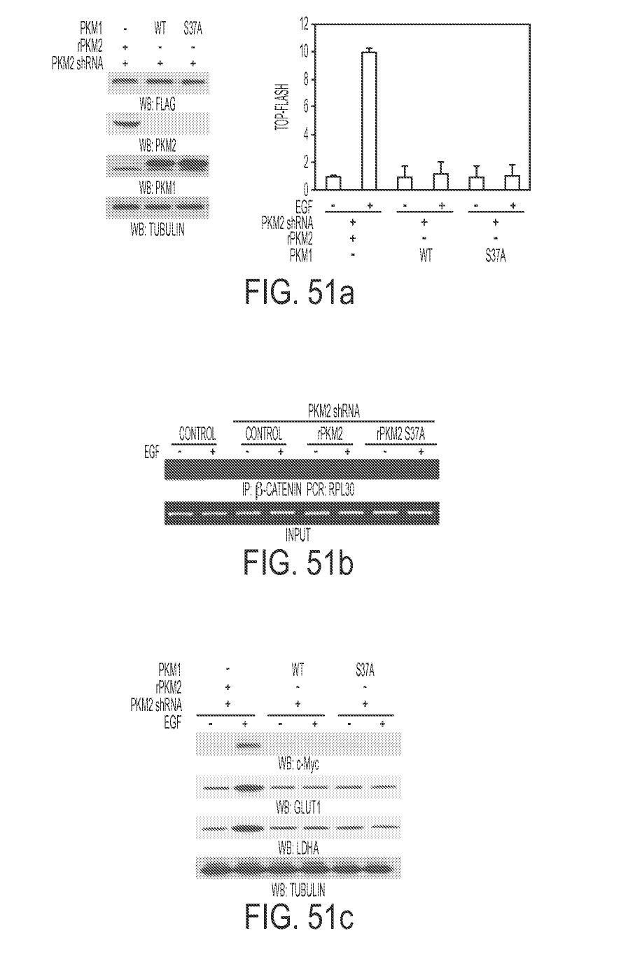

Previously, the inventors demonstrated that nuclear PKM2 interacts with phosphorylated β-catenin Y333 for β-catenin transactivation (Yang et al., 2011). The TOP-FLASH TCF/LEF-1 luciferase reporter analyses showed that PKM2 depletion significantly inhibited EGF-induced β-catenin transactivation, which was largely rescued by reconstituted expression of RNAi-resistant WT rPKM2 but not those of rPKM2 S37A (FIG. 46a), WT FLAG-PKM1, or FLAG-PKM1 S37A (FIG. 51A). In addition, chromatin immunoprecipitation (ChIP) with an anti-β-catenin antibody showed that EGFR activation resulted in the binding of β-catenin to the MYC promoter, which was inhibited by PKM2 depletion (FIG. 46b; FIG. 51B). Notably, this inhibition was abrogated by expression of WT rPKM2 but not that of rPKM2 S37A. In addition, PIN1 depletion by PIN1 shRNA also blocked EGF-induced β-catenin transactivation (FIG. 46c). These results indicate that PIN1-dependent nuclear translocation of PKM2 plays a critical role in EGF-induced β-catenin transactivation.

c-Myc expression is known to be upregulated by PKM2-dependent β-catenin transactivation (Lu et al., 2003; Yang et al., 2012). c-Myc transcriptionally induces expression of GLUT1 and lactate dehydrogenase A (LDHA) (DeBerardinis et al., 2008; Dang et al., 2008) and upregulates PTB expression, thereby regulating PKM pre-mRNA splicing for generation of PKM2 mRNA (David et al., 2010; Clower et al., 2010). The inventors next examined whether nuclear translocation of PKM2 plays a role in c-Myc-dependent expression of downstream genes. As shown in FIG. 46d, PKM2 depletion blocked EGF-enhanced expression of c-Myc, PTB, GLUT1, and LDHA, which was rescued by reconstituted expression of WT rPKM2 but not those of rPKM2 S37A, WT FLAG-PKM1, or FLAG-PKM1 S37A (FIG. 51C). Of note, EGF treatment resulted in PKM2 upregulation, which is in agreement with the enhanced expression of PTB by EGF (FIG. 46e). Expression of FLAG-PKM2 S37A or R399/400A blocked EGF-induced upregulation of c-Myc, PTB, PKM2, GLUT1, and LDHA. In line with these findings, c-Myc depletion blocked EGF-induced upregulation of PTB, PKM2, GLUT1, and LDHA (FIG. 46f). These results indicate that nuclear translocation of PKM2 plays a pivotal role in EGF-induced β-catenin transactivation, which results in a subsequent upregulation of PTB, PKM2, LDHA, and GLUT 1 expression in a c-Myc-dependent manner.

### Nuclear PKM2 is Required for the Warburg Effect

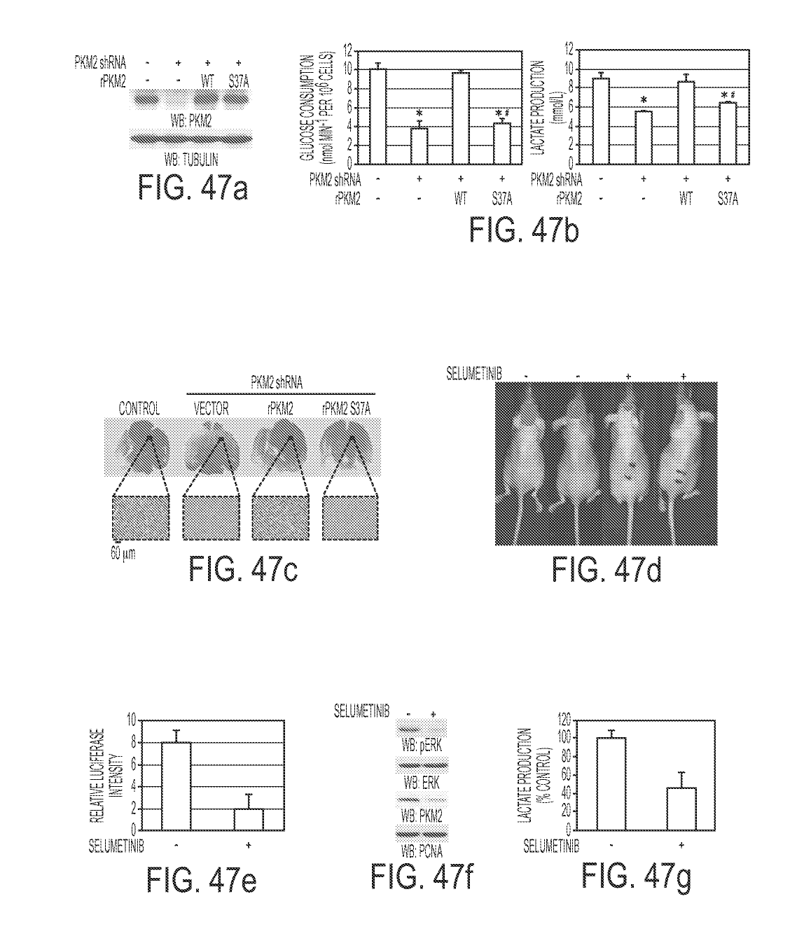

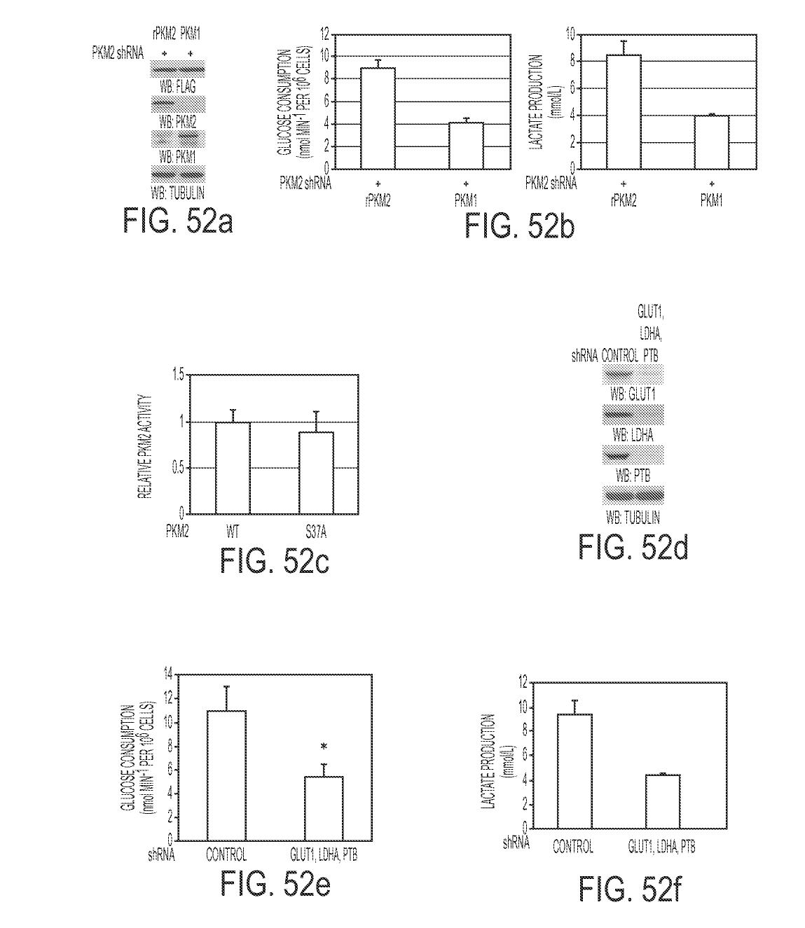

GLUT1 and LDHA are required for glucose uptake and the conversion of pyruvate to lactate, respectively (Christofk et al., 2008). To investigate the role of nuclear translocation of PKM2 in EGFR-regulated tumor cell glycolysis, the inventors depleted PKM2 with PKM2 shRNA in EGFRvIII-expressing U87 cells and reconstituted these cells with WT rPKM2, rPKM2 S37A (FIG. 47a), and WT FLAG-PKM1 (FIG. 52a). PKM2 depletion significantly reduced glucose consumption and lactate production (FIG. 47b), which were rescued by reconstituted expression of WT PKM2, but not that of PKM2 S37A (FIG. 47b) or WT FLAG-PKM1 (FIG. 52b). Because the S37A mutation did not affect PKM2 kinase activity (FIG. 52c), these results indicate that nuclear translocation of PKM2, which results in upregulation of PKM2 itself, GLUT 1, and LDHA, is essential for the EGFR-induced Warburg effect. These findings were further supported by the results showing that depletion of GLUT1, LDHA, and PTB largely reduced glucose consumption and lactate production (FIG. 44d-f).

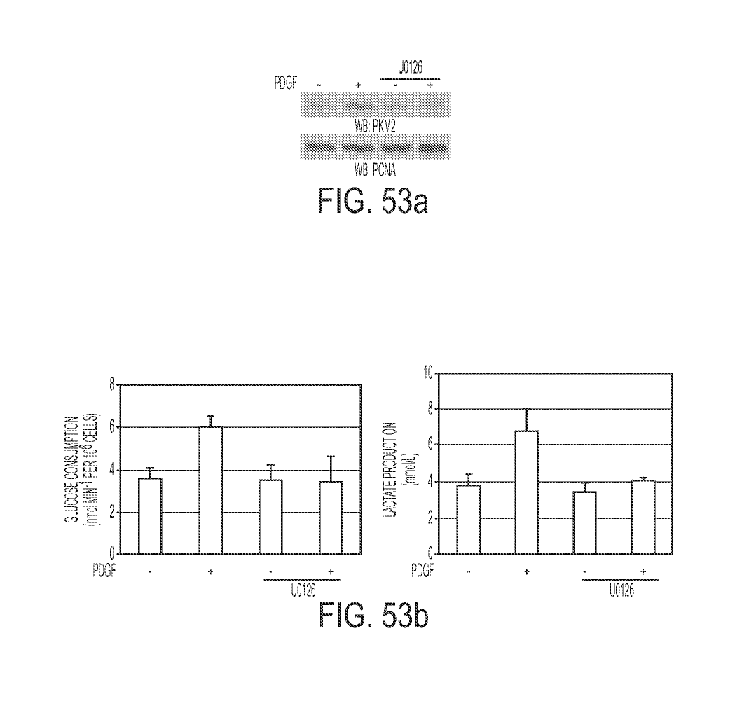

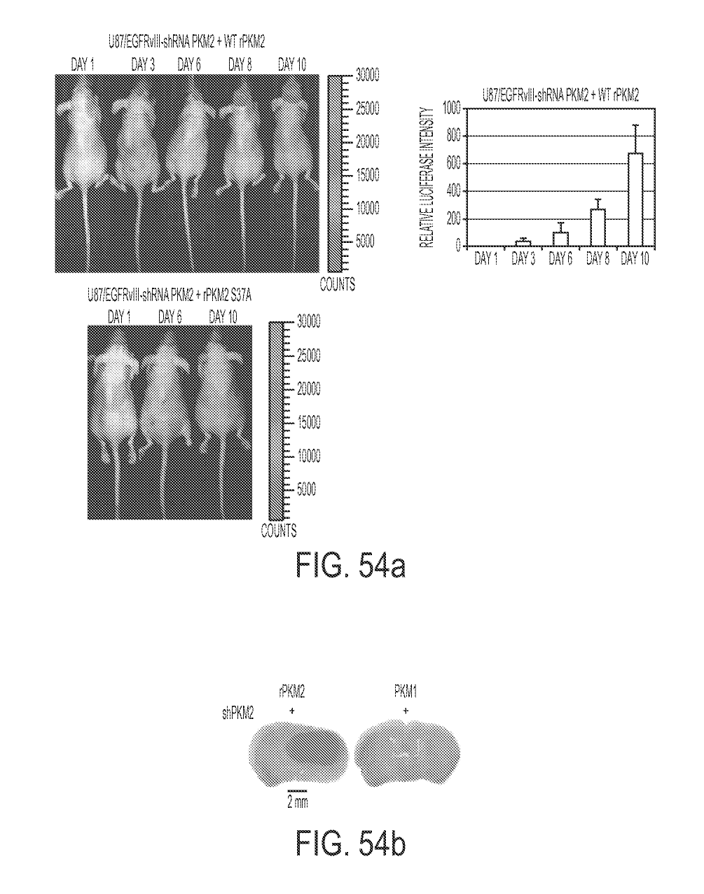

Acting like EGF, platelet-derived growth factor (PDGF) induced nuclear translocation of PKM2, which was blocked by pretreatment with U0126 (FIG. 53a). In addition, U0126 treatment blocked PDGF-induced glucose uptake and lactate production (FIG. 53b), suggesting that activation of both EGFR and PDGFR promote the Warburg effect mediated by ERK1/2-dependent nuclear translocation of PKM2.

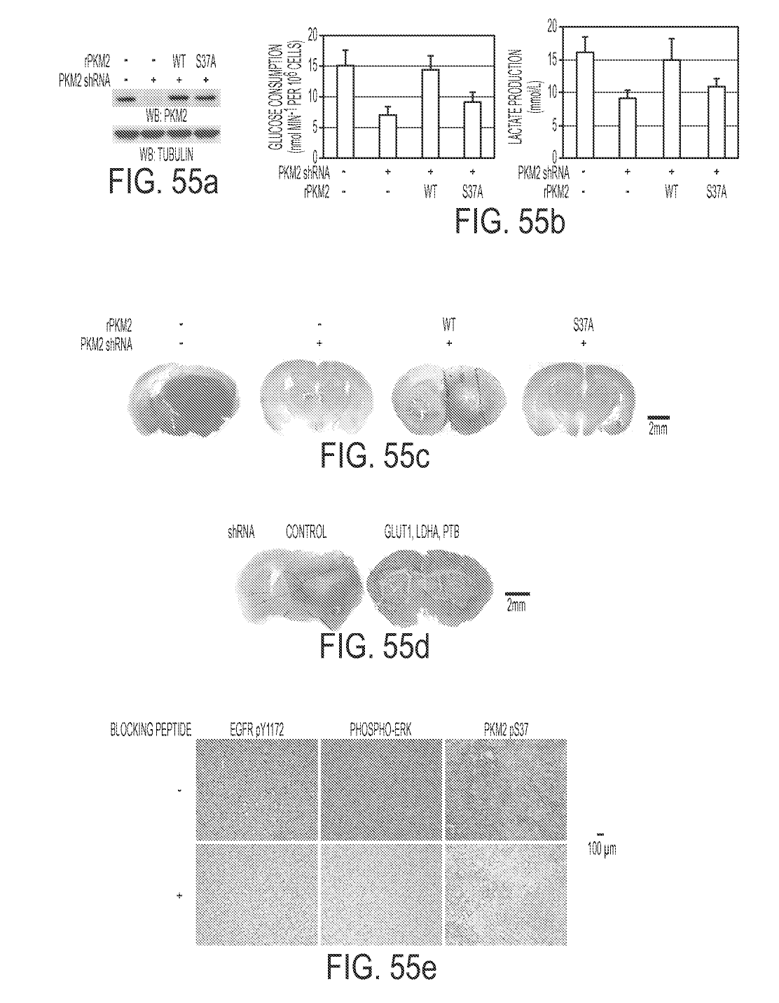

To determine the role of nuclear translocation of PKM2 in brain tumorigenesis, the inventors intracranially injected U87/EGFRvIII, U87/EGFRvIII-PKM2 shRNA, and U87/EGFRvIII-PKM2 shRNA cells with reconstituted expression of WT rPKM2, rPKM2 S37A, WT FLAG-PKM1, and FLAG-PKM1 S37A into athymic nude mice. Depletion of PKM2 abrogated the growth of brain tumors, which was rescued by reconstituted expression of WT rPKM2, but not that of rPKM2 S37A (FIG. 47c, FIG. 54a) or WT FLAG-PKM1 (FIG. 54b). Similar results on PKM2-dependent Warburg effect and tumorigenesis were also obtained using human primary GSC11 GBM cells (FIG. 55a-c). In addition, depletion of GLUT1, LDHA, and PTB inhibited EGFRvIII-induced brain tumor growth (FIG. 55d). Furthermore, intratumoral injection of the MEK inhibitor selumetinib inhibited tumor growth (FIG. 47d,e) and reduced ERK1/2 phosphorylation, PKM2 expression (FIG. 47f), and lactate production in tumor tissue (FIG. 47g). These results highlight the significance of ERK-dependent nuclear translocation of PKM2 and PKM2-dependent gene transcription in the Warburg effect and brain tumor development.

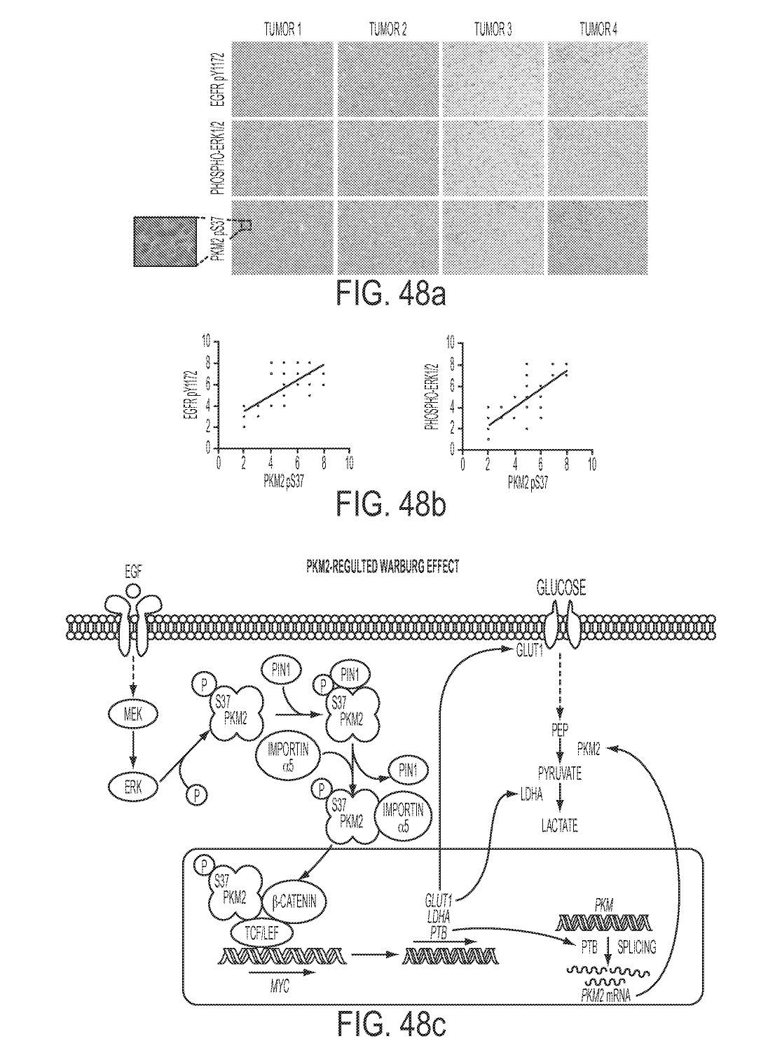

To further determine whether these findings have clinical relevance, the inventors examined the activity of EGFR and ERK1/2 with PKM2 S37 phosphorylation in serial sections of 48 human primary GBM specimens by immunohistochemical (IHC) analyses by using antibodies with validated specificities (FIG. 55e). As shown in FIG. 48a, the levels of EGFR and ERK1/2 activities correlated with the levels of PKM2 S37 phosphorylation. Quantification of the staining showed that this correlation was statistically significant among different specimens (FIG. 48b: r=0.77, P<0.001, top panel; r=0.78, P<0.001, bottom panel). These results strongly suggest that EGFR- and ERK1/2-induced PKM2 S37 phosphorylation occurs in human GBM.

### Discussion

Studies presented above demonstrate that nuclear translocation of PKM2 is a cause of the Warburg effect. The studies reveal an important mechanism underlying the Warburg effect: EGFR activation promotes aerobic glycolysis by means of a PKM2-dependent positive feedback loop on its own expression as well as the expression of GLUT1 and LDHA. EGFR activation results in nuclear translocation of PKM2, which is mediated by the ERK1/2-dependent phosphorylation of PKM2 S37 and consequently PIN1-catalized cis-trans isomerization of PKM2 for binding to importin α5. Nuclear PKM2 regulates β-catenin transactivation-dependent MYC transcription and, subsequently, the expression of GLUT1, LDHA, and PTB-mediated PKM2 expression. The elevated expression of these rate-limiting glycolytic genes plays a critical role in EGF-induced Warburg effect, featured by elevated glucose uptake and higher lactate production in the presence of oxygen, which leads to enhanced brain tumor development (FIG. 48c). Moreover, PKM2 S37 phosphorylation correlates with EGFR and ERK1/2 activity in human GBM, implying that PKM2 nuclear translocation play a significant role in the progression of these tumors in humans.

The mechanisms that accounted for the finding that PKM1 could not compensate for the loss of PKM2 in the Warburg effect and tumorigenesis were not well understood (Christofk et al., 2008). The inventors demonstrate here that PKM1, which lacks both a D domain and the NLS encoded by exon 10, did not bind activated ERK2 and was not phosphorylated at S37. PKM1 remained in cytosol after EGF treatment. Although PKM1 and PKM2 are both capable of converting phosphoenolpyruvate (PEP) to pyruvate, only PKM2 is able to translocate to the nucleus to conduct its unique nuclear functions. These functions include, but may not be limited to, transactivation of β-catenin phosphorylated at Y333; histone H3 phosphorylation and subsequent transcription of its downstream genes, such as MYC and CCND1; and promotion of cell cycle progression (Yang et al., 2011; Yang et al., 2012) and the Warburg effect. Given that c-Myc and cyclin D1 play instrumental roles in cell proliferation, survival, and metabolism, the inventor's findings underscore the significant role of the metabolic and nonmetabolic functions of PKM2 in a very broad area of cellular activities. These findings also highlight the distinct functions of PKM2 relative to those of PKM1 in tumor development. Compared with the relatively well-studied cytosolic functions of PKM2, the illustrated nuclear functions of PKM2 in tumor development and its regulatory mechanisms for expression of glycolic enzymes and cyclin D1 provide important insights into PKM2-promoted tumor progression and targets for treating human cancer.

### Materials and Methods

### Cells and Cell Culture Conditions

U87, U87/EGFR, and U251 GBM cells as well as 293T, PIN1^{+/+}, PIN1^{-/-}, c-myc^{+/+}, and c-myc^{-/-} cells were maintained in Dulbecco's modified Eagle's medium (DMEM) supplemented with 10% bovine calf serum (HyClone, Logan, Utah). These cell cultures were made quiescent by growing them to confluence and then replacing the medium with fresh medium containing 0.5% serum for 1 d. GSC11 human primary GBM cells were maintained in DMEM/F-12 50/50 supplemented with B27, EGF (10 ng/mL), and bFGF (10 ng/mL). GSC11 cell cultures were made quiescent similarly, by growing them to confluence and then replacing the medium with fresh medium containing 0.5% serum for 1 d.

### Materials

Rabbit polyclonal antibodies recognizing phospho-PKM2 S37, PKM2, EGFR, phospho-EGFR-Y1172, and c-Myc were obtained from Signalway Biotechnology (Pearland, Tex.) and Signalway Antibody (College Park, Md.). Polyclonal antibodies for GST, ERK, PCNA, MEK1, PTB, and LDHA and monoclonal antibody for phospho-ERK1/2 (sc-7383, clone E-4; 1:1000 dilution for immunoblotting) were purchased from Santa Cruz Biotechnology (Santa Cruz, Calif.). EGF, rabbit polyclonal antibody for FLAG (F7425; 1 μg for immunoprecipitation), monoclonal secondary anti-rabbit IgG (R3155; Native-Peroxidase reacts specifically with non-reduced rabbit IgG and does not react with reduced rabbit IgG), and mouse monoclonal antibodies for FLAG (F3165, clone M2; 1:5000 dilution for immunoblotting), His (H1029, clone HIS-1; 1:2000 dilution for immunoblotting), and tubulin (T9026, clone DM1A; 1:2000 dilution for immunoblotting) were purchased from Sigma (St. Louis, Mo.). Hygromycin, puromycin, G418, LY290042, SU6656, SP600125, and U0126 were purchased from EMD Biosciences (San Diego, Calif.). Active ERK2 was obtained from Signalchem (Richmond, Canada). Hoechst 33342 and Alexa Fluor 488 goat anti-rabbit antibody were from Molecular Probes (Eugene, Oreg.). HyFect transfection reagents were from Denville Scientific (Metuchen, N.J.). GelCode Blue Stain Reagent was obtained from Pierce (Rockford, Ill.).

### Transfection

Cells were plated at a density of 4×10⁵/60-mm dish 18 h prior to transfection. Transfection was performed using HyFect reagents according to the vendor's instructions. Transfected cultures were selected with puromycin (5 μg/mL), hygromycin (200 μg/mL), or G418 (400 μg/mL) for 10-14 d. At that time, antibiotic-resistant colonies were picked, pooled, and expanded for further analysis under selective conditions.

### Immunoprecipitation and Immunoblotting Analyses

Extraction of proteins with a modified buffer from cultured cells was followed by immunoprecipitation and immunoblotting with corresponding antibodies, as described previously (Lu et al., 1998).

### DNA Constructs and Mutagenesis

PCR-amplified human importin a1, a3, a5 and a7 were cloned into pcDNA3.1/hygro (+) vector between BamH I and Not I. PCR-amplified human importin a4 and a6 were cloned into pcDNA3.1/hygro (+) vector between EcoR V and Not I. PCR-amplified human PKM2 was cloned into either pCold I vector (TaKaRa, Shiga, Japan) or pcDNA3.1/hygro (+) vector between BamH I and Xba I. pcDNA 3.1/hygro (+)-PKM2 S37A, S37D, I428R/V430A, I429R/L431R, R399/400A, and R443/445/447A; pcDNA 3.1/hygro (+)-rPKM2; and pBabe-PIN1 L60/61A and C113A were made using the QuikChange site-directed mutagenesis kit (Stratagene, La Jolla, Calif.). pCDNA 3.1 rPKM2 contains non-sense mutations of C1170T, C1173T, T1174C, and G1176T.

pGIPZ control was generated with a control oligonucleotide (5′-CTTCTAACACCGGAGGTCTT-3′) (SEQ ID NO: 27). pGIPZ PKM2 shRNA was generated with 5′-CATCTACCACTTGCAATTA-3′ (SEQ ID NO: 14) oligonucleotide targeting the transcript of the PKM2 exon 10 pGIPZ importin α5 shRNA was generated with 5′-GGCCTTTGATCTTATTGAGCA-3′ oligonucleotide.

### Measurements of Glucose Consumption and Lactate Production

Cells were seeded in culture dishes, and the medium was changed after 6 h with no-serum DMEM. Cells were incubated for 12-16 h, and the culture medium was then collected for measurement of glucose and lactate concentrations. Glucose levels were determined using a glucose (GO) assay kit (Sigma). Glucose consumption was the difference in glucose concentration compared with DMEM. Lactate levels were determined using a lactate assay kit (Eton Bioscience, San Diego, Calif.).

### Purification of Recombinant Proteins

WT and mutant His-PKM2, GST-PIN1, and GST-importin α5 proteins were expressed in bacteria and purified, as described previously (Xia et al., 2007).

### In Vitro Kinase Assays

Kinase reactions were performed as described previously (Fang et al., 2007).

### Luciferase Reporter Gene Assay

Transcriptional activation of β-catenin in 293T cells was measured as described previously (Fang et al., 2007).

### In Vitro Isomerization Assay

The isomerization rate was shown with the cis-peptide content, which was determined by isomer-specific proteolysis. Cis-peptides were prepared by incubating the peptides with α-chymotrypsin at 0° C. for 2 min to completely hydrolyze the trans isomer at the 4-nitroanilide bond in order to obtain the pure cis-peptides. The pure cis-peptides were allowed to re-equilibrate. As the isomerization proceeded, aliquots were taken at the indicated time. Chymostatin was added to inactivate chymotrypsin. The absorbance of the released 4-nitroaniline was measured at 405 nm.

### Immunofluorescence Analysis

Cells were fixed and incubated with primary antibodies, Alexa Fluor dye-conjugated secondary antibodies, and Hoechst 33342 according to standard protocols. Cells were examined using a deconvolution microscope (Zeiss, Thornwood, N.Y.) with a 63-Å oil immersion objective. Axio Vision software from Zeiss was used to deconvolute Z-series images.

### Subcellular Fractionation

Nuclei, cytosol, and cell membrane were isolated using a nuclear extract kit from Active Motif North America (Carlsbad, Calif.) and the ProteoExtract subcellular proteome extraction kit from Calbiochem (San Diego, Calif.). Nuclear proteins (60 μg) and cytosolic proteins (14 μg) were used in immunoblotting analyses.

### ChIP Assay

ChIP was performed using an Upstate Biotechnology kit. Chromatin prepared from cells (in a 10-cm dish) was used to determine total DNA input and for overnight incubation with the specific antibodies or with normal rabbit or mouse immunoglobulin G. The human MYC promoter-specific primers used in PCR were 5′-CAGCCCGAGACTGTTGC-3′ (SEQ ID NO: 20) (forward) and 5′-CAGAGCGTGGGATGTTAG-3′ (SEQ ID NO: 21) (reverse).

### Pyruvate Kinase Assay

The activity of bacterially purified WT PKM2 (0.1 μg) and PKM2 S37A (0.1 μg) toward PEP was measured with a pyruvate kinase assay (BioVision, Mountain View, Calif.) according to the manufacturer's instruction. Data represent the mean±SD of three independent experiments.

### Immunohistochemical Analysis

Mouse tumor tissues were fixed and prepared for staining as previously described (Zheng et al., 2009). The specimens were stained with Mayer's hematoxylin and subsequently with eosin (Biogenex Laboratories, San Ramon, Calif.). Afterward, the slides were mounted using Universal Mount (Research Genetics, Huntsville, Ala.).

The tissue sections from paraffin-embedded human GBM specimens were stained with an antibody against phospho-EGFR-Y1172, phospho-PKM2-S37 (Signalway Biotechnology), or phospho-ERK (Santa Cruz, Calif.) or with nonspecific immunoglobulin G as a negative control. The inventors quantitatively scored the tissue sections according to the percentage of positive cells and the staining intensity, as previously defined (Zheng et al., 2009). The inventors assigned the following proportion scores: 0 if 0% of the tumor cells showed positive staining, 1 if 0% to 1%, 2 if 1% to 10%, 3 if 11% to 30%, 4 if 31% to 70%, and 5 if 71% to 100%. The inventors rated the intensity of staining on a scale of 0 to 3: 0 for negative, 1 for weak, 2 for moderate, and 3 for strong. The proportion and intensity scores were combined to obtain a total score (range, 0-8), as described previously (Yang et al., 2011). Scores were compared with overall survival, defined as the time from date of diagnosis to death or last known date of follow-up. All patients had received standard adjuvant radiotherapy after surgery, which had been followed by treatment with an alkylating agent (temozolomide in the majority of cases). The use of human brain tumor specimens and the database was approved by the institutional review board at the MD Anderson Cancer Center.

Bioluminescence Imaging with IVIS

Mice were anesthetized with isoflurane inhalation, and were subsequently intraperitoneally (i.p.) injected with 100 μL of 7.5 mg/mL D-luciferin (Xenogen). Bioluminescence imaging with a CCD camera (IVIS, Xenogen) was initiated 10 min after injection with 2 min exposure time. Bioluminescence from the region of interest (ROI) was defined manually. Background was defined using an ROI from a mouse that was not given an i.p. injection of D-luciferin. All bioluminescent data were collected and analyzed using IVIS.

### Intracranial Injection

The inventors intracranially injected 5×10⁵ U87/EGFRvIII or GSC11 cells with or without RNAi-depleted PKM2 and reconstitution of rPKM2 or rPKM2 S37A expression (in 5 μL of DMEM per mouse) into 4-week-old female athymic nude mice. The intracranial injections were performed as described in a previous publication (Yang et al., 2011). Seven mice per group were used in each experiment. Animals were sacrificed two weeks (for U87/EGFRvIII cells) or four weeks (for GSC11 cells) after the glioma cell injection. The brain of each mouse was harvested, fixed in 4% formaldehyde, and embedded in paraffin. Tumor formation and the phenotype were determined by histologic analysis of H & E-stained sections.

Three days after intracranial injections of U87/EGFRvIII cells, selumetinib (50 mg/mL in 5 μL of DMSO) or DMSO was injected into the tumor of mice (seven mice for each group). The treatment was repeated every three days. Bioluminescence imaging in both groups were collected and analyzed using IVIS at indicated time.

### Example 4

### EGFR-Induced and PKCε Monoubiquitylation-Dependent NF-κB Activation Upregulates PKM2 Expression and Promotes Turmorigenesis

Studies presented below demonstrate that activation of EGFR in human cancer cells results in increased glucose uptake and lactate production in a PKM2-dependent manner. Intriguingly, EGFR activation leads to NF-κB-dependent upregulation of PKM2 expression; NF-κB activation, in turn, is mediated by PLCγ1 and PKCε monoubiquitylation-dependent IKKβ activation. This EGFR-initiated signaling cascade promotes tumor development.

### Results

### EGFR Activation Results in Upregulation of PKM2 Expression

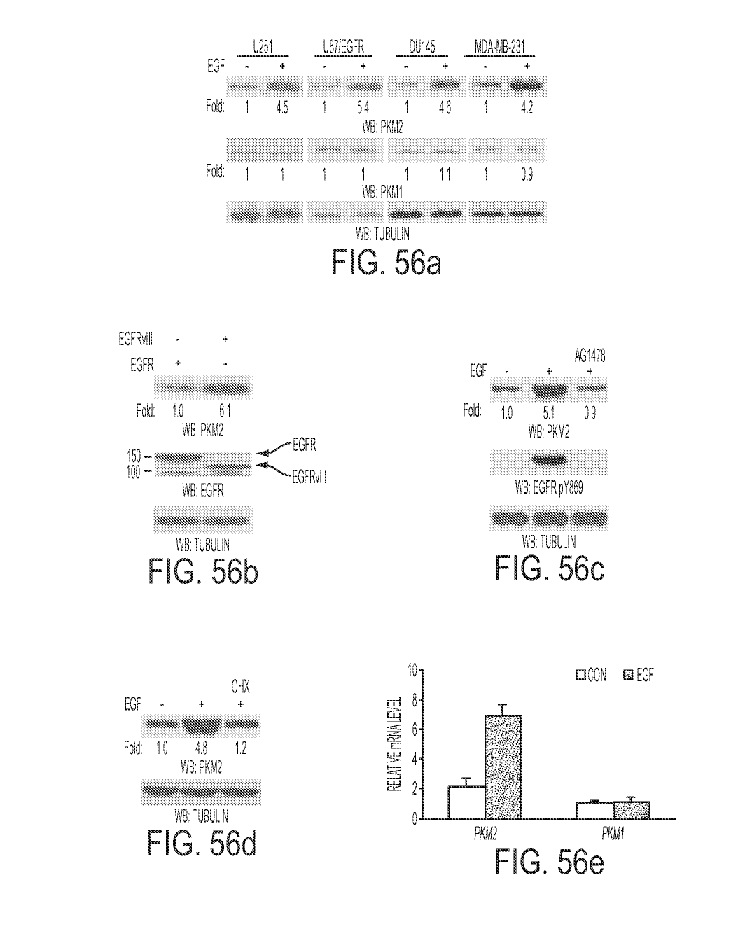

EGFR activation and PKM2 upregulation have been detected separately in many cancer types; however, the connection between these two tumorigenesis-related alterations remains unknown. To examine whether EGFR activation regulates PKM2 expression, the inventors used EGF to stimulate DU145 human prostate cancer cells, MDA-MB-231 human breast carcinoma cells, and U251 and EGFR-overexpressed U87 (U87/EGFR) human glioblastoma (GBM) cells. EGF treatment increased expression of PKM2, but not PKM1 (FIG. 56A). In addition, U87 cells expressing constitutively active EGFRvIII mutant, which lacks 267 amino acids from its extracellular domain and is commonly found in GBM as well as in breast, ovarian, prostate, and lung carcinomas (Kuan et al., 2001), had significantly higher levels of PKM2 expression compared to U87/EGFR cells without EGF treatment (FIG. 56B). EGF-induced PKM2 upregulation was blocked by pretreatment with AG1478, an EGFR inhibitor (FIG. 56C), which indicates that EGFR activation is required for PKM2 upregulation. Pretreatment with cycloheximide, which blocks protein translation, inhibited EGF-induced PKM2 upregulation (FIG. 56D), suggesting that PKM2 expression is not primarily regulated by altering PKM2 stability. Real-time quantitative reverse transcriptase-polymerase chain reaction (RT-PCR) analysis using primers specific for mRNA of PKM2 or PKM1 showed an increase in the mRNA levels of PKM2, but not of PKM1, upon EGF treatment (FIG. 56E). These results suggest that EGFR activation enhances PKM2 protein expression by increasing its mRNA level.

### EGF Increases PKM2 Expression in a PKC- and NF-κB-Dependent Manner

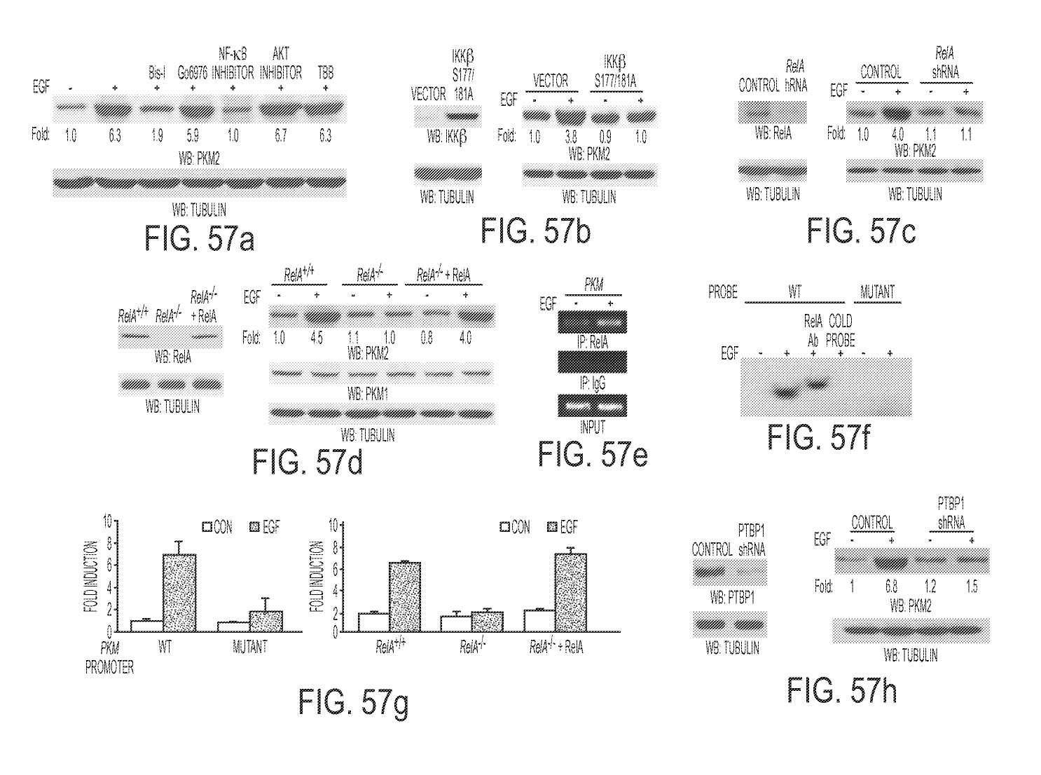

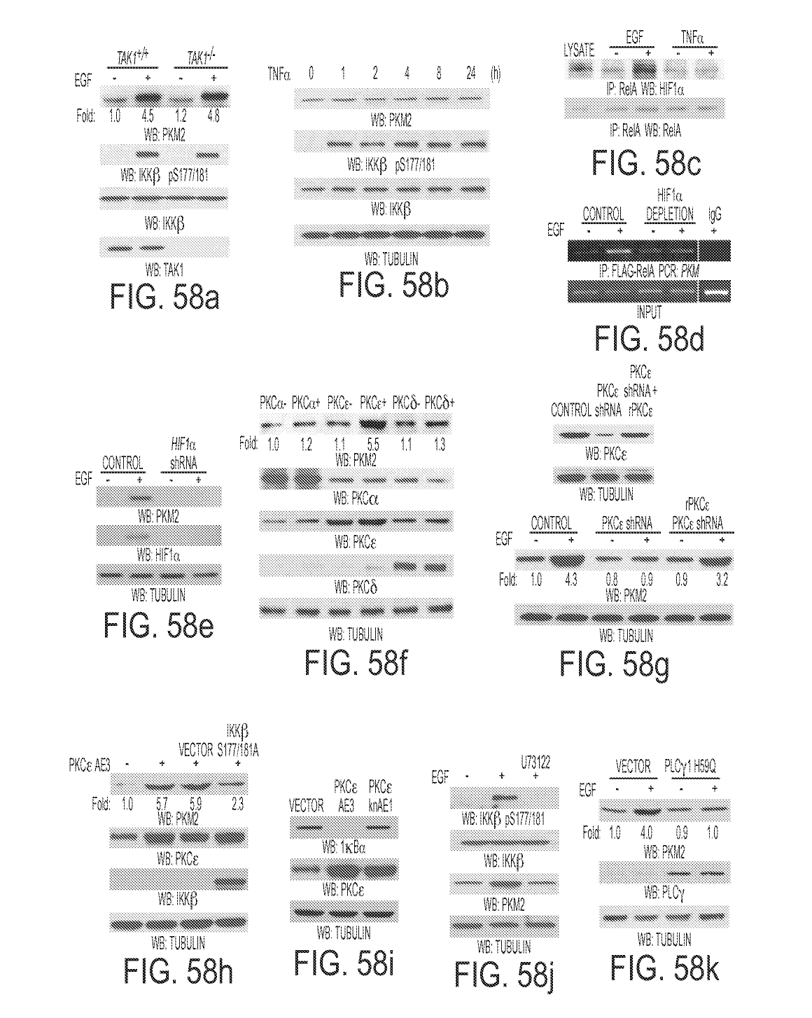

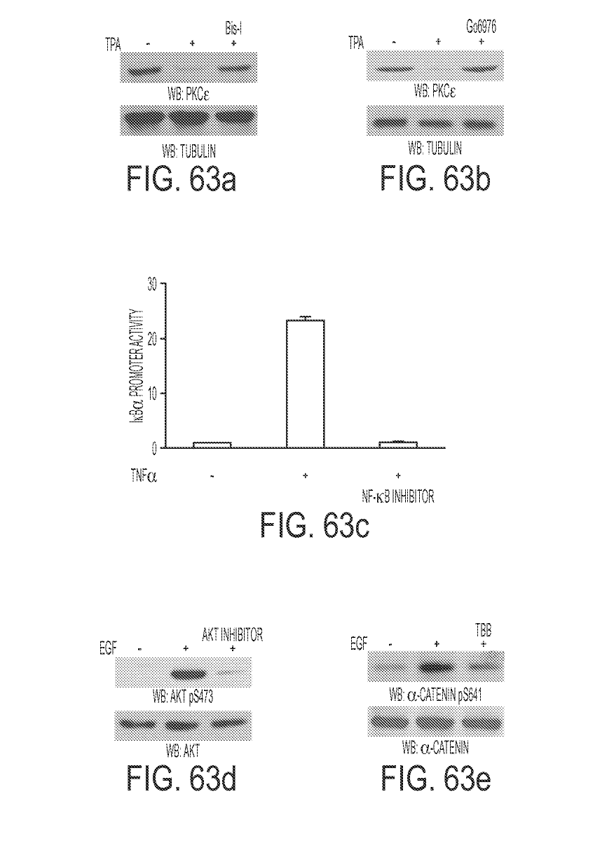

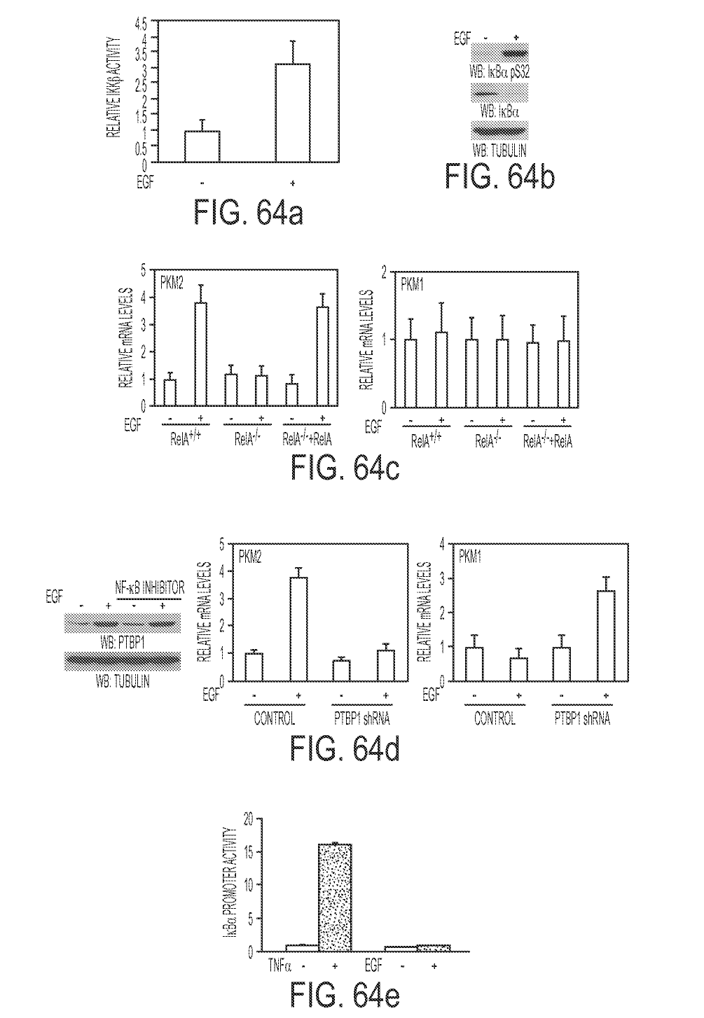

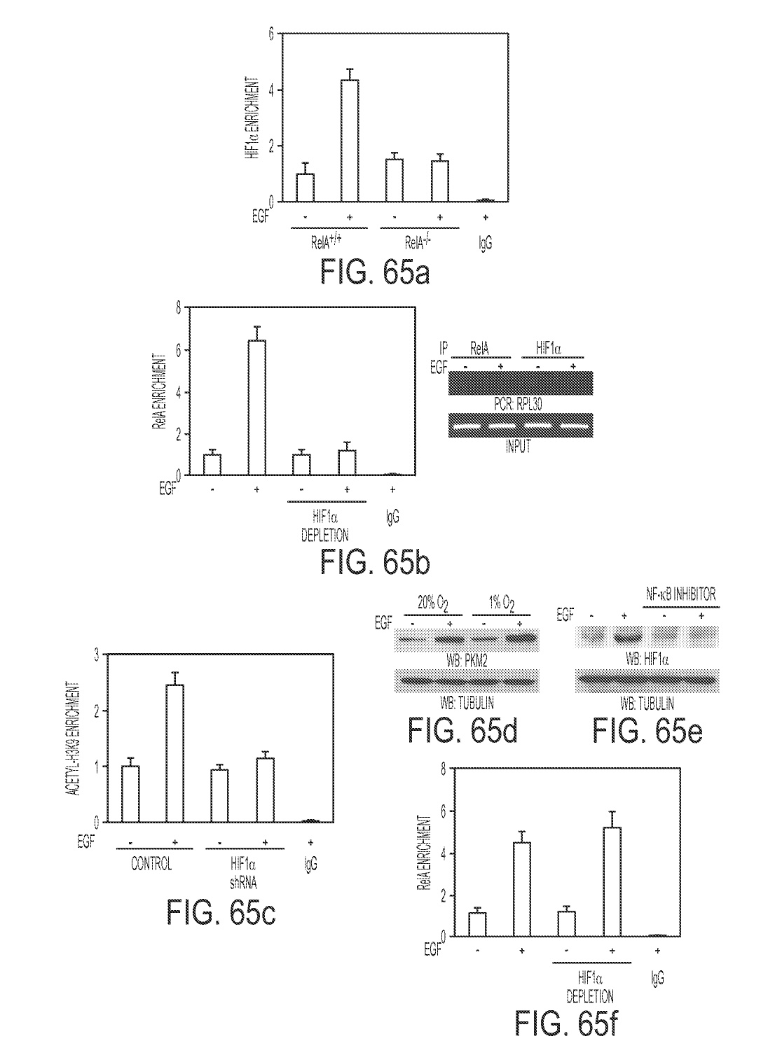

To determine how PKM2 expression is regulated by EGFR activation, the inventors pretreated U87/EGFR cells with the following inhibitors: general PKC inhibitor Bis-I, PKCα/β inhibitor Go6976, NF-κB activation inhibitor II, an AKT inhibitor, and CK2 inhibitor TBB, which successfully blocked 12-O-tetradecanoylphorbol-13-acetate (TPA)-induced and PKC kinase activity-dependent degradation of PKCε (FIG. 63A) and PKCα(FIG. 63B) (Lu et al., 1998), TNFα-induced and NF-κB-dependent IκBα promoter activation (FIG. 63C), EGF-induced AKT phosphorylation (FIG. 63D), and EGF-induced and CK2-dependent α-catenin S641 phosphorylation (Ji et al., 2009) (FIG. 63E), respectively. Inhibition of general PKC and NF-κB, but not of AKT or CK2 (FIG. 57A), largely abrogated EGF-induced PKM2 upregulation. These results implicate a role for PKC and NF-κB activation in the regulation of PKM2 expression. This finding was further validated by the observation that the stable expression of the dominant-negative kinase-dead IKKβ S177/181A mutant blocked EGF-induced PKM2 upregulation, which indicates that IKKβ activation is essential for this upregulation (FIG. 57B). Consistent with these findings, EGF treatment resulted in increased IKKβ activity (FIG. 64A) and enhanced cellular IkBα S32 phosphorylation and degradation of IkBα (FIG. 64B). In addition, RelA depletion by expressing RelA shRNA in U87/EGFR cells (FIG. 57C) or RelA deficiency blocked EGF-enhanced PKM2 expression without affecting PKM1 expression (FIG. 57D), whereas reconstituted expression of RelA in RelA^{-/-} mouse embryonic fibroblasts (MEFs) restored the ability of EGF to induce PKM2 expression (FIG. 57D).

Analysis of the PKM promoter using TFSEARCH software (http://www.cbrc.jp/research/db/TFSEARCH.html) identified a single putative NF-κB binding sequence, −291 GCGACTTTCC-300, which is similar to the NF-κB binding consensus sequence GGGRNNYYCC (N, any base; R, purine; and Y, pyrimidine) (Hayden and Ghosh, 2004). Chromatin immunoprecipitation (ChIP) with an anti-RelA antibody showed that EGFR activation results in the binding of RelA to the PKM promoter (FIG. 57E). To more directly assess an EGF-dependent NF-κB regulation of PKM, the inventors performed electrophoretic mobility shift assays (EMSA) with an oligonucleotide containing the putative wild-type (WT) or mutated NF-κB binding sequence (GCTACTTGTTT, highlighting the mutated nucleotides). The use of lysate derived from EGF-treated cells resulted in a marked increase in the NF-κB binding activity of the WT oligonucleotide, and the inclusion of an anti-RelA antibody resulted in a supershifted NF-κB-binding band (FIG. 57F). The inclusion of excess unlabelled oligonucleotide blocked the NF-κB binding activity, and the mutated oligonucleotide failed to bind to NF-κB. To examine the effect of NF-κB binding on the PKM promoter activity, the inventors transiently expressed a luciferase reporter vector containing the PKM promoter (from −1959 to −11 nucleotide) with either the WT or mutated NF-κB binding sequence into U87/EGFR cells, RelA^{+/+} MEFs, or RelA^{-/-} MEFs. As demonstrated in FIG. 57G, the activity of the WT, but not mutated, PKM promoter was significantly enhanced in EGF-treated U87/EGFR cells (left panel). Deficiency of RelA blocked EGF-induced PKM promoter activity, which was rescued by reconstituted expression of RelA in RelA^{-/-} MEFs (right panel). Real-time quantitative RT-PCR analysis showed that RelA deficiency inhibited an EGF-induced increase in mRNA levels of PKM2, but not of PKM1 (FIG. 64C). These results support a mechanism whereby EGFR activation results in NF-κB binding to GCGACTTTCC in the PKM promoter and activation of transcription.

EGF treatment increased the mRNA levels of PKM2 but not of PKM1 (FIG. 56E), suggesting that predominantly isoform-specific splicing of PKM pre-mRNA may occur co-transcriptionally. PTBP1, which is associated with gliomagenesis (Cheung et al., 2006), binds repressively to PKM sequences flanking exon 9, resulting in exon 10 inclusion (Clower et al., 2010; David et al., 2010). EGF treatment significantly increased PTBP1 expression (FIG. 64D, left panel), and RNAi-mediated PTBP1 depletion (FIG. 57H, left panel) blocked EGF-enhanced mRNA (FIG. 64D, middle panel) and protein expression of PKM2 (FIG. 57H, right panel), which was accompanied by upregulated PKM1 mRNA levels (FIG. 64D, right panel). PTBP1 protein expression was not affected by treatment with NF-κB inhibitor (FIG. 64D), indicating that NF-κB is not involved in the regulation of PTBP1 expression in response to EGF stimulation. These results suggest that EGF-induced upregulation of PKM2, but not PKM1, is regulated by both EGF-induced NF-kB activation and upregulated PTBP1 expression, which subsequently increase PKM transcription and generation of PKM2 mRNA by splicing, respectively.

PKCε Downstream from PLCγ1, Rather than TAK1, Activates IKKβ and Subsequently Increases PKM2 Expression

TAK1, which phosphorylates and activates IKKβ, is essential for canonical activation of RelA/p50 in response to inflammatory stimuli (Skaug et al., 2009). Nevertheless, the deficiency of TAK1 did not affect EGF-induced PKM2 expression or IKKβ activation, as reflected by its phosphorylation levels (FIG. 58A). These results indicate that EGF activates IKKβ/RelA via a mechanism that differs from the inflammatory stimuli-induced activation of IKKβ. In line with these findings, although both EGF and TNFα induced IKKβ activation, TNFα had no effect on PKM2 expression (FIG. 58B), whereas EGF induced significant PKM2 upregulation. In addition, a luciferase reporter assay showed that the promoter activity of IκBα, which is TNFα-induced and NF-κB-regulated, was enhanced by treatment with TNFα, but not EGF (FIG. 64E). These results suggest that EGFR activates IKKβ, thereby enhancing PKM2 expression, through a distinct signaling transmission, and NF-κB activation induced by EGF and TNFα regulates the expressions of different sets of downstream genes.

NF-κB activation in response to different extracellular stimuli likely enables NF-κB to be in complex with different transcriptional coregulators and to induce different sets of gene expression (Ghosh and Hayden, 2008; Hoffmann et al., 2006). Given that HIF1α is implicated as a transcriptional factor that regulates PKM2 transcription (Luo et al., 2011; Sun et al., 2011), the inventors have tested whether HIF1α is a coactivator with RelA in the regulation of PKM2. FIG. 58C shows that EGF, but not TNFα, induced an interaction between endogenous HIF1α and endogenous RelA. This interaction is required for HIF1α and RelA to bind to the PKM promoter, as demonstrated by ChIP and real-time quantitative PCR analysis, which showed that HIF1α increased its binding to the PKM promoter, which was blocked by RelA deficiency (FIG. 65A). Immunodepletion of HIF1α blocked EGF-induced binding of RelA to the PKM promoter (FIGS. 58D and 65B) and histone H3 acetylation at this promoter (FIG. 65C). In addition, depletion of HIF1α inhibited EGF-induced PKM2 expression (FIG. 58E). These results indicate that HIF1α is a distinct coactivator for RelA in response to EGF, but not TNFα, to induce PKM2 expression.

EGF-induced PKM2 expression in normoxic conditions could be further enhanced by creating a hypoxic condition that increased HIF1α expression (FIG. 65D). Intriguingly, upregulation of HIF1α expression was also induced upon EGF stimulation, which could be largely blocked by NF-κB inhibition (FIG. 65E). These results are in line with a previous report of IKKβ-dependent HIF1α transcription (Rius et al., 2008). Immunodepletion of HIF1α does not affect the binding of RelA to the HIF1α promoter upon EGF treatment (FIG. 65F), suggesting that RelA regulates HIF1α expression in an HIF1α-independent manner and that HIF1α is involved in some, but not all, NF-kB-dependent gene expression.

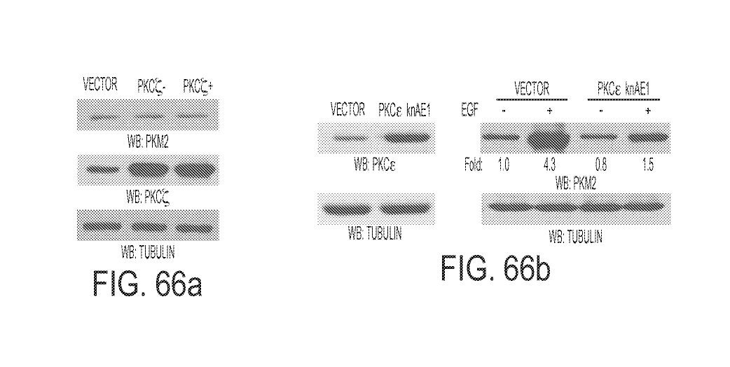

The general PKC inhibitor Bis-I, but not the PKCα/β inhibitor Go6976, blocked EGF-induced PKM2 upregulation (FIG. 57A), suggesting that a non-α/β isoform of PKC is involved in PKM2 regulation. Transient expression of constitutively active or kinase-dead mutants of PKCα, PKCε, PKCδ (FIG. 58F), or PKCζ (FIG. 66A) in U87/EGFR cells showed that only expression of a constitutively active mutant of PKCε (PKCε AE3) enhanced PKM2 expression. In addition, expression of the dominant-negative kinase-dead PKCε knAE1 (FIG. 66B) or shRNA depletion of PKCε (FIG. 58G) blocked EGF-induced PKM2 upregulation in U87/EGFR or U251 cells, and the effect of PKCε depletion was rescued by the expression of RNAi-resistant (r) PKCε (rPKCε). Furthermore, expression of a dominant-negative kinase-dead mutant of IKKβ (IKKβ S177/181A) largely blocked active PKCε-induced PKM2 expression (FIG. 58H). That PKCε activates IKKβ was further evidenced by enhanced IκBα degradation by an active, but not inactive, mutant of PKCε (FIG. 58I). These results indicate that PKCε, which is upstream from IKKβ activation, is responsible for EGF-induced PKM2 upregulation. In addition, inhibition of PLCγ with the PLCγ inhibitor U73122 or through stable expression of a dominant-negative PLCγ1 H59Q mutant in U87/EGFR cells abrogated EGF-induced IKKβ activation and PKM2 expression (FIGS. 58J and 58K). These results indicate that PLCγ1, which is downstream from EGFR and upstream from PKC activation, plays a key role in PKM2 upregulation.

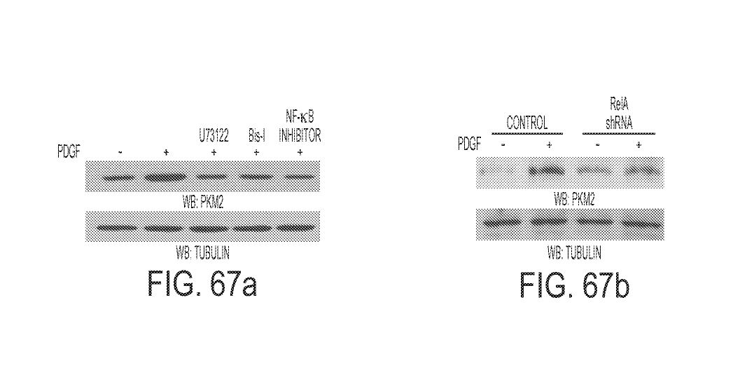

To test whether other growth factors regulate PKM2 expression, the inventors treated U87/EGFR cells with the platelet-derived growth factor (PDGF). FIG. 67 shows that PDGF induced PKM2 expression, which was blocked by inhibition of PLCγ, PKC, and NF-κB and depletion of RelA. These results indicate that both EGF and PDGF induce PKM2 expression in a PLCγ/PKC/NF-κB signal pathway-dependent manner.

### PKCε Phosphorylates IKKβ at Ser177 and Activates IKKβ

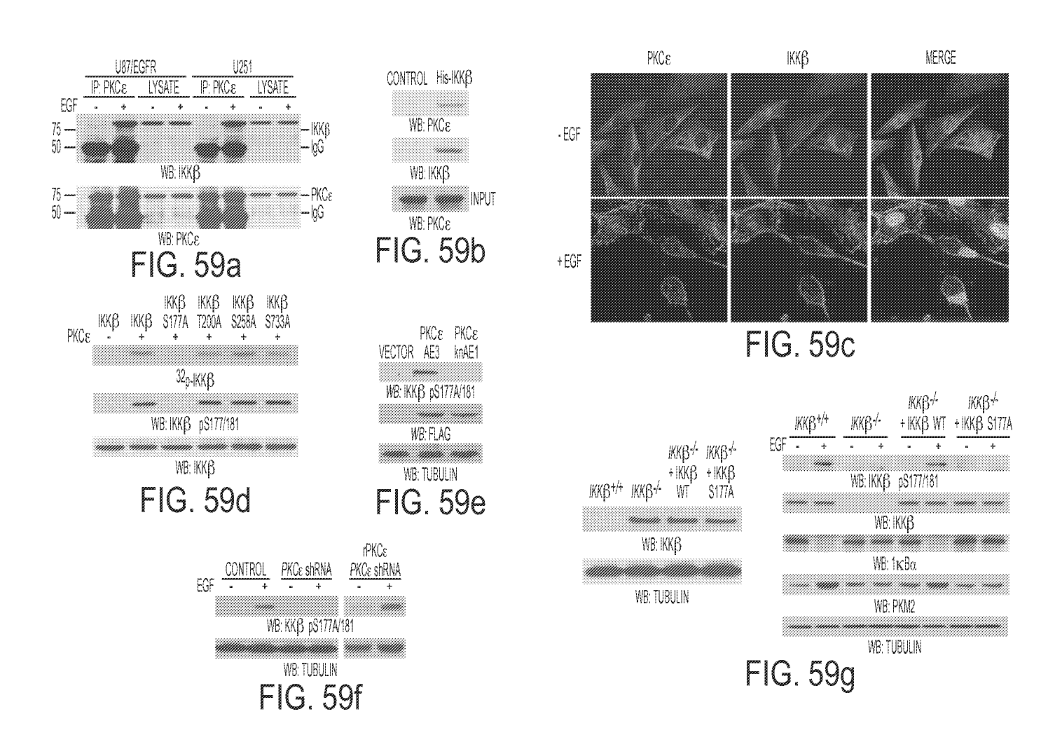

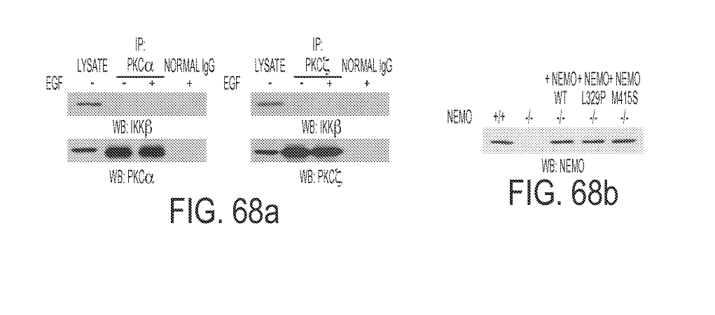

To determine whether PKCε directly activates IKKβ, the inventors immunoblotted the immunoprecipitated PKCε, PKCα, or PKCζ from U87/EGFR or U251 cells with an IKKβ antibody. This experiment showed that EGF induces an increased binding of endogenous IKKβ to PKCε (FIG. 59A), but not to PKCα or PKCζ (FIG. 68A). Furthermore, bacterially purified His-IKKβ pulled down purified active GST-PKCε, indicating that these two protein kinases directly bind each other (FIG. 59B). Immunofluorescent studies showed that both PKCε and IKKβ translocated from the cytosol to the membrane and co-localized with each other upon EGF treatment (FIG. 59C), and co-immunoprecipitation analyses of membrane fractions showed that the proteins interact with each other on the membrane.

To examine whether PKCε phosphorylates IKKβ, the inventors conducted an in vitro kinase assay, which showed that purified active PKCε phosphorylates bacterially expressed His-IKKβ (FIG. 59D). Analysis of IKKβ amino acid sequences by the motif-based profile-scanning ScanSite program revealed that IKKβ has several potential PKC phosphorylation motifs, at S177, T200, S258, and S733. Mutation of S177, but not T200, S258, or S733, into Ala largely reduced IKKβ phosphorylation by PKCε in vitro (FIG. 59D, upper panel). Immunoblotting analysis with an anti-phospho-IKKβ S177/181 antibody detected PKCε-phosphorylated WT IKKβ, but not IKKβ S177A mutant, indicating that S177, but not S181, is phosphorylated by PKCε (FIG. 59D, middle panel). In addition, expression of constitutively active PKCε AE3, but not the kinase-dead PKCε knAE1 mutant, resulted in phosphorylation of IKKβ at S177 (FIG. 59E). Furthermore, PKCε depletion (FIG. 58G) blocked EGF-induced IKKβ phosphorylation at S177 in U87/EGFR cells, which was rescued by reconstituted expression of rPKCε (FIG. 59F). Given that TAK1 activates IKKβ via phosphorylation of S177 in the activation loop of IKKβ (Wang et al., 2001), phosphorylation of IKKβ at S177 by PKCε might activate IKKβ, thereby inducing PKM2 expression. To test this hypothesis, the inventors reconstituted the expression of WT or S177A mutant IKKβ in IKKβ^{-/-} fibroblasts (FIG. 59G, left panel). IKKβ deficiency abrogated EGF-induced IKKβ phosphorylation at S177, IκBα degradation, and PKM2 upregulation, which were rescued by re-expression of WT IKKβ, but not IKKβ S177A mutant (FIG. 59G, right panel). These results indicate that PKCε phosphorylates IKKβ at S177 and activates IKKβ, which in turn induces PKM2 upregulation.

### Binding of NEMO Zinc Finger to Monoubiquitylated PKCε at Lys321 Regulates the Interaction Between PKCε and IKKβ

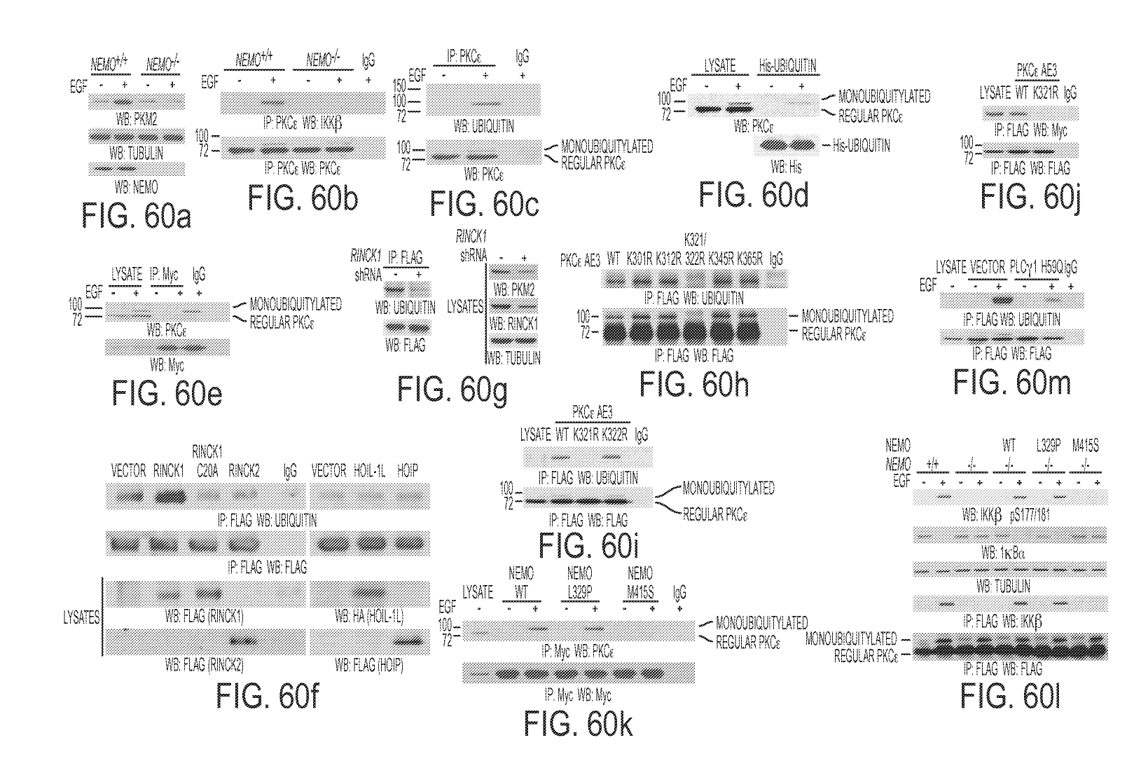

NEMO, functioning as an adaptor protein via binding of ubiquitylated proteins, is essential for TNFα-induced IKKβ phosphorylation and activation mediated by TAK1 (Skaug et al., 2009). NEMO deficiency completely blocked EGF-induced PKM2 expression (FIG. 60A), indicating its indispensable role in EGF-regulated PKM2 upregulation. Although IKKβ binds to PKCε directly in vitro (FIG. 59B), the membrane translocation of cytosolic IKKβ (FIG. 59C) and interaction with PKCε on the plasma membrane might need NEMO acting as a recruiting protein. To examine this hypothesis, the inventors immunoprecipitated PKCε from both NEMO^{+/+} and NEMO^{-/-} fibroblasts and immunoblotted it with an IKKβ antibody. As shown in FIG. 60B, NEMO deficiency abolished the EGF-induced association between endogenous PKCε and IKKβ. These results indicate that NEMO is required for PKCε binding to IKKβ in vivo. To determine whether NEMO or PKCε is ubiquitylated, the inventors used an anti-ubiquitin antibody to immunoblot immunoprecipitated NEMO or PKCε from EGF-treated or -untreated U87/EGFR cells. EGF stimulation induced monoubiquitylation of PKCε (FIG. 60C, top panel), whereas no ubiquitylation of NEMO was detected. Consistently, immunoblotting analyses showed that EGF treatment induced a slower migrating PKCε that was about 7 kD (a size of monoubiquitin) bigger than WT PKCε (FIG. 60C, bottom panel). Furthermore, His-protein pull-down analysis of 293T cells transiently expressing His-ubiquitin, which was followed by immmunoblotting with a PKCε antibody, showed that EGF treatment induced monoubiquitylated PKCε (FIG. 60D). In addition, the immunoblotting of immunoprecipitated Myc-tagged NEMO with a PKCε antibody showed that EGF treatment resulted in NEMO binding to monoubiquitylated PKCε but not to non-modified PKCε (FIG. 60E).

RING-finger protein that interacts with C kinase (RINCK)1 and linear ubiquitin assembly complex (LUBAC) composed of HOIL-1L and HOIP are known E3 ubiquitin ligases for PKC (Chen et al., 2007; Nakamura et al., 2006). Immunoblotting analysis of immunoprecipitated FLAG-PKCε AE3 with an anti-ubiquitin antibody showed that expressing WT RINCK1, but not inactive RINCK1 C20A, RINCK2, HOIL-1L, or HOIP, resulted in enhanced monoubiquitination of PKCε (FIG. 60F). In addition, RINCK1 depletion blocked EGF-induced FLAG-PKCε mono-ubiquitination and PKM2 expression (FIG. 60G). These results indicate that RINCK1 mediates PKCε monoubiquitination upon EGFR activation. To identify the ubiquitylated residue, the inventors treated the immunoprecipitated FLAG-PKCε from EGF-stimulated U87/EGFR cells with cyanogen bromide, which hydrolyzes peptide bonds at the C-terminus of methionine residues (Schroeder et al., 1969). Immmunoblotting analysis with an anti-ubiquitin antibody suggested that the −278 to −387 fragment contains the ubiquitylated residue. Mutation of K321/322, but not K301, K312, K345, or K365, into Arg abolished EGF-induced monoubiquitylation of FLAG-tagged PKCε (FIG. 60H). A single mutation at K321 or K322 showed that mutation at K321, but not K322, abrogated monoubiquitylation of PKCε upon EGF stimulation (FIG. 60I). In addition, co-immunoprecipitation analyses showed that a K321R mutant of FLAG-PKCε AE3 lost its binding to Myc-tagged NEMO (FIG. 60J), indicating that EGF-induced PKCε monoubiquitylation at K321 provides a binding motif for NEMO.

To determine whether the UBD domains of NEMO bind to monoubiquitylated PKCε, the inventors stably expressed Myc-tagged NEMO WT, L329P mutant abrogated ubiquitin-binding ability of LZ motif (NOA/UBAN/NUB domain) of NEMO (Wu et al., 2006), and M415S mutant interrupted the UBD domain in ZF domain of NEMO (Cordier et al., 2009) in U87/EGFR cells. Immunoblotting of immunoprecipitated Myc-NEMO with a PKCε antibody showed that the mutation at M415, but not at L329, abolished the EGF-induced interaction between NEMO and PKCε (FIG. 60K). Furthermore, NEMO^{-/-} fibroblasts with reconstituted expression of NEMO M415S, but not WT or L329P mutant (FIG. 68B), failed to rescue NEMO-deficiency-induced inhibition of EGF-enhanced IKKβ S177 phosphorylation, IκBα degradation, and binding of IKKβ to FLAG-PKCε (FIG. 60L). These results indicate that NEMO zinc finger binding to monoubiquitylated PKCε plays a pivotal role in IKKβ activation by PKCε.

Inhibition of PLCγ1 abrogated EGF-induced IKKβ activation and PKM2 expression (FIGS. 58J and 58K). To further investigate whether PLCγ1 regulates PKM2 expression via PKCε, the inventors treated U87/EGFR cells stably expressing PLCγ1 H59Q mutant (FIG. 58K) with EGF, showing that expression of the dominant-negative PLCγ1 mutant significantly blocked EGF-induced PKCε monoubiquitylation (FIG. 60M). These results further support that PLCγ1 downstream from EGFR upregulates PKM2 expression via regulation of PKCε.

### EGF Promotes Glycolysis and Tumorigenesis by PKCε- and NF-κB-dependent PKM2 Upregulation

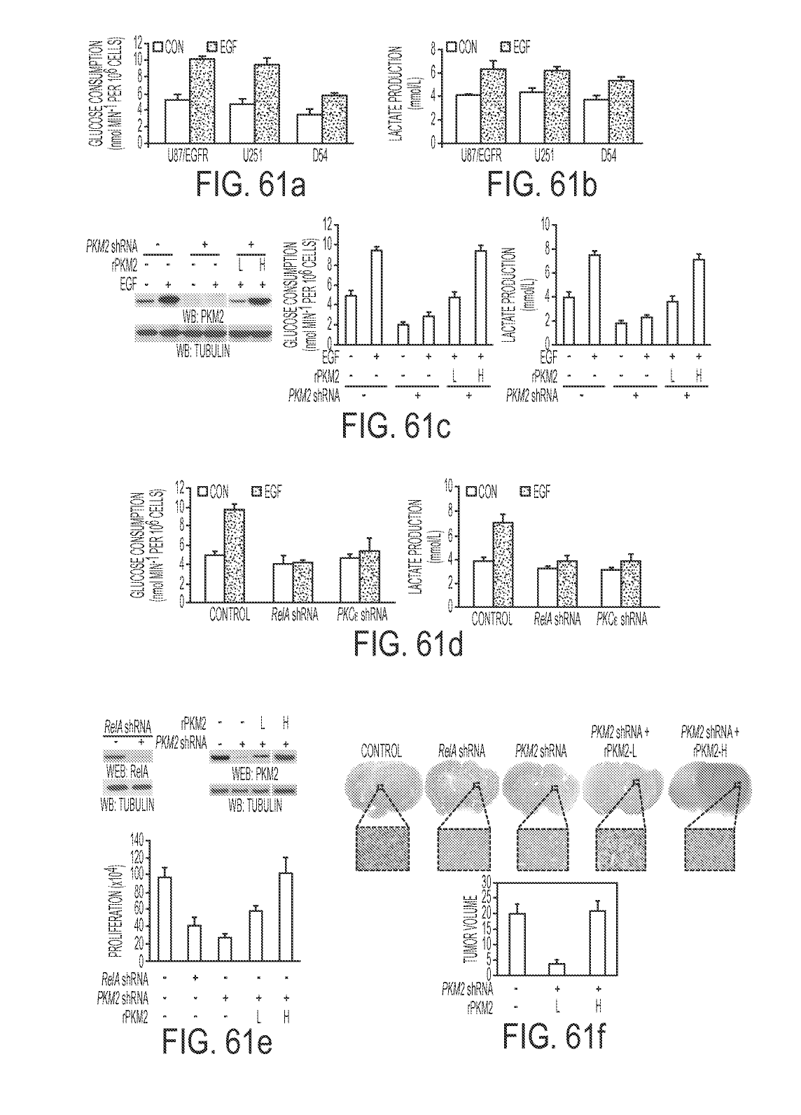

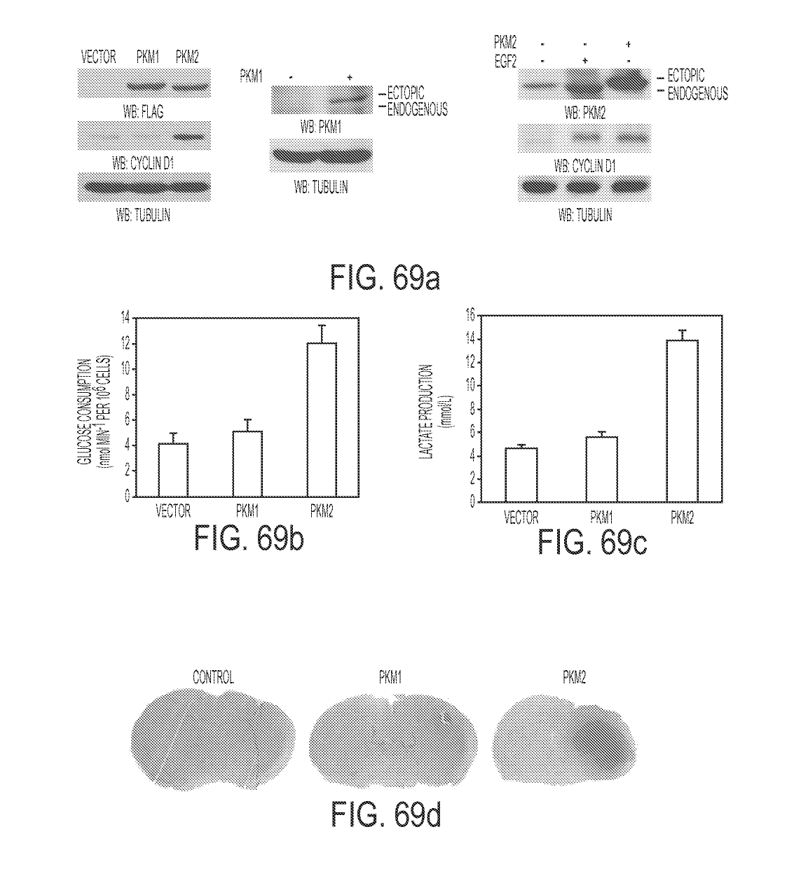

EGF treatment of U87/EGFR, U251, and D54 human GBM cells enhanced glucose consumption (FIG. 61A) and lactate production (FIG. 61B), which was also observed by overexpression of PKM2, but not PKM1, in U87/EGFR cells (FIG. 69A-C). In addition, overexpression of PKM2, but not PKM1, enhanced cyclin D1 expression (FIG. 69A), which is consistent with the finding that PKM2 regulates β-catenin transactivation and expression of downstream CCND1 gene (encoding cyclin D1) (Yang et al., 2011). In contrast to PKM2 overexpression, PKM2 depletion (FIG. 61C, left panel) largely reduced both basal and EGF-enhanced glucose consumption and lactate production (FIG. 61C, middle and right panels). Reconstituted expression of RNAi-resistant PKM2 (rPKM2) restored EGF-promoted glycolysis in an rPKM2 expression level-dependent manner (FIG. 61C), indicating that increased PKM2 expression upon EGF stimulation plays an instrumental role in EGFR-promoted glycolysis. Consistently, depletion of RelA or PKCε, which largely reduced EGF-induced PKM2 upregulation (FIGS. 57C and 58G), significantly reduced EGF-enhanced glucose consumption and lactate production (FIG. 61D).

Depletion of RelA or PKM2 from U87/EGFRvIII cells inhibited proliferation of cells, which were in culture for seven days (FIG. 61E). Reconstituted expression of rPKM2 in U87/EGFRvIII-PKM2 shRNA cells restored EGFRvIII-promoted cell proliferation in an rPKM2 expression level-dependent manner (FIG. 61E). These results indicate that EGFR-increased PKM2 expression is required for EGFR-promoted cell proliferation.

To determine the roles of RelA and PKM2 in brain tumor development, the inventors intracranially injected U87/EGFRvIII cells, with or without depleted RelA or PKM2, or U87/EGFRvIII cells with depleted endogenous PKM2 and reconstituted expression of rPKM2 into athymic nude mice. Dissection of the mice two weeks after injection revealed that all of the animals injected with U87/EGFRvIII cells had rapid tumor growth (FIG. 61F). In contrast, no tumors were detected in the mice injected with U87/EGFRvIII cells with depleted RelA or PKM2. Reconstituted expression of rPKM2 in U87/EGFRvIII-PKM2 shRNA cells restored EGFRvIII-promoted tumor growth in an rPKM2 expression level-dependent manner (FIG. 61F). U87/EGFR cells injected into mice for two weeks did not lead to tumor formation. However, overexpression of PKM2, but not PKM1, in U87/EGFR cells elicited significant tumor growth (FIG. 69D). These results elucidate the significance of RelA-dependent PKM2 upregulation in EGFR-promoted brain tumor development.

Levels of PKM2 Correlate with Levels of EGFR Activity in Human GBM and with Grades of Glioma Malignancy and Prognosis

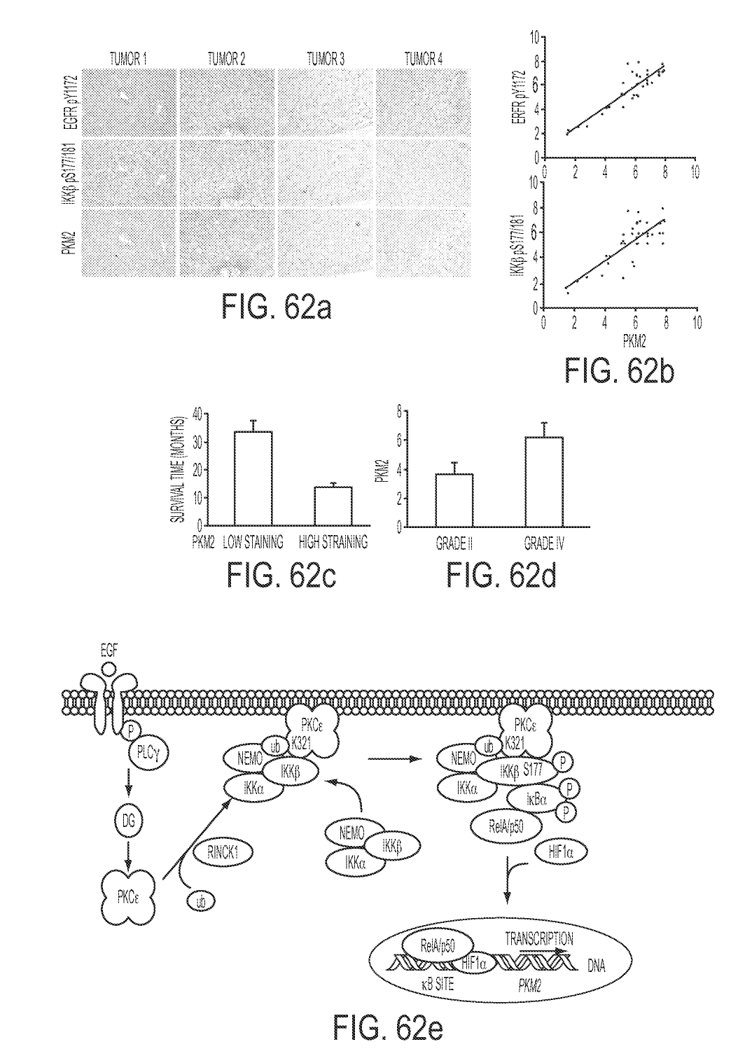

The inventors demonstrated that EGFR activation results in IKKβ-dependent PKM2 upregulation. To further determine whether the findings have clinical relevance, the inventors examined the activity of EGFR and IKKβ and PKM2 expression levels in serial sections of 55 human primary GBM specimens by immunohistochemical (IHC) analyses. As shown in FIG. 62A, the activity levels of EGFR and IKKβ reflected by their phosphorylation levels correlated with the levels of PKM2 expression. Quantification of the staining on a scale of 0-8 showed that the correlation between PKM2 expression levels and EGFR (FIG. 62B, upper panel: r=0.80, P<0.001) or IKKβ activity (FIG. 62B, bottom panel: r=0.88, P<0.001) was significant in different specimens. The survival durations for the 55 patients, all of whom received standard adjuvant radiotherapy after surgery, followed by treatment with an alkylating agent (temozolomide in the majority of cases), were analyzed with respect to low (2-5 staining) versus high (5.1-8 staining) expression of PKM2. Patients with low PKM2-expressing tumors (20 cases) had a median survival of 34.5 months, compared with 13.6 months for patients with high PKM2-expressing tumors (35 cases) (P<0.001) (FIG. 62C). These results strongly support a role for EGFR activation in the IKKβ-dependent upregulation of PKM2 in human GBM and reveal a strong relationship between PKM2 expression and patient prognosis.

To examine whether the level of PKM2 expression correlated with the grade of glioma malignancy, the inventors compared PKM2 expression levels in low-grade diffuse astrocytoma (WHO grade II) and high-grade GBM (WHO grade IV) (Furnari et al., 2007). IHC analyses of 27 human low-grade diffuse astrocytoma specimens showed significantly lower levels of PKM2 in these low-grade gliomas than in the GBM specimens (FIG. 62D). Thus, the level of PKM2 correlated with the grade of glioma malignancy.

### Discussion

PKM2 plays an essential role in aerobic glycolysis and tumor growth (Christofk et al., 2008). Nevertheless, how PKM2 expression is regulated during tumor development remains largely unclear. Although the mechanisms and the role of NF-κB activation during inflammatory response have been reported (Skaug et al., 2009), answers to the questions of how NF-κB is regulated in response to growth factor stimulation and whether this regulation contributes to cancer cell metabolism remain elusive (Brown et al., 2008). Studies detailed here revealed an important mechanism underlying the upregulation of PKM2 and the activation of NF-κB by EGFR activation in tumor cells.

In stark contrast to TNFα-induced NF-κB activation, EGF-induced NF-κB activation is TAK1 independent, and IKKβ S177, which is phosphorylated by TAK1 in inflammatory response, is phosphorylated by PKCε instead. In addition, monoubiquitylated PKCε provides a docking site for binding of the NEMO zinc finger. The binding of NEMO to PKCε creates a direct interaction between PKCε and IKKβ, which results in IKKβ phosphorylation by PKCε and subsequent NF-κB activation. EGFR activation results in the plasma membrane translocation of cytosolic PKCε and IKKβ and in the interaction of these proteins on the membrane, strongly indicates that PKCε phosphorylates and activates IKKβ on the plasma membrane. In addition, EGF, but not TNFα, induced HIF1α expression and an interaction between RelA and HIF1α, which is required for the binding of RelA to PKM promoter and PKM2 expression. Although RelA by itself is sufficient to bind a nucleosome-unassociated oligonucleotide containing the NF-κB binding sequence, it needs HIF1α to act as a co-activator to induce PKM2 transcription, and HIF1α may facilitate and stabilize the transcription factor complexes at PKM2 promoter regions. These results indicate that EGF and TNFα activate NF-κB via distinct mechanisms and subsequently induce different sets of gene expression. NF-κB-dependent PKM transcription acts coordinately with splicing of the pre-mRNA, which is mediated by EGF-induced upregulation of PTBP1, leading to increased expression of PKM2, but not PKM 1.

Aberrantly higher activity of EGFR due to gene amplification or mutation of EGFR has been detected in approximately 40% of GBM tumors, which are the most common and biologically aggressive types of brain tumors (Voldborg et al., 1997; Wykosky et al., 2011). The activity levels of EGFR and IKKβ in human GBM cell lines correlate with the levels of PKM2 expression. In addition, the level of PKM2 in human glioma tissue correlates with the level of EGFR activity, grade of glioma malignancy, and patient prognosis, suggesting that PKM2 expression levels can serve as a biomarker for brain tumor malignancy and prognosis Depletion of PKM2 or blocking PKM2 upregulation by expression of RelA shRNA largely inhibited EGFR-enhanced aerobic glycolysis in GBM cells and brain tumor growth, indicating that NF-κB activation-dependent PKM2 plays a crucial role in EGFR-promoted GBM cell metabolism and brain tumor growth.

In view of the studies presented here a mechanistic model of tumor metabolism is proposed that integrates these different components. The findings demonstrate that activation of EGFR in human cancer cells results in increased glucose uptake and lactate production in a PKM2 expression-dependent manner. Furthermore, EGF-induced PKM2 upregulation is dependent on activation of a PLCγ1-PKCε-IKKβ-RelA signaling cascade (FIG. 62E) in which NEMO zinc finger binds to monoubiquitylated PKCε at K321, mediated by RINCK1, leading to the direct interaction between IKKβ and PKCε and phosphorylation and activation of IKKβ by PKCε. Activated RelA in complex with its co-activator HIF1α is required for PKM2 expression.

Increased PKM2 expression enhanced cyclin D1 expression, glycolysis, cell proliferation, and tumorigenesis, highlighting the essential role of PKM2 expression levels in tumor development. The inventor's studies unearthed important mechanisms underlying EGFR-induced NF-κB activation and upregulated PKM2 expression during tumor development. The demonstration of a mechanistic interplay between the EGFR and NF-κB pathways in cancer metabolism provides an important insight for further understanding tumor development and may provide a molecular basis for treating activated EGFR- and upregulated PKM2-related tumors by interfering with this EGFR-induced signaling transmission at multiple levels.

In response to TNFα stimulation, PKC phosphorylates p65 at S311 and promotes p65 transcriptional activity (Duran et al., 2003; Moscat et al., 2006) whereas PKCα is involved in NF-κB activation induced by TPA, but not TNFα (Lallena et al., 1999). In response to EGF, PKCε, but not PKCα or PKCζ, interacts with and phosphorylates IKKβ. In addition, expression of constitutively active or dominant-negative mutant of PKCα or PKCζ does not affect EGF-induced PKM2 upregulation, indicating that EGFR activation regulates PKM2 expression via a distinct signaling pathway.

Inhibition of EGFR activation by the EGFR-specific inhibitor AG1478 abrogated EGF-induced PKM2 upregulation, indicating that the enhanced PKM2 transcription is EGFR kinase activity dependent. p53 deficiency, via an unknown mechanism, upregulates NF-κB activity, which depends on the expression of the glucose transporter GLUT3. The activated NF-κB, in turn, further enhances GLUT3 expression, thereby forming a positive feedback loop (Kawauchi et al., 2008). EGFR activation also leads to NF-κB activation and PKM2 upregulation in WT p53-expressing U87/EGFR cells (Badie et al., 1999), indicating that EGFR-regulated NF-κB/PKM2 is p53 status independent.

### Materials and Methods

### Materials

Rabbit polyantibodies recognizing PKM1, EGFR, phospho-a-catenin S641, phospho-EGFR-Y1172, and RelA were obtained from Signalway Biotechnology (Pearland, Tex.), and rabbit polyantibodies recognizing PKM2, IKKβ, and phospho-IKKβ-S177/181 were obtained from Cell Signaling Technology (Danvers, Mass.). Polyclonal antibodies for PKCα, PKCε, PKCδ, PTBP1, TAK1, PLCγ, phospho-IkBα S32, RINCK1, NEMO, and IkBα were purchased from Santa Cruz Biotechnology (Santa Cruz, Calif.). Monoclonal antibody for ubiquitin was acquired from Invitrogen (Carlsbad, Calif.). EGF and mouse monoclonal antibodies for FLAG, Myc, His, and tubulin were purchased from Sigma (St. Louis, Mo.). Polyclonal antibody for HIF1α was from BD Biosciences (San Jose, Calif.). Hygromycin, puromycin, G418, Bis-I, Go6976, NF-kB inhibitor, AKT inhibitor, TBB, U73122, and U0126 were purchased from EMD Biosciences (San Diego, Calif.). Active PKCε was obtained from Signalchem (Richmond, Canada). Hoechst 33342, Alexa Fluor 488 goat anti-mouse antibody, and Alexa Fluor 594 goat anti-rabbit antibody were from Molecular Probes (Eugene, Oreg.). HyFect transfection reagents were from Denville Scientific (Metuchen, N.J.). GelCode Blue Stain Reagent was obtained from Pierce (Rockford, Ill.).

### In Vitro Kinase Assays

The kinase reactions were done by mixing purified active PKCε and bacterially purified WT His-IKKβ or different His-IKKβ mutants in 20 μL kinase assay buffer containing 10 μCi of [gamma-³²P] ATP, 25 mM MOPS (pH 7.2), 12.5 mM β-glycerol-phosphate, 25 mM MgCl₂, 5 mM EGTA, 2 mM EDTA, 0.25 mM DTT, and 2.5 μL PKC lipid activator (SignalChem, Richmond, BC, Canada) for 20 min at 30° C. Reactions were stopped by adding an equal volume of 2×SDS-polyacrylamide gel electrophoresis (PAGE) sample buffer and boiling for 5 min. Samples were then separated by 6% SDS-PAGE and transferred onto nitrocellulose membranes for exposing to X-ray film. Biotinylated IκBα (Ser32) peptide was used for measuring IKKβ activity (HTScan IKKβ kinase assay kit, Cell Signaling Technology, Danvers, Mass.).

### Luciferase Reporter Gene Assay

The luciferase reporter vector pGL3-promoter containing either the WT or a mutated PKM promoter fragment or a luciferase reporter vector containing the IκBα promoter was transfected into U87/EGFR cells, RelA+/+, or RelA−/− fibroblasts seeded in 24-well plates at 1.5×10⁴ cells/well. Twelve hours after transfection, the medium was replaced with 0.1% serum for another 12-24 h, and EGF (100 ng/mL) was added 12 h before harvesting. Ten milliliters out of the 100 mL cell extract were used for measuring luciferase activity. The relative levels of luciferase activity were normalized to the levels of untreated cells and to the levels of luciferase activity of the Renilla control plasmid. Data represent the mean±standard deviation of three independent experiments.

### Cells and Cell Culture Conditions

U87, U87/EGFR, U251, and D54 GBM cells; DU145 prostate cancer cells; MDA-MB-231 breast cancer cells; and 293T, RelA, RelA^{-/-}, NEMO^{+/+}, NEMO^{-/-}, IKKβ^{+/+}, and IKKβ^{-/-} cells were maintained in Dulbecco's modified Eagle's medium (DMEM) supplemented with 10% bovine calf serum (HyClone, Logan, Utah). Cell cultures were made quiescent by growing them to confluence and then replacing the medium with fresh medium containing 0.5% serum for 1 d.

### Transfection

Cells were plated at a density of 4×10⁵/60-mm dish 18 h prior to transfection. Transfection was performed using HyFect reagents (Denville Scientific) according to the vendor's instructions. Transfected cultures were selected with puromycin (5 ug/mL), hygromycin (200 μg/mL), or G418 (400 μg/mL) for 10-14 d at 37° C. At that time, antibiotic-resistant colonies were picked, pooled, and expanded for further analysis under selective conditions.

### Immunoprecipitation and Immunoblotting Analysis

Extraction of proteins with a modified buffer from cultured cells was followed by immunoprecipitation and immunoblotting with corresponding antibodies, as described previously (Lu et al., 1998).

### DNA Constructs and Mutagenesis

The PKM2 promoter region (−1959 to −11) acquired by PCR was constructed into a luciferase reporter system (pGL3-PKM2). PCR-amplified human IKKβ was cloned into either pCold I vector (TaKaRa, Shiga, Japan) between Hind III and Xba I or pcDNA3.1/hygro (+) vector between Hind III and Kpn I. PKCε was subcloned into pcDNA3.1/hygro (+)-FLAG between BamH I and Not I. pGL3-PKM2 with a mutation at the NF-kB binding site; pCold I-IKKβ S177A, -IKKβ T200A, -IKKβ S258A, and -IKKβ S733A; pcDNA3.1/hygro (+)-IKKβ S177A, -IKKβ T200A, -IKKβ S258A, and -IKKβ S733A; and pcDNA3.1/hygro (+)-FLAG-PKCε K301R, -PKCε K312R, -PKCε K321/322R, -PKCεK321R, -PKCε K322R, -PKCε K345R, and -PKCε K365R were made using the QuikChange site-directed mutagenesis kit (Stratagene, La Jolla, Calif.). pCDNA 3.1 rPKM2 contains mutations at C1170T, C1173T, T1174C, and G1176T. pCDNA 3.1 PKCε contains mutations at C1090A and G1192A.

pGIPZ control was generated with a control oligonucleotide GCTTCTAACACCGGAGGTCTT (SEQ ID NO:1). pGIPZ PKM2 shRNA was generated with CATCTACCACTTGCAATTA (SEQ ID NO: 14) oligonucleotide targeting transcript of exon 10 of the PKM2 gene. pGIPZ PKCε shRNA and RelA shRNA were generated with CAACATTCGGAAAGCCTTGTC (SEQ ID NO: 28) and GAGCATCATGAAGAAGAGTCC (SEQ ID NO: 29), respectively. PTBP1 knockdown was performed through transduction with commercially prepared PTBP1 shRNA (Cat #sc-38280-V) or control (Cat #sc-108080) lentiviral lysates (Santa Cruz Biotechnology, Ic, Santa Cruz, Calif.).

### RT-PCR and Quantitative Real-Time PCR

Total RNA was extracted using a DNAfree kit (Qiagen Valencia, Calif.) and purified using an RNeasy kit (Qiagen). cDNA was prepared using oligonucleotide (dT), random primers, and Superscript III (Invitrogen). RT-PCR analysis of PKM2, PKM1, and β-actin as a control was carried out using the following primer pairs: PKM2, 5′-GGGTTCGGAGGTTTGATG-3′ (SEQ ID NO: 30) (forward) and 5′-ACGGCGGTGGCTTCTGT-3′ (SEQ ID NO: 31) (reverse); PKM1, 5′-CTGGAGAAACAGCCAAAGG-3′ (SEQ ID NO: 32) (forward) and 5′-GCCAGACTCCGTCAGAACTA-3′ (SEQ ID NO: 33) (reverse); β-actin, 5′-ATGGATGACGATATCGCTGCGC-3′ (SEQ ID NO: 12) (forward) and 5′-GCAGCACAGGGTGCTCCTCA-3′ (SEQ ID NO: 13) (reverse). Quantitative real-time PCR analysis was performed using IQ™ SYBR GREEN SUPERMIX (Bio-Rad, Hercules, Calif.) under the following conditions: 5 min at 95° C. followed by 40 cycles at 95° C. for 30 s, 55° C. for 40 s, and 72° C. for 1 min using an ABI Prism 7700 sequence detection system. Data show mRNA expression levels relative to those of β-actin; the former was then normalized to control expression levels for each experiment.

### Purification of Recombinant Proteins

The WT and mutants of His-IKKβ proteins were expressed in bacteria and purified, as described previously (Xia et al., 2007). Briefly, the vectors expressing WT and mutants of His-IKKβ were used to transform BL21/DE3 bacteria. Transformants were used to inoculate 50 mL cultures of LB/ampicillin, which were grown overnight at 37° C. to stationary phase. A measure of 5 mL preculture was then used to inoculate 200 mL LB/ampicillin. The cultures were grown at 37° C. to an OD₆₀₀ of ˜0.6 before inducing with 0.5 mM IPTG at 16° C. for 24 h. Cell pellets were collected, resuspended in 10 mL Bugbuster® protein extraction reagent (buffer) (EMD, San Diego, Calif.) with the addition of 20 μL protease cocktail inhibitor (EMD), and incubated at room temperature for 20 min, before centrifugation at 10,000 rpm for 10 min (4° C.). Cleared lysates were then bound to Ni-NTA His•Bind® Resins (EMD) for 3 h, with rolling at 4° C. Beads were washed extensively with the extraction buffer before eluting for 1 h in extraction buffer (pH 7.5) plus 500 mM imidazole. Eluted proteins were then dialyzed extensively against 20 mM Tris-Cl pH 8.0, 50 mM NaCl, 10% glycerol, and 1 mM DTT.

### Electrophoretic Mobility Shift Assay (EMSA)

Cells were solubilized in buffer (10 mM HEPES pH 7.2, 10 mM KCl, 0.1 mM EDTA, 0.1 mM EGTA, 0.4% NP-40, protease inhibitor cocktail, 1 mM DTT) and centrifuged at 10,000 g for 10 min. Pellets were resuspended in buffer (20 mM HEPES pH 7.9, 400 mM NaCl, 1 mM EDTA, 1 mM EGTA, protease inhibitor cocktail, 1 mM DTT) and then centrifuged at 20,000 g for 15 min. The supernatant was used as the nuclear extract. ³²P-labelled DNA probes for the WT NF-κB binding site (underlined),

(SEQ ID NO: 34)
CCCCGGAGCGACTTTCCTCCCAG,

and for the mutant of the NF-κB binding site,

(SEQ ID NO: 35)
CCCCGGAGCTACTTGTTTCCCAG,

were prepare. Nuclear extracts (10 μg of protein) were incubated with the ³²P-labelled probes (100,000 cpm) in 20 μL of buffer (20 mM HEPES pH 7.9, 5% glycerol, 1 mM EDTA, 100 μg/mL poly dI-dC) for 20 min at room temperature. Samples were subjected to 5% polyacrylamide gel electrophoresis.

### Measurements of Glucose Consumption and Lactate Production

Cells were seeded in culture dishes and the medium changed after 6 h. Cells were incubated for 20 h, and the culture medium was then collected for measurement of glucose and lactate concentrations. Glucose levels were determined using a glucose (GO) assay kit (Sigma). Glucose consumption was the difference in glucose concentration compared with control. Lactate levels were determined using a lactate assay kit (Eton Bioscience, Inc., San Diego, Calif.). Cells were collected and counted, and glucose consumption and lactate production were normalized by cell numbers (per 10⁶).

### Immunofluorescence Analysis

Cells were fixed and incubated with primary antibodies, Alexa Fluor dye-conjugated secondary antibodies, and Hoechst 33342 according to standard protocols. Cells were examined using a deconvolution microscope (Zeiss, Thornwood, N.Y.) with a 63-Å oil immersion objective. Axio Vision software from Zeiss was used to deconvolute Z-series images.

### Immunohistochemical Analysis

Mouse tumor tissues were fixed and prepared for staining as previously described (Zheng et al., 2009). The specimens were stained with Mayer's hematoxylin and subsequently with eosin (Biogenex Laboratories, San Ramon, Calif.). Afterward, the slides were mounted using Universal Mount (Research Genetics).

The tissue sections from paraffin-embedded human GBM specimens were stained with an antibody against phospho-EGFR-Y1172 (Signalway Antibody), PKM2 antibody (Cell Signaling Technology), or nonspecific IgG as a negative control. The inventors quantitatively scored the tissue sections according to the percentage of positive cells and staining intensity, as previously defined (Zheng et al., 2009). The inventors assigned the following proportion scores: 0 if 0% of the tumor cells showed positive staining, 1 if 0% to 1% of cells were stained, 2 if 1% to 10% stained, 3 if 11% to 30% stained, 4 if 31% to 70% stained, and 5 if 71% to 100% stained. The inventors rated the intensity of staining on a scale of 0 to 3: 0, negative; 1, weak; 2, moderate; and 3, strong. The inventors then combined the proportion and intensity scores to obtain a total score (range, 0-8), as described previously (Allred et al., 1998). Scores were compared with overall survival, defined as the time from date of diagnosis to death or last known date of follow-up. All patients received standard adjuvant radiotherapy after surgery, followed by treatment with an alkylating agent (temozolomide in the majority of cases). The use of human brain tumor specimens and the database was approved by the institutional review board at MD Anderson Cancer Center.

### Intracranial Injection

The inventors intracranially injected 5×10⁵ U87/EGFRvIII or U87/EGFRvIII cells expressing different shRNAs (in 0.15 mL of DMEM per mouse) into 4-week-old female athymic nude mice. The intracranial injections were performed as described in a previous publication (Gomez-Manzano et al., 2006). Seven mice per group in each experiment were included. Animals were killed three weeks after glioma cell injection. The brain of each mouse was harvested, fixed in 4% formaldehyde, and embedded in paraffin. Tumor formation and the phenotype were determined by histologic analysis of H & E-stained sections. Tumor volumes were defined as (longest diameter)×(shortest diameter)²×0.5.

### Example 5

### PKM2 Regulates Chromosome Segregation and Mitosis Progression

Studies detailed below demonstrate that PKM2 binds to Bub3 during mitosis and phosphorylates Bub3 at Y207. This phosphorylation event is shown to be required for recruitment of the Bub3-Bub1 complex to Blinkin and kinetochores and the subsequent regulation of chromosome segregation, cell proliferation, and tumorigenesis.

### Results

### PKM2 is Required for the Fidelity of Chromosome Segregation and Kinetochore Localization of Bub3 and Bub1

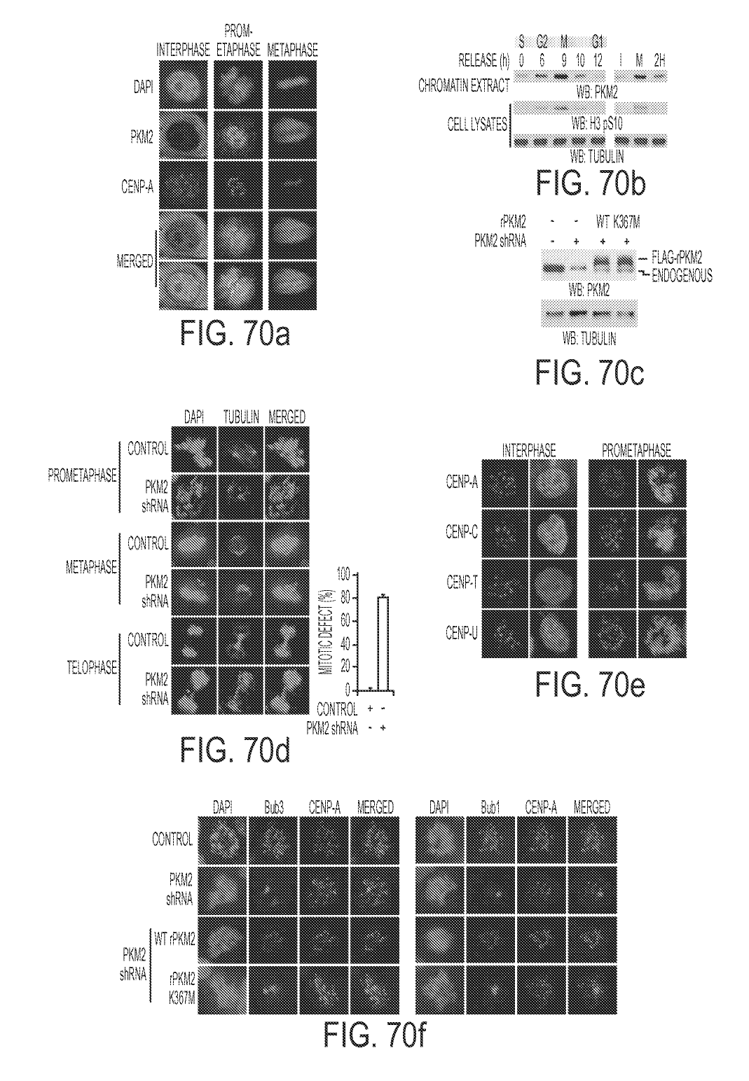

To examine whether PKM2 plays a role in mitosis, the inventors synchronized HeLa human cervical cancer cells in the G1 phase with a double-thymidine block and then released the block by removing thymidine for 12 h. Immunofluorescence analyses showed that PKM2 co-localized with chromatin and CENP-A, a centromere-specific histone H3 variant and a marker of kinetochore, primarily in prometaphase (and to a lesser extent in metaphase), but not in interphase (Cheeseman and Desai, 2008) (FIG. 70A). In line with this finding, immunoblotting studies revealed that PKM2 were enriched in chromatin extract of mitotic cells that were indicated by the mitosis marker phospho-histone H3-S10 (Cheung et al., 2000) (FIG. 70B, left panel). PKM2 association with chromatin was also observed in cells treated with nocodazole after a double-thymidine block that arrested the cells at mitosis (FIG. 70B, right panel). The amount of chromatin-associated PKM2 was reduced after removal of nocodazole for 2 h that prompted mitotic exit, suggesting a role for PKM2 in mitosis progression. This hypothesis was supported by the finding that PKM2 depletion (FIG. 70C) disrupted the microtubule spindle attachment to kinetochores and resulted in abnormal chromosome segregation and lagging chromatids in anaphase (FIG. 70D), which was rescued by reconstituted expression of RNAi-resistant wild-type (WT) rPKM2, but not rPKM2 K367M inactive mutant (FIGS. 70C and 76A). These results indicate that PKM2 kinase activity is required for the fidelity of chromosome segregation.

PKM2 depletion had no effect on the localization of CENP-A and other kinetochore proteins including CENP-C, -T, and -U, in the interphase and prometaphase of HeLa cells (FIG. 70E) and U87 human glioblastoma (GBM) cells expressing an active EGFRvIII mutant (U87/EGFRvIII) (FIG. 76B), but it blocked the recruitment of the SAC proteins Bub3 and Bub1 to kinetochores during the prometaphase; this defect of Bub3 and Bub1 recruitment was rescued by reconstituted expression of WT rPKM2, but not by rPKM2 K367M kinase-dead mutant in HeLa cells (FIGS. 70F and 76C), U87/EGFRvIII cells (FIGS. 76D and 76E), and GSC11 human primary GBM cells. These results indicated that PKM2 kinase activity is essential for the translocation of Bub3 and Bub1 to kinetochores, the establishment of correct K-MT attachments, and proper chromosome segregation.

PKM2, but not PKM1, Interacts with and Phosphorylates Bub3 at Y207

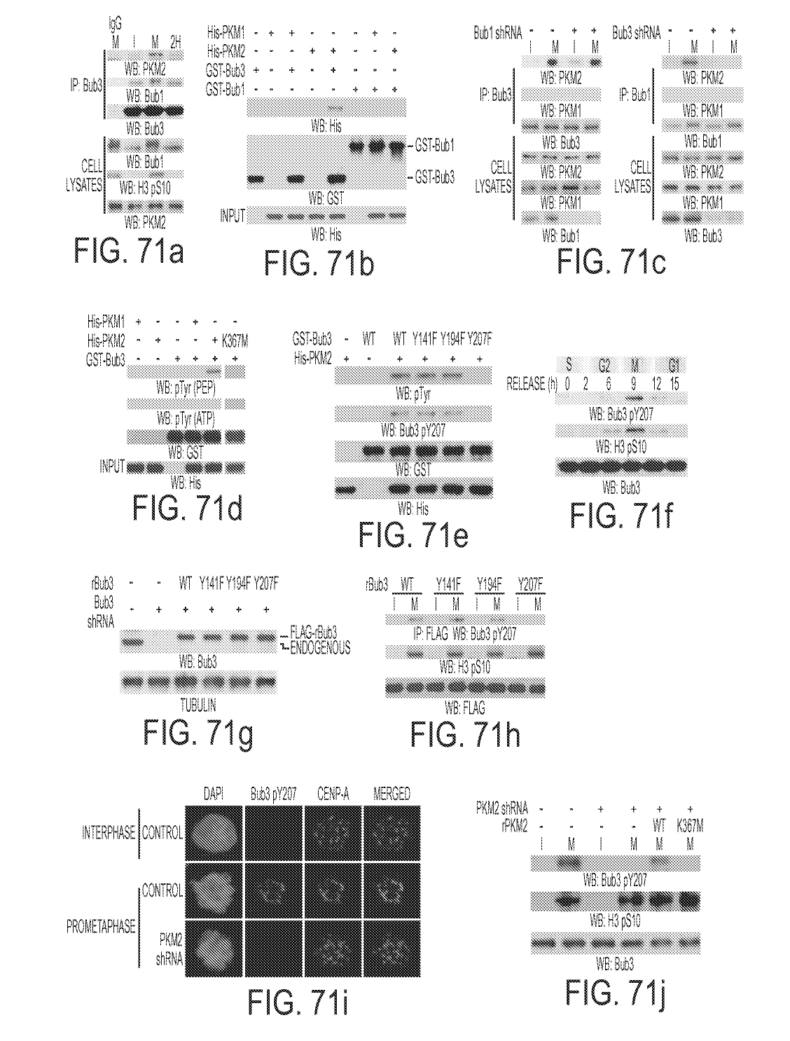

To determine the relationship between PKM2 and the Bub3-Bub1 complex, the inventors synchronized HeLa cells with a double-thymidine block followed with or without nocodazole treatment. Immunoblotting of immunoprecipitated Bub3 with an anti-PKM2 antibody showed that, in contrast to the constant association between Bub3 and Bub1 (FIG. 71A, second panel), Bub3 interacts with PKM2 during mitosis, but not in interphase or after mitosis exit (FIG. 71A, top panel). In line with a previous observation (Tang et al., 2004), immunoblotting analyses also detected a mobility shift of Bub1 during mitosis. To examine whether PKM2 directly binds to Bub3 or Bub1, the inventors incubated bacterially purified recombinant GST-Bub3 or GST-Bub1 with purified recombinant His-PKM2 or His-PKM1. PKM2, but not PKM1, binds to Bub3, but not Bub1 (FIG. 71B). Consistent with these in vitro results, co-immunoprecipitation analyses revealed that Bub1 depletion did not block the interaction between PKM2 and Bub3 (FIG. 71C, left panel). In contrast, Bub3 depletion blocked the interaction between PKM2 and Bub1 (FIG. 71C, right panel), further supporting a direct interaction between PKM2 and Bub3, but not Bub1. In addition, PKM1 was not detected in the Bub3-Bub1 complex.

An in vitro protein kinase assay of recombinant PKM2 or PKM1 mixed with recombinant Bub3 showed that PKM2, but not PKM2 K367M or PKM1, phosphorylated Bub3; Bub3 phosphorylation was detected by anti-phospho-Tyr (FIG. 71D), but not anti-phospho-Ser or -phospho-Thr antibodies. Notably, this phosphorylation occurred in the presence of PEP, the physiological phosphate group donor of PKM2, but not in the presence of ATP. Mutations of Bub3 Y141, Y194, and Y207 into phenylalanines showed that Bub3 Y207F, but not WT Bub3, Bub3 Y141F, or Bub3 Y194F, was resistant to phosphorylation by PKM2, as detected by anti-phospho-Tyr antibody and an antibody that specifically recognizes phosphorylated Bub3 Y207 (FIG. 71E). These results indicated that PKM2 interacts with and phosphorylates Bub3 at Y207 in vitro.

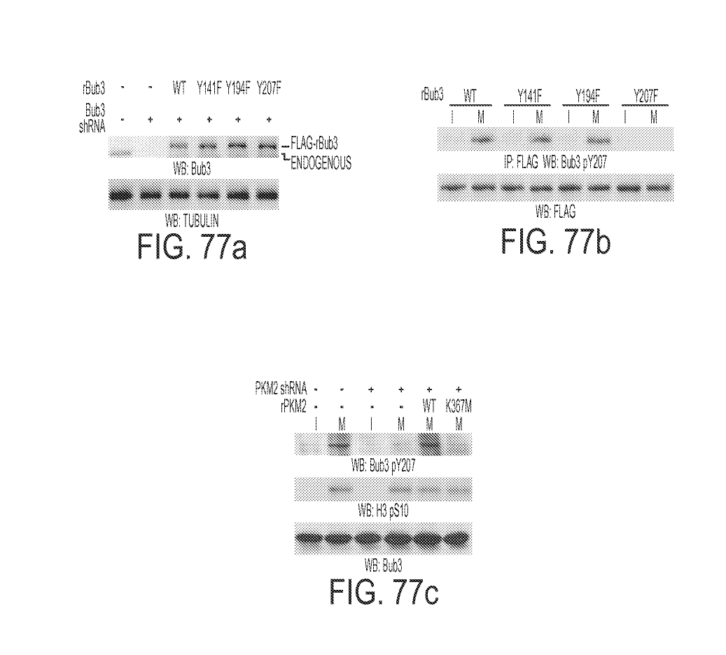

To test whether PKM2 phosphorylates Bub3 in cells, the inventors synchronized HeLa cells with a double-thymidine block and showed that Bub3 Y207 was phosphorylated during mitosis (FIG. 71F). Depletion of endogenous Bub3 and reconstituted expression of RNAi-resistant WT rBub3, rBub3 Y141F, rBub3 Y194F, or rBub3 Y207F in HeLa cells (FIG. 71G) or of WT rBub3 or rBub3 Y207F in U87/EGFRvIII (FIG. 77A) showed that only rBub3 Y207F was resistant to phosphorylation during mitosis (FIG. 71H; FIG. 77B). These results were further supported by immunofluorescence analyses showing that phosphorylated Bub3 Y207 co-localized with CENP-A and was detected in prometaphase, but not in interphase, which were blocked by PKM2 depletion (FIG. 71I). In addition, depletion of endogenous PKM2 and reconstituted expression of WT rPKM2 or rPKM2 K367M in HeLa cells (FIG. 70C) and U87/EGFRvIII cells (FIG. 76D) revealed that PKM2 depletion blocked Bub3 Y207 phosphorylation during mitosis, which was rescued by expression of WT rPKM2 but not rPKM2 K367M mutant (FIG. 71J; FIG. 77C). These results indicated that PKM2 phosphorylates Bub3 Y207 during mitosis.

### PKM2-Dependent Bub3 Y207 Phosphorylation is Required for Recruitment of Bub3 and Bub1 to Kinetochores and Accurate Chromosome Segregation

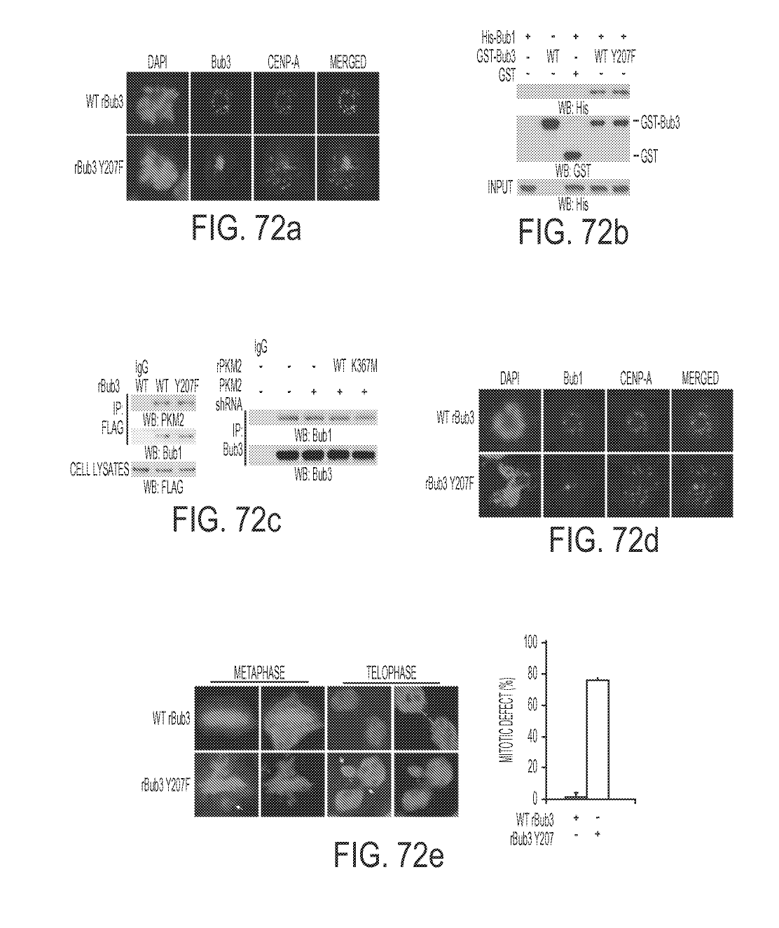

Immunostaining of HeLa cells with depleted endogenous Bub3 and reconstituted expression of WT rBub3 or rBub3 Y207F (FIG. 71G) showed that rBub3 Y207F, unlike its WT counterpart, failed to co-localize with CENP-A during prometaphase (FIG. 72A), indicating that PKM2-dependent Bub3 Y207 phosphorylation is required for kinetochore recruitment of Bub3. Nevertheless, like its WT counterpart, purified recombinant GST-Bub3 Y207F bound to purified recombinant His-Bub1, indicating that Bub3 Y207 phosphorylation is dispensable for the association between Bub3 and Bub1 (FIG. 72B). In agreement with these findings, a co-immunoprecipitation assay showed that Bub3 Y207F and its WT counterpart bound similarly to PKM2 and Bub1 (FIG. 72C, left panel), and reconstituted expression of rPKM2 K367M, in contrast to re-expression of WT rPKM2, did not significantly affect the interaction between Bub3 and Bub1 (FIG. 72C, right panel), indicating that Bub3 Y207 phosphorylation does not alter formation of the Bub3, Bub1, and PKM2 complex.

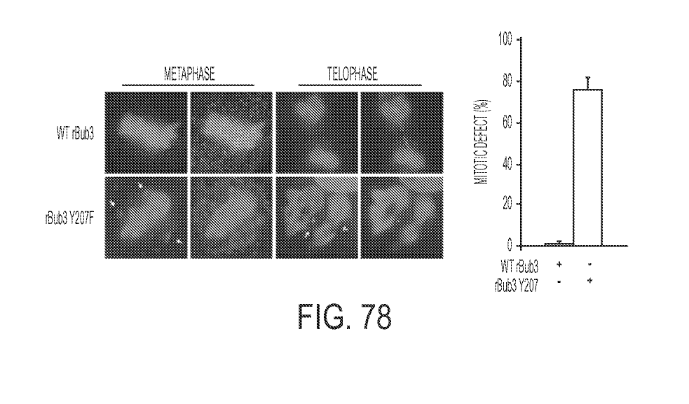

In sharp contrast, immunofluorescence analysis revealed that Bub1 failed to co-localize with CENP-A in the cells with reconstituted expression of rBub3 Y207F, but not its WT counterpart (FIG. 72D). In addition, HeLa (FIG. 72E) or U87/EGFRvIII (FIG. 78) cells with reconstituted expression of rBub3 Y207F exhibited increased incidences of mitotic defects, as reflected by the misalignment of chromosomes in the metaphase plate and defective chromosome segregation represented by the increased incidence of lagging chromosomes and micronuclei in telophase. These results indicated that PKM2-dependent Bub3 Y207 phosphorylation is required for kinetochore recruitment of Bub3 and Bub1, correct K-MT attachments, and proper chromosome segregation.

### PKM2-Dependent Bub3 Y207 Phosphorylation is Required for Recruitment of Bub3 and Bub1 to Blinkin

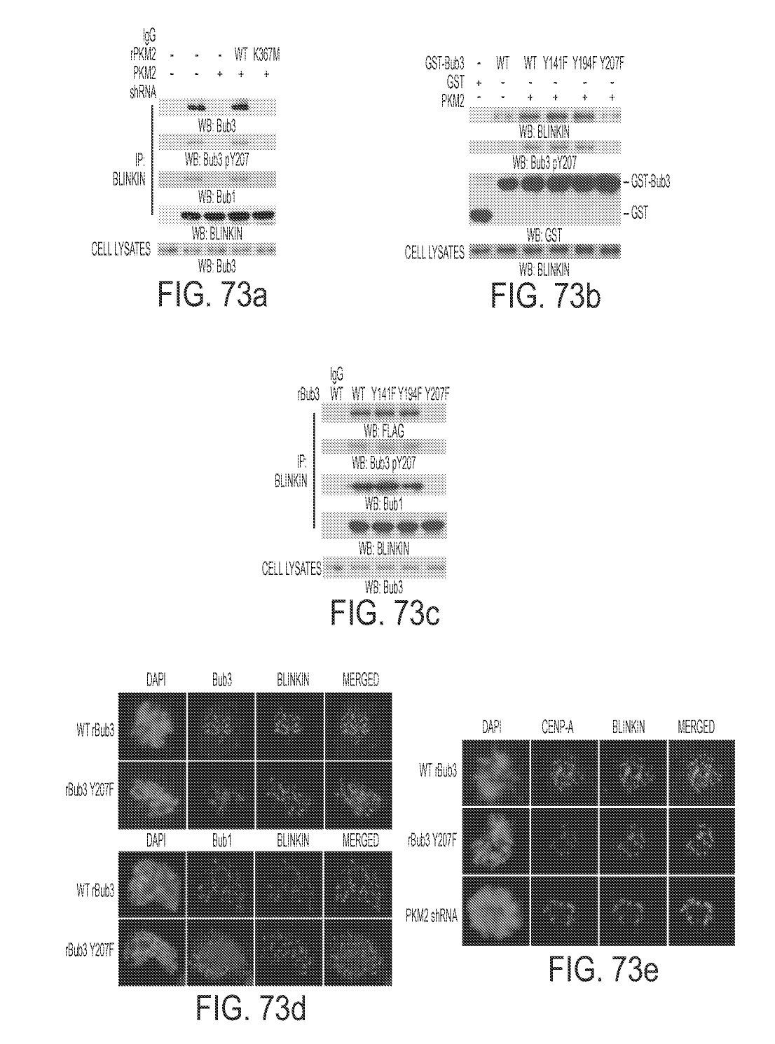

Blinkin interaction with Bub1 is required for recruitment of Bub1 to kinetochores (Kiyomitsu et al., 2007). To examine whether Bub3 Y207 phosphorylation regulates the binding of the Bub3-Bub1 complex to Blinkin, the inventors performed a double-thymidine block followed by co-immunoprecipitation analyses with an anti-Blinkin antibody. FIG. 73A shows that Blinkin interacted with phosphorylated Bub3 Y207 and Bub1 during mitosis. These interactions were blocked by PKM2 depletion and rescued by reconstituted expression of WT rPKM2, but not rPKM2 K367M, indicating that PKM2 kinase activity is required for Blinkin-Bub3-Bub1 complex formation.

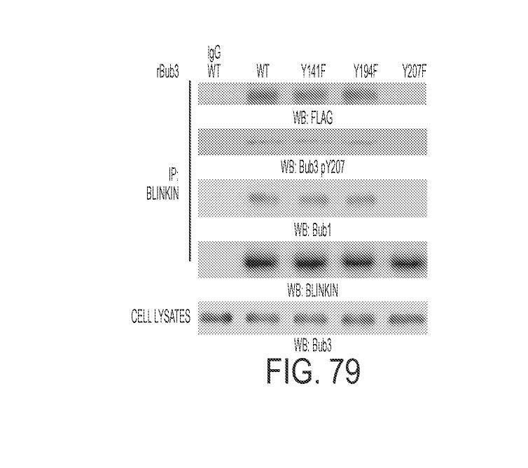

In line with these observations, a GST pull-down assay showed that purified GST-Bub3 interacted with a limited amount of Blinkin from mitotic cells with endogenous PKM2 depletion (FIG. 73B). However, incubation of purified WT Bub3, Bub3 Y141F, or Bub3 Y194F, but not Bub3 Y207F, with purified PKM2, which phosphorylated Bub3 Y207, significantly enriched the association between Bub3 and Blinkin. In addition, co-immunoprecipitation with an anti-Blinkin antibody showed that Blinkin interacted with Bub1, WT rBub3, rBub3 Y141F, and rBub3 Y194F, but not rBub3 Y207F, in HeLa (FIG. 73C) and U87/EGFRvIII (FIG. 79) cells with reconstituted expression of these Bub3 proteins (FIG. 71G and FIG. 77A). Furthermore, Bub3 Y207F, unlike its WT counterpart, failed to co-localize with Blinkin in kinetochores during prometaphase (FIG. 73D, upper panels), and Bub3 Y207F expression blocked recruitment of Bub1 to Blinkin at kinetochores (FIG. 73D, lower panels). In contrast, reconstituted expression of rBub3 Y207F or PKM2 depletion did not affect the co-localization of Blinkin with CENP-A at the centromere (FIG. 73E). These results indicated that PKM2-dependent Bub3 Y207 phosphorylation is required for recruitment of Bub3 and Bub1 to kinetochores to interact with Blinkin.

### PKM2-Dependent Bub3 Y207 Phosphorylation is Required for Spindle Assembly Checkpoint, Cell Survival and Proliferation, and Tumorigenesis

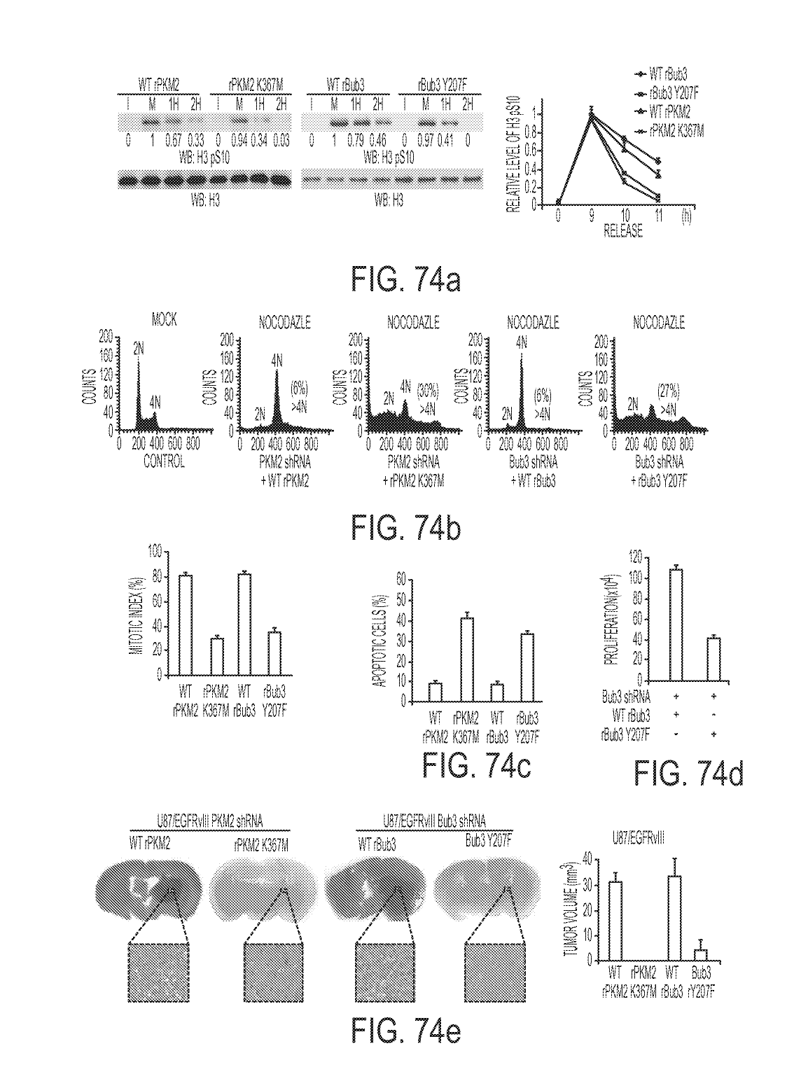

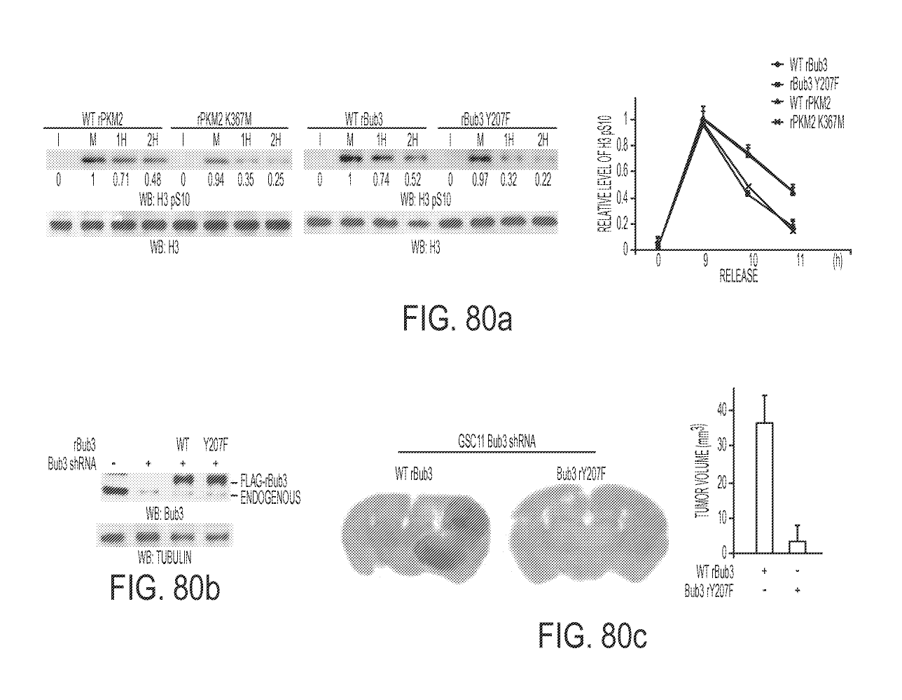

Bub3 and Bub1 are required for SAC and delay the onset of anaphase; failure of SAC leads to an accelerated mitosis exit (Bolanos-Garcia and Blundell, 2011). A double-thymidine block and release of HeLa (FIG. 74A) and U87/EGFRvIII (FIG. 80A) cells with depleted PKM2 (left panel) or Bub3 (right panel) and reconstituted expression of WT rPKM2, rPKM2 K367M, WT rBub3, or rBub3 Y207F showed that expression of rPKM2 K367M or rBub3 Y207F, in contrast to expression of their WT counterparts, resulted in a rapid downregulation of histone H3-S10 phosphorylation, suggesting an accelerated mitosis exit. These observations were supported by flow cytometric analyses showing that about 80% of the HeLa cells with reconstituted expression of WT rPKM2 or WT rBUb3 were arrested in mitosis in the presence of nocodazole treatment for 36 h. In contrast, only about 30% of the cells with reconstituted expression of rPKM2 K367M or rBub3 Y207F were arrested in mitosis, and a significant fraction of these cells failed to undergo mitotic arrest (FIG. 74B).

With exposure to nocodazole (36 hours), about 27%-30% of the cells with reconstituted expression of PKM2 K367M or Bub3 Y207F underwent another round of DNA replication in the absence of cell division and had DNA content more than 4N (FIG. 74B, top panel), indicating a defective SAC. Notably, a significantly higher fraction of these cells underwent cell death compared with their WT counterparts (FIG. 74C), which were arrested at mitosis with 4N DNA content (FIG. 74B, top panel). The inventors also found a higher percentage of apoptosis among the cells with reconstituted expression of PKM2 K367M or Bub3 Y207F than among their WT counterparts in the absence of nocodazole treatment. These results indicated that PKM2 kinase activity and PKM2-dependent Bub3 Y207 phosphorylation are required for SAC to prevent abnormal mitosis exit and apoptosis. In line with the mitosis defect, cells with reconstituted expression of Bub3 Y207F exhibited inhibited cell growth in contrast to the WT control cells (FIG. 74D).

To determine the role of PKM2-dependent Y207F phosphorylation in brain tumor development, the inventors intracranially injected endogenous PKM2- or Bub3-depleted U87/EGFRvIII cells with reconstituted expression of WT rPKM2, rPKM2 K367M, WT rBub3, or rBub3 Y207F into athymic nude mice. U87/EGFRvIII cells expressing WT rPKM2 or WT rBub3 elicited rapid tumorigenesis (FIG. 74E). In contrast, rPKM2 K367M and rBub3 Y207F expression abrogated EGFRvIII-driven tumor growth. Similar tumorigenesis results were obtained by using GSC11 human primary GBM cells with endogenous Bub3 depletion and reconstituted expression of WT rBub3 or rBub3 Y207F (FIGS. 80B and 80C). These results indicated that PKM2-dependent Bub3 Y207 phosphorylation is instrumental in EGFR-promoted tumor development.

Bub3 Y207 Phosphorylation Positively Correlates with the Level of H3-S10 Phosphorylation

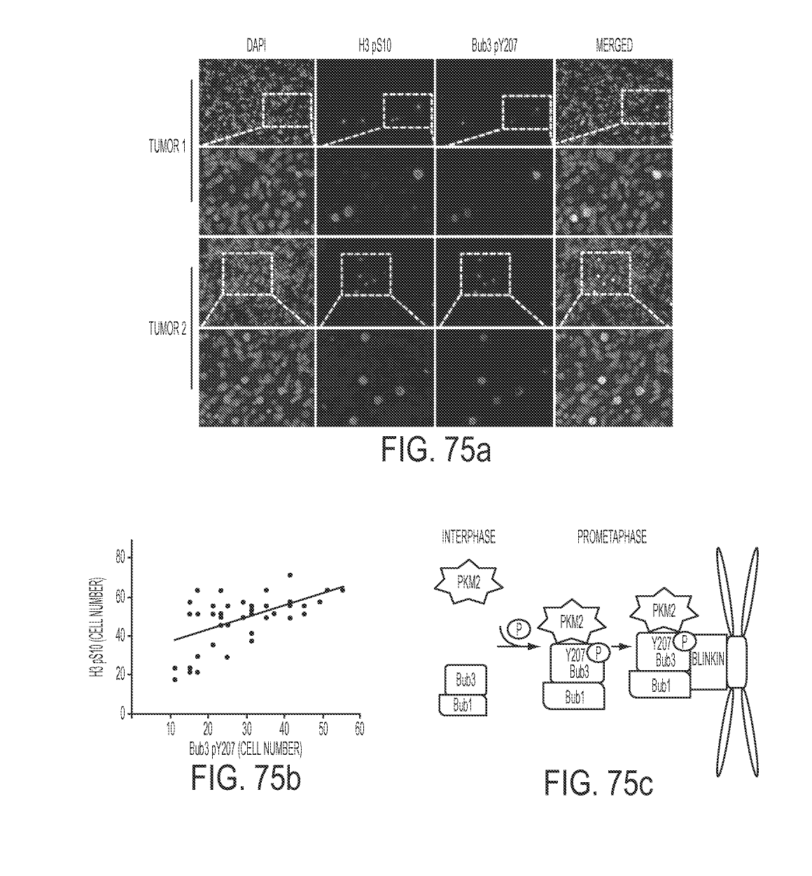

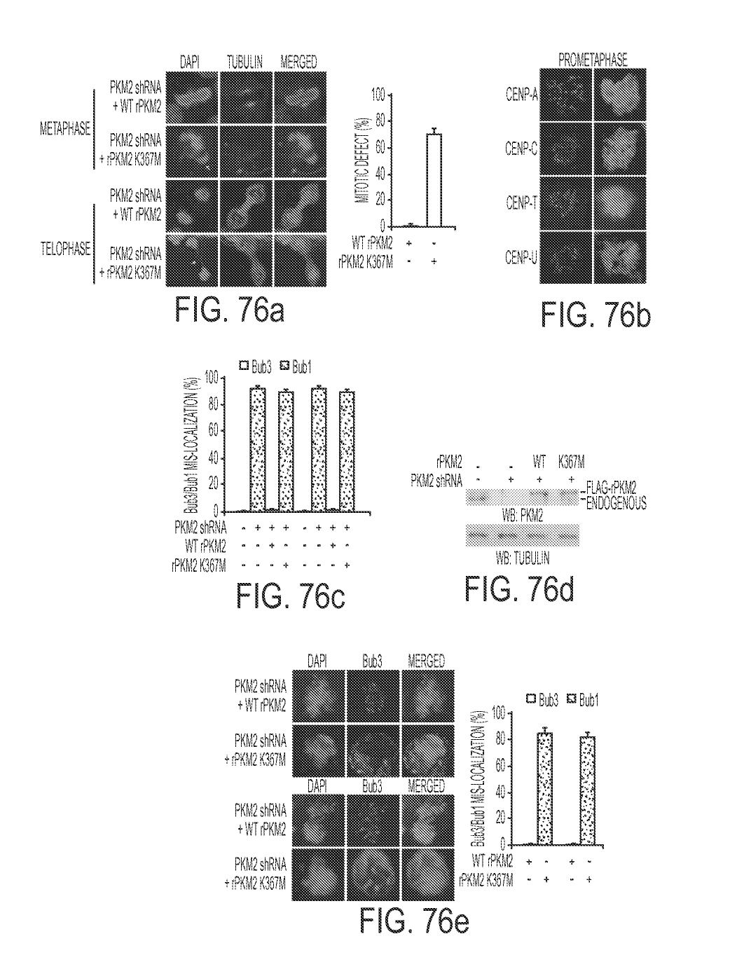

Bub3 Y207 phosphorylation correlates with H3-S10 phosphorylation during mitosis (FIG. 71H). To further define the clinical relevance of the inventor's finding that PKM2 phosphorylates Bub3 Y207, the inventors used immunohistochemical (IHC) analyses to examine the levels of Bub3 Y207 phosphorylation and H3-S10 phosphorylation in serial sections of 50 human primary GBM specimens (World Health Organization [WHO] grade IV). The antibody specificities were validated by using IHC analyses with specific blocking peptides. As shown in FIG. 75A, Bub3 Y207 phosphorylation co-localized with H3-S10 phosphorylation. In addition, levels of Bub3 Y207 and H3-S10 phosphorylation were correlated with each other. Quantification of the staining showed that these correlations were significant (FIG. 75B; r=0.78, P<0.0001). These results support a role for PKM2-dependent Bub3 Y207 phosphorylation in the clinical behavior of human GBM and reveal a relationship between Bub3 Y207 phosphorylation and mitotic progression of tumor cells.

### Discussion

The mitotic checkpoint is a major cell cycle control mechanism that guards against chromosome mis-segregation and the subsequent production of aneuploid daughter cells (Holland and Cleveland, 2009). PKM2 plays a key role as a glycolytic enzyme in the Warburg effect (Christofk et al., 2008; Mellati et al., 1992). PKM2 also processes nonmetabolic functions and plays a critical role in regulating gene transcription (Yang et al., 2011). However, whether PKM2 directly regulates cell cycle progression by mediating mitosis process is not known. The inventors demonstrated that PKM2 interacts with Bub3 and phosphorylates Bub3 Y207, which leads to the recruitment of the Bub3-Bub1 complex to Blinkin in kinetochores, precise control of kinetochore-spindle microtubule attachment and SAC, and subsequently, accurate chromosome segregation and cell proliferation (FIG. 75C).

Aneuploidy is associated with cancer and tumorigenesis, but it also adversely affects cell proliferation and the growth of organisms, which results from the gain or loss of hundreds or thousands of genes and the disruption of a large array of cellular activities. Thus, aneuploidy can either promote or suppress tumor formation, depending on the genetic and cellular context, including the specific genes on the abnormal chromosome, the extent of the aneuploidy, the already-accumulated genetic errors, and specific features unique to the cell type (Holland and Cleveland, 2009). In mammals, complete inactivation of the mitotic checkpoint leads to massive chromosome mis-segregation, cell death, and early embryonic lethality (Williams et al., 2008; Michel et al., 2001; Dobles et al., 2000). Depleting the SAC proteins BubR1 or Mad2 or inhibiting BubR1 kinase activity causes apoptotic cell death in human cancer cells (Kops et al., 2004). Depletion of Bub1, Bub3, and Blinkin all lead to chromosome mis-segregation and mitosis defects (Kiyomitsu et al., 2007; Logarinho and Bousbaa, 2008). Consistent with the critical role of SAC proteins in mitosis, Bub1-null mice are embryonically lethal (Jeganathan et al., 2007). Similarly, Bub3-null embryos accumulate mitotic errors in the form of micronuclei, chromatin bridging, lagging chromosomes, and irregular nuclear morphology that result in failure to survive. Bub3-null embryos treated with a spindle-depolymerizing agent fail to arrest in metaphase and show an increase in mitotic defects (Kalitsis et al., 2000). In line with these evidences of the essential role of Bub1, Bub3, and Blinkin in kinetochore-spindle microtubule attachment and mitotic checkpoint, reconstituted expression of PKM2 kinase-dead mutant in endogenous PKM2-depleted cancer cells displayed a similar mitotic defect, aneuoploid formation, and cell apoptosis. Importantly, the inventor's findings support that PKM2-dependent Bub3 Y207 phosphorylation regulates the mitotic functions of the Bub3-Bub1-Blinkin complex and governs the integrity of chromosome segregation and cell survival and proliferation.

Abnormally high expression of SAC protein, such as MAD2, kinetochore component HEC1, and PKM2, is common in human cancers, and elevated levels of these proteins are often associated with a poor prognosis (Mazurek, 2007; Yang et al., 2011; Holland and Cleveland, 2009). In contrast, reduced expression of SAC proteins such as CENP-E and BubR1, resulting from CENP-E haploinsufficiency and BubR1 heterozygosity, respectively, lowered the tumor incidence in mice (Holland and Cleveland, 2009; Rao et al., 2005). The findings that interruption of Bub3 Y207 phosphorylation results in increased cell apoptosis and inhibition of tumor cell proliferation and EGFR-promoted tumorigenesis and that Bub3 Y207 phosphorylation correlates with mitotic progression of tumor cells in GBM specimens highlight the nonmetabolic function of PKM2 as a protein kinase in controlling the mitotic process and may provide a molecular basis for improving the diagnosis and treatment of tumors with upregulated PKM2.

In addition to the essential role of PKM2 in controlling G1-S phase transition and chromatid segregation/mitotic check point by phosphorylating histone H3 at T11 and Bub3 Y207, respectively, it was also found that PKM2 phosphorylates MLC2 at position Y118 and directly controls cytokinese. Inhibition of PKM2-dependent MLC2 Y118 phosphorylation resulted in inhibition of cell division and multinucleate cells and subsequent inhibition of cell growth and proliferation.

### Materials and Methods

### Cell Culture and Synchronization

Hela, U87/EGFRvIII, and GSC11 cancer cells were maintained in Dulbecco's modified Eagle's medium (DMEM) supplemented with 10% bovine calf serum (HyClone, Logan, Utah).

Double-thymidine block: 30%-40% confluent cells were washed twice with phosphate buffered saline (PBS), treated with 2 mM thymidine for 17 h, washed twice in PBS again, released in complete medium containing 10 μM deoxycytidine for 9 h, treated with 2 mM thymidine for 17 h, and then released into complete medium with 10 μM deoxycytidine and assayed.

Double-thymidine-nocodazole block: after the double-thymidine block, cells were washed twice with PBS, released in complete medium for 6 h, and then treated with 100 ng/mL nocodazole for different periods of time.

### Materials

Rabbit polyclonal antibodies recognizing phospho-Bub3 Y207, PKM1, PKM2, and phospho-histone H3 S10 were obtained from Signalway Biotechnology (Pearland, Tex.). A mouse antibody recognizing Bub3 was obtained from BD Biosciences (San Jose, Calif.). CENP-A, CENP-C, and Bub1 were purchased from Abcam (Boston, Mass.). A polyclonal antibody against Blinkin was purchased from Bioss (Woburn, Mass.). A rabbit polyclonal antibody against tubulin was from Cell Signaling Technology (Beverly, Mass.). A polyclonal antibody for acetylated histone H3 was obtained from Upstate Biotechnology (Billerica, Mass.). Mouse monoclonal antibodies for FLAG, GST, and His were purchased from Sigma (St. Louis, Mo.). Hygromycin, puromycin, DNase-free RNase A, and propidium iodide were purchased from EMD Biosciences (San Diego, Calif.). Thymidine and nocodazole were from Sigma. DAPI, Alexa Fluor 488, 594 goat anti-rabbit antibody, and Alexa Fluor 488, 594 goat anti-mouse antibody were from Molecular Probes (Eugene, Oreg.). HyFect transfection reagents were from Denville Scientific (Metuchen, N.J.). GelCode Blue Stain Reagent was obtained from Pierce (Rockford, Ill.).

### Transfection

Cells were plated at a density of 4×10⁵/60-mm dish 18 h prior to transfection. Transfection was performed using HyFect reagents (Denville Scientific) according to the vendor's instructions.

### Immunoprecipitation and Immunoblotting Analysis

Extraction of proteins from cultured cells using a modified buffer was followed by immunoprecipitation and immunoblotting with corresponding antibodies, as described previously (Lu et al., 1998).

### Cell Proliferation Assay

Cells (2×10⁴) were plated and counted seven days after seeding in DMEM with 0.5% bovine calf serum. Data represent the mean±standard deviation (S.D.) of three independent experiments.

### DNA Constructs and Mutagenesis

Bub3 was cloned into pcDNA3.1/hygro (+) vector between BamHI and XhoI. pcDNA 3.1/hygro (+) Bub3 Y141F, Y194F, and Y207F were made using the QuikChange site-directed mutagenesis kit (Stratagene, Santa Clara, Calif.). The pGIPZ controls were generated with control oligonucleotide GCTTCTAACACCGGAGGTCTT (SEQ ID NO: 1) or GCCCGAAAGGGTTCCAGCTTA (SEQ ID NO: 36). pGIPZ PKM2 shRNA was generated with CATCTACCACTTGCAATTA (SEQ ID NO: 14) oligonucleotide targeting exon 10 of the PKM2 transcript. pGIPZ Bub3 shRNA was generated with AAGGCCGAGTGGCAGTTGAGT (SEQ ID NO: 37).

### In Vitro Kinase Assays

The kinase reactions were performed as described previously (Fang et al., 2007). In brief, bacterially purified recombinant PKM2 (200 ng) was incubated with Bub3 (100 ng) in kinase buffer (50 mM Tris-HCl [pH 7.5], 100 mM KCl, 50 mM MgCl₂, 1 mM Na₃VO₄, 1 mM dithiothreitol [DTT], 5% glycerol, 0.5 mM PEP, 0.05 mM FBP) in 25 μL at 25° C. for 1 h. The reactions were terminated by the addition of sodium dodecyl sulfate-polyacrylamide gel electrophoresis (SDS-PAGE) loading buffer and heated to 100° C. The reaction mixtures were then subjected to SDS-PAGE analyses.

### Flow Cytometry Analysis

Cells (1×10⁶) were fixed in 70% ethanol on ice for 3 h, spun down, and incubated for 1 h at 37° C. in PBS with DNase-free RNase A (100 μg/mL) and propidium iodide (50 μg/mL). Cells were then analyzed by fluorescence-activated cell sorting (FACS).

### Purification of Recombinant Proteins

Wild-type and mutant GST-PKM2, His-PKM2, His-PKM1, GST-Bub1, His-Bub1, GST-Bub3, and His-Bub3 were expressed in bacteria purified as described previously (Xia et al., 2007).

### Immunofluorescence Analysis

Cells were fixed and incubated with primary antibodies, Alexa Fluor dye-conjugated secondary antibodies, and DAPI according to standard protocols. Cells were examined using a deconvolution microscope (Zeiss, Thornwood, N.Y.) with a 63-Å oil immersion objective. Axio Vision software from Zeiss was used to deconvolute Z-series images.

### Chromatin Fractionation

Cells were first lysed with buffer A (50 mM HEPES [pH 7.9], 10 mM KCl, 1.5 mM MgCl₂, 0.34 M sucrose, 10% glycerol, 1 mM DTT, protease inhibitor cocktail, 0.1% Trition X-100) on ice for 15 min. After centrifugation at 6600 g, pellets, including the nucleus, were washed in buffer A and further lysed with buffer B (3 mM EDTA, 0.2 mM EGTA, 1 mM DTT, protease inhibitor cocktail) on ice for 30 min. After centrifugation at 6600 g, pellets containing the chromatin were washed with buffer B and sonicated in RIPA lysis buffer (50 mM Tris-HCl [pH 7.4], 150 mM NaCl, 1% Trition x-100, 1% sodium deoxycholate, 0.1% SDS, 1 mM EDTA, protease inhibitor) for Western blot analysis.

### Immunohistochemical Analysis

Mouse tumor tissues were fixed and then stained with Mayer's hematoxylin and eosin (H & E) (Biogenex Laboratories, Fremont Calif.). The slides were mounted using Fluorogel with Tris buffer (Electron Microscopy Sciences, Hatfield, Pa.). The tissue sections from paraffin-embedded human GBM specimens were stained with antibodies against phospho-Bub3 Y207, phospho-histone H3 S10, or nonspecific IgG as a negative control. The tissue sections were quantitatively scored by counting positively-stained cells in 10 microscopic fields. The use of human brain tumor specimens and the database was approved by the institutional review board of The University of Texas MD Anderson Cancer Center.

### Intracranial Injection

The inventors intracranially injected 5×10⁵ U87/EGFRvIII or GSC11 cells with PKM2 or PKM2 K367M and reconstitution of rPKM2, rPKM2 S37A, Bub3, or Bub3 Y207F expression (in 5 μL of DMEM per mouse) into 4-week-old female athymic nude mice. The intracranial injections were performed as described in a previous publication (Yang et al., 2011). Seven mice per group in each experiment were included. The mice were sacrificed two weeks after glioma cell injection. The brain of each mouse was harvested, fixed in 4% formaldehyde, and embedded in paraffin. Tumor formation and phenotype were determined by histological analysis of H & E-stained sections.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. Publn. 2010/0099726
PCT Pat. Publn. WO 2012/056319
Allred et al., Prognostic and predictive factors in breast cancer by immunohistochemical analysis, Mod. Pathol., 11:155-168, 1998.
Altenberg and Greulich, Genes of glycolysis are ubiquitously overexpressed in 24 cancer classes, Genomics, 84:1014-1020, 2004.
Anastasiou et al., Pyruvate kinase M2 activators promote tetramer formation and suppress tumorigenesis, Nat. Chem. Biol. Aug. 26, 2012.
Badie et al., Combined radiation and p53 gene therapy of malignant glioma cells, Cancer Gene Ther., 6:155-162, 1999.
Baek, When signaling kinases meet histones and histone modifiers in the nucleus, Mol. Cell, 42:274-284, 2011.
Bolanos-Garcia and Blundell, BUB1 and BUBR1: Multifaceted kinases of the cell cycle, Trends Biochem. Sci., 36:141-150, 2011.
Breitkreutz et al., Protein kinase C family: on the crossroads of cell signaling in skin and tumor epithelium, J. Cancer Res. Clin. Oncol., 133:793-808, 2007.
Brown et al., NF-kappaB in carcinoma therapy and prevention, Expert Opin. Ther. Targets, 12:1109-1122, 2008.
Burke et al., J Biol Chem. 278:1450-6, 2003.
BMC Bioinformatics. 2010; Suppl 7:S15
Cairns et al., Regulation of cancer cell metabolism, Nat. Rev. Cancer, 11:85-95, 2011.
Cancer Res. 2008; 68:9519-24
Cheeseman and Desai, Nat. Rev. Mol. Cell Biol., 9:33-46, 2008.
Chen et al., Amplitude control of protein kinase C by RINCK, a novel E3 ubiquitin ligase, J. Biol. Chem., 282:33776-33787, 2007.
Chen et al., Shikonin and its analogs inhibit cancer cell glycolysis by targeting tumor pyruvate kinase-M2. Oncogene, 30:4297-4306, 2011
Cheung et al., Signaling to chromatin through histone modifications, Cell, 103:263-271, 2000.
Cheung et al., Polypyrimidine tract binding protein and Notch1 are independently re-expressed in glioma, Mod. Pathol. 19:1034-1041, 2006.
Choi et al., On/off-regulation of phospholipase C-gamma 1-mediated signal transduction, Adv. Enzyme Regul., 47:104-116. 2007.
Chou and Howard, Inhibition by Wnt-1 or Wnt-3a of nerve growth factor-induced differentiation of PC12 cells is reversed by bisindolylmaleimide-I but not by several other PKC inhibitors, Oncogene, 21:6348-6355, 2002.
Christofk et al., Pyruvate kinase M2 is a phosphotyrosine-binding protein, Nature, 452:181-186, 2008a.
Christofk et al., The M2 splice isoform of pyruvate kinase is important for cancer metabolism and tumour growth, Nature, 452:230-233, 2008b.
Clower et al., The alternative splicing repressors hnRNP A1/A2 and PTB influence pyruvate kinase isoform expression and cell metabolism, Proc. Natl. Acad. Sci. U.S.A., 107:1894-1899, 2010.
Coluccia et al., Bcr-Abl stabilizes beta-catenin in chronic myeloid leukemia through its tyrosine phosphorylation, EMBO J., 26:1456-1466, 2007.
Cordier et al., The zinc finger of NEMO is a functional ubiquitin-binding domain, J. Biol. Chem., 284:2902-2907, 2009.
Dang et al., The interplay between MYC and HIF in cancer, Nat. Rev. Cancer, 8:51-56, 2008.
David et al., HnRNP proteins controlled by c-Myc deregulate pyruvate kinase mRNA splicing in cancer, Nature, 463:364-368, 2010.
DeBerardinis et al., The biology of cancer: metabolic reprogramming fuels cell growth and proliferation, Cell. Metab., 7:11-20, 2008.
Dobles et al., Chromosome mis-segregation and apoptosis in mice lacking the mitotic checkpoing protein Mad2, Cell, 101:635-646, 2000.
Dombrauckas et al., Structural basis for tumor pyruvate kinase M2 allosteric regulation and catalysis, Biochemistry, 44:9417-9429, 2005.
Duncan et al. J. Med. Chem. 54:3854-3865, 2011
Duran et al., Essential role of RelA Ser311 phosphorylation by zetaPKC in NF-kappaB transcriptional activation, EMBO J., 22:3910-3918, 2003.
Ea et al., Activation of IKK by TNFalpha requires site-specific ubiquitination of RIP1 and polyubiquitin binding by NEMO, Mol. Cell, 22:245-257, 2006.
Eur J Med Chem. 2011; 46:1245-53
Fang et al., Phosphorylation of beta-catenin by AKT promotes beta-catenin transcriptional activity, J. Biol. Chem., 282:11221-11229, 2007.
Freije et al., Gene expression profiling of gliomas strongly predicts survival, Cancer Res., 64:6503-6510, 2004.
Furnari et al., Malignant astrocytic glioma: genetics, biology, and paths to treatment, Genes Dev., 21:2683-2710, 2007.
Gao et al., Pyruvate kinase m2 regulates gene transcription by acting as a protein kinase, Mol. Cell, 45:598-609, 2012.
Ghosh and Hayden, New regulators of NF-kappaB in inflammation, Nat. Rev. Immunol., 8:837-848, 2008.
Gomez-Manzano et al., Delta-24 increases the expression and activity of topoisomerase I and enhances the antiglioma effect of irinotecan, Clin. Cancer Res., 12:556-562, 2006.
Gravendeel et al., Intrinsic gene expression profiles of gliomas are a better predictor of survival than histology, Cancer Res., 69:9065-9072, 2009.
Hayden and Ghosh, Signaling to NF-kappaB, Genes Dev., 18:2195-2224, 2004.
Hoffmann et al., Transcriptional regulation via the NF-kappaB signaling module, Oncogene, 25:6706-6716, 2006.
Holland and Cleveland, Boveri revisited: chromosomal instability, aneuploidy and tumorigenesis, Nat. Rev. Mol. Cell Biol., 10:478-487, 2009.
Hornia et al., Antagonistic effects of protein kinase C alpha and delta on both transformation and phospholipase D activity mediated by the epidermal growth factor receptor, Mol. Cell. Biol., 19:7672-7680, 1999.
Hoshino et al., Regulation of cell proliferation by interleukin-3-induced nuclear translocation of pyruvate kinase. J. Biol. Chem., 282:17706-17711, 2007.
Hsu and Sabatini, Cancer cell metabolism: Warburg and beyond, Cell, 134:703-707, 2008.
Jans et al., Nuclear targeting signal recognition: a key control point in nuclear transport? Bioessays, 22:532-544, 2000.
Jean et al., Epidermal growth factor receptor/beta-catenin/T-cell factor 4/matrix metalloproteinase 1: a new pathway for regulating keratinocyte invasiveness after UVAb irradiation, Cancer Res., 69:3291-3299, 2009.
Jeganathan et al., J. Cell. Biol., 179:255-267, 2007.
Ji et al., EGF-induced ERK activation promotes CK2-mediated disassociation of alpha-Catenin from beta-Catenin and transactivation of beta-Catenin, Mol. Cell, 36:547-559, 2009.
J Pharmacol Exp Ther. 2005; 312:373-81
J Pharmacol Exp Ther. 2009; 330:377-88
J Biol Chem. 2005; 280:20442-8
J Natl Cancer Inst. 2008; 100:862-75
Jurica et al., The allosteric regulation of pyruvate kinase by fructose-1,6-bisphosphate, Structure, 6:195-210, 1998.
Kalitsis et al., Genes Dev., 14:2277-2282, 2000.
Karin, Nuclear factor-kappaB in cancer development and progression, Nature, 441:431-436, 2006.
Kawauchi et al., p53 regulates glucose metabolism through an IKK-NF-kappaB pathway and inhibits cell transformation, Nat. Cell Biol., 10:611-618, 2008.
Kiyomitsu et al., Dev. Cell, 13:663-676, 2007.
Koppenol et al., Otto Warburg's contributions to current concepts of cancer metabolism, Nat. Rev. Cancer, 11:325-337, 2011.
Kops et al., Lethality to human cancer cells through massive chromosome loss by inhibition of the mitotic checkpoint, Proc. Natl. Acad. Sci. U.S.A., 101:8699-8704, 2004.
Kuan et al., EGF mutant receptor viii as a molecular target in cancer therapy, Endocr. Relat. Cancer, 8:83-96, 2001.
Lallena et al., Activation of IkappaB kinase beta by protein kinase C isoforms, Mol. Cell. Biol., 19:2180-2188, 1999.
Le Mellay et al., Regulation of glycolysis by Raf protein serine/threonine kinases, Adv. Enzyme Regul., 42:317-332, 2002.
Lee et al., Pyruvate kinase isozyme type M2 (PKM2) interacts and cooperates with Oct-4 in regulating transcription, Int. J. Biochem. Cell. Biol., 40:1043-1054, 2008.
Lee et al., The language of histone crosstalk, Cell, 14: 682-685, 2010.
Lilien and Balsamo, The regulation of cadherin-mediated adhesion by tyrosine phosphorylation/dephosphorylation of beta-catenin, Curr. Opin. Cell. Biol., 17:459-465, 2005.
Logarinho et al., The human spindle assembly checkpoint protein Bub3 is required for the establishment of efficient kinetochore-microtubule attachments, Mol. Biol. Cell., 19:1798-1813, 2008.
Logarinho and Bousbaa, Cell Cycle, 7:1763-1768, 2008.
Lu, Nonmetabolic functions of pyruvate kinase isoform M2 in controlling cell cycle progression and tumorigenesis, Chin. J. Cancer, 31:5-7, 2012.
Lu et al., Activation of protein kinase C triggers its ubiquitination and degradation, Mol. Cell. Biol., 18:839-845, 1998.
Lu et al., Epidermal growth factor-induced tumor cell invasion and metastasis initiated by dephosphorylation and downregulation of focal adhesion kinase, Mol. Cell. Biol., 21:4016-4031, 2001.
Lu et al., Downregulation of caveolin-1 function by EGF leads to the loss of E-cadherin, increased transcriptional activity of beta-catenin, and enhanced tumor cell invasion, Cancer Cell, 4:499-515, 2003.
Lu and Hunter, Wnt-independent beta-catenin transactivation in tumor development, Cell Cycle, 3:571-573, 2004.
Lu and Xu, ERK1/2 MAP kinases in cell survival and apoptosis, IUBMB Life, 58:621-631, 2006.
Lu and Zhou, The prolyl isomerase PIN1: a pivotal new twist in phosphorylation signalling and disease, Nat. Rev. Mol. Cell Biol., 8:904-916, 2007.
Lufei and Cao, Nuclear import of Pin1 is mediated by a novel sequence in the PPIase domain, FEBS Lett., 583:271-276, 2009.
Luo et al., Pyruvate Kinase M2 Is a PHD3-Stimulated Coactivator for Hypoxia-Inducible Factor 1, Cell, 145:732-744, 2011.
Majumder et al., mTOR inhibition reverses Akt-dependent prostate intraepithelial neoplasia through regulation of apoptotic and HIF-1-dependent pathways, Nat. Med., 10:594-601, 2004.
Mason et al., Evolution of the metazoan-specific importin alpha gene family, J. Mol. Evol., 68:351-365, 2009.
Mazurek et al., Pyruvate kinase type M2: A key regulator of the metabolic budget system in tumor cells, Int. J. Biochem. Cell. Biol., 43:969-980, 2011.
Mazurek, Pyruvate kinase type M2: a key regulator within the tumour metabolome and a tool for metabolic profiling of tumours, Ernst Schering Found. Symp. Proc., 99-124, 2007.
Mazurek et al., Pyruvate kinase type M2 and its role in tumor growth and spreading. Semin. Cancer Biol., 15:300-308, 2005.
Mellati et al., Regulation of M2-type pyruvate kinase from human meningioma by allosteric effectors fructose 1,6 diphosphate and L-alanine, Cancer Biochem. Biophys., 13:33-41, 1992.
Meraldi and Sorger, A dual role for Bub1 in the spindle checkpoint and chromosome congression, EMBO J., 24:1621-1633, 2005.
Metzger et al., Phosphorylation of histone H3 at threonine 11 establishes a novel chromatin mark for transcriptional regulation, Nat. Cell Biol., 10:53-60, 2008.
Michel et al., MAD2 haplo-insufficiency causes premature anaphase and chromosome instability in mammalian cells, Nature, 409:355-359, 2001.
Miravet et al., Tyrosine phosphorylation of plakoglobin causes contrary effects on its association with desmosomes and adherens junction components and modulates beta-catenin-mediated transcription, Mol. Cell. Biol., 23:7391-7402, 2003.
Mori et al., A dual inhibitor against prolyl isomerase Pin1 and cyclophilin discovered by a novel real-time fluorescence detection method. BBRC, 406:439-443, 2011.
Mol Cancer Ther. 2009; 8:571-81
Moscat et al., PKCzeta at the crossroad of NF-kappaB and Jak1/Stat6 signaling pathways, Cell Death Differ., 13:702-711, 2006.
Moscatello et al., Frequent expression of a mutant epidermal growth factor receptor in multiple human tumors, Cancer Res., 55:5536-5539, 1995.
Musacchio and Salmon, The spindle-assembly checkpoint in space and time, Nat. Rev. Mol. Cell Biol., 8:379-393, 2007.
Nagashima et al., Blood. 107:4266-73, 2006.
Nakamura et al., Mutual regulation of conventional protein kinase C and a ubiquitin ligase complex, Biochem. Biophys. Res. Commun., 351:340-347, 2006.
Nicholson et al., EGFR and cancer prognosis, Eur. J. Cancer, 37(Suppl 4):S9-15, 2001.
Nogami et al., Requirement of autophosphorylated tyrosine 992 of EGF receptor and its docking protein phospholipase C gamma 1 for membrane ruffle formation, FEBS Lett., 536:71-76, 2003.
Noguchi et al., The M1- and M2-type isozymes of rat pyruvate kinase are produced from the same gene by alternative RNA splicing, J. Biol. Chem., 261:13807-13812, 1986.
Palka-Hamblin et al., Identification of beta-catenin as a target of the intracellular tyrosine kinase PTK6, J. Cell Sci., 123:236-245, 2010.
Perez-Cadahia et al., H3 phosphorylation: dual role in mitosis and interphase, Biochem. Cell Biol., 87:695-709, 2009.
Petalidis et al., Improved grading and survival prediction of human astrocytic brain tumors by artificial neural network analysis of gene expression microarray data, Mol. Cancer Ther., 7:1013-1024, 2008.
Phillips et al., Molecular subclasses of high-grade glioma predict prognosis, delineate a pattern of disease progression, and resemble stages in neurogenesis, Cancer Cell, 9:157-173, 2006.
Preuss et al., Novel mitosis-specific phosphorylation of histone H3 at Thr11 mediated by Dlk/ZIP kinase, Nucleic Acids Res., 31:878-885, 2003.
Rahighi et al., Specific recognition of linear ubiquitin chains by NEMO is important for NF-kappaB activation, Cell, 136:1098-1109, 2009.
Rao et al., Proc. Natl. Acad. Sci. U.S.A., 102:4365-4370, 2005.
Resnitzky and Reed, Different roles for cyclins D1 and E in regulation of the G1-to-S transition, Mol. Cell Biol., 15:3463-3469, 1995.
Rius et al., NF-kappaB links innate immunity to the hypoxic response through transcriptional regulation of HIF-1alpha, Nature, 453:807-811, 2008.
Roura et al., Regulation of Ecadherin/Catenin association by tyrosine phosphorylation, J. Biol. Chem., 274:36734-36740, 1999.
Schroeder et al., An examination of conditions for the cleavage of polypeptide chains with cyanogen bromide: application to catalase, Arch. Biochem. Biophys., 130:551-556, 1969.
Shimada et al., Chk1 is a histone H3 threonine 11 kinase that regulates DNA damage-induced transcriptional repression, Cell, 132:221-232, 2008.
Shimada and Nakanishi, Checkpoints meet the transcription at a novel histone milestone (H3-T11), Cell Cycle, 7:1555-1559, 2008.
Skaug et al., The role of ubiquitin in NF-kappaB regulatory pathways, Annu. Rev. Biochem., 78:769-796, 2009.
Stetak et al., Nuclear translocation of the tumor marker pyruvate kinase M2 induces programmed cell death, Cancer Res., 67:1602-1608, 2007.
Suganuma and Workman, Crosstalk among Histone Modifications, Cell, 135:604-607, 2008.
Sun et al., Mammalian target of rapamycin up-regulation of pyruvate kinase isoenzyme type M2 is critical for aerobic glycolysis and tumor growth, Proc. Natl. Acad. Sci. U.S.A., 108:4129-4134, 2011.
Tanaka et al., Kinetochore capture and bi-orientation on the mitotic spindle, Nat. Rev. Mol. Cell Biol., 6:929-942, 2005.
Tanaka et al., Blood. 105:2324-31, 2005.
Tang et al., Mol. Cell, 16:387-397, 2004.
Taus et al., Universal and confident phosphorylation site localization using phosphoRS, J. Proteome Res., 10:5354-5362, 2011.
Tetsu et al., Beta-catenin regulates expression of cyclin D1 in colon carcinoma cells, Nature, 398:422-426, 1999.
Tokunaga et al., Involvement of linear polyubiquitylation of NEMO in NF-kappaB activation, Nat. Cell Biol., 11:123-132, 2009.
Vander Heiden et al., Biochem. Pharmacol., 79(8): 1118-1124, 2010.
Vander Heiden et al., Understanding the Warburg effect: the metabolic requirements of cell proliferation, Science, 324:1029-1033, 2009.
Vander Heiden et al., Evidence for an alternative glycolytic pathway in rapidly proliferating cells, Science, 329:1492-1499, 2010.
Voldborg et al., Epidermal growth factor receptor (EGFR) and EGFR mutations, function and possible role in clinical trials, Ann. Oncol., 8:1197-1206, 1997.
Wang et al., TAK1 is a ubiquitin-dependent kinase of MKK and IKK, Nature, 412:346-351, 2001.
Warburg, On the origin of cancer cells, Science, 123:309-314, 1956.
Williams et al., Aneuploidy affects proliferation and spontaneous immortalization in mammalian cells, Science, 322:703-709, 2008.
Wu et al., Sensing of Lys 63-linked polyubiquitination by NEMO is a key event in NF-kappaB activation, Nat. Cell Biol., 8:398-406, 2006.
Wykosky et al., Therapeutic targeting of epidermal growth factor receptor in human cancer: successes and limitations, Chin. J. Cancer, 30:5-12, 2011.
Xia et al., c-Jun downregulation by HDAC3-dependent transcriptional repression promotes osmotic stress-induced cell apoptosis, Mol. Cell, 25:219-232, 2007.
Xu et al. Biochem. 50:9545-9550, 2011.
Xu et al., PLOS One, 7(9):e44226, 2012.
Yang et al., Nuclear PKM2 regulates beta-catenin transactivation upon EGFR activation, Nature, 480:118-122, 2011.
Yang et al., PKM2 Phosphorylates Histone H3 and Promotes Gene Transcription and Tumorigenesis, Cell, 150:685-696, 2012.
Yochum et al., Serial analysis of chromatin occupancy identifies beta-catenin target genes in colorectal carcinoma cells, Proc. Natl. Acad. Sci. U.S.A., 104:3324-3329, 2007.
Yoon et al., Cancer Letters, 316:97-104, 2012
Zheng et al., FAK phosphorylation by ERK primes ras-induced tyrosine dephosphorylation of FAK mediated by PIN 1 and PTP-PEST, Molecular Cell, 35:11-25, 2009.

1-3. (canceled)

4. The method of claim 19, wherein the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, neuroendocrine tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

5. The method of claim 19, comprising a PKM2 inhibitor.

6. The method of claim 5, wherein the PKM2 inhibitor is a small molecule PKM2 inhibitor.

7. (canceled)

8. The method of claim 5, wherein the PKM2 inhibitor comprises an inhibitory polynucleotide complementary to all or part of a PKM2 gene.

9. The method of claim 8, wherein the inhibitory polynucleotide is a siRNA.

10. The method claim 5, further comprising at least a second therapeutic.

11. The method of claim 10, wherein the second therapy is a MEK/ERK inhibitor therapy or a Src inhibitor therapy.

12. The method of claim 19, comprising a MEK/ERK inhibitor.

13. The method of claim 12, wherein the MEK/ERK inhibitor is U0126, AZD6244, PD98059, GSK1120212, GDC-0973, RDEA119, PD18416, CI1040 or FR180204.

14-18. (canceled)

19. A method for treating a patient having a cancer comprising:
(i) selecting a patient whose cancer cells have been determined to comprise an elevated level of histone H3 T11 phosphorylation; an elevated level of PKM2 S37 phosphorylation; an elevated level of nuclear PKM2 expression; an elevated level of Bub3 Y207 phosphorylation; an elevated level of MLC2 Y118 phosphorylation; or an elevated level of histone H3 K9 acetylation compared to a reference level; and
(ii) treating the patient with a MEK/ERK inhibitor therapy; a Src inhibitor therapy; a PKM2 inhibitor therapy; a NF-κB inhibitor therapy; a PKCε inhibitor therapy; or a Pin1 inhibitor therapy.

20-39. (canceled)

40. A method for screening candidate PKM2 inhibitors or anti-cancer agents comprising determining the binding of PKM2 to histone H3; Bub3; or MLC2 and/or the phosphorylation of histone H3; Bub3; or MLC2 by PKM2 in the presence or absence of an agent, wherein an agent that disrupts binding of PKM2 to histone H3; Bub3; or MLC2 and/or disrupts phosphorylation of histone H3; Bub3; or MLC2 by PKM2 is a candidate PKM2 inhibitor or anti-cancer agent.

41-74. (canceled)

75. An in vitro method of identifying a cancer patient that is a candidate for a therapy comprising:
(i) determining a level of β-catenin activity in a patient sample; and
(ii) identifying a cancer patient that is a candidate for a Src inhibitor o therapy based on the level of β-catenin activity, wherein an elevated level of β-catenin activity relative to a reference level indicates that the patient is a candidate for said therapy.

76-102. (canceled)
